(19) United States
(12) Patent Application Publication
(10) Pub. No.: US 2024/0376496 A1
(43) Pub. Date: Nov. 14, 2024

(54) AAV CAPSID COMPOSITIONS AND METHODS FOR DELIVERY

(71) Applicant: BIOMARIN PHARMACEUTICAL INC., Novato, CA (US)

(72) Inventors: Peter COLOSI, San Anselmo, CA (US); Vincent LEONARD, Alameda, CA (US); Silvia RAMIREZ, Windsor, CA (US); Justin ISHIDA, San Rafael, CA (US); Yu-Shan TSENG, San Rafael, CA (US); Teague STERLING, San Anselmo, CA (US)

(73) Assignee: BIOMARIN PHARMACEUTICAL INC., Novato, CA (US)

(21) Appl. No.: 18/688,685

(22) PCT Filed: Sep. 2, 2022
(86) PCT No.: PCT/US2022/075950

§ 371 Date: Mar. 1, 2024

Related U.S. Application Data
(60) Provisional application No. 63/240,774, filed on Sep. 3, 2021.

(51) Int. Cl. C12N 15/86; A61K 48/00; C07K 14/005

(52) U.S. Cl. CPC C12N 15/86; A61K 48/0058; C07K 14/005; C12N 2750/14122; C12N 2750/14143

(57) ABSTRACT

The disclosure provides various compositions comprising novel adeno-associated virus (AAV) capsid sequences and functional fragments thereof. Also provided, are methods of delivery, treatment and manufacture using the compositions provided by the disclosure.

### 1. CROSS-REFERENCE TO RELATED APPLICATIONS AND INCORPORATION BY REFERENCE

This application claims the benefit of U.S. Provisional Patent Application No. 63/240,774, filed Sep. 3, 2021, which is incorporated herein in its entirety.

All of the patents, patent applications and publications referred to herein are incorporated by reference herein in their entireties. Citation or identification of any reference in this application is not an admission that such reference is available as prior art to this application.

### 2. SEQUENCE LISTING

This application contains a computer readable Sequence Listing which has been submitted in XML file format with this application, the entire content of which is incorporated by reference herein in its entirety. The Sequence Listing XML file submitted with this application is entitled “11808-473-228_SEQ_LISTING.xml”, was created on Aug. 24, 2022 and is 913,813 bytes in size.

### 3. BACKGROUND

Gene therapy approaches using recombinant adeno-associated virus (AAV) based vectors for delivery have demonstrated safety and long-term efficacy in a number of clinical trials. Following these successes, a handful of AAV-based gene therapy products have been approved by the Food and Drug Administration and/or European Medicine Agency, such as, Glybera (which uses, AAV-1), Luxturna (which uses, AAV-2), and Zolgensma (which uses, AAV-9).

Despite these advances in AAV-based gene therapy, these studies also revealed several technical hurdles to using AAV-based vectors as a therapeutic modality more widely such as, the need for AAV vectors with increased evasion of AAV-neutralizing antibodies present in the blood, and the need for AAV vectors with enhanced tissue specificity, referred to as tropism.

Thus, there is still an ongoing unmet need for AAV-based vectors with improved functional characteristics that will allow for the broader application of AAV-based vectors as a therapeutic modality.

### 4. SUMMARY

The present disclosure provides various compositions comprising novel AAV capsid sequences that have enhanced ability to evade neutralizing antibodies, enhanced tissue specificity, and/or increased cell transduction, thereby permitting broader use of AAV-based vectors for delivery and/or treatment of disease. The embodiments described herein relate to novel AAV capsid sequences and/or their functional fragments, AAV clades, AAV branches (i.e., a group of AAV clades), recombinant AAV viral particles, vectors, rAAV vector genome constructs, host cells, and pharmaceutical compositions for delivering a biomolecule (e.g., a therapeutic biomolecule). In particular, the compositions of the disclosure can be used for in vitro, in vivo, and/or ex vivo delivery to the muscle, heart, brain, plasma, kidney, liver and/or cancer cell(s). In some embodiments, the compositions of the disclosure can be used for in vitro, in vivo, and/or ex vivo delivery to the muscle, heart, brain, plasma, kidney, liver, ear and/or cancer cell(s). The embodiments described herein also relate to methods of treatment comprising administering to a subject in need of treatment any of the novel AAV capsid sequences, rAAV vector genomes, recombinant AAV (rAAV) viral particles, host cells, or pharmaceutical compositions provided herein. The embodiments described herein also relate to methods of treatment comprising administering to a subject in need of treatment any of the novel AAV capsid sequences, rAAV vector genomes, recombinant AAV (rAAV) viral particles, host cells, or pharmaceutical compositions provided herein and a biomolecule (e.g., a therapeutic biomolecule). Methods of manufacturing a novel rAAV viral particle of the disclosure are also provided.

In one aspect, provided herein is a member of an adeno-associated virus (AAV) clade. In a specific embodiment, provided herein is a member of a clade in any one of Table 2. In a specific aspect, provided herein is a member of an AAV clade, comprising: (a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362. In some embodiments, the AAV clade member comprises: (a) a VP1 amino acid sequence that is at least 91%, at least 92%, at least 93%, at least 94%, at least 95%, at least 96%, at least 97%, at least 98%, or at least 99% identity to the VP1 amino acid sequence of any one of SEQ ID NOs: 12-127 and 361-362, (b) a VP2 amino acid sequence that is at least 91%, at least 92%, at least 93%, at least 94%, at least 95%, at least 96%, at least 97%, at least 98%, or at least 99% identity to the VP2 amino acid sequence of any one of SEQ ID NOs: 12-127 and 361-362, or (c) a VP3 amino acid sequence that is at least 91%, at least 92%, at least 93%, at least 94%, at least 95%, at least 96%, at least 97%, at least 98%, or at least 99% identity to the VP3 amino acid sequence of any one of SEQ ID NOs: 12-127 and 361-362. In one embodiment, the AAV clade member comprises: (a) a VP1 amino acid sequence that has at least 95% identity to the VP1 amino acid sequence of any one of SEQ ID NOs: 12-127 and 361-362, (b) a VP2 amino acid sequence that has at least 95% identity to the VP2 amino acid sequence of any one of SEQ ID NOs: 12-127 and 361-362, or (c) a VP3 amino acid sequence that has at least 95% identity to the VP3 amino acid sequence of any one of SEQ ID NOs: 12-127 and 361-362. In certain embodiments, the AAV clade member comprises a VP1 amino acid sequence that has at least 98% identity to the VP1 amino acid sequence of any one of SEQ ID NOs: 12-127 and 361-362. In certain embodiments, the VP1, VP2, or VP3 amino acid sequence comprises a variable region sequence, wherein the variable region sequence is selected from the variable region of at least one of SEQ ID NOs: 12-127 and 361-362. In a specific embodiment, the VP1 amino acid sequence further comprises a GBS region sequence, wherein the GBS region sequence is selected from the GBS region sequence of at least one of: SEQ ID NOs: 12-127 and 361-362. In another embodiment, the VP1 amino acid sequence further comprises a GH loop sequence, wherein the GH loop sequence is selected from the GH loop of at least one of: SEQ ID NOs: 12-127 and 361-362. In some embodiments, the AAV clade member comprises a VP2 amino acid sequence that is the VP2 amino acid sequence of any one of SEQ ID NOs: 12-127 and 361-362. In certain embodiments, the AAV clade member comprises a VP3 amino acid sequence that is the VP3 amino acid sequence of any one of SEQ ID NOs: 12-127 and 361-362. In some embodiments, the AAV clade member comprises a VP1 amino acid sequence that is the VP1 amino acid sequence of any one of SEQ ID NOs: 12-127 and 361-362. In specific embodiments, the VP1, VP2, or VP3 amino acid sequence of the AAV clade member comprises one or more of the amino acid modifications listed in Table 2. In specific embodiments, the one or more of the amino acid modifications of the VP1, VP2, or VP3 amino acid sequence of the AAV clade member are limited to the ones listed in Table 2. In specific embodiments, the AAV clade member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In some embodiments, provided herein is a member of an AAV clade, comprising: (a) a VP1 amino acid sequence that has at least 90% identity to a VP1 amino acid sequence set forth in Table 9 which is identified by a “BCD_” prefix, (b) a VP2 amino acid sequence that has at least 90% identity to the VP2 amino acid sequence of the amino acid sequence set forth in Table 9 which is identified by a “BCD_” prefix, or (c) a VP3 amino acid sequence that has at least 90% identity to the VP3 amino acid sequence of the amino acid sequence set forth in Table 9 which is identified by a “BCD_” prefix. In some embodiments, the AAV clade member comprises: (a) a VP1 amino acid sequence that is at least 91%, at least 92%, at least 93%, at least 94%, at least 95%, at least 96%, at least 97%, at least 98%, or at least 99% identity to a VP1 amino acid sequence set forth in Table 9 which is identified by a “BCD_” prefix, (b) a VP2 amino acid sequence that is at least 91%, at least 92%, at least 93%, at least 94%, at least 95%, at least 96%, at least 97%, at least 98%, or at least 99% identity to the VP2 amino acid sequence of the amino acid sequence set forth in Table 9 which is identified by a “BCD_” prefix, or (c) a VP3 amino acid sequence that is at least 91%, at least 92%, at least 93%, at least 94%, at least 95%, at least 96%, at least 97%, at least 98%, or at least 99% identity to the VP3 amino acid sequence of the amino acid sequence set forth in Table 9 which is identified by a “BCD_” prefix. In some embodiments, the AAV clade member comprises: (a) a VP1 amino acid sequence that has at least 95% identity to a VP1 amino acid sequence set forth in Table 9 which is identified by a “BCD_” prefix, (b) a VP2 amino acid sequence that has at least 95% identity to the VP2 amino acid sequence of the amino acid sequence set forth in Table 9 which is identified by a “BCD_” prefix, or (c) a VP3 amino acid sequence that has at least 95% identity to the VP3 amino acid sequence of the amino acid sequence set forth in Table 9 which is identified by a “BCD_” prefix. In certain embodiments, the AAV clade member comprises a VP1 amino acid sequence that has at least 98% identity to a VP1 amino acid sequence set forth in Table 9 which is identified by a “BCD_” prefix. In certain embodiments, the VP1, VP2, or VP3 amino acid sequence comprises a variable region sequence, wherein the variable region sequence is selected from the variable region of an amino acid sequence set forth in Table 9 which is identified by a “BCD_” prefix. In a specific embodiment, the VP1 amino acid sequence comprises a GBS region sequence, wherein the GBS region sequence is selected from the GBS region sequence of an amino acid sequence set forth in Table 9 which is identified by a “BCD_” prefix. In another embodiment, the VP1 amino acid sequence comprises a GH loop sequence, wherein the GH loop sequence is selected from the GH loop of an amino acid sequence set forth in Table 9 which is identified by a “BCD_” prefix. For example, in some embodiments, a member of specific clade in any one of Table 2 (e.g., Table 2.6, 2.7, 2.8, 2.9, 2.10, 2.11, 2.12, 2.13, 2.14, or 2.15) that comprises a VP1 amino acid sequence that is at least 90%, at least 95%, at least 98%, or at least 99% identical to a VP1 amino acid sequence of a capsid protein with a “BCD_” prefix in the same table, comprises a variable region sequence (e.g., GBS region or GH loop) that is identical to the GBS region or GH loop found in the VP1 amino acid sequence of the capsid protein with the “BCD_” prefix in the same table. In some embodiments, the AAV clade member comprises a VP2 amino acid sequence that is the VP2 amino acid sequence of an amino acid sequence set forth in Table 9 which is identified by a “BCD_” prefix. In certain embodiments, the AAV clade member comprises a VP3 amino acid sequence that is the VP3 amino acid sequence of an amino acid sequence set forth in Table 9 which is identified by a “BCD_” prefix. In some embodiments, the AAV clade member comprises a VP1 amino acid sequence of a VP1 amino acid sequence set forth in Table 9 which is identified by a “BCD_” prefix. In specific embodiments, the VP1, VP2, or VP3 amino acid sequence of the AAV clade member comprises one or more of the amino acid modifications listed in Table 2. In specific embodiments, the one or more of the amino acid modifications of the VP1, VP2, or VP3 amino acid sequence of the AAV clade member are limited to the ones listed in Table 2. For example, in some embodiments, a member of specific clade in any one of Table 2 (e.g., Table 2.6, 2.7, 2.8, 2.9, 2.10, 2.11, 2.12, 2.13, 2.14, or 2.15) that comprises a VP1 amino acid sequence that is at least 90%, at least 95%, at least 98%, or at least 99% identical to a VP1 amino acid sequence of a capsid protein with a “BCD_” prefix in the same table, comprises one or more of the amino acid modifications identified in that table. In specific embodiments, the AAV clade member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC50, and wherein the NC50 increases from about 1-fold to about 600-fold as compared to a reference AAV.

In another aspect, provided herein is a member of an AAV clade, comprising: a VP1 amino acid sequence that has a least 90% sequence identity to a representative VP1 amino acid sequence of a AAV clade, and wherein the representative sequence is selected from any one of: SEQ ID NOs: 28, 10, 91, 7, 9, 4, 27, 3, 1, and 23. In one embodiment, the VP1 amino acid sequence has at least 95% identity to any one of: SEQ ID NOs: 28, 10, 91, 7, 9, 4, 27, 3, 1, and 23. In another embodiment, the VP1 amino acid sequence has at least 98% identity to any one of: SEQ ID NOs: 28, 10, 91, 7, 9, 4, 27, 3, 1, and 23. In another embodiment, the VP1 amino acid sequence has at least 99% identity to any one of: SEQ ID NOs: 28, 10, 91, 7, 9, 4, 27, 3, 1, and 23. In specific embodiments, the VP1 amino acid sequence comprises one or more of the amino acid modifications listed in Table 2. In specific embodiments, the VP1 amino acid sequence modifications are limited to the ones listed in Table 2. In specific embodiments, the AAV clade member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another aspect, provided herein is a member of an adeno-associated virus (AAV) clade, comprising: a VP1 amino acid sequence that has a variable region amino acid sequence, wherein the variable region amino acid sequence has substantial sequence similarity or identity to a variable region amino acid sequence in any one of: SEQ ID NOs: 12-127 and 361-362. In one embodiment, the variable region amino acid sequence is selected from any one of VRI-VRIX, a GBS region, or a GH loop, or a combination thereof. In certain embodiments, the any one of VRI-VRIX sequence has at least 90% sequence similarity or identity to any one of VRI-VRIX of any one of SEQ ID NOs: 12-127 and 361-362. In some embodiments, the GBS region sequence has at least 90% sequence similarity or identity to the GBS region of any one of SEQ ID NOs: 12-127 and 361-362. In certain embodiments, the GH loop sequence has at least 90% sequence similarity or identity to the GH loop of any one of SEQ ID NOs: 12-127 and 361-362. In specific embodiments, the AAV clade member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another aspect, provided herein is a member of an AAV clade, comprising: a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3. In one embodiment, the genetic distance is the mean genetic distance within the same AAV clade, as provided in Table 3. In another embodiment, the genetic distance is a range from about the min genetic distance within the same clade to about the max genetic distance within the same clade, as provided in Table 3. In certain embodiments, the second VP1 amino acid sequence comprises a VP1 amino acid sequence of any one of: SEQ ID NOs: 1-180 and 361-362. In specific embodiments, the AAV clade member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another aspect, provided herein is a member of an AAV branch, comprising: a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3. In one embodiment, the genetic distance is the mean genetic distance within the same branch as provided in Table 3. In another embodiment, the genetic distance is a range from about the min genetic distance within the same branch to about the max genetic distance within the same branch as provided in Table 3. In certain embodiments, the second VP1 amino acid sequence comprises a VP1 amino acid sequence of any one of SEQ ID NOs: 1-180 and 361-362. In specific embodiments, the AAV branch member further comprises the ability to evade AAV humoral immunity as determined in an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a neutralizing antibody (Nab) titer, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another aspect, provided herein is an AAV capsid protein, comprising: (a) a VP1 amino acid sequence that has at least 90% identity to a VP1 amino acid sequence set forth in Table 9 which is identified by a “BCD_” prefix, (b) a VP2 amino acid sequence that has at least 90% identity to the VP2 amino acid sequence of the amino acid sequence set forth in Table 9 which is identified by a “BCD_” prefix, or (c) a VP3 amino acid sequence that has at least 90% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 91% identity to a VP1 amino acid sequence set forth in Table 9 which is identified by a “BCD_” prefix, (b) a VP2 amino acid sequence that has at least 91% identity to the VP2 amino acid sequence of the amino acid sequence set forth in Table 9 which is identified by a “BCD_” prefix, or (c) a VP3 amino acid sequence that has at least 91% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 92% identity to a VP1 amino acid sequence set forth in Table 9 which is identified by a “BCD_” prefix, (b) a VP2 amino acid sequence that has at least 92% identity to the VP2 amino acid sequence of the amino acid sequence set forth in Table 9 which is identified by a “BCD_” prefix, or (c) a VP3 amino acid sequence that has at least 92% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 93% identity to a VP1 amino acid sequence set forth in Table 9 which is identified by a “BCD_” prefix, (b) a VP2 amino acid sequence that has at least 93% identity to the VP2 amino acid sequence of the amino acid sequence set forth in Table 9 which is identified by a “BCD_” prefix, or (c) a VP3 amino acid sequence that has at least 93% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 94% identity to a VP1 amino acid sequence set forth in Table 9 which is identified by a “BCD_” prefix, (b) a VP2 amino acid sequence that has at least 94% identity to the VP2 amino acid sequence of the amino acid sequence set forth in Table 9 which is identified by a “BCD_” prefix, or (c) a VP3 amino acid sequence that has at least 94% identity to the VP3 amino acid sequence of the amino acid sequence set forth in Table 9 which is identified by a “BCD_” prefix. In some embodiments, the amino acid set forth in Table 9 is one discussed in the Example section, infra. In some embodiments, the amino acid sequence set forth in Table 9 is BCD_0126, BCD_0282, BCD_0176, BCD_0446, BCD_0160, BCD_0195, BCD_0180, BCD_0192, BCD_0185, BCD_0454, BCD_0277, BCD_0174, BCD_0167, BCD_0126, BCD_0125, BCD_0193, BCD_0286, BCD_0182, BCD_0283, BCD_0106, or BCD_0361.

In some embodiments, provided herein is an AAV capsid protein, comprising: (a) a VP1 amino acid sequence that has at least 95% identity to a VP1 amino acid sequence set forth in Table 9 which is identified by a “BCD_” prefix, (b) a VP2 amino acid sequence that has at least 95% identity to the VP2 amino acid sequence of the amino acid sequence set forth in Table 9 which is identified by a “BCD_” prefix, or (c) a VP3 amino acid sequence that has at least 95% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 96% identity to a VP1 amino acid sequence set forth in Table 9 which is identified by a “BCD_” prefix, (b) a VP2 amino acid sequence that has at least 96% identity to the VP2 amino acid sequence of the amino acid sequence set forth in Table 9 which is identified by a “BCD_” prefix, or (c) a VP3 amino acid sequence that has at least 96% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 97% identity to a VP1 amino acid sequence set forth in Table 9 which is identified by a “BCD_” prefix, (b) a VP2 amino acid sequence that has at least 97% identity to the VP2 amino acid sequence of the amino acid sequence set forth in Table 9 which is identified by a “BCD_” prefix, or (c) a VP3 amino acid sequence that has at least 97% identity to the VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 98% identity to a VP1 amino acid sequence set forth in Table 9 which is identified by a “BCD_” prefix, (b) a VP2 amino acid sequence that has at least 98% identity to a VP2 amino acid sequence of the amino acid sequence set forth in Table 9 which is identified by a “BCD_” prefix, or (c) a VP3 amino acid sequence that has at least 98% identity to a VP3 amino acid sequence of the amino acid sequence set forth in Table 9 which is identified by a “BCD_” prefix. In some embodiments, provided herein is an AAV capsid protein, comprising: (a) a VP1 amino acid sequence that has at least 99% identity to a VP1 amino acid sequence set forth in Table 9 which is identified by a “BCD_” prefix, (b) a VP2 amino acid sequence that has at least 99% identity to a VP2 amino acid sequence of the amino acid sequence set forth in Table 9 which is identified by a “BCD_” prefix, or (c) a VP3 amino acid sequence that has at least 99% identity to a VP3 amino acid sequence of the amino acid sequence set forth in Table 9 which is identified by a “BCD_” prefix. In some embodiments, provided herein is an AAV capsid protein, comprising: (a) a VP1 amino acid sequence comprising a VP1 amino acid sequence set forth in Table 9 which is identified by a “BCD_” prefix, (b) a VP2 amino acid sequence that comprising a VP2 amino acid sequence of the amino acid sequence set forth in Table 9 which is identified by a “BCD_” prefix, or (c) a VP3 amino acid sequence comprising a VP3 amino acid sequence of the amino acid sequence set forth in Table 9 which is identified by a “BCD_” prefix. In some embodiments, the amino acid set forth in Table 9 is one discussed in the Example section, infra. In some embodiments, the amino acid sequence set forth in Table 9 is BCD_0126, BCD_0282, BCD_0176, BCD_0446, BCD_0160, BCD_0195, BCD_0180, BCD_0192, BCD_0185, BCD_0454, BCD_0277, BCD_0174, BCD_0167, BCD_0126, BCD_0125, BCD_0193, BCD_0286, BCD_0182, BCD_0283, BCD_0106, or BCD_0361.

In another aspect, provided herein is an AAV capsid protein, comprising: (a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of the VP2 sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362. In one embodiment, the AAV capsid protein comprises: (a) a VP1 amino acid sequence that has at least 95% identity to the VP1 amino acid sequence of any one of SEQ ID NOs: 12-127 and 361-362, (b) a VP2 amino acid sequence that has at least 95% identity to the VP2 amino acid sequence of ay one of SEQ ID NOs: 12-127 and 361-362, or (c) a VP3 amino acid sequence that has at least 95% identity to the VP3 amino acid sequence of any one of SEQ ID NOs: 12-127 and 361-362. In another embodiment, the AAV capsid protein comprises: (a) a VP1 amino acid sequence that has at least 98% identity to the VP1 amino acid sequence of any one of SEQ ID NOs: 12-127 and 361-362, (b) a VP2 amino acid sequence that has at least 98% identity to the VP2 amino acid sequence of any one of SEQ ID NOs: 12-127 and 361-362, or (c) a VP3 amino acid sequence that has at least 98% identity to the VP3 amino acid sequence of any one of SEQ ID NOs: 12-127 and 361-362. In another embodiment, the AAV capsid protein comprises a VP1, VP2, or VP3 amino acid sequence that is a VP1, VP2 or VP3 amino acid sequence of any one of SEQ ID NOs: 12-127 and 361-362. In certain embodiments, the VP1, VP2, or VP3 amino acid sequence comprises a variable region amino acid sequence, and wherein the variable region amino acid sequence is a VRI-VRIX of any one of: SEQ ID NOs: 12-127 and 361-362. In a specific embodiment, the VP1, VP2, or VP3 amino acid sequence comprises a GBS region amino acid sequence, and wherein the GBS region amino acid sequence is a GBS region of any one of: SEQ ID NOs: 12-127 and 361-362. In another specific embodiment, the VP1, VP2, or VP3 amino acid sequence comprises a GH loop amino acid sequence, and wherein the GH loop amino acid sequence is a GH loop selected from any one of: SEQ ID NOs: 12-127 and 361-362. In specific embodiments, the AAV capsid protein further comprises the ability to evade AAV humoral immunity as determined by an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a NC₅₀, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another embodiment, provided herein is an AAV capsid protein, comprising: (a) a VP1 amino acid sequence that has at least 90% identity to the VP1 amino acid sequence of any one of SEQ ID NOs12-127 and 361-362, (b) a VP2 amino acid sequence that has at least 90% identity to the VP2 amino acid sequence of the VP2 sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362, wherein one or more variable regions of the VP1 amino acid sequence, VP2 amino acid sequence, or VP3 amino acid sequence is identical to the one or more variable regions of the amino acid sequence to which the VP1 amino acid sequence, VP2 amino acid sequence, or VP3 amino acid sequence has 90% identity. In one embodiment, the AAV capsid protein comprises: (a) a VP1 amino acid sequence that has at least 95% identity to the VP1 amino acid sequence of any one of SEQ ID NOs: 12-127 and 361-362, (b) a VP2 amino acid sequence that has at least 95% identity to the VP2 amino acid sequence of any one of SEQ ID NOs: 12-127 and 361-362, and (c) a VP3 amino acid sequence that has at least 95% identity to the VP3 amino acid sequence of any one of SEQ ID NOs: 12-127 and 361-362, wherein one or more variable regions of the VP1 amino acid sequence, VP2 amino acid sequence, or VP3 amino acid sequence is identical to the one or more variable regions of the amino acid sequence to which the VP1 amino acid sequence, VP2 amino acid sequence, or VP3 amino acid sequence has 95% identity. In another embodiment, the AAV capsid protein comprises: (a) a VP1 amino acid sequence that has at least 98% identity to the VP1 amino acid sequence of any one of SEQ ID NOs: 12-127 and 361-362, (b) a VP2 amino acid sequence that has at least 98% identity to the VP2 amino acid sequence of any one of SEQ ID NOs: 12-127 and 361-362, and (c) a VP3 amino acid sequence that has at least 98% identity to the VP3 amino acid sequence of any one of SEQ ID NOs: 12-127 and 361-362, wherein one or more variable regions of the VP1 amino acid sequence, VP2 amino acid sequence, or VP3 amino acid sequence is identical to the one or more variable regions of the amino acid sequence to which the VP1 amino acid sequence, VP2 amino acid sequence, or VP3 amino acid sequence has 98% identity. In some embodiments, the one or more variable regions is one or more of VRI-VRIX. In some embodiments, the one or more variable regions is two, three, four, five, or more of VRI-VRIX. In some embodiments, the one or more variable regions is six, seven, or eight of VRI-VRIX. In some embodiments, the one or more variable regions is VRI-VRIX. In some embodiments, the one or more variable regions is a GBS region. In some embodiments, the one or more variable regions is a GH loop. In some embodiments, the one or more variable regions is a GBS region and a GH loop. In some embodiments, the one or more variable regions is one or more of VRI-VRIX, and a GBS region. In some embodiments, the one or more variable regions is one or more of VRI-VRIX, and a GH loop. In some embodiments, the one or more variable regions is one or more of VRI-VRIX, GBS loop, and a GH loop. In specific embodiments, the AAV capsid protein comprises the ability to evade AAV humoral immunity as determined by an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a NC₅₀, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In some embodiments, an AAV capsid protein provided herein comprises a VP1 amino acid sequence, a VP2 amino acid sequence, and a VP3 amino acid sequence.

In another aspect, provided herein is a nucleotide sequence encoding an AAV clade member described herein, AAV branch member described herein, or AAV capsid protein described herein. In specific embodiments, an AAV clade member, AAV branch member, or AAV capsid protein comprises a VP1 amino acid sequence, a VP2 amino acid sequence, or a VP3 amino acid sequence of a VP1 amino acid sequence, a VP2 amino acid sequence, or a VP3 amino acid sequence of a capsid protein provided herein (e.g., in Table 9) with the “BCD” prefix.

In another aspect, provided herein is a vector comprising a nucleotide sequence encoding an AAV clade member described herein, AAV branch member described herein, or AAV capsid protein described herein. In specific embodiments, the vector further comprises a heterologous regulatory sequence that controls expression of the capsid protein in a host cell. In one embodiment, provided herein is a vector comprising: (a) a nucleotide sequence encoding a VP1 amino acid sequence of the AAV clade member described herein; and (b) a heterologous regulatory sequence that controls expression of the capsid protein in a host cell. In another embodiment, provided herein is a vector, comprising: (a) a nucleotide sequence encoding a VP1 amino acid sequence of the AAV branch member described herein; and (b) a heterologous regulatory sequence that controls expression of the capsid protein in a host cell. In another embodiment, provided herein is a vector, comprising: (a) a nucleotide sequence encoding a VP1, VP2, or VP3 capsid protein that has at least 90% identity to the VP1, VP2, or VP3 of any one of SEQ ID NOs: 12-127 and 361-362; and (b) a heterologous regulatory sequence that controls expression of the capsid protein in a host cell. In another embodiment, provided herein is a vector, comprising: (a) a nucleotide sequence encoding a VP1, VP2, or VP3 capsid protein that has at least 91%, at least 92%, at least 93%, at least 94%, at least 95%, at least 96%, at least 97%, at least 98%, or at least 99% identity to the VP1, VP2, or VP3 of any one of SEQ ID NOs: 12-127 and 361-362; and (b) a heterologous regulatory sequence that controls expression of the capsid protein in a host cell. In another embodiment, provided herein is a vector, comprising: (a) a nucleotide sequence encodes a VP1, VP2, or VP3 capsid protein that has at least 95% identity to the VP1, VP2, or VP3 of any one of SEQ ID NOs: 12-127 and 361-362; and (b) a heterologous regulatory sequence that controls expression of the capsid protein in a host cell. In another embodiment, provided herein is a vector, comprising: (a) a nucleotide sequence encodes a VP1, VP2, or VP3 capsid protein that has at least 98% identity to the VP1, VP2, or VP3 of any one of SEQ ID NOs: 12-127 and 361-362; and (b) a heterologous regulatory sequence that controls expression of the capsid protein in a host cell. In certain embodiments, the vector further comprises a transgene comprising a nucleotide sequence encoding a biomolecule operably linked to a heterologous regulatory sequence that controls expression of the capsid protein in a host cell.

In another aspect, provided herein are host cells comprising a nucleotide sequence encoding a VP1, VP2, or VP3 capsid protein described herein, or a vector described herein. In a specific embodiment, provided herein are in vitro or ex vivo host cells comprising a nucleotide sequence encoding a VP1, VP2, or VP3 capsid protein described herein, or a vector described herein.

In another aspect, provided herein are AAV viral particles comprising an AAV clade member described herein, an AAV branch member, or an AAV capsid protein described herein. In one embodiment, provided herein is a recombinant AAV viral particle, comprising: (a) a capsid, wherein the capsid comprises a VP1 amino acid sequence of the AAV clade member described herein; and (b) an rAAV vector genome comprising a nucleotide sequence encoding a biomolecule operably linked to a heterologous regulatory sequence that controls expression of the nucleotide sequence in a host cell. In another embodiment, provided herein is a recombinant AAV viral particle, comprising: (a) a capsid, wherein the capsid comprises a VP1 amino acid sequence of the AAV branch member described herein; and (b) an rAAV vector genome comprising a nucleotide sequence encoding a biomolecule operably linked to a heterologous regulatory sequence that controls expression of the nucleotide sequence in a host cell. In another embodiment, provided herein is a recombinant AAV viral particle, comprising: (a) the AAV capsid protein described herein; and (b) an rAAV vector genome comprising a nucleotide sequence encoding a biomolecule operably linked to a heterologous regulatory sequence that controls expression of the nucleotide sequence in a host cell. In some embodiments, a recombinant AAV viral particle comprises (a) a capsid, wherein the capsid comprises a VP1 amino acid sequence, a VP2 amino acid sequence, or a VP3 amino acid sequence of the AAV clade member described herein; and (b) a rAAV vector genome comprising a nucleotide sequence encoding a biomolecule operably linked to a heterologous regulatory sequence that controls expression of the nucleotide sequence in a host cell. In specific embodiments, a recombinant AAV viral particle comprises (a) a capsid, wherein the capsid comprises a VP1 amino acid sequence, a VP2 amino acid sequence, and a VP3 amino acid sequence of the AAV clade member described herein; and (b) a rAAV vector genome comprising a nucleotide sequence encoding a biomolecule operably linked to a heterologous regulatory sequence that controls expression of the nucleotide sequence in a host cell. In specific embodiments, the rAAV vector genome comprises an AAV inverted terminal repeat or a fragment thereof. In a specific embodiment, the AAV inverted terminal repeat is a 5′ AAV inverted terminal repeat selected from Table 4. In another specific embodiment, the AAV inverted terminal repeat is a 3′ AAV inverted terminal repeat selected from Table 4. In certain embodiments, the rAAV vector genome comprises a 5′ AAV inverted terminal repeat or a fragment thereof, and a 3′ AAV terminal repeat or a fragment thereof. In a specific embodiment, the 5′ AAV inverted terminal repeat and the 3′ AAV inverted terminal repeat are selected from a 5′ AAV terminal repeat and a 3′ AAV terminal repeat, respectively, provided in Table 4. In certain embodiments, the biomolecule is selected from a therapeutic protein, an enzyme, a peptide, an RNA, a component of CRISPR gene editing system, an antisense oligonucleotides (AONs), an AON-mediated exon skipping, a poison exon, or a dominant negative mutant protein. In some embodiments, the therapeutic protein is endogenously expressed in one or more of a muscle, heart, brain, plasma, kidney, liver, ear, or cancer cell of a subject. In a specific embodiment, the therapeutic protein is a functional version of the endogenously expressed protein. In certain embodiments, the recombinant AAV viral particle has enhanced tropism to the muscle cell as compared to a reference AAV. In some embodiments, the recombinant AAV viral particle has enhanced tropism to the heart cell as compared to a reference AAV. In certain embodiments, the recombinant AAV viral particle has enhanced tropism to the brain cell as compared to a reference AAV. In some embodiments, the recombinant AAV viral particle has enhanced tropism to the plasma cell as compared to a reference AAV. In certain embodiments, the recombinant AAV viral particle has enhanced tropism to the kidney cell as compared to a reference AAV. In some embodiments, the recombinant AAV viral particle has enhanced tropism to the liver cell as compared to a reference AAV. In certain embodiments, the recombinant AAV viral particle de-targets cells in a subject other than the cell for which the rAAV has enhanced tropism. In specific embodiments, the de-targeted cell is selected from one or more of a muscle, heart, brain, plasma, kidney, or liver cell. In specific embodiments, the recombinant AAV viral particle has the ability to evade AAV humoral immunity as determined by an in vitro assay. In a specific embodiment, the in vitro assay is an IVIg assay that determines a percent (%) transduction, and wherein the % transduction is about 2% to about 500% greater transduction as compared to a reference AAV at a given IVIg concentration. In another specific embodiment, the in vitro assay is an IVIg assay that determines a NC₅₀, and wherein the NAb titer is reduced to about 1-fold to about 4,000-fold as compared to a reference AAV. In another specific embodiment, the in vitro assay is an IVIg assay that determines a NC₅₀, and wherein the NC₅₀ increases from about 1-fold to about 600-fold as compared to a reference AAV.

In another aspect, provided herein is an in vitro cell(s) or tissue comprising a recombinant AAV viral particle described herein. In another aspect, provided herein is an ex vivo cell(s) or tissue comprising a recombinant AAV viral particle described herein.

In another aspect, provided herein are cultured host cells comprising a recombinant nucleic acid molecule encoding an AAV capsid protein described herein. In one embodiment, provided herein is a cultured host cell comprising a recombinant nucleic acid molecule encoding an AAV VP1 capsid protein comprising a sequence comprising: (a) the full length VP1 protein of any one of SEQ ID NOs: 12-127 and 361-362; or (b) an amino acid sequence with at least 95% identity to the full length VP1 capsid protein of any one of SEQ ID NOs: 12-127 and 361-362, wherein the recombinant nucleic acid molecule further comprises a heterologous sequence. In another embodiment, provided herein is a cultured host cell, comprising a recombinant nucleic acid molecule encoding an AAV VP2 capsid protein comprising: (a) a sequence comprising the full length VP2 protein of any one of SEQ ID NOs: 12-127 and 361-362; or (b) an amino acid sequence with at least 95% identity to the full length VP2 capsid protein of any one of SEQ ID NOs: 12-127 and 361-362, wherein the recombinant nucleic acid molecule further comprises a heterologous sequence. In another embodiment, provided herein is a cultured host cell, comprising a recombinant nucleic acid molecule encoding an AAV VP3 capsid protein comprising: (a) a sequence comprising the full length VP3 protein of any one of SEQ ID NOs: 12-127 and 361-362; or (b) an amino acid sequence with at least 95% identity to the full length VP3 capsid protein of any one of SEQ ID NOs: 12-127 and 361-362, wherein the recombinant nucleic acid molecule further comprises a heterologous sequence. In specific embodiments, the amino acid residues varied in the AAV VP1, VP2, or VP3 capsid protein with at least 95% identity to the full length VP1, VP2, or VP3 capsid protein of any one of SEQ ID NOs: 12-127 and 361-362 are selected from Table 2. In specific embodiments, the heterologous sequence is a heterologous nucleotide sequence, which is heterologous to the nucleotide sequence encoding the AAV capsid protein. In specific embodiments, the heterologous sequence is a heterologous nucleotide sequence, which encodes an amino acid sequence that is heterologous to the AAV capsid protein.

In another embodiment, provided herein is a cultured host cell containing a recombinant nucleic acid molecule, comprising: (a) nucleotides of a full length AAV VP1 capsid protein of any one of SEQ ID NOs: 192-307 and 363-364; or (b) a sequence at least 95% identical to nucleotides of the full length VP1 capsid protein of any one of SEQ ID NOs: 192-307 and 363-364, wherein the recombinant nucleic acid molecule further comprises a heterologous sequence. In another embodiment, provided herein is a cultured host cell containing a recombinant nucleic acid molecule, comprising: (a) nucleotides of a full length AAV VP2 capsid protein of any one of SEQ ID NOs: 192-307 and 363-364; or (b) a sequence at least 95% identical to nucleotides of the full length VP2 capsid protein of any one of SEQ ID NOs: 192-307 and 363-364, wherein the recombinant nucleic acid molecule further comprises a heterologous sequence. In another embodiment, provided herein is a cultured host cell containing a recombinant nucleic acid molecule, comprising: nucleotides of a full length AAV VP3 capsid protein of any one of SEQ ID NOs: 192-307 and 363-364; or a sequence at least 95% identical to nucleotides of the full length VP3 capsid protein of any one of SEQ ID NOs: 192-307 and 363-364, wherein the recombinant nucleic acid molecule further comprises a heterologous sequence. In specific embodiments, nucleic acids varied in the AAV VP1, VP2, or VP3 capsid protein with at least 95% identity to the full length VP1, VP2, or VP3 capsid protein of any one of SEQ ID NOs: 192-307 and 363-364 are selected from nucleic acids encoding the amino acid residues that vary in Table 2. In specific embodiments, the heterologous sequence is a heterologous nucleotide sequence, which is heterologous to the nucleotide sequence encoding the AAV capsid protein. In specific embodiments, the heterologous sequence is a heterologous nucleotide sequence, which encodes an amino acid sequence that is heterologous to the AAV capsid protein.

In another aspect, provided herein are compositions comprising a recombinant AAV viral particle described herein. In one embodiment, provided herein is a composition, comprising: (a) a recombinant AAV viral particle described herein; and (b) a physiologically acceptable carrier.

In another aspect, provided herein is a method of delivering a biomolecule to a cell in vitro, comprising: transducing the cell with a recombinant AAV viral particle described herein. In some embodiments, the cell is one or more of a muscle cell, heart cell, brain cell, plasma cell, kidney cell, liver cell, ear cell, or cancer cell. In certain embodiments, the cell is one or more of a muscle, heart, brain, plasma, kidney, liver, or cancer cell.

In another aspect, provided herein is a method of delivering a biomolecule to a cell ex vivo, comprising transducing the cell with a recombinant AAV viral particle described herein. In some embodiments, the cell is one or more of a muscle cell, heart cell, brain cell, plasma cell, kidney cell, liver cell, ear cell, or cancer cell. In certain embodiments, the cell is one or more of a muscle, heart, brain, plasma, kidney, liver, or cancer cell.

In another aspect, provided herein is a method of delivering a biomolecule to a cell in a subject, comprising: administering a recombinant AAV viral particle described herein to the cell in the subject. In some embodiments, the cell is one or more of a muscle cell, heart cell, brain cell, plasma cell, kidney cell, liver cell, ear cell, or cancer cell. In certain embodiments, the cell is one or more of a muscle, heart, brain, plasma, kidney, liver, or cancer cell. In certain embodiments, the subject is a human subject. In other embodiments, the subject is a non-human subject.

In another aspect, provided herein is a method of treating a disease or disorder, comprising administering a recombinant AAV viral particle described herein to a subject. In certain embodiments, the subject is a human subject. In other embodiments, the subject is a non-human subject.

In another aspect, provided herein are methods for producing a recombinant AAV (rAAV) viral particle described herein. In one embodiment, provided herein is a method for producing a recombinant AAV viral particle, comprising: culturing a host cell comprising one or more vectors or rAAV vector genomes for generating the rAAV viral particle, wherein the one or more vectors or rAAV vector genomes comprises a nucleotide sequence encoding an AAV capsid protein described herein. In another embodiment, provided herein is a method for producing a rAAV viral particle, comprising: culturing a host cell comprising one or more vectors or rAAV vector genomes for generating the rAAV viral particle, wherein the one or more vectors or rAAV vector genomes comprises a nucleotide sequence encoding a VP1 protein of an AAV clade member described herein. In another embodiment, provided herein is a method for producing a rAAV viral particle, comprising: culturing a host cell comprising one or more vectors or rAAV vector genomes for generating the rAAV viral particle, wherein the one or more vectors or rAAV vector genomes comprises a nucleotide sequence encoding a VP1 protein of an AAV branch member described herein. In certain embodiments, the one or more vectors further comprises a nucleotide sequence used by the host cell to generate a rAAV viral particle, and wherein the nucleotide sequence is operably linked to a heterologous regulatory sequence that controls expression of the nucleotide sequence in a host cell. In some embodiments, prior to the culturing step the host cell is transfected with the one or more vector or rAAV vector genomes. In certain embodiments, the rAAV viral particle is isolated from the host cell.

### 5. BRIEF DESCRIPTION OF THE DRAWINGS

FIG. 1 shows an overview of the approaches used to discover and/or produce the novel AAV capsid sequences.

FIG. 2 shows a diagram of the workflow used in the identification and characterization of novel AAV capsid proteins and viral particles.

FIGS. 3A-3C show a phylogenetic tree diagram of branch 2 constructed using the AAV VP1 capsid protein and Neighbor-Joining method, grouped in clades based on their common ancestry as determined by the Jukes-Cantor model.

FIGS. 4A-4L show an alignment of the VP1 protein for AAV clade 0. VP1 protein of BCD_0454 (SEQ ID NO:114); BCD_0446 (SEQ ID NO:109); BCD_0455 (SEQ ID NO: 115); BCD_0190 (SEQ ID NO:51); BCD_0192 (SEQ ID NO:52); BCD_0160 (SEQ ID NO: 28); BCD_0161 (SEQ ID NO:29); BMN_0273 (SEQ ID NO:160); BMN_0271 (SEQ ID NO: 158); BMN_0265 (SEQ ID NO:152); BMN_0272 (SEQ ID NO:159); BCD_0299 (SEQ ID NO: 84); BCD_0300 (SEQ ID NO:85); BCD_0301 (SEQ ID NO:86); BMN_0261 (SEQ ID NO: 148); BMN_0266 (SEQ ID NO:153); BMN_0262 (SEQ ID NO:149); BCD_0285 (SEQ ID NO: 70); BCD_0281 (SEQ ID NO:66); BMN_0269 (SEQ ID NO:156); BMN_0268 (SEQ ID NO: 155); BMN_0267 (SEQ ID NO:154); BMN_0264 (SEQ ID NO:151); BMN_0263 (SEQ ID NO: 150); BCD_0283 (SEQ ID NO:68); BCD_0282 (SEQ ID NO:67); BCD_0280 (SEQ ID NO: 65); BCD_0284 (SEQ ID NO:69); BMN_0270 (SEQ ID NO:157); BCD_0291 (SEQ ID NO: 76); BCD_0298 (SEQ ID NO:83); BCD_0295 (SEQ ID NO:80); BCD_0293 (SEQ ID NO: 78); BCD_0290 (SEQ ID NO:75); BCD_0287 (SEQ ID NO:72); BCD_0294 (SEQ ID NO: 79); BCD_0289 (SEQ ID NO:74); BCD_0286 (SEQ ID NO:71); BCD_0292 (SEQ ID NO: 77); BCD_0288 (SEQ ID NO:73); BCD_0165 (SEQ ID NO:30); BCD_0174 (SEQ ID NO: 37); BCD_0175 (SEQ ID NO:38); BCD_0278 (SEQ ID NO:63); BCD_0274 (SEQ ID NO: 59); BCD_0279 (SEQ ID NO:64); BCD_0296 (SEQ ID NO:81); BCD_0276 (SEQ ID NO: 61); BCD_0297 (SEQ ID NO:82); BCD_0275 (SEQ ID NO:60); and BCD_0277 (SEQ ID NO: 62) are aligned.

FIGS. 5A-5J show an alignment of the VP1 protein for AAV clade 1. VP1 protein of BCD_0386 (SEQ ID NO:94); BCD_0125 (SEQ ID NO:20); BCD_0193 (SEQ ID NO:53); BCD_0195 (SEQ ID NO:55); BMN_0305 (SEQ ID NO:164); BMN_0302 (SEQ ID NO:161); BMN_0303 (SEQ ID NO:162); BMN_0304 (SEQ ID NO:163); BMN_0310 (SEQ ID NO:169); BMN_0309 (SEQ ID NO:168); BMN_0307 (SEQ ID NO:166); BMN_0311 (SEQ ID NO:170); BMN_0306 (SEQ ID NO:165); BMN_0308 (SEQ ID NO:167); BMN_0314 (SEQ ID NO:173); BMN_0312 (SEQ ID NO:171); BMN_0313 (SEQ ID NO:172); BCD_0180 (SEQ ID NO:43); BMN_0315 (SEQ ID NO:174); BCD_0182 (SEQ ID NO:45); BCD_0185 (SEQ ID NO:46); BCD_0448 (SEQ ID NO:111); BCD_0198 (SEQ ID NO:58); BCD_0146 (SEQ ID NO:25); BCD_0127 (SEQ ID NO:22); AAV-10 (SEQ ID NO:10); BCD_0126 (SEQ ID NO:21); BCD_0449 (SEQ ID NO:112); BCD_0450 (SEQ ID NO:113); BCD_0181 (SEQ ID NO:44); BCD_0177 (SEQ ID NO:40); BCD_0197 (SEQ ID NO:57); BCD_0488 (SEQ ID NO:125); AAV-8 (SEQ ID NO:8); BCD_0486 (SEQ ID NO:123); BCD_0487 (SEQ ID NO:124); BCD_0186 (SEQ ID NO:47); BCD_0187 (SEQ ID NO:48); BMN_0218 (SEQ ID NO:138); BMN_0208 (SEQ ID NO:128); BMN_0214 (SEQ ID NO:134); BMN_0212 (SEQ ID NO:132); BMN_0211 (SEQ ID NO:131); BMN_0216 (SEQ ID NO:136); BMN_0210 (SEQ ID NO:130); BMN_0213 (SEQ ID NO:133); BMN_0209 (SEQ ID NO:129); BMN_0215 (SEQ ID NO:135); and BMN_0217 (SEQ ID NO:137) are aligned.

FIGS. 6A-6F show an alignment of the VP1 protein for AAV clade 4. VP1 protein of BCD_0361 (SEQ ID NO:92); BCD_0461 (SEQ ID NO:117); BCD_0389 (SEQ ID NO:95); BCD 0392 (SEQ ID NO:98); BCD_0413 (SEQ ID NO:103); BCD_0467 (SEQ ID NO:120); BCD_0354 (SEQ ID NO:89); BCD_0492 (SEQ ID NO:127); BCD_0390 (SEQ ID NO:96); BCD_0462 (SEQ ID NO:118); BCD_0468 (SEQ ID NO:121); BCD_0359 (SEQ ID NO:90); BCD_0396 (SEQ ID NO:102); BCD_0360 (SEQ ID NO:91); BCD_0391 (SEQ ID NO:97); BCD_0505 (SEQ ID NO:361); BCD_0106 (SEQ ID NO:13); BCD_0415 (SEQ ID NO:105); BCD_0466 (SEQ ID NO:119); AAV-po.6 (SEQ ID NO:2); BCD_0443 (SEQ ID NO:108); BCD_0353 (SEQ ID NO:88); BCD_0414 (SEQ ID NO:104); BCD_0351 (SEQ ID NO:87); BCD_0459 (SEQ ID NO: 116); BCD_0395 (SEQ ID NO:101); BCD_0393 (SEQ ID NO:99); BCD_0394 (SEQ ID NO:100); BCD_0362 (SEQ ID NO:93); and BCD_0506 (SEQ ID NO:362) are aligned.

FIGS. 7A-7D show an alignment of the VP1 protein for AAV clade 6. VP1 protein of AAV-7 (SEQ ID NO:7); BCD_0188 (SEQ ID NO:49); BCD_0176 (SEQ ID NO:39); BCD_0179 (SEQ ID NO:42); BMN_0254 (SEQ ID NO:142); BMN_0258 (SEQ ID NO:146); BMN_0253 (SEQ ID NO:141); BMN_0260 (SEQ ID NO:147); BMN_0257 (SEQ ID NO:145); BMN_0256 (SEQ ID NO:144); BMN_0255 (SEQ ID NO:143); BCD_0194 (SEQ ID NO:54); BMN_0251 (SEQ ID NO:140); BMN_0250 (SEQ ID NO:139); BCD_0485 (SEQ ID NO:122); BCD_0489 (SEQ ID NO: 126); BCD_0196 (SEQ ID NO:56); BCD_0120 (SEQ ID NO:19); BCD_0178 (SEQ ID NO:41); and BCD_0189 (SEQ ID NO:50) are aligned.

FIGS. 8A-8B show an alignment of the VP1 protein for AAV clade 11. VP1 protein of AAV-9 (SEQ ID NO:9); BCD_0112 (SEQ ID NO:14); BMN_0319 (SEQ ID NO:178); BMN_0316 (SEQ ID NO:175); BMN_0321 (SEQ ID NO:180); BMN_0317 (SEQ ID NO:176); BMN_0318 (SEQ ID NO:177); BMN_0320 (SEQ ID NO:179) are aligned.

FIGS. 9A-9B show an alignment of the VP1 protein for AAV clade 12. VP1 protein of BCD_0105 (SEQ ID NO:12); AAV-2 (SEQ ID NO:4); BCD_0137 (SEQ ID NO:24); and BCD_0447 (SEQ ID NO:110) are aligned.

FIGS. 10A-10B show an alignment of the VP1 protein for AAV clade 13. VP1 protein of BCD_0168 (SEQ ID NO:32); BCD_0169 (SEQ ID NO:33); BCD_0171 (SEQ ID NO: 35); BCD_0172 (SEQ ID NO:36); BCD_0170 (SEQ ID NO:34); BCD_0159 (SEQ ID NO: 27); and BCD_0167 (SEQ ID NO:31) are aligned.

FIGS. 11A-11B show an alignment of the VP1 protein for AAV clade 16. VP1 protein of BCD_0441 (SEQ ID NO:106); BCD_0442 (SEQ ID NO:107); AAV-1 (SEQ ID NO: 3); and AAV-6 (SEQ ID NO:6) are aligned.

FIGS. 12A-12B show an alignment of the VP1 protein for AAV clade 21. VP1 protein of BCD_0115 (SEQ ID NO:17); BCD_0113 (SEQ ID NO:15); BCD_0117 (SEQ ID NO: 18); BCD_0114 (SEQ ID NO:16); AAV-13 (SEQ ID NO:11); AAV-LK03 (SEQ ID NO:1); and AAV-3b (SEQ ID NO:5) are aligned.

FIG. 13 shows an alignment of the VP1 protein for AAV clade 22. VP1 protein of BCD_0135 (SEQ ID NO:23); and BCD_0157 (SEQ ID NO:26) are aligned.

FIGS. 14A-14B show in vitro IVIg neutralization data of selected rAAVs, including novel rAAV viral particles.

FIG. 15 shows a representative image of in vivo BCD_0160 expression in the brain.

### 6. DETAILED DESCRIPTION

### 6.1 General Overview

The present disclosure provides novel AAV capsid sequences (nucleic and amino acid sequences) and functional fragments thereof. Also provided herein, are novel AAV isolates, clades, and branches for broader use of AAV-based vectors in biomedical applications, such as gene therapy, which provide improved functional characteristics over previously described AAV-based vectors.

The disclosure also provides rAAV viral particles, vectors, rAAV vector genome constructs, host cells, and pharmaceutical compositions. The novel AAV capsid based vectors and/or rAAV viral particles provide enhanced evasion of AAV humoral immunity, enhanced tropism, enhanced cell transduction, and/or enhanced transgene expression as compared to a reference AAV.

Also provided herein are methods of delivery of a biomolecule (e.g., a therapeutic biomolecule). In some embodiments, the method is in vivo, in vitro, or ex vivo delivery. In some embodiments, the method delivers the biomolecule (e.g., a therapeutic biomolecule) to one or more cells, particularly enhanced delivery/tropism to a muscle, heart, liver, plasma, kidney, brain, and/or cancer cell, while detargeting other cells.

The present disclosure also provides methods of treatment including administering to a subject in need any of the novel AAV capsid sequences/functional fragments, rAAV vector genome constructs, rAAV particles, host cells, or pharmaceutical compositions provided herein. The methods of treatment can be used for a disease or disorder capable of being treated by delivery to muscle, heart, liver, plasma, kidney, brain, or/and cancer cell.

Further provided herein, are methods of manufacturing a novel rAAV viral particle of the disclosure and producing a biomolecule (e.g., a therapeutic biomolecule) using a novel rAAV viral particle.

### 6.2 Definitions

Unless specifically stated or apparent from context, as used herein, the terms “a”, “an”, and “the” are understood to be singular or plural.

The terms “include(s)” or “such as,” and the like are intended to convey inclusion without limitation, unless otherwise specifically indicated

The terms “or” and “and” can be used interchangeably and can be understood to mean “and/or.”

The term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “heterologous gene” or “heterologous regulatory sequence” means that the referenced gene or regulatory sequence is not naturally present in the AAV vector or particle and has been artificially introduced therein.

The term “heterologous transgene” or “transgene” refers to a nucleic acid that comprises both a heterologous gene and a heterologous regulatory sequence that are operably linked to the heterologous gene that control expression of that gene in a host cell. It is contemplated that the transgene herein can encode a biomolecule (e.g., a therapeutic biomolecule), such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., a CRISPR gene editing system, antisense oligonucleotides (AONs), antisense oligonucleotide (AON)-mediated exon skipping, a poison exon(s) that triggers nonsense mediated decay (NMD), or a dominant negative mutant.

The term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells.

An “AAV vector genome” or “rAAV vector genome” refers to nucleic acids, either single-stranded or double-stranded, comprising an AAV inverted terminal repeat (ITR) (e.g., an AAV 5′ inverted terminal repeat (ITR) sequence and an AAV 3′ ITR) flanking a biomolecule (e.g., a therapeutic biomolecule) or transgene operably linked to a transcription regulatory element(s) that is heterologous to the AAV viral genome, e.g., one or more promoters and/or enhancers and, optionally, a polyadenylation sequence and/or one or more introns inserted between exons of the protein-coding sequence. A single-stranded AAV vector genome refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector genome can be provided by a double-stranded vector or virus, e.g., baculovirus, used to express or transfer the AAV vector genome nucleic acids. The size of such double-stranded nucleic acids in provided in base pairs (bp). In a specific embodiment, the AAV vector genome is a recombinant AAV vector genome.

The “AAV rep gene” or “rep” as used herein refers to the art-recognized region of the AAV genome which encodes the replication proteins of the virus which are required to replicate the viral genome and to insert the viral genome into a host genome during latent infection. For a further description of the AAV rep coding region, see, e.g., Muzyczka et al., Current Topics in Microbiol. and Immunol. 158:97-129 (1992); Kotin et al., Human Gene Therapy 5:793-801 (1994), the disclosures of which are incorporated herein by reference in their entireties. The rep coding region, as used herein, can be derived from any viral serotype, such as the AAV serotypes described herein. The region need not include all of the wild-type genes of an AAV serotype but may be altered, e.g., by the insertion, deletion and/or substitution of nucleotides, so long as the rep genes retain the desired functional characteristics when expressed in a suitable recipient cell (e.g., the ability to provide viral genome replication and packaging during infection).

The “AAV cap gene” or “cap” as used herein refers to the art-recognized region of the AAV genome which encodes the coat proteins of the virus which are required for packaging the viral genome. For a further description of the cap coding region, see, e.g., Muzyczka et al., Current Topics in Microbiol. and Immunol. 158:97-129 (1992); Kotin et al., Human Gene Therapy 5:793-801 (1994), the disclosures of which are incorporated herein by reference in their entireties. The AAV cap coding region, as used herein, can be derived from any AAV serotype, as described herein. The region need not include all of the wild-type cap genes of an AAV serotype but may be altered, e.g., by the insertion, deletion or substitution of nucleotides, so long as the genes provide for sufficient packaging functions when present in a host cell along with an AAV vector.

An “AAV virion” or “AAV viral particle” or “AAV particle” or “AAV vector particle” or “AAV virus” refers to a viral particle composed of at least one AAV capsid protein (e.g., VP1, VP2, or VP3, or a combination thereof). In a specific embodiment, an “AAV virion” or “AAV viral particle” or “AAV vector particle” or “AAV virus” refers to a virus composed of at least one AAV capsid protein and an encapsidated polynucleotide AAV vector genome. If the particle comprises a heterologous nucleotide sequence (e.g., an AAV vector genome) (i.e., a polynucleotide other than a wild-type AAV genome such as a transgene to be delivered to a mammalian cell), it is typically referred to as an “AAV vector particle”. Thus, production of AAV vector particle necessarily includes production of AAV vector genome, as such a vector genome is contained within an AAV vector particle. In a specific embodiment, the AAV viral particle is a recombinant AAV viral particle.

A “variable region” or “VR” or “VRs” refer to amino acids region(s) that vary within a capsid viral protein (“VP”, VP1, VP2, or VP3) and that are not a part of the conserved core structure. Generally, the variable regions contain surface loops conformations within the capsid viral proteins. The VR exhibit the highest sequence and structural variation within the AAV capsid sequences and may also have roles in receptor attachment, transcriptional activation of transgenes, tissue transduction and antigenicity. Table 8 provides examples of variable regions VRI-VRIX, GBS region, and GH loop. In certain embodiments, the location of the N-terminal and/or C-terminal ends of those regions may vary by from up to 1 amino acid, 2 amino acids, 3 amino acids, 4 amino acids or 5 amino acids from the amino acid locations of those regions as they are explicitly described herein (particularly in Table 8).

The “glycan binding sequence (GBS)” or “GBS domain” or “GBS region” refer to the amino acid sequence located between VR IV and VR V that governs the glycan binding specificity of the viral capsid. The locations of the GBS regions in various AAV VP1 amino acid sequences are herein described, and those from other AAV VP1 amino acid sequences are known in the art and/or may be routinely identified. Table 8 provides examples of GBS regions. In certain embodiments, the location of the N-terminal and/or C-terminal ends of the GBS region may vary by from up to 1 amino acid, 2 amino acids, 3 amino acids, 4 amino acids, or 5 amino acids from the amino acid locations of the GBS region explicitly described herein (particularly in Table 8).

The term “GH loop” refers to a loop sequence that is flanked by β-strand G and β-strand H within the internal β-barrel of the capsid protein. The “GH loop” sequence comprises variable region VR IV through VR VIII, including the encompassed GBS sequence and all interspersed conserved backbone sequence from the donor. The locations of the GH loop regions in various AAV VP1 amino acid sequences are herein described and those from other AAV VP1 amino acid sequences may be routinely identified. Table 8 provides examples of GH loops. In certain embodiments, the location of the N-terminal and/or C-terminal ends of the GH loop may vary by from up to 1 amino acid, 2 amino acids, 3 amino acids, 4 amino acids, or 5 amino acids from the amino acid locations of the GH loop explicitly described herein (particularly in Table 8).

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. In specific embodiments, a fragment is at least about 8 amino acids in length, and may be up to about 700 amino acids. Examples of suitable functional fragments are described herein (e.g., in Section 6.3.1.3, infra).

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In a specific embodiment, the percent identity between at least two sequences (e.g., amino acid sequences or nucleic acid sequences) is accomplished using ClustalW. In certain specific embodiments, ClustalW with cost matrix BLOSUM, a gap open cost of 10, and a gap extend cost of 0.1 is used to determine the percent identity between at least two sequences (e.g., amino acid sequences or nucleic acid sequences).

The term “substantial identity” when referring to amino acids or fragments thereof, indicates that, when optimally aligned with appropriate amino acid insertions or deletions with another amino acid (or its complementary strand), there is amino acid sequence identity in at least about 90 to 99% of the aligned sequences using a technique described herein (e.g., ClustalW). Preferably, the identity is over the full-length of the two sequences being compared or a fragment thereof which is at least 8 amino acids, or more desirably, at least 15 amino acids in length. Examples of suitable fragments are described herein.

A “fragment” of a protein, polypeptide or peptide refers to a sequence of at least 8 amino acids in length. In specific embodiments, a fragment of a protein, polypeptide or peptide is at least about 15 amino acids in length, at least about 18 amino acids in length, at least about 20 amino acids in length, at least about 25 amino acids in length, at least about 30 amino acids in length, at least about 35 amino acids in length, at least about 40 amino acids in length, at least about 18 amino acids in length, at least about 45 amino acids in length, at least about 50 amino acids in length, at least about 55 amino acids in length, or at least about 60 amino acids in length. In specific embodiments, a fragment of a protein, polypeptide or peptide is at least about 65 amino acids in length, at least about 70 amino acids in length, at least about 80 amino acids in length, at least about 85 amino acids in length, at least about 90 amino acids in length, at least about 95 amino acids in length, at least about 100 amino acids in length, at least about 105 amino acids in length, at least about 110 amino acids in length, at least about 115 amino acids in length, at least about 120 amino acids in length, or at least about 125 amino acids in length. In specific embodiments, a fragment of a protein, polypeptide or peptide is at least about 150 amino acids in length, at least about 200 amino acids in length, at least about 250 amino acids in length, at least about 300 amino acids in length, at least about 350 amino acids in length, at least about 400 amino acids in length, at least about 450 amino acids in length, at least about 500 amino acids in length, at least about 550 amino acids in length, or at least about 600 amino acids in length. In specific embodiments, a fragment of a protein, polypeptide or peptide is about 9 to about 25 amino acids in length, about 15 to about 25 amino acids in length, about 20 to about 50 amino acids in length, about 25 to about 50 amino acids in length, about 50 to about 75 amino acids in length, about 50 to about 100 amino acids in length, or about 75 to about 100 amino acids in length. In specific embodiments, a fragment of a protein, polypeptide, or peptide is about 100 to about 150 amino acids in length, about 100 to about 200 amino acids in length, about 150 to about 200 amino acids in length, about 150 to about 300 amino acids in length, about 200 to about 300 amino acids in length, about 250 to about 300 amino acids in length, or about 300 to about 400 amino acids in length. In specific embodiments, a fragment comprises a portion of consecutive amino acid residues of a protein, polypeptide, or peptide.

A “fragment” of a nucleic acid sequence refers to a sequence of at least 9 nucleotides in length. In specific embodiments, a fragment of a nucleic acid sequence is at least about 15 nucleotides in length, at least about 18 nucleotides in length, at least about 20 nucleotides in length, at least about 25 nucleotides in length, at least about 30 nucleotides in length, at least about 35 nucleotides in length, at least about 40 nucleotides in length, at least about 18 nucleotides in length, at least about 45 nucleotides in length, at least about 50 nucleotides in length, at least about 55 nucleotides in length, or at least about 60 nucleotides in length. In specific embodiments, a fragment of a nucleic acid sequence is at least about 65 nucleotides in length, at least about 70 nucleotides in length, at least about 80 nucleotides in length, at least about 85 nucleotides in length, at least about 90 nucleotides in length, at least about 95 nucleotides in length, at least about 100 nucleotides in length, at least about 105 nucleotides in length, at least about 110 nucleotides in length, at least about 115 nucleotides in length, at least about 120 nucleotides in length, or at least about 125 nucleotides in length. In specific embodiments, a fragment of a nucleic acid sequence is at least about 150 nucleotides in length, at least about 200 nucleotides in length, at least about 250 nucleotides in length, at least about 300 nucleotides in length, at least about 350 nucleotides in length, at least about 400 nucleotides in length, at least about 450 nucleotides in length, at least about 500 nucleotides in length, at least about 550 nucleotides in length, or at least about 600 nucleotides in length. In specific embodiments, a fragment of a nucleic acid sequence is about 9 to about 25 nucleotides in length, about 15 to about 25 nucleotides in length, about 20 to about 50 nucleotides in length, about 25 to about 50 nucleotides in length, about 50 to about 75 amino acids in length, about 50 to about 100 amino acids in length, or about 75 to about 100 amino acids in length. In specific embodiments, a fragment of a nucleic acid sequence is about 100 to about 150 amino acids in length, about 100 to about 200 amino acids in length, about 150 to about 200 amino acids in length, about 150 to about 300 amino acids in length, about 200 to about 300 amino acids in length, about 250 to about 300 amino acids in length, or about 300 to about 400 amino acids in length. In specific embodiments, a fragment comprises a portion of consecutive nucleotides of a nucleic acid sequence.

The term “functional version” in the context of endogenous nucleic acid or protein means it has a functionality of a reference nucleic acid sequence or protein in vitro, when expressed in cultured cells, or in vivo, when expressed in cells or body tissues. For example, a functional version of a protein may retain one, two, three or more functions of an endogenous protein. In a particular example, a functional version of an enzyme would retain its enzymatic activity, protein binding/signaling, transport, or structural properties in a cell or organ. In an alternatively example, a functional version can also be a codon-optimized gene, a mini-gene that removes segments of the gene such as introns or codons not required for the function of interest.

The term “AAV clade” or “clade” means a group of AAV isolates defined by one or more common structural features of their capsid viral protein (VP1, VP2, and/or VP3), such as those provided in Sections 6.3.2.1-6.3.2.4. An AAV clade may be further defined by one or more common functional features such as tropism or ability to evade AAV humoral immunity. Alternatively, example a clade can be described as in Gao et al. J Virol. (2004) June; 78 (12): 6381-6388 by neighbor-joining tree analysis with maximum parsimony and likelihood that show phylogenetic groups containing nonredundant but phylogenetically similar AAV members (e.g., isolates) from different tissue sources (e.g., two or more, three or more AAV members).

### 6.3 Compositions

### 6.3.1 Novel AAV Capsid Sequences

The disclosure provides novel AAV capsid sequences including nucleic acid sequences (DNA, cDNA, and RNA) and amino acid sequences encoded by the nucleic acid sequences as well as fragments thereof, individually referred to herein as “novel AAV capsid nucleic acid sequences” and “novel AAV capsid amino acid sequences”, respectively, and collectively referred herein as “novel AAV capsid sequences.” The disclosure also provides for modified AAV capsid sequences. See Section 6.3.1.4. Unless explicitly clear from the context, the phrase “novel AAV capsid sequences” encompasses modified novel AAV capsid sequences.

In specific embodiments, provided herein are novel AAV capsid sequences comprising a VP1 sequence, a VP2 sequence, or a VP3 sequence that is at least 90%, at least 91%, at least 92%, at least 93%, at least 94%, at least 95%, at least 96%, at least 97%, at least 98%, or at least 99% identical to a VP1 sequence, a VP2 sequence, or a VP3 sequence of a capsid provided herein (e.g., in Table 9) with the “BCD_” prefix. In specific embodiments, provided herein are novel AAV capsid sequences comprising a VP1 sequence, a VP2 sequence, or a VP3 sequence identical to a VP1 sequence, a VP2 sequence, or a VP3 sequence of a capsid provided herein (e.g., in Table 9) with the “BCD_” prefix. In specific embodiments, a novel AAV capsid sequence comprises a VP1 sequence, a VP2 sequence, and a VP3 sequence.

An AAV capsid comprises three capsid proteins, VP1, VP2, and VP3. Often, a novel AAV capsid nucleotide sequence of the disclosure comprises a nucleotide sequence that encodes or codes for three proteins, referred to as viral proteins: VP1, VP2, and VP3. VP2 and VP3, are smaller than VP1. VP2 and VP3 coding regions are derived from the VP1 coding region and comprise a subset of the VP1 coding region AAV capsids may also be described as comprising constant regions and variable regions. The novel AAV capsid amino acid sequences include VP1, VP2, and VP3, as well as constant regions and variable regions. The novel AAV capsid sequences provided in the figures and sequence listing are of VP1 and VP1 coding regions.

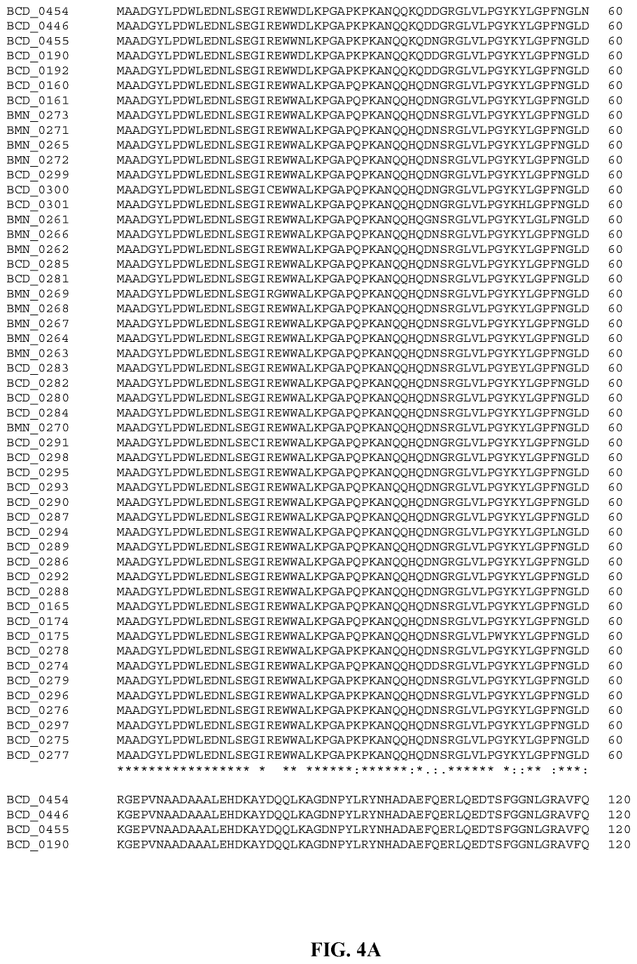

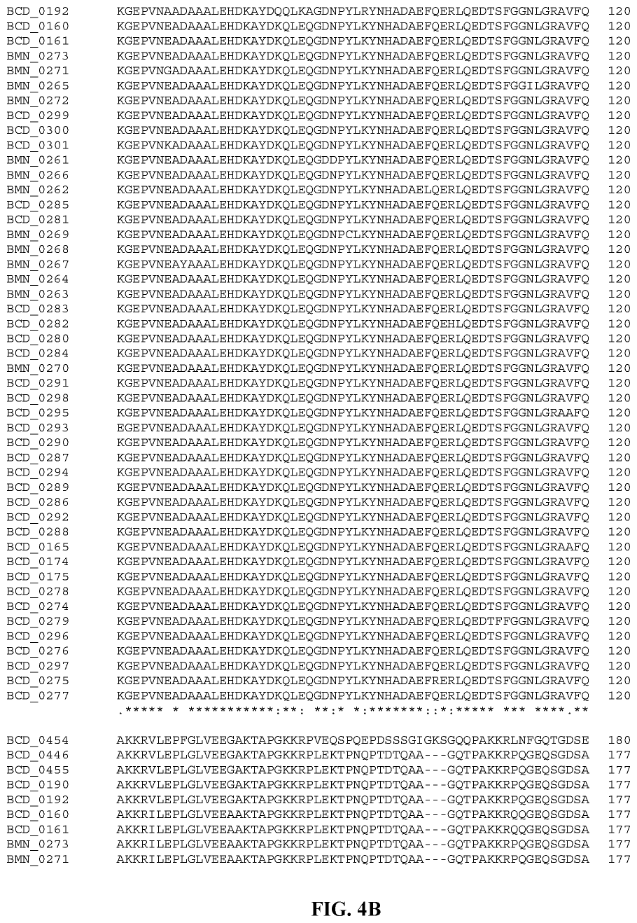

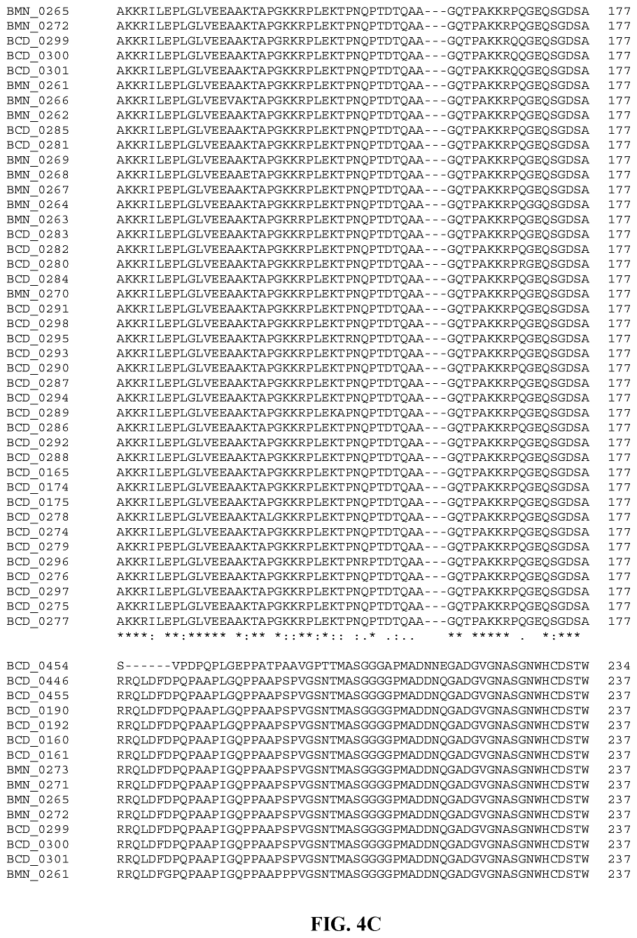

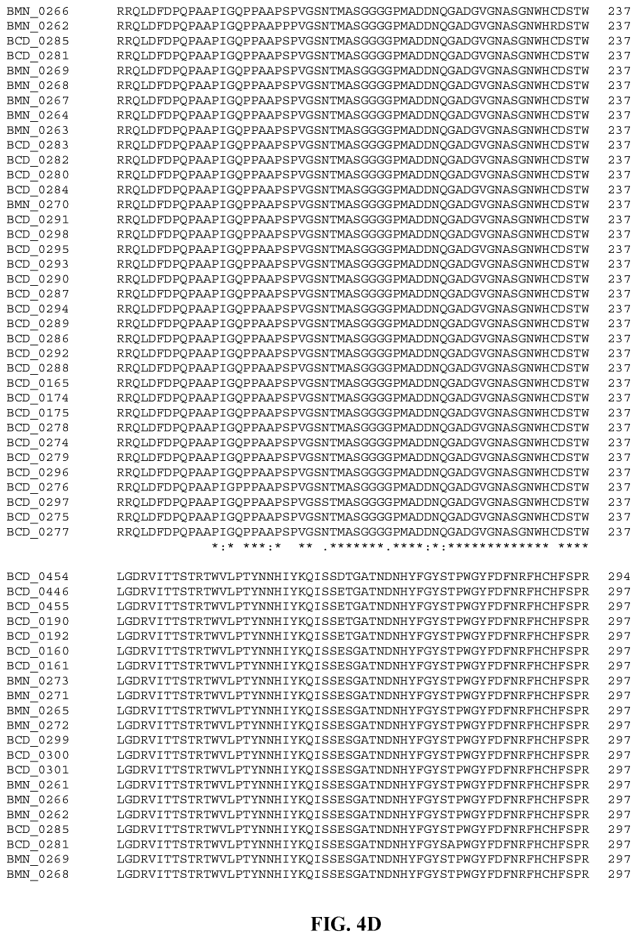

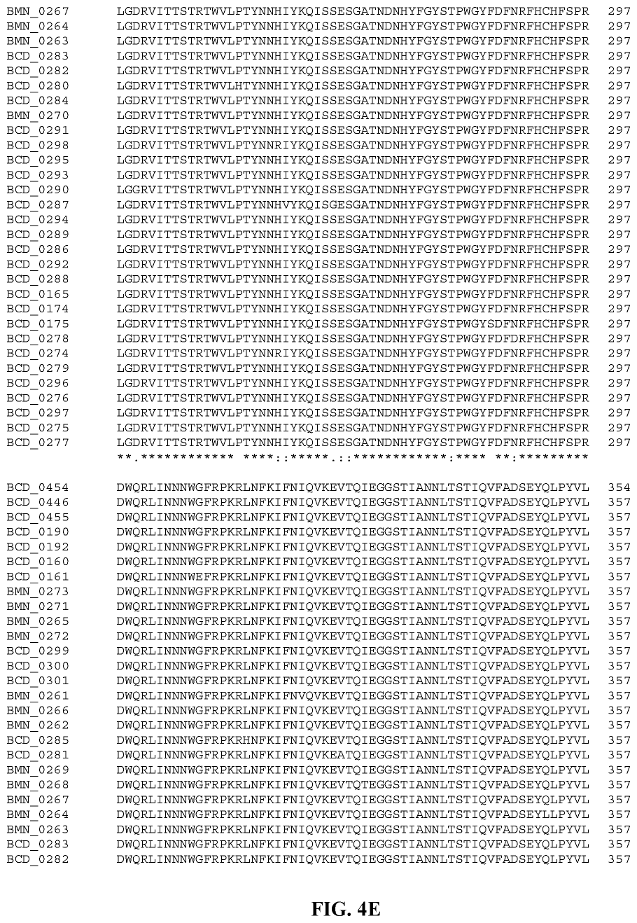

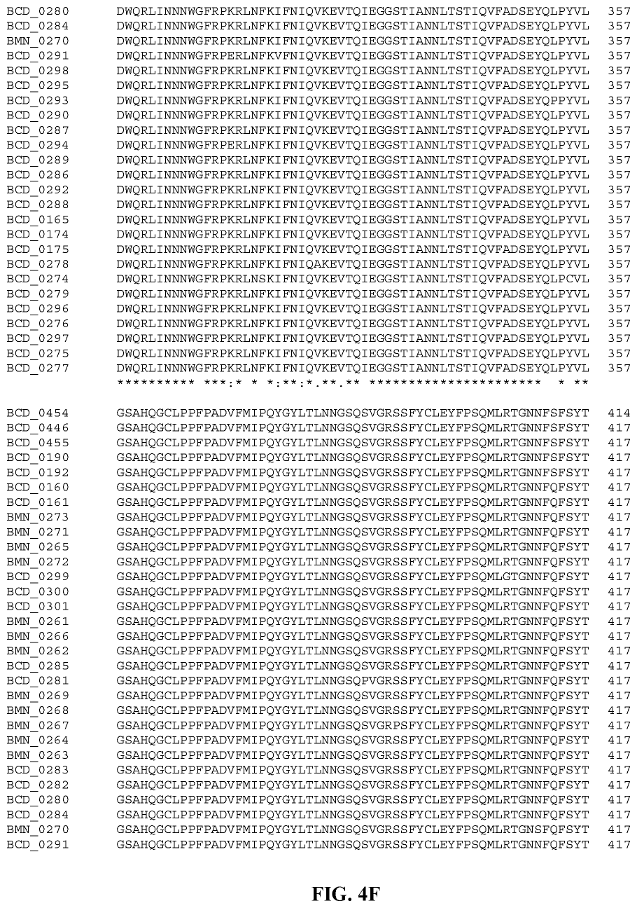

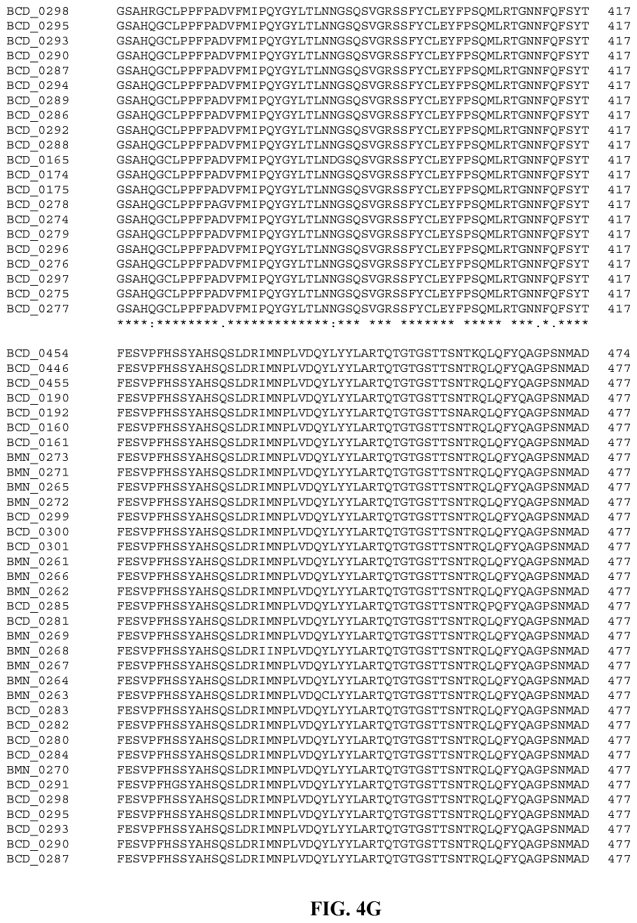

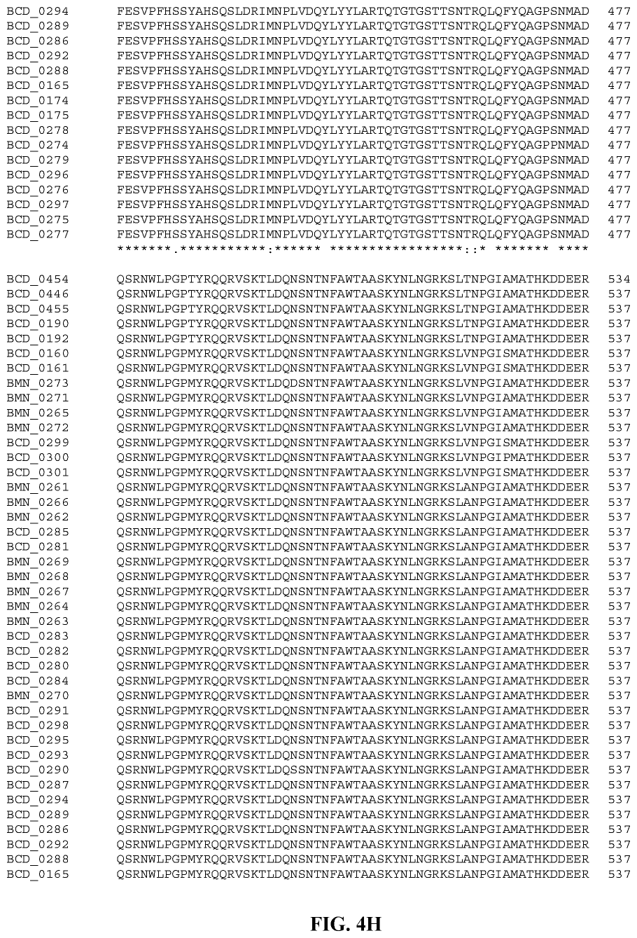

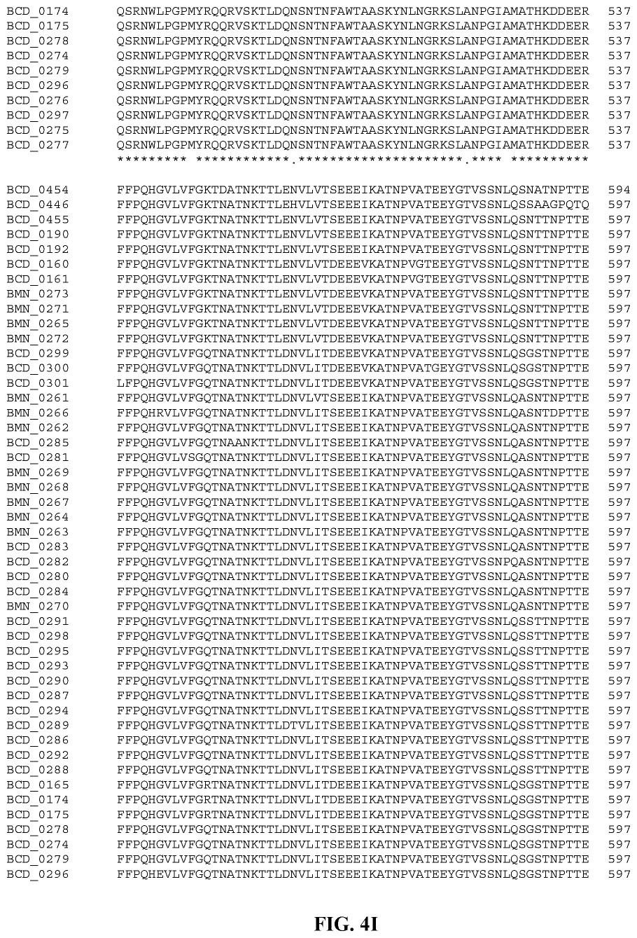

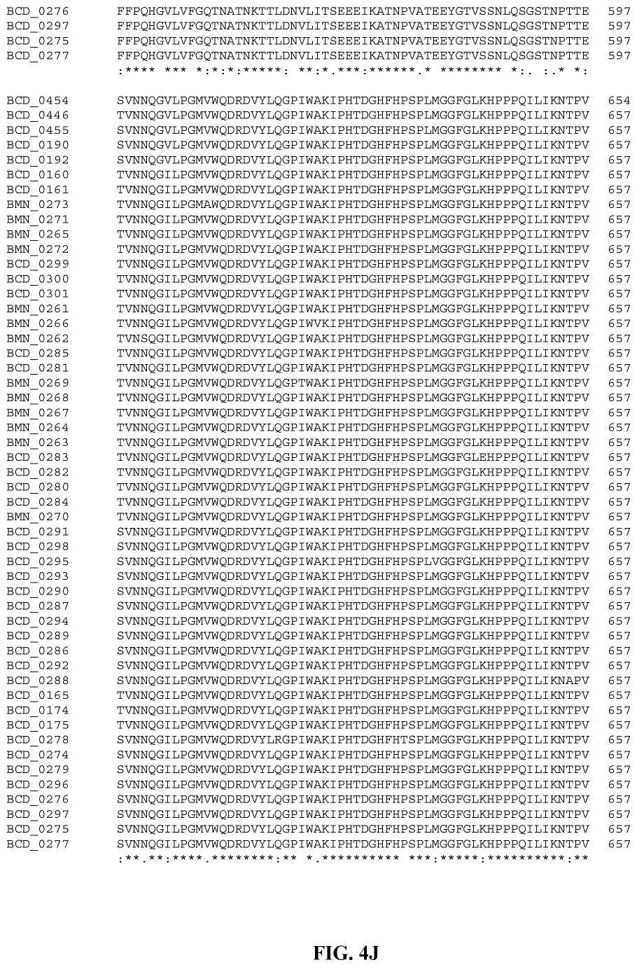

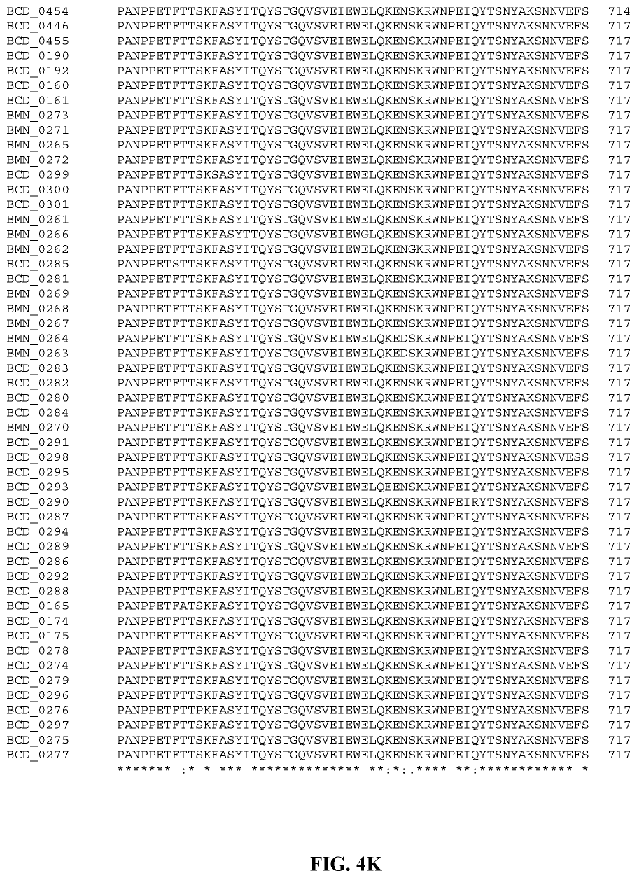

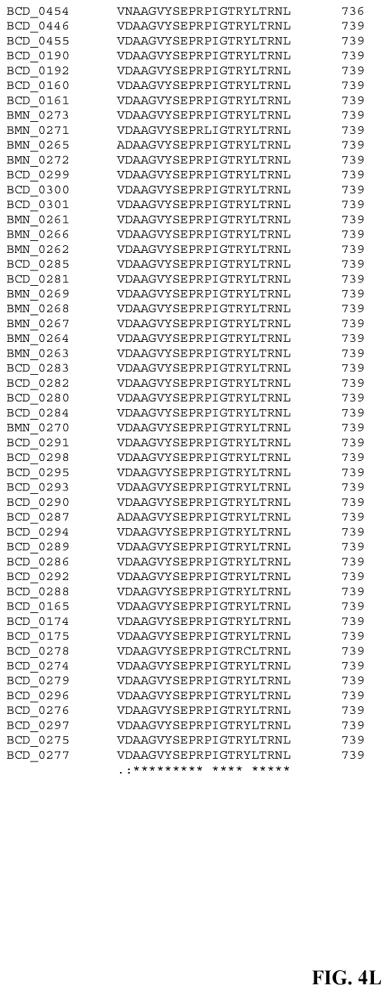

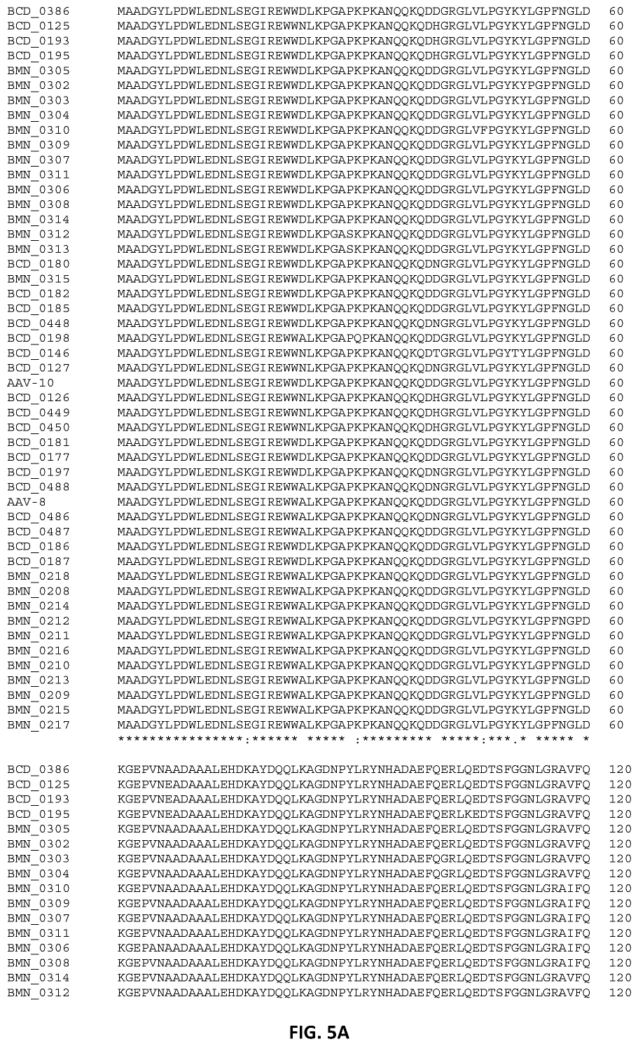

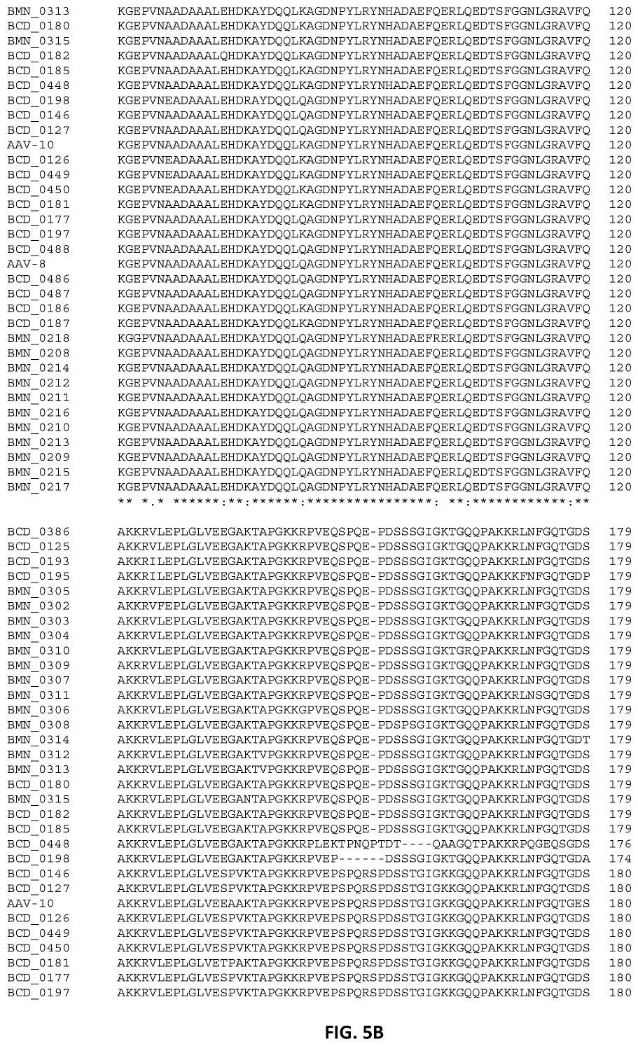

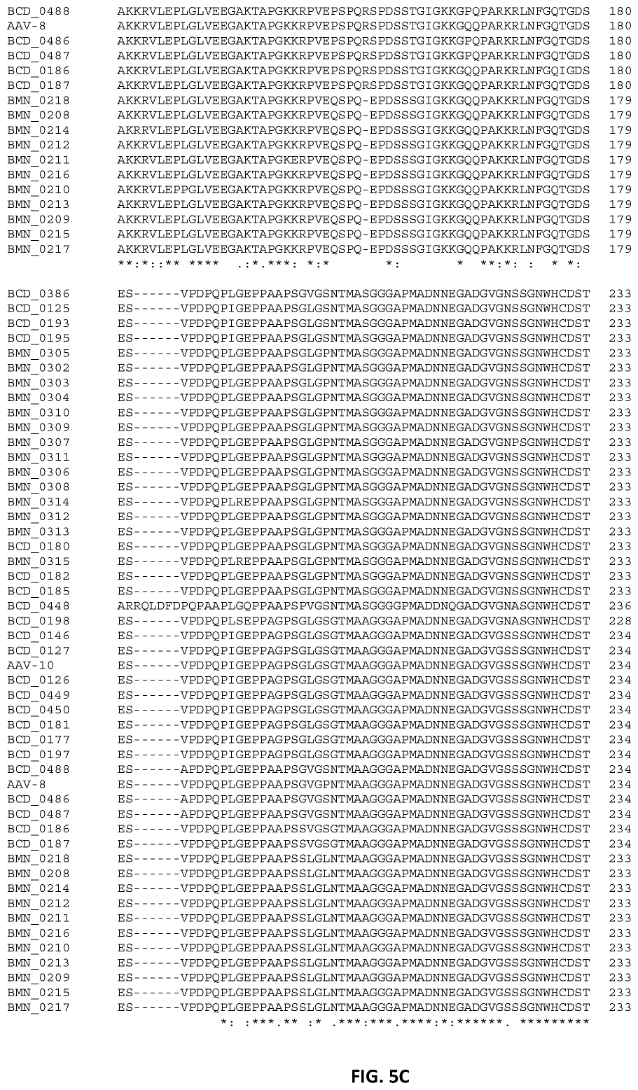

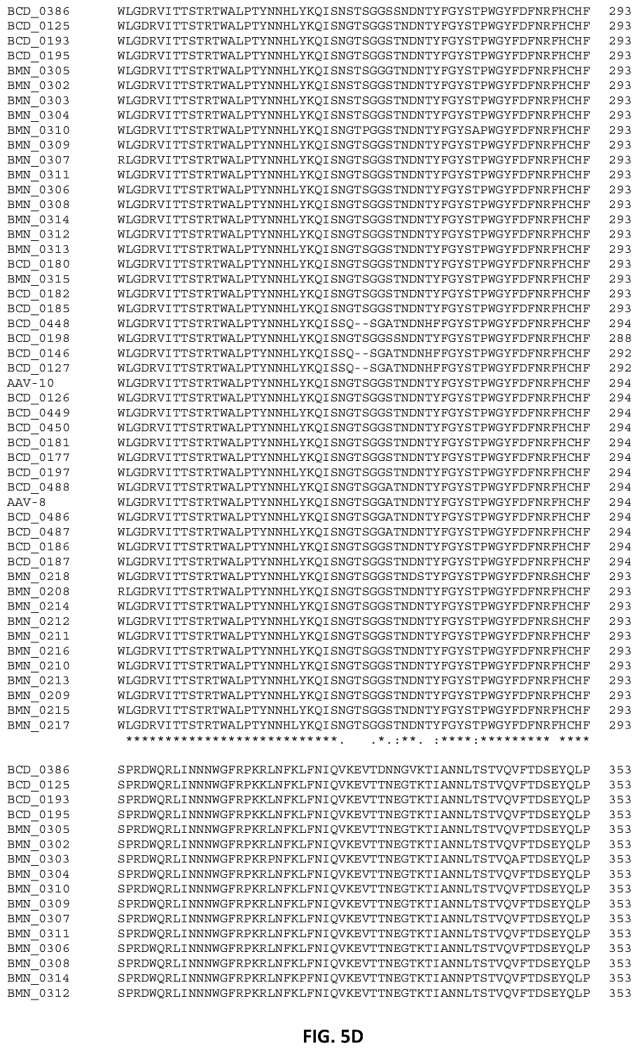

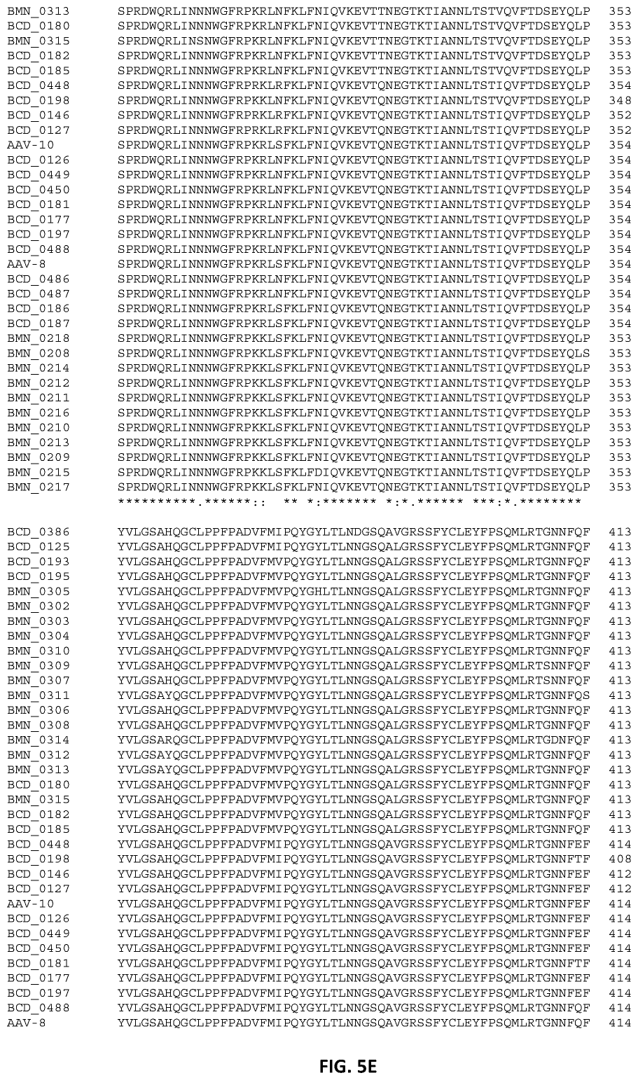

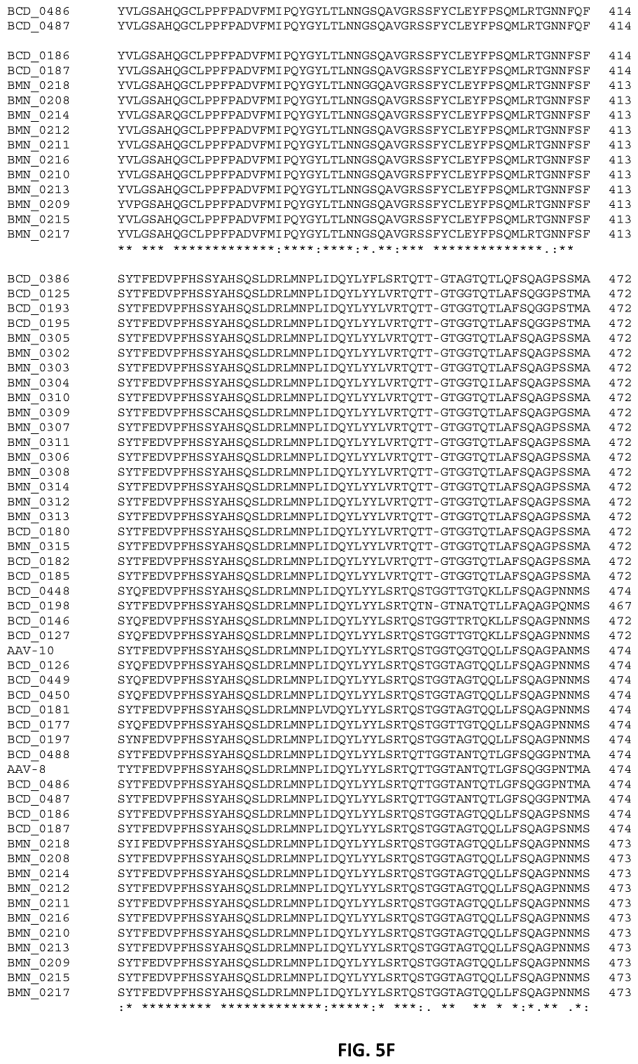

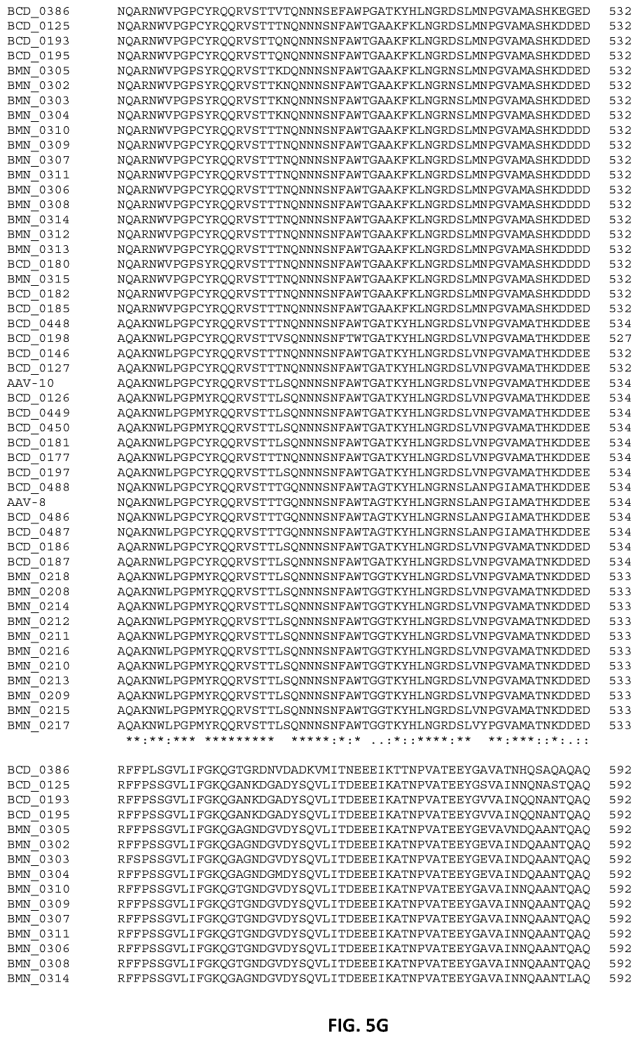

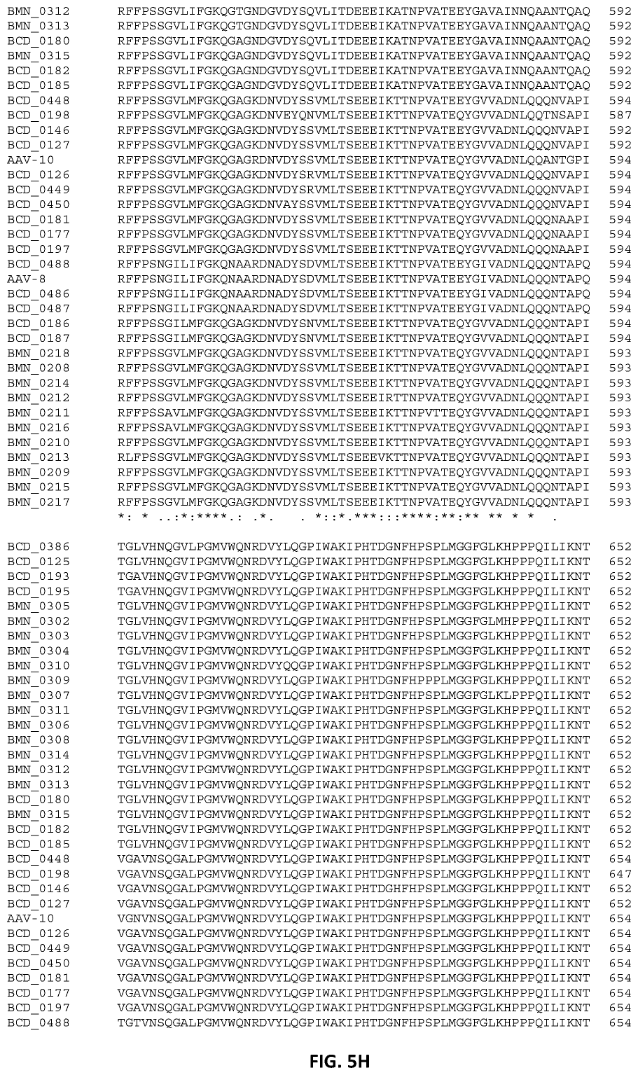

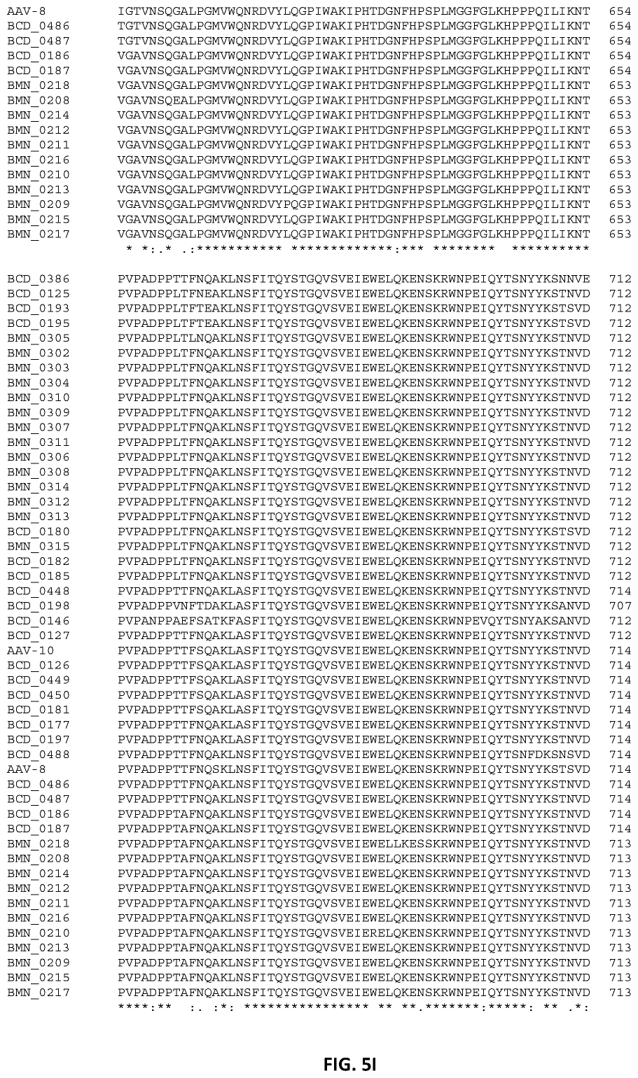

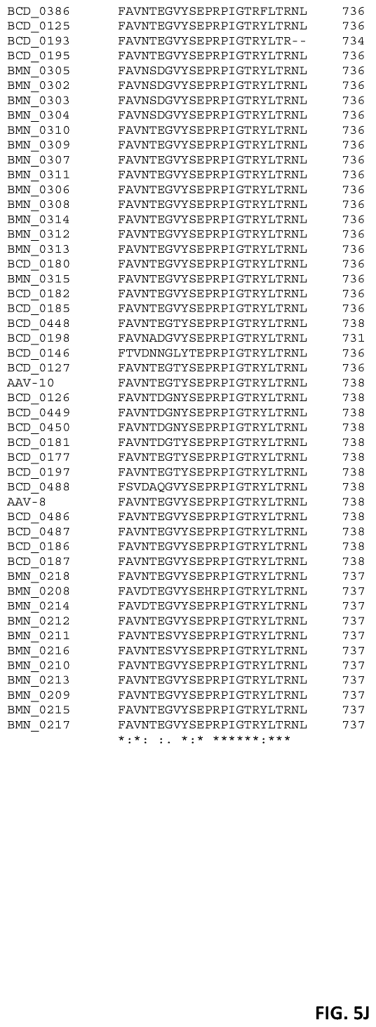

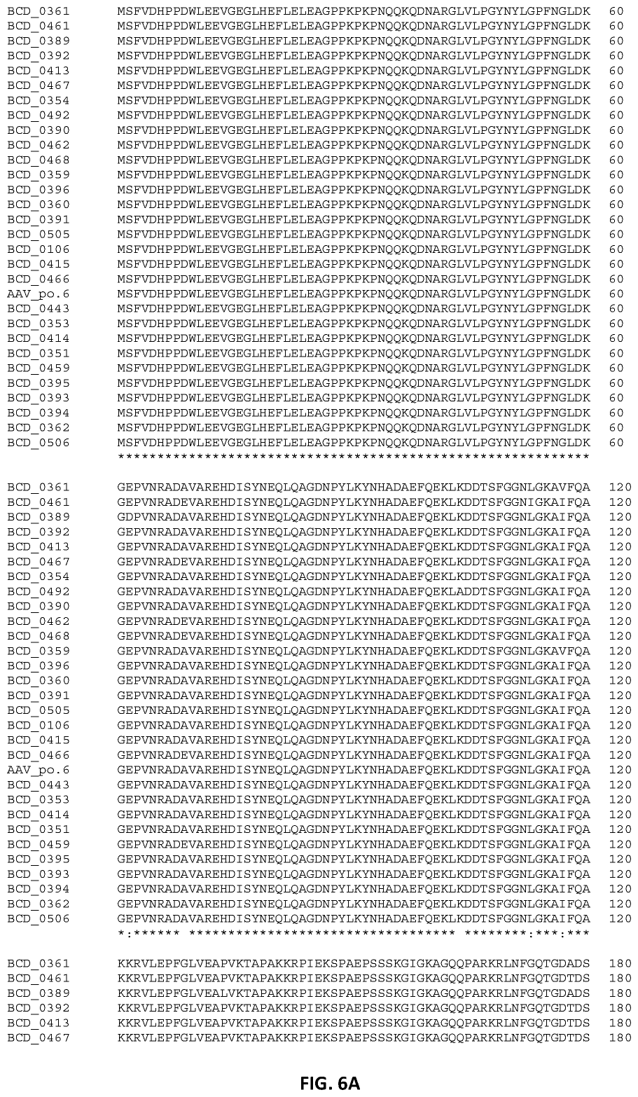

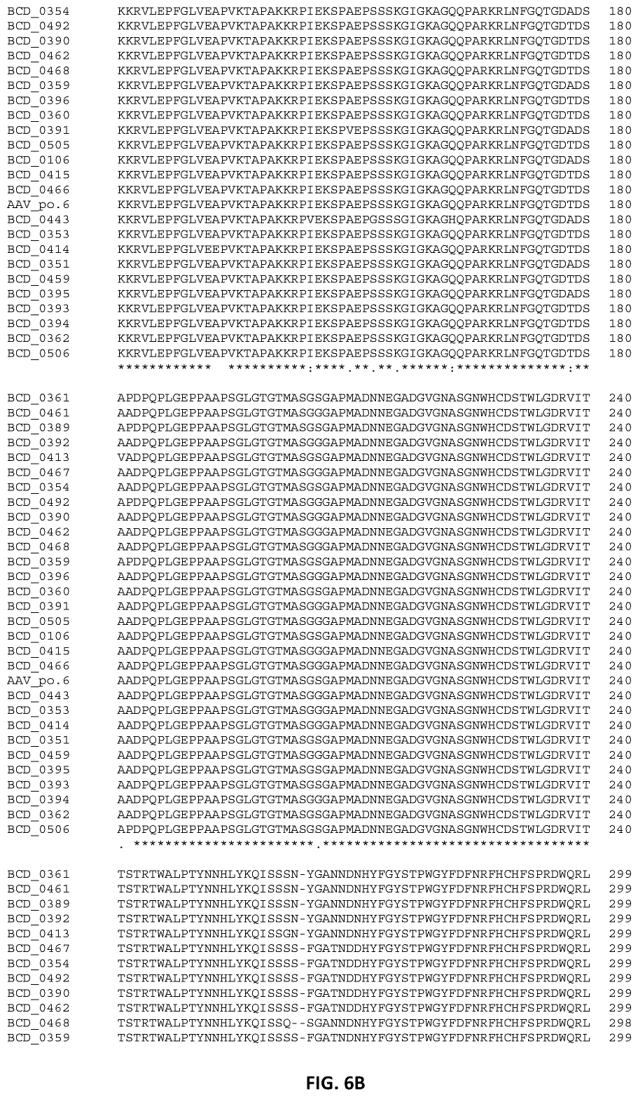

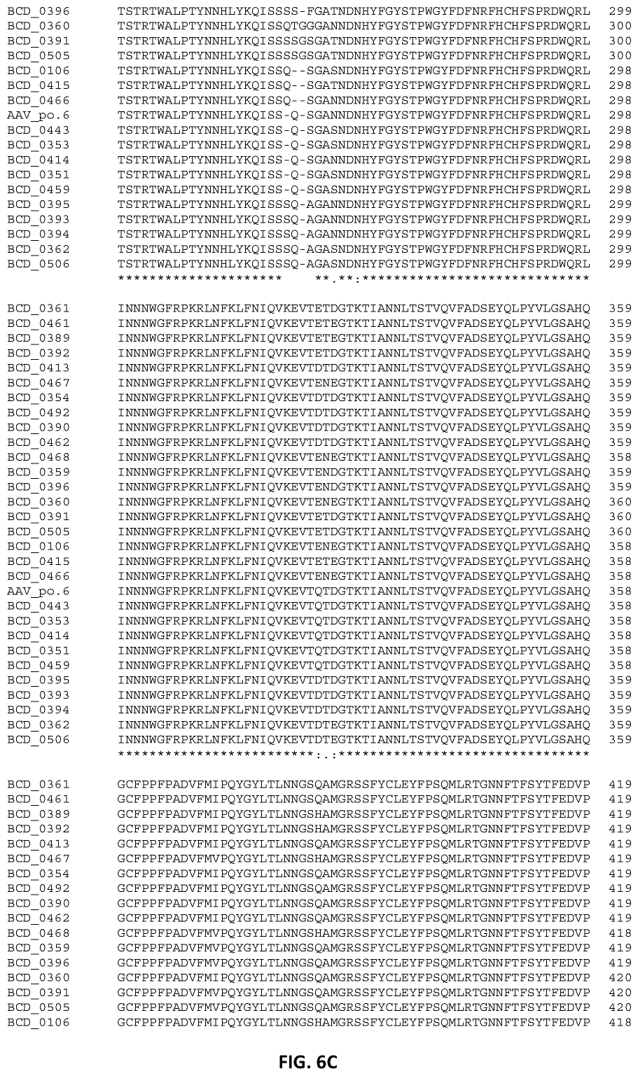

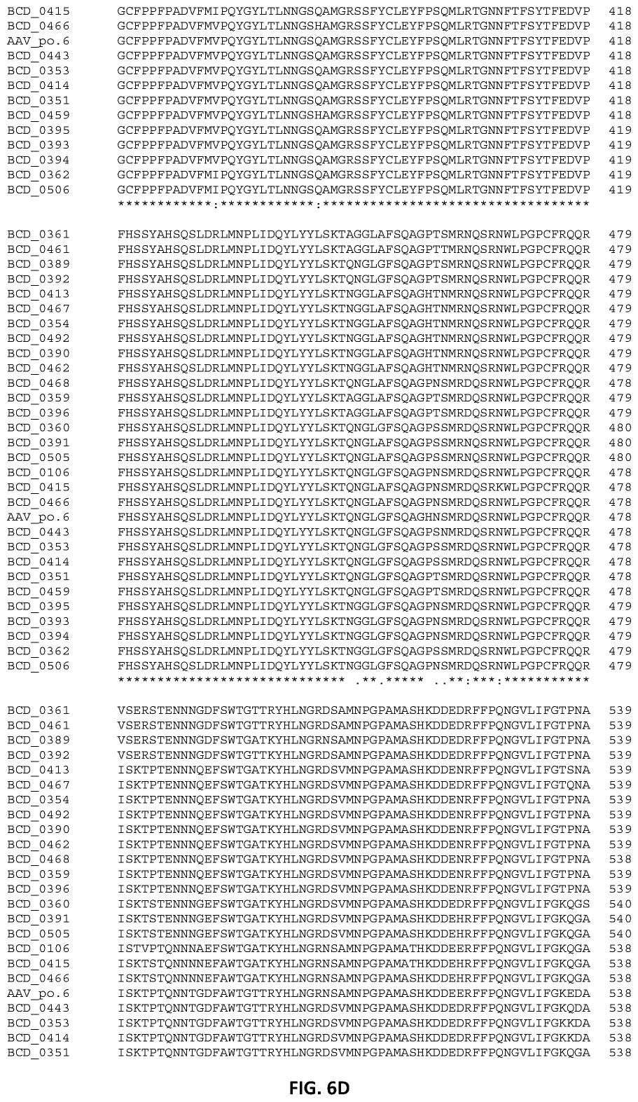

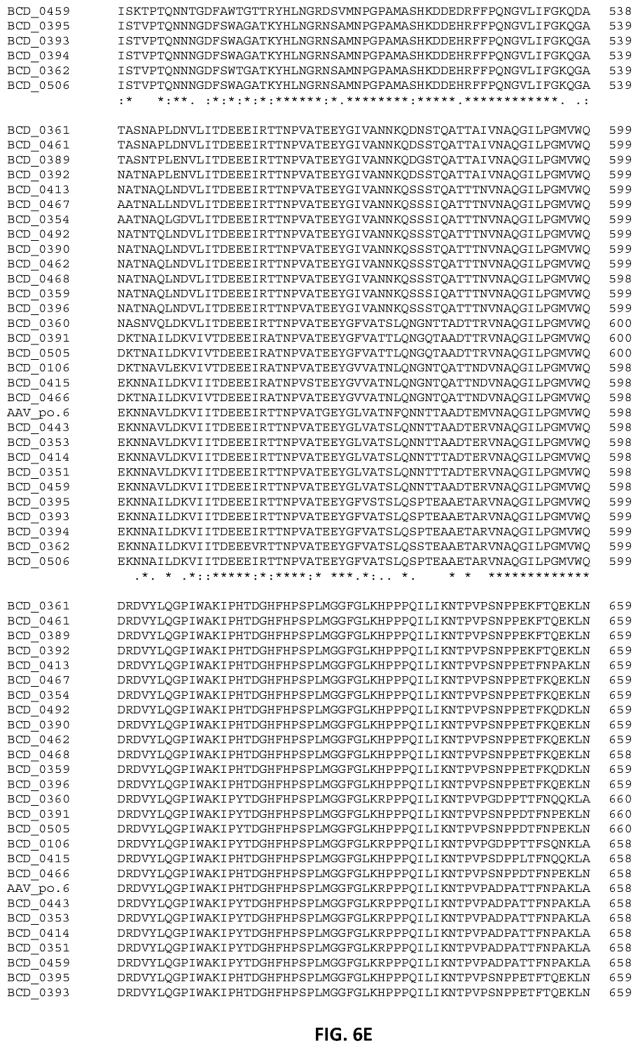

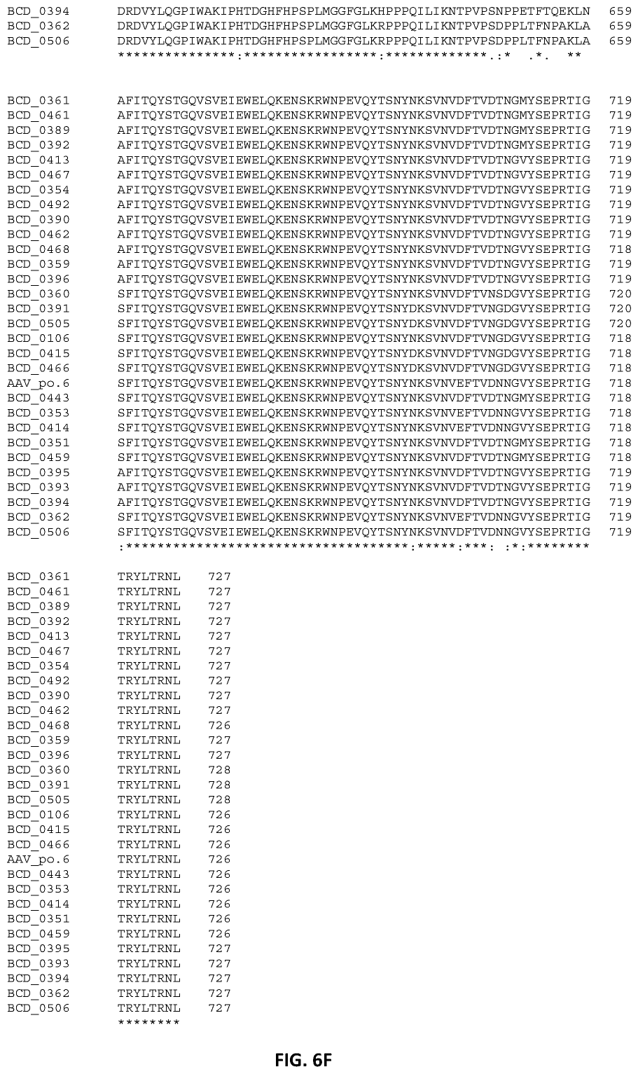

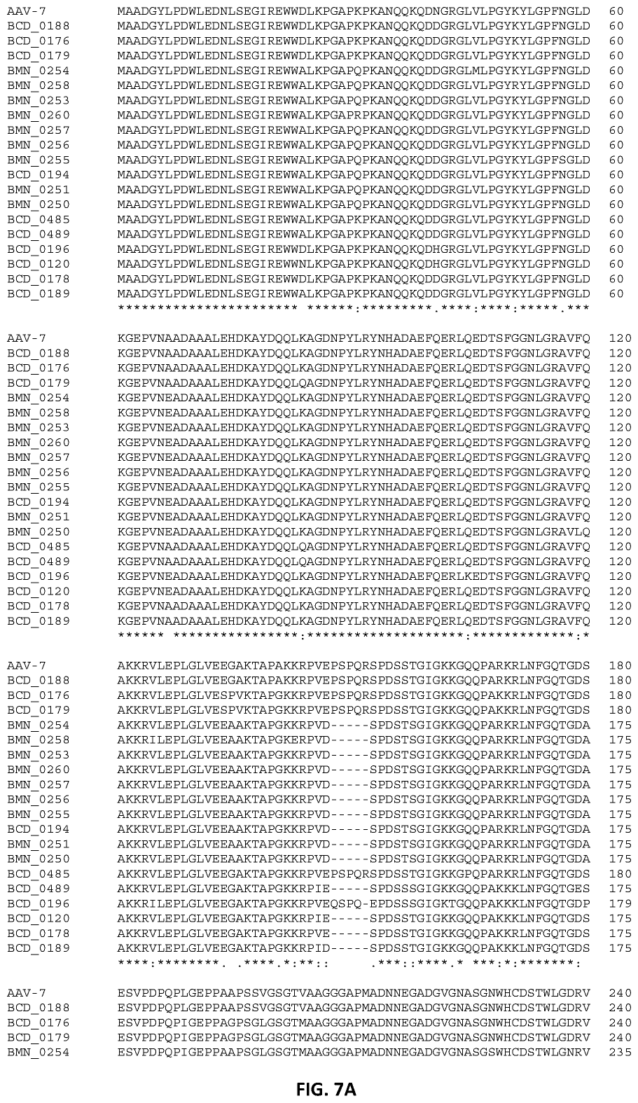

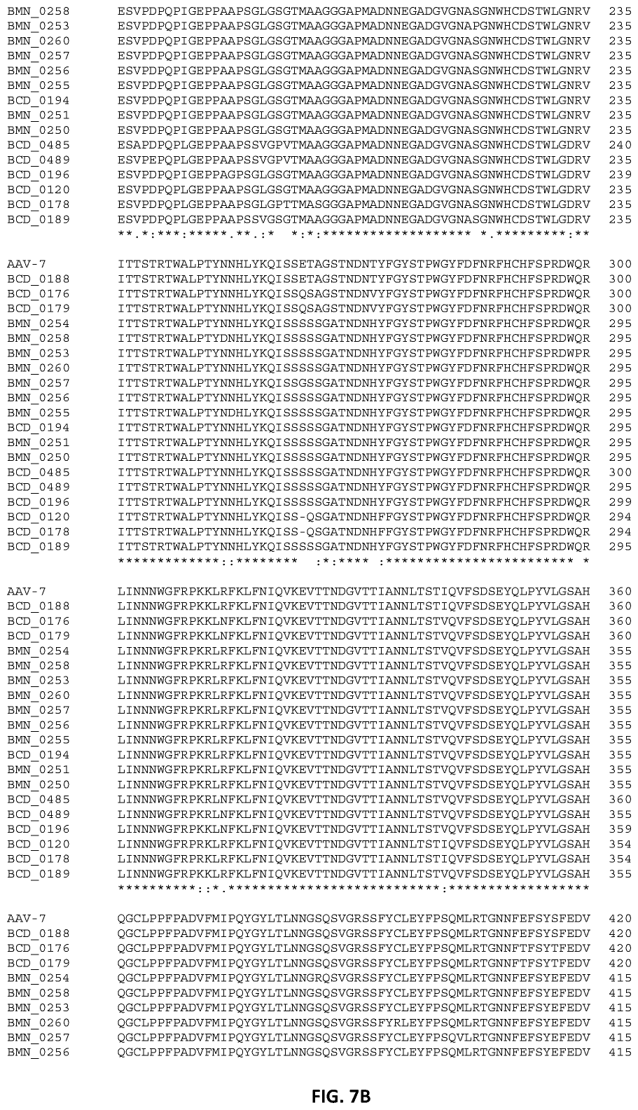

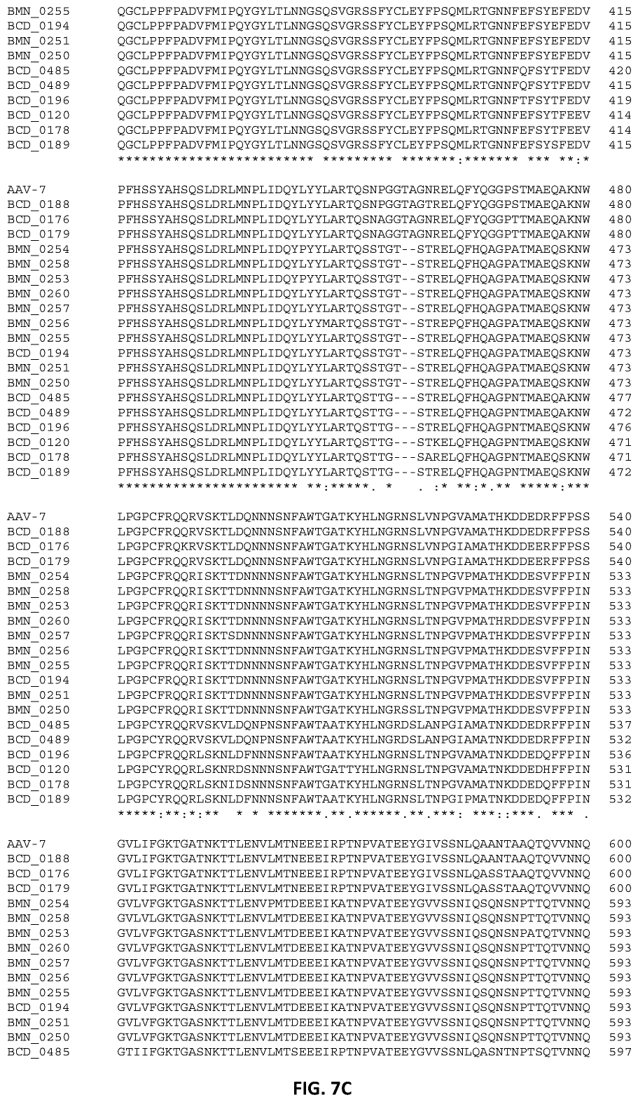

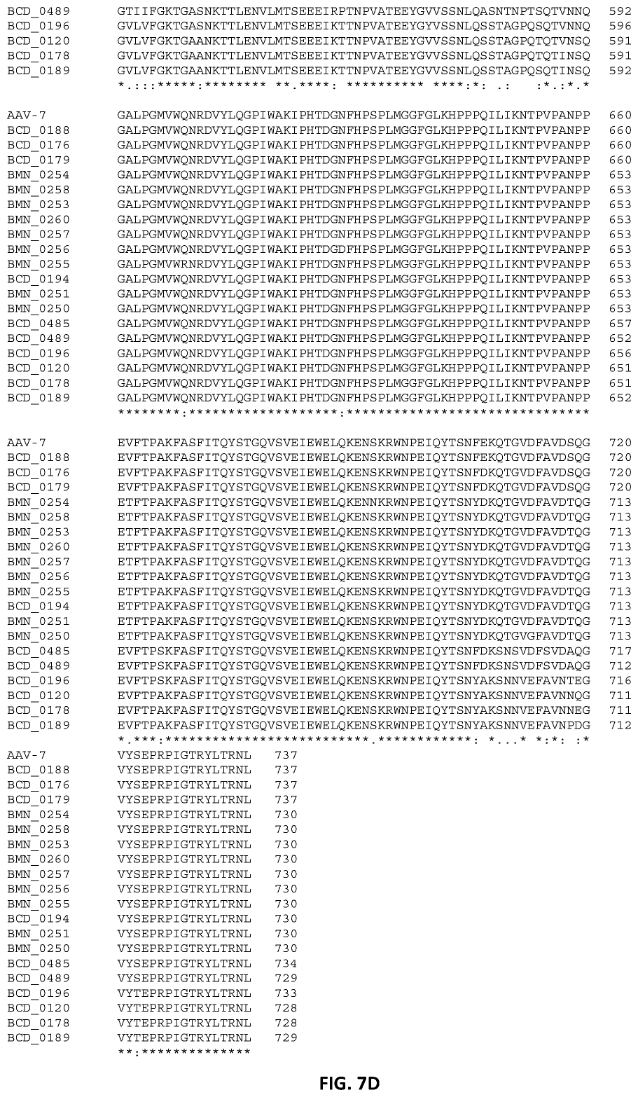

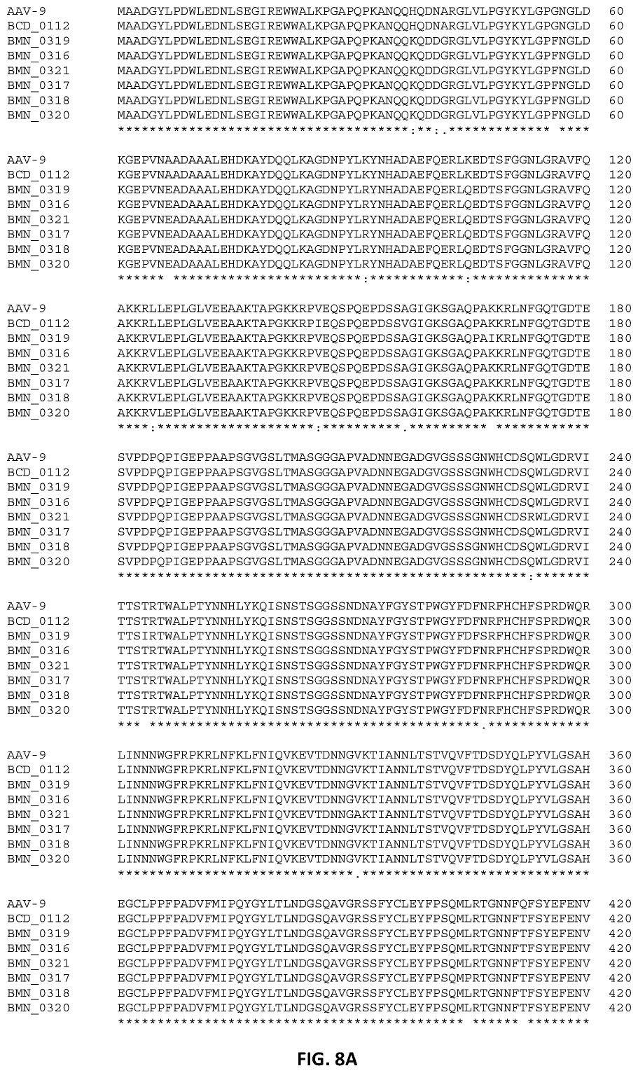

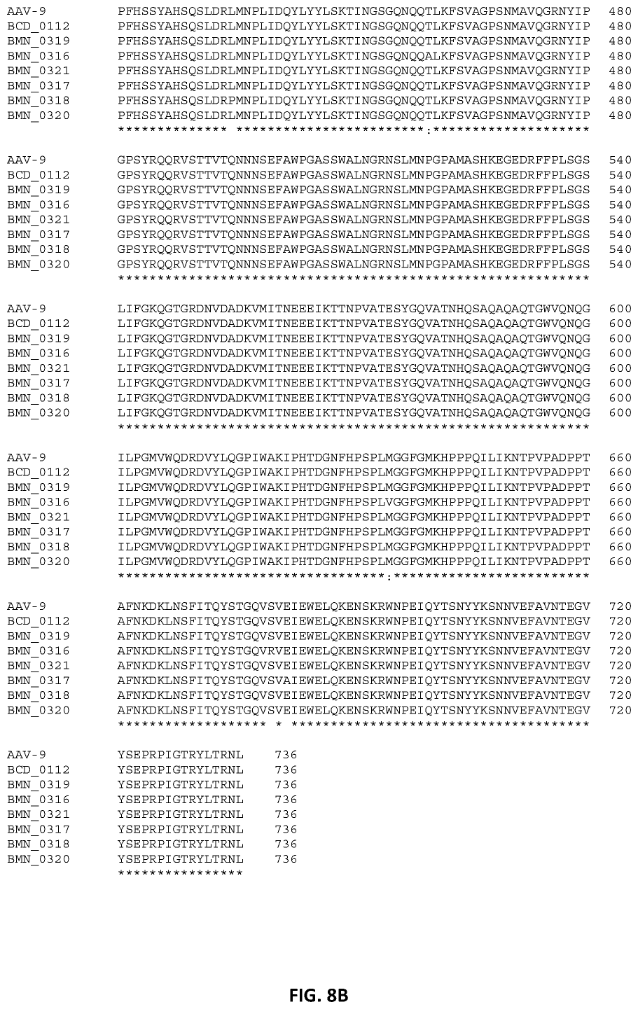

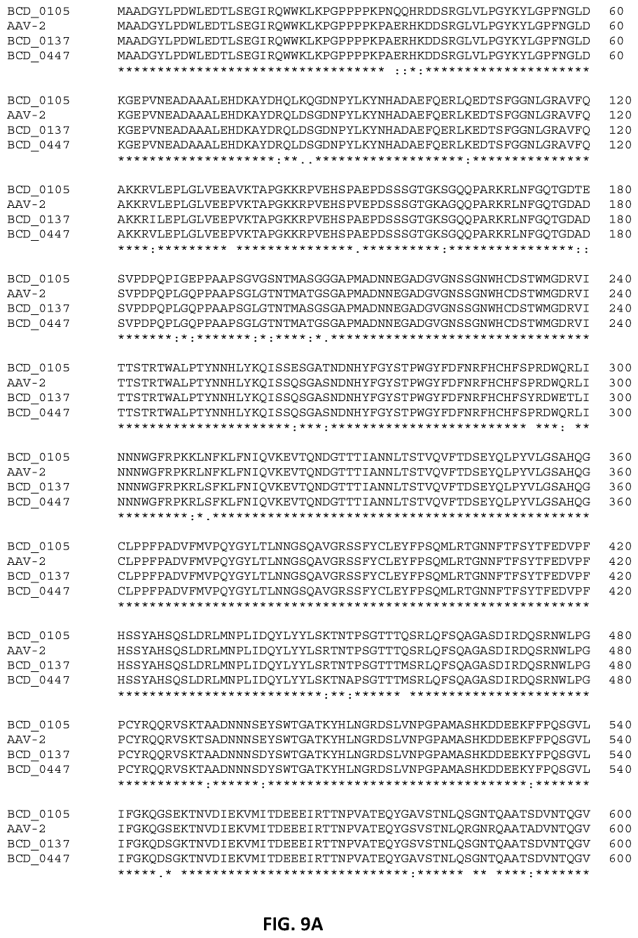

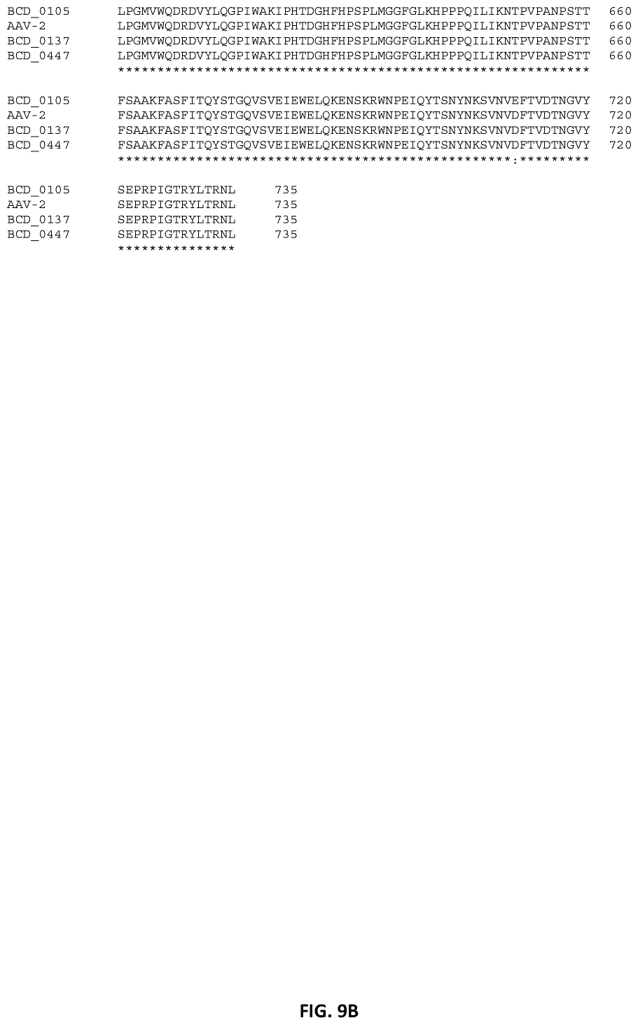

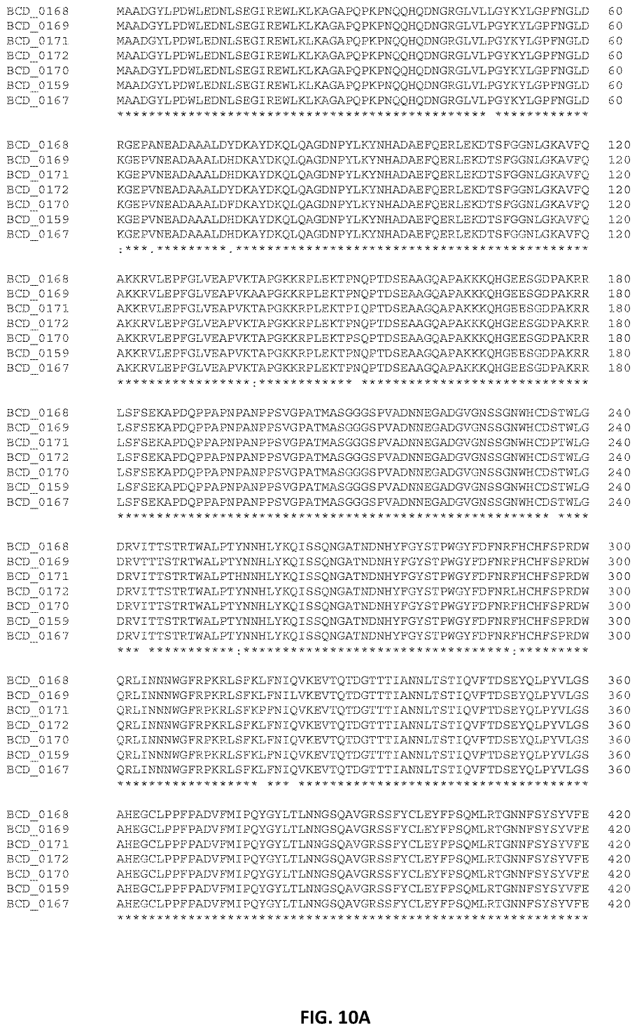

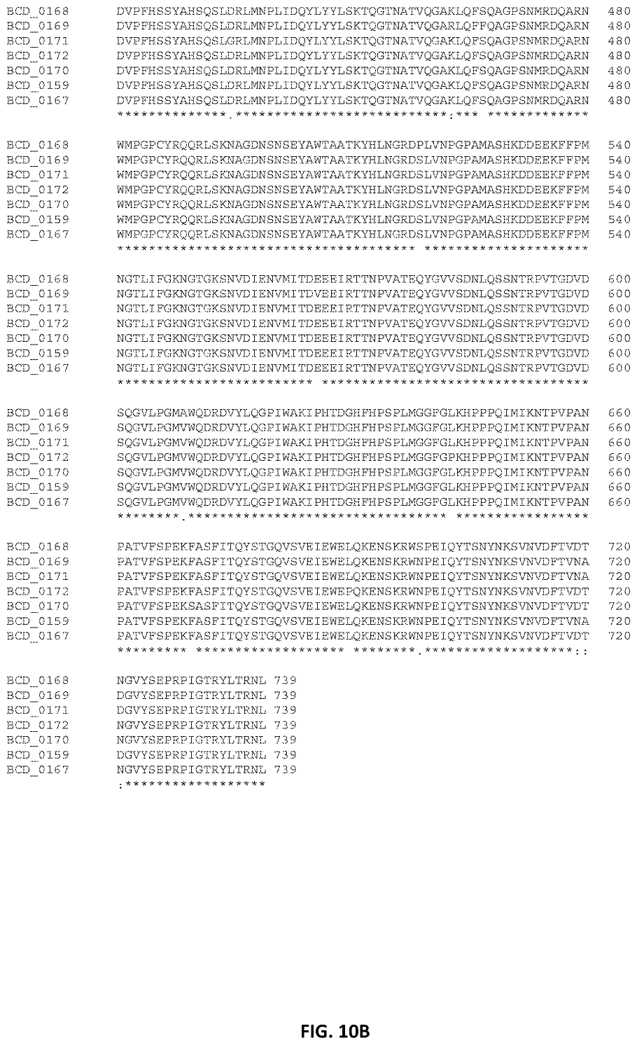

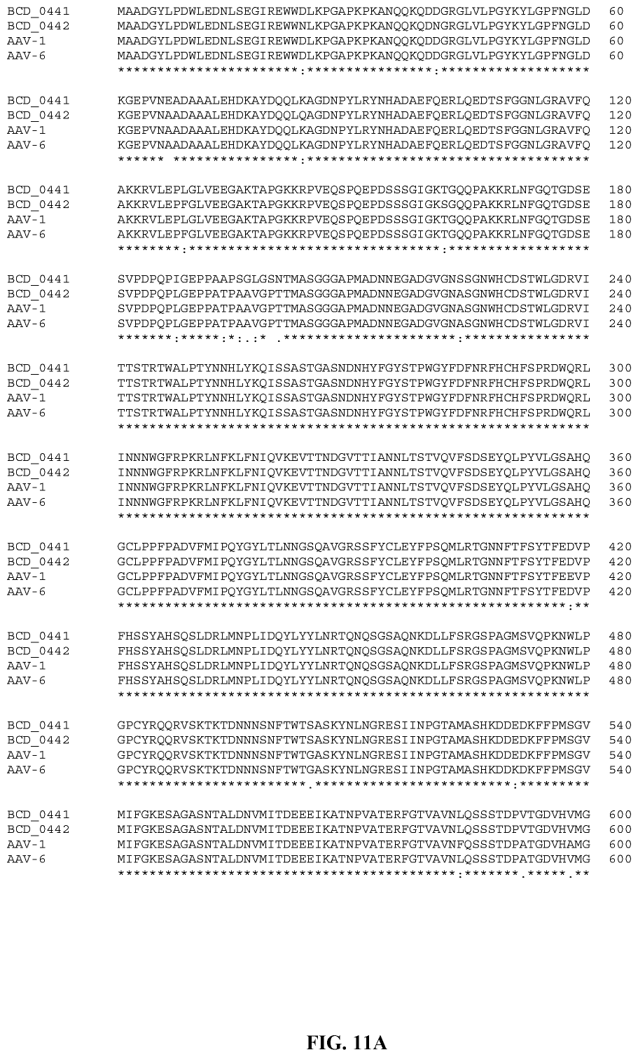

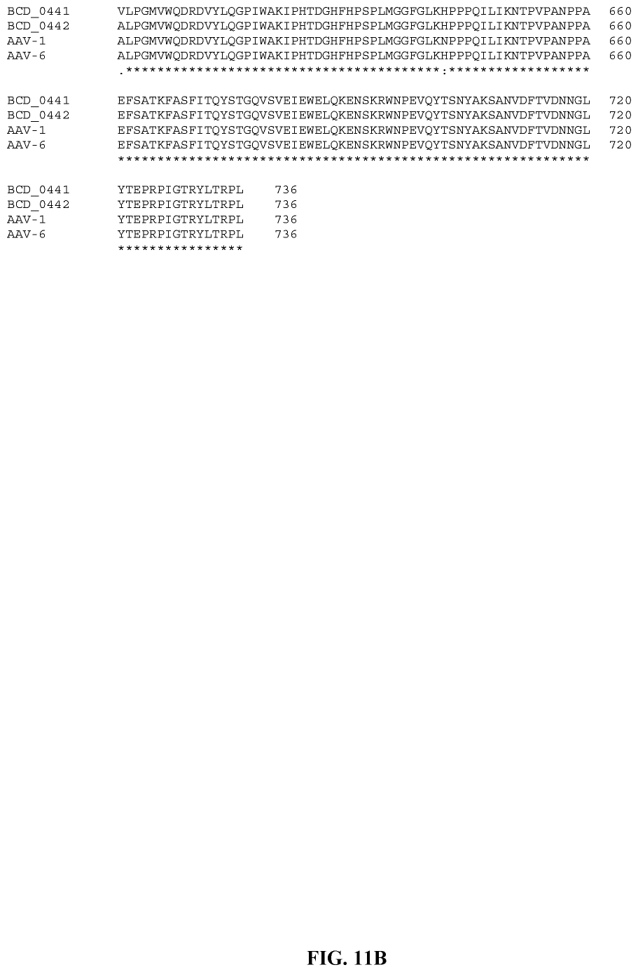

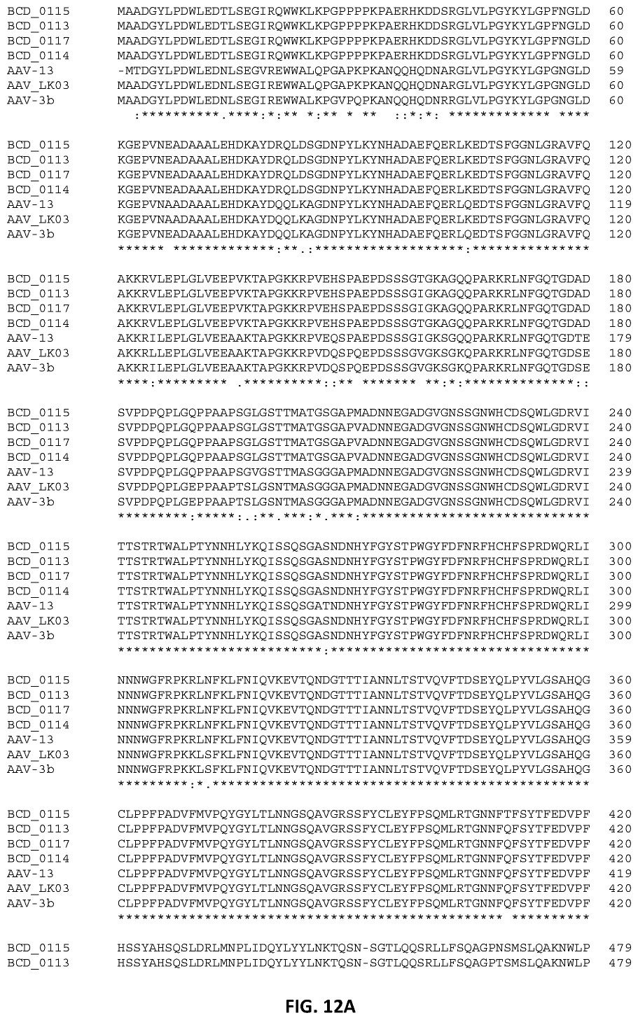

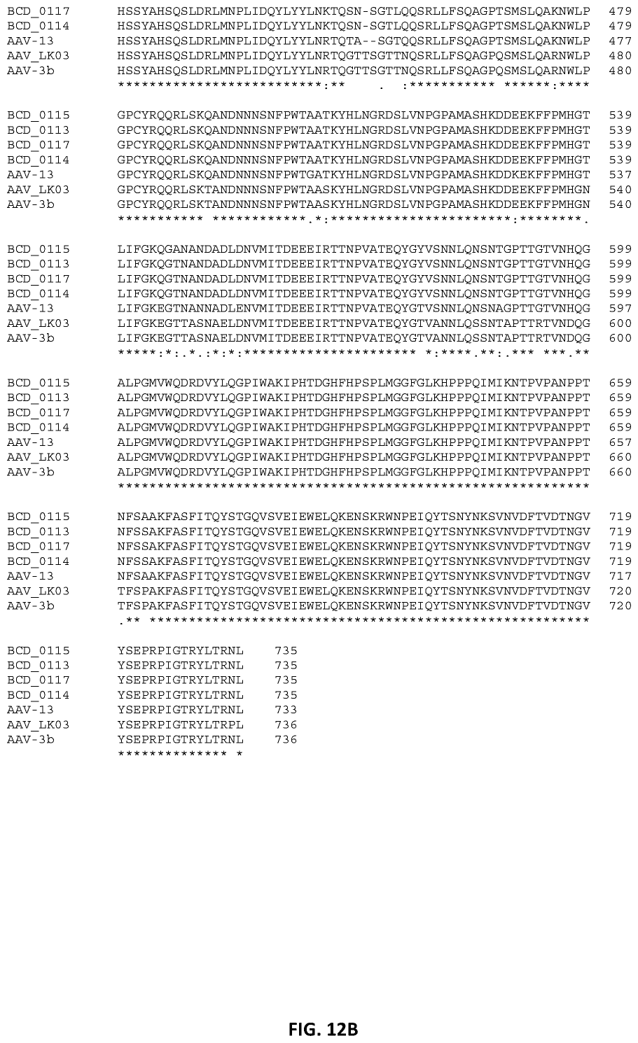

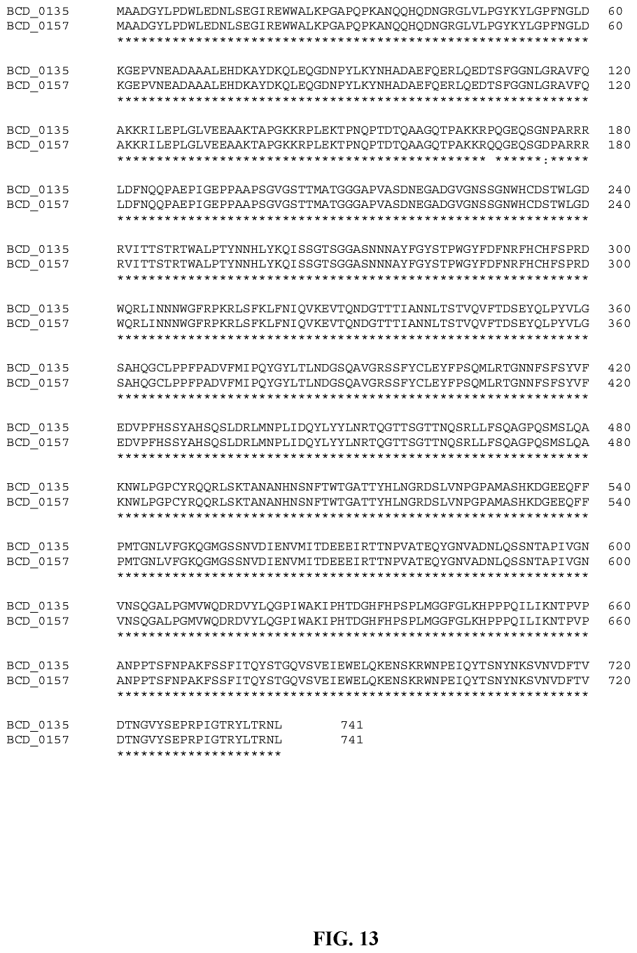

One of skill in the art can readily determine the location of the VP2 and VP3 regions, variable regions, and constant regions by using, for example, the provided VP1 sequences of the novel AAV capsid proteins and comparing them to the VP1 regions of closely related AAVs. For example, the location of the VP2 and VP3 regions of a novel AAV capsid sequence may be determined by comparing the VP1 region(s) of the novel AAV capsid sequence to the VP2 and VP3 regions of an AAV with a VP1 region closely related to the VP1 of the novel AAV capsid sequence (e.g., known VP2 and VP3 regions). See, Example 9 and FIGS. 4-13.

### 6.3.1.1 Novel AAV Capsid Amino Acid Sequences

The novel AAV capsid VP1 amino acid sequences presented herein are described in Table 9 identified with a “BCD_” prefix and SEQ ID NOs: 12-127 and 361-362. See Example 1, infra, for a discussion of the identification of the novel AAV capsid VP1 sequences. As disclosed herein, one can determine the location of the novel VP2 and VP3 proteins of the disclosure, when present, by comparing the VP2 and VP3 regions with an AAV that is closely related to the novel capsid VP1 region.

In some embodiments, provided herein are amino acid sequences of a VP1 region of a capsid protein described in the Examples, infra. In one embodiment, provided herein is the VP1 amino acid sequence of BCD_0126. In another embodiment, provided herein is the VP1 amino acid sequence of BCD_0282. In another embodiment, provided herein is the VP1 amino acid sequence of BCD_0176. In another embodiment, provided herein is the VP1 amino acid sequence of BCD_0446. In another embodiment, provided herein is the VP1 amino acid sequence of BCD_0160. In another embodiment, provided herein is the VP1 amino acid sequence of BCD_0195. In another embodiment, provided herein is the VP1 amino acid sequence of BCD_0180. In another embodiment, provided herein is the VP1 amino acid sequence of BCD_0192. In another embodiment, provided herein is the VP1 amino acid sequence of BCD_0185. In another embodiment, provided is the VP1 amino acid sequence of BCD_0454. In another embodiment, provided herein is the VP1 amino acid sequence of BCD_0277. In another embodiment, provided herein is the VP1 amino acid sequence of BCD_0174. In another embodiment, provided herein is the VP1 amino acid sequence of BCD_0167. In another embodiment, provided herein is the VP1 amino acid sequence of BCD_0126. In another embodiment, provided herein is the VP1 amino acid sequence of BCD_0125. In another embodiment, provided herein is the VP1 amino acid sequence of BCD_0193. In another embodiment, provided herein is the VP1 amino acid sequence of BCD_0286. In another embodiment, provided herein is the VP1 amino acid sequence of BCD_0182. In another embodiment, provided herein is the VP1 amino acid sequence of BCD_0283. In another embodiment, provided herein is the VP1 amino acid sequence of BCD_0106. In another embodiment, provided herein is the VP1 amino acid sequence of BCD_0361.

The disclosure also provides for functional fragments of the novel AAV capsid amino acid sequences. Examples of functional fragments of the novel AAV capsid amino acid sequences of the disclosure include, for example, the constant region and the variable region sequences (i.e., VR, GBS, and/or GH Loop) of the VP1, VP2, and/or VP3 proteins as provided in the Examples (e.g., Example 9).

The amino acid sequences, proteins, peptides, and fragments of the disclosure can be produced by any suitable means, including recombinant production, chemical synthesis production, synthetic production, or any other means known in the art.

### 6.3.1.2 Novel AAV Capsid Nucleic Acid Sequences

The novel AAV VP1 nucleic acid sequences presented herein are described in Table 9 identified with a “BCD_” prefix an SEQ ID NOs: 192-307 and 363-364. See Example 1, infra, for a discussion of the identification of the novel AAV capsid VP1 sequences. The AAV capsid nucleic acid sequences of the disclosure encompass the strand which is the complementary nucleic acid sequence, as well as the RNA and cDNA sequences corresponding to sequences, and its complementary strand. Due the degeneracy of codons, multiple codons may encode for the same amino acid. Accordingly, provided herein are nucleic acid sequences encoding each of the novel AAV VP1 amino acid sequences in SEQ ID NOs: 12-127 and 361-362. In certain embodiments, a nucleic acid sequence encoding a novel AAV VP1 amino acid sequence presented herein is codon optimized for the intended host cell, e.g., codon optimized for human cells.

In some embodiments, provided herein are nucleic acid sequences encoding a VP1 region of a capsid protein described in the Examples, infra. In one embodiment, provided herein are nucleic acid sequences encoding the VP1 amino acid sequence of BCD_0126. In another embodiment, provided herein are nucleic acid sequences encoding the VP1 amino acid sequence of BCD_0282. In another embodiment, provided herein are nucleic acid sequences encoding the VP1 amino acid sequence of BCD_0176. In another embodiment, provided herein are nucleic acid sequences encoding the VP1 amino acid sequence of BCD_0446. In another embodiment, provided herein are nucleic acid sequences encoding the VP1 amino acid sequence of BCD_0160. In another embodiment, provided herein are nucleic acid sequences encoding the VP1 amino acid sequence of BCD_0195. In another embodiment, provided herein are nucleic acid sequences encoding the VP1 amino acid sequence of BCD_0180. In another embodiment, provided herein are nucleic acid sequences encoding the VP1 amino acid sequence of BCD_0192. In another embodiment, provided herein are nucleic acid sequences encoding the VP1 amino acid sequence of BCD_0185. In another embodiment, provided herein are nucleic acid sequences encoding the VP1 amino acid sequence of BCD_0454. In another embodiment, provided herein are nucleic acid sequences encoding the VP1 amino acid sequence of BCD_0277. In another embodiment, provided herein are nucleic acid sequences encoding the VP1 amino acid sequence of BCD_0174. In another embodiment, provided herein are nucleic acid sequences encoding the VP1 amino acid sequence of BCD_0167. In another embodiment, provided herein are nucleic acid sequences encoding the VP1 amino acid sequence of BCD_0126. In another embodiment, provided herein are nucleic acid sequences encoding the VP1 amino acid sequence of BCD_0125. In another embodiment, provided herein are nucleic acid sequences encoding the VP1 amino acid sequence of BCD_0193. In another embodiment, provided herein are nucleic acid sequences encoding the VP1 amino acid sequence of BCD_0286. In another embodiment, provided herein are nucleic acid sequences encoding the VP1 amino acid sequence of BCD_0182. In another embodiment, provided herein are nucleic acid sequences encoding the VP1 amino acid sequence of BCD_0283. In another embodiment, provided herein are nucleic acid sequences encoding the VP1 amino acid sequence of BCD_0106. In another embodiment, provided herein are nucleic acid sequences encoding the VP1 amino acid sequence of BCD_0361.

As disclosed herein, one can readily determine the location of the VP2 and VP3 nucleic acid sequences encoding the proteins, if present, by comparing the VP2 and VP3 regions with an AAV that has substantial identity or similarity to the novel capsid VP1 region. Comparison can be conducted as provided herein. Accordingly, provided herein are nucleic acid sequences encoding the VP2 region of the novel AAV VP1 amino acids sequences in SEQ ID NOs: 12-127 and 361-362. Also provided herein are nucleic acid sequences encoding the VP3 region of the novel AAV VP1 amino acid sequences in SEQ ID NOs: 12-127 and 361-362.

In some embodiments, provided herein are nucleic acid sequences encoding a VP2 region of a capsid protein described in the Examples, infra. In one embodiment, provided herein are nucleic acid sequences encoding the VP2 region of the VP1 amino acid sequence of BCD_0126. In another embodiment, provided herein are nucleic acid sequences encoding the VP2 region of the VP1 amino acid sequence of BCD_0282. In another embodiment, provided herein are nucleic acid sequences encoding the VP2 region of the VP1 amino acid sequence of BCD_0176. In another embodiment, provided herein are nucleic acid sequences encoding the VP2 region of the VP1 amino acid sequence of BCD_0446. In another embodiment, provided herein are nucleic acid sequences encoding the VP2 region of the VP1 amino acid sequence of BCD_0160. In another embodiment, provided herein are nucleic acid sequences encoding the VP2 region of the VP1 amino acid sequence of BCD_0195. In another embodiment, provided herein are nucleic acid sequences encoding the VP2 region of the VP1 amino acid sequence of BCD_0180. In another embodiment, provided herein are nucleic acid sequences encoding the VP2 region of the VP1 amino acid sequence of BCD_0192. In another embodiment, provided herein are nucleic acid sequences encoding the VP2 region of the VP1 amino acid sequence of BCD_0185. In another embodiment, provided herein are nucleic acid sequences encoding the VP2 region of the VP1 amino acid sequence of BCD_0454. In another embodiment, provided herein are nucleic acid sequences encoding the VP2 region of the VP1 amino acid sequence of BCD_0277. In another embodiment, provided herein are nucleic acid sequences encoding the VP2 region of the VP1 amino acid sequence of BCD_0174. In another embodiment, provided herein are nucleic acid sequences encoding the VP2 region of the VP1 amino acid sequence of BCD_0167. In another embodiment, provided herein are nucleic acid sequences encoding the VP2 region of the VP1 amino acid sequence of BCD_0126. In another embodiment, provided herein are nucleic acid sequences encoding the VP2 region of the VP1 amino acid sequence of BCD_0125. In another embodiment, provided herein are nucleic acid sequences encoding the VP2 region of the VP1 amino acid sequence of BCD_0193. In another embodiment, provided herein are nucleic acid sequences encoding the VP2 region of the VP1 amino acid sequence of BCD_0286. In another embodiment, provided herein are nucleic acid sequences encoding the VP2 region of the VP1 amino acid sequence of BCD_0182. In another embodiment, provided herein are nucleic acid sequences encoding the VP2 region of the VP1 amino acid sequence of BCD_0283. In another embodiment, provided herein are nucleic acid sequences encoding the VP2 region of the VP1 amino acid sequence of BCD_0106. In another embodiment, provided herein are nucleic acid sequences encoding the VP2 region of the VP1 amino acid sequence of BCD_0361.

In some embodiments, provided herein are nucleic acid sequences encoding a VP3 region of a capsid protein described in the Examples, infra. In one embodiment, provided herein are nucleic acid sequences encoding the VP3 region of the VP1 amino acid sequence of BCD_0126. In another embodiment, provided herein are nucleic acid sequences encoding the VP3 region of the VP1 amino acid sequence of BCD_0282. In another embodiment, provided herein are nucleic acid sequences encoding the VP3 region of the VP1 amino acid sequence of BCD_0176. In another embodiment, provided herein are nucleic acid sequences encoding the VP3 region of the VP1 amino acid sequence of BCD_0446. In another embodiment, provided herein are nucleic acid sequences encoding the VP3 region of the VP1 amino acid sequence of BCD_0160. In another embodiment, provided herein are nucleic acid sequences encoding the VP3 region of the VP1 amino acid sequence of BCD_0195. In another embodiment, provided herein are nucleic acid sequences encoding the VP3 region of the VP1 amino acid sequence of BCD_0180. In another embodiment, provided herein are nucleic acid sequences encoding the VP3 region of the VP1 amino acid sequence of BCD_0192. In another embodiment, provided herein are nucleic acid sequences encoding the VP3 region of the VP1 amino acid sequence of BCD_0185. In another embodiment, provided herein are nucleic acid sequences encoding the VP3 region of the VP1 amino acid sequence of BCD_0454. In another embodiment, provided herein are nucleic acid sequences encoding the VP3 region of the VP1 amino acid sequence of BCD_0277. In another embodiment, provided herein are nucleic acid sequences encoding the VP3 region of the VP1 amino acid sequence of BCD_0174. In another embodiment, provided herein are nucleic acid sequences encoding the VP3 region of the VP1 amino acid sequence of BCD_0167. In another embodiment, provided herein are nucleic acid sequences encoding the VP3 region of the VP1 amino acid sequence of BCD_0126. In another embodiment, provided herein are nucleic acid sequences encoding the VP3 region of the VP1 amino acid sequence of BCD_0125. In another embodiment, provided herein are nucleic acid sequences encoding the VP3 region of the VP1 amino acid sequence of BCD_0193. In another embodiment, provided herein are nucleic acid sequences encoding the VP3 region of the VP1 amino acid sequence of BCD_0286. In another embodiment, provided herein are nucleic acid sequences encoding the VP3 region of the VP1 amino acid sequence of BCD_0182. In another embodiment, provided herein are nucleic acid sequences encoding the VP3 region of the VP1 amino acid sequence of BCD_0283. In another embodiment, provided herein are nucleic acid sequences encoding the VP3 region of the VP1 amino acid sequence of BCD_0106. In another embodiment, provided herein are nucleic acid sequences encoding the VP3 region of the VP1 amino acid sequence of BCD_0361.

Included within the scope of the AAV capsid nucleic acid sequences of the disclosure are nucleic acid sequences that hybridize under stringent conditions to the nucleotide sequences encoding the novel AAV capsids. In some embodiments, provided herein is a nucleic acid sequence(s) that hybridizes under stringent conditions across the entire length of a nucleotide sequence encoding a novel AAV capsid. In some embodiments, provided herein is a nucleic acid sequence that hybridizes under stringent conditions across the entire length of a nucleotide sequence encoding a VP1 capsid protein set forth in Table 9, which has a “BCD_” prefix. In a specific embodiment, provided herein is a nucleic acid sequence that hybridizes under stringent conditions across the entire length of any one of the nucleotide sequences set forth in SEQ ID NOs: 192-307 and 363-364. In some embodiments, provided herein is a nucleic acid sequence that hybridizes under stringent conditions across the entire length of a nucleotide sequence encoding a capsid protein described in the Examples. In specific embodiments, provided herein a nucleic acid sequence that hybridizes under stringent conditions across the entire length of the nucleotide sequence provided in Table 9, infra, which encodes a capsid protein of BCD_0126, BCD_0282, BCD_0176, BCD_0446, BCD_0160, BCD_0195, BCD_0180, BCD_0192, BCD_0185, BCD_0454, BCD_0277, BCD_0174, BCD_0167, BCD_0126, BCD_0125, BCD_0193, BCD_0286, BCD_0182, BCD_0283, BCD_0106, or BCD_0361. In other embodiments, provided herein is a nucleic acid sequence(s) that hybridizes under stringent conditions across a fragment of a nucleotide sequence encoding a novel AAV capsid. In some embodiments, provided herein is a nucleic acid sequence that hybridizes under stringent conditions across a fragment of a nucleotide sequence encoding a VP1 capsid protein set forth in Table 9, which has a “BCD_” prefix. In a specific embodiment, provided herein is a nucleic acid sequence that hybridizes under stringent conditions across a fragment of any one of the nucleotide sequences set forth in SEQ ID NOs: 192-307 and 363-364. In some embodiments, provided herein is a nucleic acid sequence that hybridizes under stringent conditions across a fragment of a nucleotide sequence encoding a capsid protein described in the Examples. In specific embodiments, provided herein a nucleic acid sequence that hybridizes under stringent conditions across a fragment of the nucleotide sequence provided in Table 9, infra, which encodes the VP1 amino acid sequence of BCD_0126, BCD_0282, BCD_0176, BCD_0446, BCD_0160, BCD_0195, BCD_0180, BCD_0192, BCD_0185, BCD_0454, BCD_0277, BCD_0174, BCD_0167, BCD_0126, BCD_0125, BCD_0193, BCD_0286, BCD_0182, BCD_0283, BCD_0106, or BCD_0361. Fragments of, e.g., 15, 16, 17, 18, 19 or 20 nucleotides or more that are selective for (i.e., specifically hybridize to any one of the polynucleotides of the invention) are contemplated. In certain embodiments, fragments are at least 9 nucleotides, at least 12 nucleotides, at least 15 nucleotides, at least 20 nucleotides, at least 25 nucleotides, at least 50 nucleotides, at least 75 nucleotides or at least 100 nucleotides in length. In some embodiments, fragments are 15 to 30 nucleotides, 25 to 50 nucleotides, 25 to 75 nucleotides, 50 to 75 nucleotides, 50 to 100 nucleotides, or 75 to 100 nucleotides in length. In certain embodiments, fragments are 100 to 125 nucleotides, 100 to 150 nucleotides, 125 to 150 nucleotides, 150 to 175 nucleotides, 150 to 200 nucleotides, or 175 to 200 nucleotides in length. In some embodiments, the fragment comprises a nucleotide sequence encoding a variable region(s) of an AAV capsid.

Included within the scope of the AAV capsid nucleic acid sequences of the disclosure are nucleic acid sequence fragments that hybridize under stringent conditions to the nucleotide sequences encoding the novel AAV capsid, which fragment is greater than about 9 nucleotides, is greater than about 12 nucleotides, greater than about 15 nucleotides, greater than about 27 nucleotides, greater than about 39 nucleotides, greater than about 51, or greater than about 462 nucleotides. Fragments of, e.g., 9, 12, 15, 27, 39, 51, or 462 nucleotides or more that are selective for (i.e., specifically hybridize to any one of the polynucleotides of the disclosure) are contemplated. Probes capable of hybridizing to a polynucleotide under stringent conditions can differentiate polynucleotide sequences of the disclosure from other polynucleotide sequences.

The term “stringent” in the context of hybridization is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015M sodium citrate at 65-68° C. or 0.015 M sodium chloride, 0.0015M sodium citrate, and 50% formamide at 42° C. See Green, M. and Sambrook, J., Molecular Cloning: A Laboratory Manual, 4th Ed., Cold Spring Harbor Laboratory (Cold Spring Harbor, N.Y. 2012).

Where more stringent conditions (such as higher temperature, lower ionic strength, higher formamide, or other denaturing agent) are used, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20- base oligos), and 60° C. (for 23-base oligos).

Other agents may be included in the hybridization and washing buffers for the purpose of reducing non-specific and/or background hybridization. Examples of other agents are 0.1% bovine serum albumin, 0.1% polyvinyl-pyrrolidone, 0.1% sodium pyrophosphate, 0.1% sodium dodecylsulfate, NaDodS04 (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art to accommodate these variables and allow DNAs of different sequence relatedness to form hybrids.

The AAV capsid nucleic acid sequences may be produced by any suitable means, including recombinant production, chemical synthesis production, synthetic production, or any other means known in the art.

### 6.3.1.3 Functional Fragments of Novel AAV Capsid Protein

The disclosure also provides functional fragments of the novel AAV VP1 capsid sequences disclosed herein.

The functional fragments of the novel AAV capsid VP1 sequences include the VP2, VP3, constant region(s), variable region(s), GBS domain, GH loop, or a combination thereof. In some embodiments the functional fragments provided by the disclosure have one or more conservative amino acid substitutions. See for example, Table 1. Some non-limiting examples of conservative amino acid substitutions in fragments of AAV capsid VP1 sequences are provided in Table 2 and FIGS. 4-13. References to Table 2 refers to subtables 2.6 to 2.15.

The variable region functional fragments of the novel VP1 amino acid sequences can be determined using the information provided herein. See, Example 9. Such functional fragments of the capsid VP1 protein may be used alone, in combination with other AAV sequences or fragments, e.g., AAV sequences or fragments from other novel AAV capsid sequences described herein, or in combination with elements from other AAVs (e.g., a reference AAV) or other viral sequences (e.g., non-AAV sequences such as a delivery vehicle).

The inclusion of one or more functional fragments of the disclosure in an AAV capsid may result in an AAV viral particle or delivery vehicle (e.g., nanoparticle, such as a lipid nanoparticle) with one, two, three or more, or more, or all of the of following: enhanced packaging yield, enhanced transduction efficiency, enhanced gene transfer efficiency, enhanced translation efficiency, enhanced tissue-specificity (i.e., tropism), and/or the enhanced ability to evade immunity compared to a non-modified AAV viral particle or naturally occurring sequence (e.g., a reference sequence, such as in Table 4, infra). The enhanced activities of the AAV particle or nanoparticle may be assessed in an in vitro or an in vivo assay. The in vitro or in vivo assay may be one described herein (e.g., in the Examples) or others known in the art to assess the yield, transduction efficiency, gene transfer efficiency, translation efficiency, tissue-specificity (i.e., tropism), and/or the ability to evade immunity of an AAV viral particle or delivery vehicle.

### 6.3.1.4 Modified Novel AAV Capsid

The disclosure also provides modifications of the novel AAV capsid sequences. In a specific embodiment, a modified novel AAV capsid sequence (i.e., VP1, VP2, or VP3) of the disclosure comprises a novel AAV capsid sequence (e.g., any one of SEQ ID NOs: 12-127 and 361-362, or SEQ ID NOs: 192-307 and 363-364) with at least one nucleic acid or amino acid residue mutation (change, e.g., substitution, insertion, and/or deletion, relative to the novel AAV capsid sequence) with no more than about 10% of the total sequence the novel AAV capsid sequence (e.g., one of SEQ ID NOs: 12-127 and 361-362, or SEQ ID NOs: 192-307 and 363-364) changed. In some embodiments, about 1% to about 4% of the total sequence of the novel AAV capsid sequence is changed. In some embodiments, about 4%-6% of the total sequence of the novel AAV capsid sequence is changed. In some other embodiments, about 6% to about 8% of the total sequence of the novel AAV capsid sequence is changed. Yet, in some other embodiments, about 8% to about 10% of the total sequence of the novel AAV capsid sequence is changed. Non-limiting substitution, insertion, and/or deletions modifications that can be made to a novel AAV capsid sequence are provided in Table 2 and FIGS. 4-13. Regarding FIGS. 4-13 the colon mark (:) indicates amino acid residue substitution with strongly similar properties, the period mark (.) indicates amino acid residue substitution with weakly similar properties, and the asterisk mark (*) indicates fully conserved amino acid residue substitution as determined by ClustalΩ (alternatively known as “Clustal O” or “Clustal Omega”).

In certain embodiments, a modified novel AAV capsid protein includes an AAV capsid protein of an AAV clade member listed in any one of Tables 2.6 to 2.15 with one or more of the amino acid residue variations (e.g., amino acid residue substitutions) listed in Tables 2.6 to 2.16, respectively. In specific embodiments, a modified novel AAV capsid protein comprises an amino acid sequence of an AAV capsid of any one of Nos. 0-50 in Table 2.6 with one or more mutations, such as one or more amino acid substitutions (e.g., 1, 2, 3, 4, 5, 6, 7, 8, 9 or more), recited in Table 2.6, provided that the mutation(s) (e.g., amino acid substitution(s)) does not result in a known AAV capsid (e.g., Nos. 38-50 in Table 2.6). In specific embodiments, a modified novel AAV capsid protein comprises an amino acid sequence of an AAV capsid of any one of Nos. 0-48 in Table 2.7 with one or more mutations, such as one or more amino acid substitutions (e.g., 1, 2, 3, 4, 5, 6, 7, 8, 9 or more), recited in Table 2.7, provided that the mutation(s) (e.g., amino acid substitution(s)) does not result in a known AAV capsid (e.g., Nos. 0, 1, and 23-48 in Table 2.7). In specific embodiments, a modified novel AAV capsid protein comprises an amino acid sequence of an AAV capsid of any one of Nos. 0-29 in Table 2.8 with one or more mutations, such as one or more amino acid substitutions (e.g., 1, 2, 3, 4, 5, 6, 7, 8, 9 or more), recited in Table 2.8, provided that the mutation(s) (e.g., amino acid substitution(s)) does not result in a known AAV capsid (e.g., No. 1 in Table 2.8). In specific embodiments, a modified novel AAV capsid protein comprises an amino acid sequence of an AAV capsid of any one of Nos. 0-19 in Table 2.9 with one or more mutations, such as one or more amino acid substitutions (e.g., 1, 2, 3, 4, 5, 6, 7, 8, 9 or more), recited in Table 2.9, provided that the mutation(s) (e.g., amino acid substitution(s)) does not result in a known AAV capsid (e.g., No. 0 and 11-19 in Table 2.9).

In specific embodiments, a modified novel AAV capsid protein comprises an amino acid sequence of an AAV capsid of any one of Nos. 0-7 in Table 2.10 with one or more mutations, such as one or more amino acid substitutions (e.g., 1, 2, 3, 4, 5, 6, 7, 8, 9 or more), recited in Table 2.10, provided that the mutation(s) (e.g., amino acid substitution(s)) does not result in a known AAV capsid (e.g., No. 0 and 2-7 in Table 2.10). In specific embodiments, a modified novel AAV capsid protein comprises an amino acid sequence of an AAV capsid of any one of Nos. 0-3 in Table 2.11 with one or more mutation(s), such as one or more amino acid substitutions (e.g., 1, 2, 3, 4, 5, 6, 7, 8, 9 or more), recited in Table 2.11, provided that the mutation(s) (e.g., amino acid substitution(s)) does not result in a known AAV capsid (e.g., No. 0 in Table 2.11). In specific embodiments, a modified novel AAV capsid protein comprises an amino acid sequence of an AAV capsid of any one of Nos. 0-6 in Table 2.12 with one or more mutations, such as one or more amino acid substitutions (e.g., 1, 2, 3, 4, 5, 6, 7, 8, 9 or more), recited in Table 2.12, provided that the mutation(s) (e.g., amino acid substitution(s)) does not result in a known AAV capsid. In specific embodiments, a modified novel AAV capsid protein comprises an amino acid sequence of an AAV capsid of any one of Nos. 0-3 in Table 2.13 with one or more mutations, such as one or more amino acid substitutions (e.g., 1, 2, 3, 4, 5, 6, 7, 8, 9 or more), recited in Table 2.13, provided that the mutation(s) (e.g., amino acid substitution(s)) does not result in a known AAV capsid (e.g., No. 0 or 1 in Table 2.13).

In specific embodiments, a modified novel AAV capsid protein comprises an amino acid sequence of an AAV capsid of any one of Nos. 0-6 in Table 2.14 with one or more mutations, such as one or more amino acid substitutions (e.g., 1, 2, 3, 4, 5, 6, 7, 8, 9 or more), recited in Table 2.14, provided that the mutation(s) (e.g., amino acid substitution(s)) does not result in a known AAV capsid (e.g., No. 0, 1, or 3 in Table 2.14). In specific embodiments, a modified novel AAV capsid protein comprises an amino acid sequence of an AAV capsid of any one of Nos. 0-1 in Table 2.15 with one or more mutation(s), such as one or more amino acid substitutions (e.g., 1, 2 or more), recited in Table 2.15, provided that the mutation(s) (e.g., amino acid substitution(s)) does not result in a known AAV capsid.

In another specific embodiment, a modified novel AAV capsid sequence of the disclosure comprises a novel AAV capsid sequence e.g., any one of SEQ ID NOs: 12-127 and 361-362, or SEQ ID NOs: 192-307 and 363-364) with at least one amino acid residue or nucleic acid substitution but with no more than about 10% of the total sequence of the novel AAV capsid sequence (e.g., any one of SEQ ID NOs: 12-127 and 361-362 or SEQ ID NOs: 192-307 and 363-364) changed. In some embodiments, about 1% to about 4% of the total sequence of the novel AAV capsid sequence is changed. In some embodiments, about 4% to about 6% of the total sequence of the novel AAV capsid sequence is changed. In some other embodiments, about 6% to about 8% of the total sequence of the novel AAV capsid sequence is changed. Yet, in some other embodiments, about 8% to about 10% of the total sequence of the novel AAV capsid sequence is changed. Some non-limiting amino acid modifications that can be made to a novel AAV capsid sequence are provided in Table 2 and FIGS. 4-13.

In another specific embodiment, a modified novel AAV capsid sequence of the disclosure comprises a novel AAV capsid amino acid sequence (e.g., any one of 12-127 and 361-362) with 1, 2, 3, 4, 5, 6, 7, 8, 9, or 10, or 1 to 10 amino acid residue substitutions in the novel AAV capsid amino acid sequence (e.g., any one of 12-127 and 361-362). In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acid residue substitutions in the novel AAV capsid amino acid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 32, 33, 34, 35, 36, 37, 38, 39, 40, 41, 42, 43, 44, 45, 46, 47, 48, 49 or 50 amino acid residue substitutions in the novel AAV capsid amino acid sequence (e.g., one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 amino acid residue substitutions in the novel AAV capsid amino acid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in the novel AAV capsid amino acid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19 or 20 conservative amino acid residue substitutions in the novel AAV capsid amino acid sequence (e.g., any one of SEQ ID NOs: 12-127 and 361-362).

In another specific embodiment, a modified novel AAV capsid amino acid sequence of the disclosure comprises an AAV capsid amino acid sequence (e.g., a VP1, VP2 or VP3 amino acid sequence) of a representative sequence in Table 2 with 1, 2, 3, 4, 5, 6, 7, 8, 9, 10, 11, 12, 13, 14 or 15 amino acid substitutions (e.g., conservative amino acid substitutions), provided that the modified novel AAV capsid amino acid sequence is not a known AAV capsid protein. In another specific embodiment, a modified novel AAV capsid amino acid sequence of the disclosure comprises an AAV capsid amino acid sequence (e.g., a VP1, VP2 or VP3 amino acid sequence) of a representative sequence in Table 2 with 16, 17, 18, 19, 20, 21, 22, 23, 24, 25, 26, 27, 28, 29, or 30 amino acid substitutions (e.g., conservative amino acid substitutions), provided that the modified novel AAV capsid amino acid sequence is not a known AAV capsid protein. In another specific embodiment, a modified novel AAV capsid amino acid sequence of the disclosure comprises an AAV capsid amino acid sequence (e.g., a VP1, VP2 or VP3 amino acid sequence) of a representative sequence in Table 2 with 31, 32, 33, 34, 35, 36, 37, 38, 39, 40 or more amino acid substitutions (e.g., conservative amino acid substitutions), provided that the modified novel AAV capsid amino acid sequence is not a known AAV capsid protein. In specific embodiments, the amino acid substitution(s) is one provided in Table 2.

In another specific embodiment, a modified novel AAV capsid amino acid sequence of the disclosure comprises an AAV VP1, VP2 or VP3 amino acid sequence that has at least 90% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 91% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 92% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 93% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 94% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 95% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 95% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 96% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 97% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 98% identical to an AAV VP1, VP2 or VP3 amino acid sequence of representative sequence in Table 2, provided that the modified novel AAV capsid amino acid sequence is not a known AAV capsid protein. In another specific embodiment, a modified novel AAV capsid amino acid sequence of the disclosure comprises an AAV VP1, VP2 or VP3 amino acid sequence that has at least 99% identical to an AAV VP1, VP2 or VP3 amino acid sequence of representative sequence in Table 2, provided that the modified novel AAV capsid amino acid sequence is not a known AAV capsid protein.

In another specific embodiment, a modified novel AAV capsid sequence of the disclosure comprises a fragment of a novel AAV capsid sequence (e.g., any one of SEQ ID NOs: 12-127 and 361-362, or SEQ ID NOs: 192-307 and 363-364) with at least one amino acid residue or nucleic acid mutation (e.g., substitution, insertion, and/or deletion) but with no more than about 10% of the total sequence of the fragment of the novel AAV sequence (e.g., the fragment of any one of SEQ ID NOs: 12-127 and 361-362, or SEQ ID NOs: 192-307 and 363-364) changed.

In another specific embodiment, a modified novel AAV capsid sequence of the disclosure comprises a fragment of a novel AAV capsid sequence (e.g., any one of SEQ ID NOs: 12-127 and 361-362, or SEQ ID NOs: 192-307 and 363-364) with at least one amino acid residue or nucleic acid substitution but with no more than about 10% of the total sequence the fragment of the novel AAV capsid sequence (e.g., the fragment of any one of SEQ ID NOs: 12-127 and 361-362, or SEQ ID NOs: 192-307 and 363-364) changed.

In another specific embodiment, a modified novel AAV capsid sequence of the disclosure comprises a fragment of a novel AAV capsid amino acid sequence (e.g., any one of SEQ ID NOs: 12-127 and 361-362, or SEQ ID NOs: 192-307 and 363-364) with 1, 2, 3, 4, 5, 6, 7, 8, 9 or 10 amino acid residue substitutions in the novel AAV capsid sequence (e.g., any one of SEQ ID NOs: 12-127 and 361-362 or SEQ ID NOs: 192-307 and 363-364) changed.

In another specific embodiment, a modified novel AAV capsid amino acid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in the novel AAV capsid amino acid sequence (e.g., any one of SEQ ID NOs: 12-127 and 361-362).

Alignment of the novel AAV VP1 amino acid sequence(s) provided herein with other VP1 amino acid sequences to various AAV references (see Table 4) can be used identify conserved and variable regions of the novel AAV VP1 capsid proteins. Using such an analysis, 1, 2, 3, 4, 5, 6, 7, 8, 9, 10, 11, 12 or more variable regions (including GBS and GH loop) in the capsid protein can be identified. In certain embodiments, presented herein are modified novel AAV capsid sequences comprising mutation(s) in one or more of a conversed and or variable region.

In some embodiments, there are 9 VRs in the capsid protein (denoted herein as VRI-VR IX). In certain embodiments, a modified novel AAV capsid sequence comprises a novel AAV capsid sequence with mutations (e.g., substitutions, insertions and/or deletions) 1, 2, 3, 4, 5, 6, 7, 8 or all 9 variable regions. In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions of the novel AAV amino acid capsid sequence (e.g., any one of SEQ ID NOs: 12-127 and 361-362). In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19, or 20 conservative amino acid residue substitutions in 1, 2, 3, 4, 5, 6, 7, 8 or all 9 variable regions of the novel AAV capsid amino acid sequence (e.g., any one of SEQ ID NOs: 12-127 and 361-362).

In another specific embodiment, a modified novel AAV capsid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions of the novel AAV capsid amino acid sequence (e.g., any one of SEQ ID NOs: 12-127 and 361-362).

In certain embodiments, a modified novel AAV capsid sequence comprises a novel AAV capsid sequence with mutations (e.g., substitutions, insertions and/or deletions) in the GBS, GH loop, or both the GBS and GH loop. In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in the GBS, GH loop, or both the GBS and GH loop of the novel AAV amino acid capsid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19 or 20 conservative amino acid residue substitutions in the GBS, GH loop, or both the GBS and GH loop of the novel AAV capsid amino acid sequence (e.g., any one of SEQ ID NOs: 12-127 and 361-362).

In another specific embodiment, a modified novel AAV capsid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in the GBS, GH loop, or both the GBS and GH loop of the novel AAV capsid amino acid sequence (e.g., any one of SEQ ID NOs: 12-127 and 361-362).

In certain embodiments, a modified novel AAV capsid sequence comprises a novel AAV capsid sequence with mutations (e.g., substitutions, insertions and/or deletions) 1, 2, 3, 4, 5, 6, 7, 8 or all 9 variable regions and GBS. In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions and GBS of the novel AAV amino acid capsid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19 or 20 conservative amino acid residue substitutions in 1, 2, 3, 4, 5, 6, 7, 8 or all 9 variable regions and GBS of the novel AAV capsid amino acid sequence (e.g., any one of SEQ ID NOs: 12-127 and 361-362).

In another specific embodiment, a modified novel AAV capsid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions and GBS of the novel AAV capsid amino acid sequence (e.g., any one of SEQ ID NOs: 12-127 and 361-362).

In certain embodiments, a modified novel AAV capsid sequence comprises a novel AAV capsid sequence with mutations (e.g., substitutions, insertions and/or deletions) 1, 2, 3, 4, 5, 6, 7, 8 or all 9 variable regions and GH loop. In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions and GH loop of the novel AAV amino acid capsid sequence (e.g., any one of SEQ ID NOs: 12-127 and 361-362). In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19 or 20 conservative amino acid residue substitutions in 1, 2, 3, 4, 5, 6, 7, 8 or all 9 variable regions and GH loop of the novel AAV capsid amino acid sequence (e.g., any one of SEQ ID NOs: 12-127 and 361-362).

In another specific embodiment, a modified novel AAV capsid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions and GH loop of the novel AAV capsid amino acid sequence (e.g., any one of SEQ ID NOs: 12-127 and 361-362). In certain embodiments, a modified novel AAV capsid sequence comprises a novel AAV capsid sequence with mutations (e.g., substitutions, insertions and/or deletions) 1, 2, 3, 4, 5, 6, 7, 8 or all 9 variable regions, GH loop and GBS. In another specific embodiment, a modified novel AAV capsid sequence of the disclosure comprises a novel AAV capsid amino acid sequence (e.g., any one of SEQ ID NOs: 12-127 and 361-362) with 1, 2, 3, 4, 5, 6, 7, 8, 9 or 10 conservative amino acid residue substitutions in 1, 2, 3, 4, 5, 6, 7, 8 or all 9 variable regions, GH loop and GBS of the novel AAV amino acid capsid sequence (e.g., any one of SEQ ID NOs: 12-127 and 361-362).

In another specific embodiment, a modified novel AAV capsid amino acid sequence of the disclosure comprises a novel AAV capsid amino acid sequence (e.g., any one of SEQ ID NOs: 12-127 and 361-362) with 11, 12, 13, 14, 15, 16, 17, 18, 19, or 20 conservative amino acid residue substitutions in 1, 2, 3, 4, 5, 6, 7, 8, or all 9 variable regions, GH loop and GBS of the novel AAV capsid amino acid sequence (e.g., any one of SEQ ID NOs: 12-127 and 361-362).

In another specific embodiment, a modified novel AAV capsid sequence of the disclosure comprises a fragment of a novel AAV capsid amino acid sequence (e.g., any one of SEQ ID NOs: 12-127 and 361-362) with 1, 2, 3, 4, 5, 6, 7, 8, 9, or 10 conservative amino acid residue substitutions in 1, 2, 3, 4, 5, 6, 7, 8, or all 9 variable regions, GH loop and GBS of the novel AAV capsid amino acid sequence (e.g., any one of SEQ ID NOs: 12-127 and 361-362).

In certain embodiments, a modified novel AAV capsid sequence of the disclosure comprises an AAV capsid amino acid sequence of a first novel AAV capsid protein (e.g., any one of SEQ ID NOs: 12-127 and 361-362) with 1, 2, 3, 4, 5, 6, 7, 8 or all 9 variable regions of a second AAV capsid protein (e.g., a different AAV serotype), wherein the first and second AAV capsid proteins are different. In specific embodiments, a modified novel AAV capsid sequence of the disclosure comprises a an AAV capsid amino acid sequence of a first novel AAV capsid protein (e.g., any one of SEQ ID NOs: 12-127 and 361-362) with 1, 2, 3, 4, 5, 6, 7, 8 or all 9 variable regions of a second novel AAV capsid protein, wherein the first and second AAV capsid proteins are different. In certain embodiments, the second AAV capsid protein (e.g., second novel AAV capsid protein) is a member of a different clade than the first novel AAV capsid protein. In some embodiments, the second AAV capsid protein (e.g., second novel AAV capsid protein) is a different AAV serotype within the same clade as the first novel AAV capsid protein. In specific embodiments, 1, 2, 3, 4, 5, 6, 7, 8, or all 9 of the variable regions in the first and second AAV capsid proteins are different.

In certain embodiments, a modified novel AAV capsid sequence of the disclosure comprises an AAV capsid amino acid sequence of a first novel AAV capsid protein (e.g., any one of SEQ ID NOs: 12-127 and 361-362) with one or more variable regions of a second AAV capsid protein (e.g., a different AAV serotype), wherein the first and second AAV capsid proteins are different. In specific embodiments, a modified novel AAV capsid sequence of the disclosure comprises an AAV capsid amino acid sequence of a first novel AAV capsid protein (e.g., any one of SEQ ID NOs: 12-127 and 361-362) with one or more variable regions of a second novel AAV capsid protein, wherein the first and second AAV capsid proteins are different. In certain embodiments, the second AAV capsid protein (e.g., second novel AAV capsid protein) is a member of a different clade than the first novel AAV capsid protein. In some embodiments, the second AAV capsid protein (e.g., second novel AAV capsid protein) is a different AAV serotype within the same clade as the first novel AAV capsid protein. In specific embodiments, the one or more variable regions in the first and second AAV capsid proteins are different.

In certain embodiments, a modified novel AAV capsid sequence of the disclosure comprises an AAV capsid amino acid sequence of a first novel AAV capsid protein (e.g., any one of SEQ ID NOs: 12-127 and 361-362) with a GBS, GH loop or both a GBS and GH loop of a second AAV capsid protein (e.g., a different AAV serotype), wherein the first and second AAV capsid proteins are different. In certain embodiments, the second AAV capsid protein is a member of a different clade than the first novel AAV capsid protein. In some embodiments, the second AAV capsid protein is a different AAV serotype within the same clade as the first novel AAV capsid protein. In specific embodiments, the GBS, GH loop, or both the GBS and GH loop in the first and second AAV capsid proteins are different.

In specific embodiments, a modified novel AAV capsid sequence of the disclosure comprises an AAV capsid amino acid sequence of a first novel AAV capsid protein (e.g., any one of SEQ ID NOs: 12-127 and 361-362) with GBS, GH loop or both a GBS and GH loop of a second novel AAV capsid protein, wherein the first and second AAV capsid proteins are different. In certain embodiments, the second novel AAV capsid protein is a member of a different clade than the first novel AAV capsid protein. In some embodiments, the second novel AAV capsid protein is a different AAV serotype within the same clade as the first novel AAV capsid protein. In specific embodiments, the GBS, GH loop, or both the GBS and GH loop in the first and second novel AAV capsid proteins are different.

In certain embodiments, a modified novel AAV capsid sequence of the disclosure comprises a an AAV capsid amino acid sequence of a first novel AAV capsid protein (e.g., any one of SEQ ID NOs: 12-127 and 361-362) with 1, 2, 3, 4, 5, 6, 7, 8 or all 9 variable regions, and GBS of a second AAV capsid protein (e.g., a different AAV serotype), wherein the first and second AAV capsid proteins are different. In certain embodiments, the second AAV capsid protein is a member of a different clade than the first novel AAV capsid protein. In some embodiments, the second AAV capsid protein is a different AAV serotype within the same clade as the first novel AAV capsid protein. In specific embodiments, the 1, 2, 3, 4, 5, 6, 7, 8 or all variable regions, and GBS in the first and second AAV capsid proteins are different.

In specific embodiments, a modified novel AAV capsid sequence of the disclosure comprises an AAV capsid amino acid sequence of a first AAV capsid protein (e.g., any one of SEQ ID NOs: 12-127 and 361-362) with 1, 2, 3, 4, 5, 6, 7, 8 or all 9 variable regions and GBS of a second novel AAV capsid protein, wherein the first and second AAV capsid proteins are different. In certain embodiments, the second novel AAV capsid protein is a member of a different clade than the first novel AAV capsid protein. In some embodiments, the second novel AAV capsid protein is a different AAV serotype within the same clade as the first novel AAV capsid protein. In specific embodiments, 1, 2, 3, 4, 5, 6, 7, 8, or all 9 of the variable regions and GBS in the first and second novel AAV capsid proteins are different.

In certain embodiments, a modified novel AAV capsid sequence of the disclosure comprises an AAV capsid amino acid sequence of a first AAV capsid protein (e.g., any one of SEQ ID NOs: 12-127 and 361-362) with one or more variable regions and GBS of a second AAV capsid protein (e.g., a different AAV serotype), wherein the first and second AAV capsid proteins are different. In certain embodiments, the second AAV capsid protein is a member of a different clade than the first novel AAV capsid protein. In some embodiments, the second AAV capsid protein is a different AAV serotype within the same clade as the first novel AAV capsid protein. In specific embodiments, the one or more variable regions and GBS in the first and second AAV capsid proteins are different.

In specific embodiments, a modified novel AAV capsid sequence of the disclosure comprises an AAV capsid amino acid sequence of a first AAV capsid (e.g., any one of SEQ ID NOs: 12-127 and 361-362) with one or more variable regions and GBS of a second novel AAV capsid protein, wherein the first and second AAV capsid proteins are different. In certain embodiments, the second novel AAV capsid protein is a member of a different clade than the first novel AAV capsid protein. In some embodiments, the second novel AAV capsid protein is a different AAV serotype within the same clade as the first novel AAV capsid protein. In specific embodiments, the one or more variable regions and GBS in the first and second novel AAV capsid proteins are different.

In certain embodiments, a modified novel AAV capsid sequence of the disclosure comprises an AAV capsid amino acid sequence of a first novel AAV capsid protein (e.g., any one of SEQ ID NOs: 12-127 and 361-362) with 1, 2, 3, 4, 5, 6, 7, 8 or all 9 variable regions (e.g., any one of, a combination thereof, or all of VRI-VRIX), and GH loop of a second AAV capsid protein (e.g., a different AAV serotype), wherein the first and second AAV capsid proteins are different. In certain embodiments, the second AAV capsid protein is a member of a different clade than the first novel AAV capsid protein. In some embodiments, the second AAV capsid protein is a different AAV serotype within the same clade as the first novel AAV capsid protein. In specific embodiments, the 1, 2, 3, 4, 5, 6, 7, 8 or all 9 variable regions, and GH loop in the first and second AAV capsid proteins are different.

In specific embodiments, a modified novel AAV capsid sequence of the disclosure comprises an AAV capsid amino acid sequence of a first novel AAV capsid (e.g., any one of SEQ ID NOs: 12-127 and 361-362) with 1, 2, 3, 4, 5, 6, 7, 8 or all 9 variable regions (e.g., any one of, a combination thereof, or all of VRI-VRIX) and GH loop of a second novel AAV capsid protein, wherein the first and second AAV capsid proteins are different. In certain embodiments, the second novel AAV capsid protein is a member of a different clade than the first novel AAV capsid protein. In some embodiments, the second novel AAV capsid protein is a different AAV serotype within the same clade as the first novel AAV capsid protein. In specific embodiments, 1, 2, 3, 4, 5, 6, 7, 8, or all 9 of the variable regions and GH loop in the first and second novel AAV capsid proteins are different.

In certain embodiments, a modified novel AAV capsid sequence of the disclosure comprises an AAV capsid amino acid sequence of a first novel AAV capsid protein (e.g., any one of SEQ ID NOs: 12-127 and 361-362) with one or more variable regions and GH loop of a second AAV capsid protein (e.g., a different AAV serotype), wherein the first and second AAV capsid proteins are different. In specific embodiments, a modified novel AAV capsid sequence of the disclosure comprises a first novel AAV capsid amino acid sequence (e.g., any one of SEQ ID NOs: 12-127 and 361-362) with one or more variable regions and GH loop of a second novel AAV capsid protein, wherein the first and second AAV capsid proteins are different. In certain embodiments, the second AAV capsid protein (e.g., second novel AAV capsid protein) is a member of a different clade than the first novel AAV capsid protein. In some embodiments, the second AAV capsid protein (e.g., second novel AAV capsid protein) is a different AAV serotype within the same clade as the first novel AAV capsid protein. In specific embodiments, the one or more variable regions and GH loop in the first and second AAV capsid proteins are different.

In certain embodiments, a modified novel AAV capsid sequence of the disclosure comprises an AAV capsid amino acid sequence of a first novel AAV capsid protein (e.g., any one of SEQ ID NOs: 12-127 and 361-362) with 1, 2, 3, 4, 5, 6, 7, 8 or all 9 variable regions (e.g., any one of, a combination thereof, or all of VRI-VRIX), GBS, and GH loop of a second AAV capsid protein (e.g., a different AAV serotype), wherein the first and second AAV capsid proteins are different. In certain embodiments, the second AAV capsid protein is a member of a different clade than the first novel AAV capsid protein. In some embodiments, the second AAV capsid protein is a different AAV serotype within the same clade as the first novel AAV capsid protein. In specific embodiments, the 1, 2, 3, 4, 5, 6, 7, 8 or all 9 variable regions, GBS, and GH loop in the first and second AAV capsid proteins are different.

In specific embodiments, a modified novel AAV capsid sequence of the disclosure comprises an AAV capsid amino acid sequence of a first AAV capsid protein (e.g., any one of SEQ ID NOs: 12-127 and 361-362) with 1, 2, 3, 4, 5, 6, 7, 8 or all 9 variable regions (e.g., any one of, a combination thereof, or all of VRI-VRIX), GBS, and GH loop of a second novel AAV capsid protein, wherein the first and second AAV capsid proteins are different. In certain embodiments, the second novel AAV capsid protein is a member of a different clade than the first novel AAV capsid protein. In some embodiments, the second novel AAV capsid protein is a different AAV serotype within the same clade as the first novel AAV capsid protein. In specific embodiments, 1, 2, 3, 4, 5, 6, 7, 8, or all 9 of the variable regions (e.g., any one of, a combination thereof, or all of VRI-VRIX), GBS, and GH loop in the first and second novel AAV capsid proteins are different.

In certain embodiments, a modified novel AAV capsid sequence of the disclosure comprises an AAV capsid amino acid sequence of a first novel AAV capsid (e.g., any one of SEQ ID NOs: 12-127 and 361-362) with one or more variable regions (e.g., any one of, a combination thereof, or all of VRI-VRIX), GBS, and GH loop of a second AAV capsid protein (e.g., a different AAV serotype), wherein the first and second AAV capsid proteins are different. In specific embodiments, a modified novel AAV capsid sequence of the disclosure comprises an AAV capsid amino acid sequence of a first novel AAV capsid (e.g., any one of SEQ ID NOS: 12-127 and 361-362) with one or more variable regions (e.g., any one of, a combination thereof, or all of VRI-VRIX), GBS, and GH loop of a second novel AAV capsid protein, wherein the first and second AAV capsid proteins are different. In certain embodiments, the second AAV capsid protein (e.g., second novel AAV capsid protein) is a member of a different clade than the first novel AAV capsid protein. In some embodiments, the second AAV capsid protein (e.g., second novel AAV capsid protein) is a different AAV serotype within the same clade as the first novel AAV capsid protein. In specific embodiments, the one or more variable regions, GBS, and GH loop in the first and second novel AAV capsid proteins are different.

Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Standardized and accepted functionally equivalent amino acid substitutions are presented in Table 1. In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

TABLE 1
Conservative Amino Acid Substitutions

| Amino Acid Group | Conservative Substitutions |
|---|---|
| Nonpolar side chains | alanine, valine, leucine, glycine, |
| | isoleucine, proline, phenylalanine, |
| | methionine, tryptophan |
| Uncharged polar side | asparagine, glutamine, serine, threonine, |
| chains | tyrosine, cysteine |
| Beta-branched side chains | threonine, valine, isoleucine |
| Aromatic side chains | tyrosine, phenylalanine, tryptophan, |
| | histidine |
| Basic side chains or | lysine, arginine, histidine |
| positively charged R groups | |
| Acidic side chains or | aspartic acid, glutamic acid |
| negatively charged R groups | |

In some embodiments, a modified novel AAV nucleic acid capsid sequence comprises a codon that encodes an amino acid that is not naturally encoded. In certain embodiments, a modified novel AAV amino acid capsid sequence comprises an amino acid mutation(s) (e.g., substitution) that allows for modification of capsids after virion assembly. In some embodiments, a modified novel AAV sequence comprises amino acid changes resulting from capsid shuffling.

In various embodiments, a modified novel AAV capsid sequence comprises one or more additional binding moieties relative to a novel AAV capsid sequence (e.g., any one of SEQ ID NOs: 12-127 and 361-362, or SEQ ID NOs: 192-307 and 363-364). Examples of binding moieties are targeting peptides (e.g., receptors), monoclonal antibodies, bispecific F(ab′)₂, and antigen-binding fragments such as Fab fragments, Fvs, scFvs, tandem scFvs, and the like. In some embodiments, a modified novel AAV capsid sequence comprises a tissue-specific targeting peptide that improves delivery of the AAV to a particular tissue in the body or cell type.

The modification of an AAV capsid may result in an AAV viral particle with one, two, three or more, or more, or all of the of following: enhanced packaging yield, enhanced transduction efficiency, enhanced gene transfer efficiency, enhanced translation efficiency, enhanced tissue-specific infectivity (i.e., tropism), and/or the enhanced ability to evade immunity compared to a non-modified AAV viral particle or naturally occurring sequence (e.g., a reference sequence, such as in Table 4, infra). The enhanced activities of the AAV particle may be assessed in an in vitro or an in vivo assay known to one of skill in the art or described herein. For example, enhanced packaging yield may be assessed by Alkaline Gel Electrophoresis, ddPCR, qPCR, SEC-MALS (see WO2021/062164, which is incorporated herein in its entirety). Enhanced transduction efficiency may be assessed, e.g., in vitro cell based assays such as Example 5, qPCR, or RNA next-generation sequencing. Enhanced translation efficiency of a transgene may be assessed by, e.g., RT-ddPCR, Liquid Chromatography-Mass Spectrometry, or by associating a transgene and/or reporter that is detectable by enzymatic, radiographic, colorimetric, fluorescence or other spectrographic assays, fluorescent activating cell sorting assays (FACS), immunological assays, including enzyme linked immunosorbent assay (ELISA), radioimmunoassay (RIA) and immunohistochemistry (IHC) assays. Enhanced tissue-specific infectivity (i.e., tropism) may be assessed by, e.g., an in vivo imaging system (IVIS), such as those described in WO2018/022608 or WO2019/222136, each of which is incorporated herein in its entirety and in particular for its tissue specific AAV infectivity assays and disclosure. Briefly, AAV comprising a test capsid and expressing one or more detectable transgenes, for example a luciferase transgene (e.g., a Fluc or Fluc2 gene) and/or a green fluorescent protein (GFP) transgene, may be generated and tested in animals, e.g., mice, by introducing the AAV into the test animals at one or mor concentrations and at an appropriate time post-infection (e.g., at 3 and 5 weeks post-infection) measurement, for example imaging, of the detectable marker or markers may be performed In the case of a luciferase marker, for example, in vivo bioluminescent imaging may be employed, utilizing standard bioluminescent substrates and imaging devices. Whole animal imaging and/or organ imaging may be analyzed using living image software. Regions of interest may be traced surrounding each animal as well as individual organs to quantify the total flux (photons/second) being released. Total flux activity is a proxy for AAV infectivity/tropism. Enhanced ability to evade immunity (pre-exisiting immunity in host) may be assessed by, e.g., cell-based in vitro TI assays, in vivo TI assays (e.g., in mice), and enzyme-linked immunosorbent assay (ELISA)-based detection of total anticapsid antibody (TAb) assays, or an IVIg cell based in vitro transduction inhibition assay tests ability of plasma to block the in vitro transduction in cultured cells. See, for example, Example 4.

### 6.3.1.4.1 Codon Optimization

In some embodiments, a novel AAV capsid nucleic acid sequence is optimized by alternative or preferred codons usage for a particular host cell or delivery cell type. AAV nucleic acid sequences can be codon optimized using any software known in the art. For example, an AAV backbone can be codon optimized using software such as//https://github.com/CMRI-TVG/AAVcodons//.

### 6.3.2 Novel AAV Clades and AAV Branches

Clades for AAV serotypes were previously proposed by Gao, G et al. J Virol. (2004) June; 78 (12): 6381-6388, which is incorporated herein by reference in its entirety, based on more than 100 AAV isolates and grouped by their viral protein (VP) phylogenetic similarity. The present disclosure provides novel AAV clades based on previously described AAV reference isolates and more than 300 new AAV isolates and grouped by various structural features as provided in Sections 6.3.2.1-6.3.2.4 and/or functional features as provided in Sections 6.3.2.5.-6.3.2.6.

A novel AAV clade encompasses all structurally related AAV members, including but not limited to, naturally-occurring AAVs, non-naturally occurring AAVs, such as for example, recombinant, modified, chimeric, hybrid (i.e., derived from two or more different AAVs), synthetic, or artificial AAVs.

In specific embodiments, the present disclosure does not encompass AAV capsid proteins that are known in the art, such as AAV VP1 sequences disclosed in any one of Table 2 with a prefix other than “BCD”, or VP2 and VP3 capsid proteins derived therefrom. In specific embodiments, the present disclosure does not encompass the AAV capsid proteins (e.g., VP1, VP2 and/or VP3) of any of the AAVs listed in Table 4 or Item A or Item B.

### 6.3.2.1 Structural Homology of VP1, VP2, and VP3 Capsid Proteins

The present disclosure also provides AAV clades grouped by their structural homology of a VP1, VP2, or VP3 capsid protein. In some embodiments, an AAV member of a novel AAV clade of the disclosure has structural homology among the VP1, VP2, or VP3 amino acid sequences to another novel AAV capsid amino acid sequence provided by the present disclosure (e.g., “a novel reference capsid”).

Homologous proteins to a novel reference capsid can be identified using sequence similarity searches, such as BLAST (Altschul et al. (1997) Nucleic Acids Res. 25:3389-3402, units 3.3 and 3.4), PSI-BLAST (id.), SSEARCH (Smith and Waterman (1981) Mol. Biol. 147:195-197; Pearson (1991) Genomics 11:635-650, unit 3.10), FASTA (Pearson and Lipman (1988) Proc. Natl. Acad. Sci. USA. 85:2444-2448, unit 3.9) and the HMMER3 (Johnson et al. (2010) BMC Bioinformatics. 11:431), which produce accurate statistical estimates, ensuring protein sequences that share significant similarity also have similar structures. Structural homology can be inferred from statistically significant similarity in, e.g., a BLAST, FASTA, SSEARCH, HMMER, or ClustalW search. Local sequence alignments calculated by BLAST, SSEARCH, FASTA, HMMER, ClustalW can identify the most similar region between two sequences. Scoring matrices, such as BLOSUM (e.g., BLOSUM62 or BLOSUM50), may be used to detect very distant similarities, and have relatively low penalties for mismatched residues. In some embodiments, the similarity of two amino acid sequences is described in terms of a similarity score. In specific embodiments, the similarity of two amino acid sequences is described in terms of the percent similarity. In some other embodiments, the similarity of two amino acid sequences is described in terms of the percent identity.

In a specific embodiment, similarity between at least two amino acid sequences is accomplished using ClustalW. In certain specific embodiments, ClustalW with cost matrix BLOSUM, a gap open cost of 10, and a gap extend cost of 0.1 is used to determine the similarity between at least two amino acid sequences.

Structural homology of a capsid protein can be determined using the methods described herein to determine if a capsid protein (e.g., VP1, VP2, or VP3) has substantial homology (e.g., substantial amino acid similarity and/or amino acid similarity). In some embodiments, substantial homology (e.g., substantial amino acid identity and/or amino acid similarity) is across the full-length of capsid proteins (e.g., two VP1 capsid proteins, two VP2 capsid proteins, or two VP3 capsid proteins). In certain embodiments, substantial homology (e.g., substantial amino acid identity and/or amino acid similarity) is across a fragment of two capsid proteins (e.g., a fragment of two VP1 capsid proteins, a fragment of two VP2 capsid proteins, or a fragment of two VP3 capsid proteins).

In a specific embodiment, a capsid protein (e.g., VP1, VP2, or VP3) has substantial homology if there is about 90% to 99% similarity and/or identity to another capsid protein (e.g., VP1, VP2 or VP3) using a technique described herein (e.g., ClustalW) or known to one of skill in the art.

In some embodiments, a VP1 capsid protein has substantial homology if there is about 90% to 99% similarity and/or identity to a VP1 capsid protein in any one of Table 9 using a technique described herein (e.g., ClustalW) or known to one of skill in the art. In specific embodiments, the VP1 capsid protein is one provided in Table 9 with a “BCD_” prefix.

In another specific embodiment, a VP1 capsid protein has substantial homology if there is about 90% to 99% similarity and/or identity to a VP1 capsid protein in any one of Table 2 using a technique described herein (e.g., ClustalW) or known to one of skill in the art. In another specific embodiment, a VP1 capsid protein has substantial homology if there is about 90% to 99% similarity and/or identity to VP1 capsid protein No. 0 in any one of Table 2 using a technique described herein (e.g., ClustalW) or known to one of skill in the art.

In some embodiments, a VP1 capsid protein has structural homology with a VP1 capsid protein in an AAV clade in any one of Table 2 if there is at least about 90% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 91% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 91% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 91% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 92% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 92% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 93% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 93% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 94% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 94% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 95% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 95% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 96% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 96% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 97% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 97% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 98% identity between the VP1 capsid amino acid sequences using a technique described herein (e.g., ClustalW) or known to one of skill in the art. In a specific embodiment, a VP1 capsid protein has structural homology with VP1 capsid protein of No. 0 in an AAV clade in any one of Table 2 if there is at least about 98% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 99% identity between the VP1 capsid amino acid sequences using a technique described herein (e.g., ClustalW) or known to one of skill in the art. In a specific embodiment, a VP1 capsid protein has structural homology with VP1 capsid protein of No. 0 in an AAV clade in any one of Table 2 if there is at least about 99% identity between the VP1 capsid amino acid sequences using a technique described herein (e.g., ClustalW) or known to one of skill in the art.

In some embodiments, a capsid protein has substantial similarity if there is about 90% to 99% similarity to another capsid protein (e.g., VP1, VP2 or VP3) using a technique described herein (e.g., ClustalW) or known to one of skill in the art. In some embodiments, a capsid protein has substantial similarity if there is about 90% similarity to another capsid protein (e.g., VP1, VP2 or VP3) using a technique described herein (e.g., ClustalW) or known to one of skill in the art. In some embodiments, a capsid protein has substantial similarity if there is about 91% similarity to another capsid protein (e.g., VP1, VP2 or VP3) using a technique described herein (e.g., ClustalW) or known to one of skill in the art. In some embodiments, a capsid protein has substantial similarity if there is about 92% similarity to another capsid protein (e.g., VP1, VP2 or VP3) using a technique described herein (e.g., ClustalW) or known to one of skill in the art. In some embodiments, a capsid protein has substantial similarity if there is about 93% similarity to another capsid protein (e.g., VP1, VP2 or VP3) using a technique described herein (e.g., ClustalW) or known to one of skill in the art. In some embodiments, a capsid protein has substantial similarity if there is about 94% similarity to another capsid protein (e.g., VP1, VP2 or VP3) using a technique described herein (e.g., ClustalW) or known to one of skill in the art. In some embodiments, a capsid protein has substantial similarity if there is about 95% similarity to another capsid protein (e.g., VP1, VP2 or VP3) using a technique described herein (e.g., ClustalW) or known to one of skill in the art. In some embodiments, a capsid protein has substantial similarity if there is about 96% similarity to another capsid protein (e.g., VP1, VP2 or VP3) using a technique described herein (e.g., ClustalW) or known to one of skill in the art. In some embodiments, a capsid protein has substantial similarity if there is about 97% similarity to another capsid protein (e.g., VP1, VP2 or VP3) using a technique described herein (e.g., ClustalW) or known to one of skill in the art. In some embodiments, a capsid protein has substantial similarity if there is about 98% similarity to another capsid protein (e.g., VP1, VP2 or VP3) using a technique described herein (e.g., ClustalW) or known to one of skill in the art. In some embodiments, a capsid protein has substantial similarity if there is about 99% similarity to another capsid protein (e.g., VP1, VP2 or VP3) using a technique described herein (e.g., ClustalW) or known to one of skill in the art.

In some embodiments, a VP1 capsid protein has substantial similarity if there is about 90% to 99% similarity to a VP1 capsid protein in any one of Table 9 using a technique described herein (e.g., ClustalW) or known to one of skill in the art. In some embodiments, a VP1 capsid protein has substantial similarity if there is about 90% similarity to a VP1 capsid protein in any one of Table 9 using a technique described herein (e.g., ClustalW) or known to one of skill in the art. In some embodiments, a VP1 capsid protein has substantial similarity if there is about 91% similarity to a VP1 capsid protein in any one of Table 9 using a technique described herein (e.g., ClustalW) or known to one of skill in the art. In some embodiments, a VP1 capsid protein has substantial similarity if there is about 92% similarity to a VP1 capsid protein in any one of Table 9 using a technique described herein (e.g., ClustalW) or known to one of skill in the art. In some embodiments, a VP1 capsid protein has substantial similarity if there is about 93% similarity to a VP1 capsid protein in any one of Table 9 using a technique described herein (e.g., ClustalW) or known to one of skill in the art. In some embodiments, a VP1 capsid protein has substantial similarity if there is about 94% similarity to a VP1 capsid protein in any one of Table 9 using a technique described herein (e.g., ClustalW) or known to one of skill in the art. In some embodiments, a VP1 capsid protein has substantial similarity if there is about 95% similarity to a VP1 capsid protein in any one of Table 9 using a technique described herein (e.g., ClustalW) or known to one of skill in the art. In some embodiments, a VP1 capsid protein has substantial similarity if there is about 96% similarity to a VP1 capsid protein in any one of Table 9 using a technique described herein (e.g., ClustalW) or known to one of skill in the art. In some embodiments, a VP1 capsid protein has substantial similarity if there is about 97% similarity to a VP1 capsid protein in any one of Table 9 using a technique described herein (e.g., ClustalW) or known to one of skill in the art. In some embodiments, a VP1 capsid protein has substantial similarity if there is about 98% similarity to a VP1 capsid protein in any one of Table 9 using a technique described herein (e.g., ClustalW) or known to one of skill in the art. In some embodiments, a VP1 capsid protein has substantial similarity if there is about 99% similarity to a VP1 capsid protein in any one of Table 9 using a technique described herein (e.g., ClustalW) or known to one of skill in the art. In specific embodiments, the VP1 capsid protein is one provided in Table 9 with a “BCD_” prefix.

In another specific embodiment, a VP1 capsid protein has substantial similarity if there is about 90% to 99% similarity to a VP1 capsid protein in any one of Table 2 using a technique described herein (e.g., ClustalW) or known to one of skill in the art. In another specific embodiment, a VP1 capsid protein has substantial similarity if there is about 90% similarity to a VP1 capsid protein in any one of Table 2 using a technique described herein (e.g., ClustalW) or known to one of skill in the art. In another specific embodiment, a VP1 capsid protein has substantial similarity if there is about 91% similarity to a VP1 capsid protein in any one of Table 2 using a technique described herein (e.g., ClustalW) or known to one of skill in the art. In another specific embodiment, a VP1 capsid protein has substantial similarity if there is about 92% similarity to a VP1 capsid protein in any one of Table 2 using a technique described herein (e.g., ClustalW) or known to one of skill in the art. In another specific embodiment, a VP1 capsid protein has substantial similarity if there is about 93% similarity to a VP1 capsid protein in any one of Table 2 using a technique described herein (e.g., ClustalW) or known to one of skill in the art. In another specific embodiment, a VP1 capsid protein has substantial similarity if there is about 94% similarity to a VP1 capsid protein in any one of Table 2 using a technique described herein (e.g., ClustalW) or known to one of skill in the art. In another specific embodiment, a VP1 capsid protein has substantial similarity if there is about 95% similarity to a VP1 capsid protein in any one of Table 2 using a technique described herein (e.g., ClustalW) or known to one of skill in the art. In another specific embodiment, a VP1 capsid protein has substantial similarity if there is about 96% similarity to a VP1 capsid protein in any one of Table 2 using a technique described herein (e.g., ClustalW) or known to one of skill in the art. In another specific embodiment, a VP1 capsid protein has substantial similarity if there is about 97% similarity to a VP1 capsid protein in any one of Table 2 using a technique described herein (e.g., ClustalW) or known to one of skill in the art. In another specific embodiment, a VP1 capsid protein has substantial similarity if there is about 98% similarity to a VP1 capsid protein in any one of Table 2 using a technique described herein (e.g., ClustalW) or known to one of skill in the art. In another specific embodiment, a VP1 capsid protein has substantial similarity if there is about 99% similarity to a VP1 capsid protein in any one of Table 2 using a technique described herein (e.g., ClustalW) or known to one of skill in the art. In specific embodiments, the VP1 capsid protein with substantial similarity to a VP1 capsid protein in any one of Table 2 is not a known AAV. Table 9 provides the sequences of the VP1 capsid protein recited in any one of Table 2.

In another specific embodiment, a VP1 capsid protein has substantial similarity if there is about 90% to 99% similarity to VP1 capsid protein No. 0 in any one of Table 2 using a technique described herein (e.g., ClustalW) or known to one of skill in the art. In another specific embodiment, a VP1 capsid protein has substantial similarity if there is about 90% similarity to VP1 capsid protein No. 0 in any one of Table 2 using a technique described herein (e.g., ClustalW) or known to one of skill in the art. In another specific embodiment, a VP1 capsid protein has substantial similarity if there is about 91% similarity to VP1 capsid protein No. 0 in any one of Table 2 using a technique described herein (e.g., ClustalW) or known to one of skill in the art. In another specific embodiment, a VP1 capsid protein has substantial similarity if there is about 92% similarity to VP1 capsid protein No. 0 in any one of Table 2 using a technique described herein (e.g., ClustalW) or known to one of skill in the art. In another specific embodiment, a VP1 capsid protein has substantial similarity if there is about 93% similarity to VP1 capsid protein No. 0 in any one of Table 2 using a technique described herein (e.g., ClustalW) or known to one of skill in the art. In another specific embodiment, a VP1 capsid protein has substantial similarity if there is about 94% similarity to VP1 capsid protein No. 0 in any one of Table 2 using a technique described herein (e.g., ClustalW) or known to one of skill in the art. In another specific embodiment, a VP1 capsid protein has substantial similarity if there is about 95% similarity to VP1 capsid protein No. 0 in any one of Table 2 using a technique described herein (e.g., ClustalW) or known to one of skill in the art. In another specific embodiment, a VP1 capsid protein has substantial similarity if there is about 96% similarity to VP1 capsid protein No. 0 in any one of Table 2 using a technique described herein (e.g., ClustalW) or known to one of skill in the art. In another specific embodiment, a VP1 capsid protein has substantial similarity if there is about 97% similarity to VP1 capsid protein No. 0 in any one of Table 2 using a technique described herein (e.g., ClustalW) or known to one of skill in the art. In another specific embodiment, a VP1 capsid protein has substantial similarity if there is about 98% similarity to VP1 capsid protein No. 0 in any one of Table 2 using a technique described herein (e.g., ClustalW) or known to one of skill in the art. In another specific embodiment, a VP1 capsid protein has substantial similarity if there is about 99% similarity to VP1 capsid protein No. 0 in any one of Table 2 using a technique described herein (e.g., ClustalW) or known to one of skill in the art. In specific embodiments, the VP1 capsid protein with substantial similarity to a VP1 capsid protein of No. 0 in any one of Table 2 is not a known AAV.

In some embodiments, a capsid protein has substantial identity if there is about 90% to 99% identity to another capsid protein (e.g., VP1, VP2 or VP3) using a technique described herein (e.g., ClustalW) or known to one of skill in the art. In some embodiments, a capsid protein has substantial identity if there is about 90% identity to another capsid protein (e.g., VP1, VP2 or VP3) using a technique described herein (e.g., ClustalW) or known to one of skill in the art. In some embodiments, a capsid protein has substantial identity if there is about 91% identity to another capsid protein (e.g., VP1, VP2 or VP3) using a technique described herein (e.g., ClustalW) or known to one of skill in the art. In some embodiments, a capsid protein has substantial identity if there is about 92% identity to another capsid protein (e.g., VP1, VP2 or VP3) using a technique described herein (e.g., ClustalW) or known to one of skill in the art. In some embodiments, a capsid protein has substantial identity if there is about 93% identity to another capsid protein (e.g., VP1, VP2 or VP3) using a technique described herein (e.g., ClustalW) or known to one of skill in the art. In some embodiments, a capsid protein has substantial identity if there is about 94% identity to another capsid protein (e.g., VP1, VP2 or VP3) using a technique described herein (e.g., ClustalW) or known to one of skill in the art. In some embodiments, a capsid protein has substantial identity if there is about 95% identity to another capsid protein (e.g., VP1, VP2 or VP3) using a technique described herein (e.g., ClustalW) or known to one of skill in the art. In some embodiments, a capsid protein has substantial identity if there is about 96% identity to another capsid protein (e.g., VP1, VP2 or VP3) using a technique described herein (e.g., ClustalW) or known to one of skill in the art. In some embodiments, a capsid protein has substantial identity if there is about 97% identity to another capsid protein (e.g., VP1, VP2 or VP3) using a technique described herein (e.g., ClustalW) or known to one of skill in the art. In some embodiments, a capsid protein has substantial identity if there is about 98% identity to another capsid protein (e.g., VP1, VP2 or VP3) using a technique described herein (e.g., ClustalW) or known to one of skill in the art. In some embodiments, a capsid protein has substantial identity if there is about 99% identity to another capsid protein (e.g., VP1, VP2 or VP3) using a technique described herein (e.g., ClustalW) or known to one of skill in the art. In specific embodiments, a capsid protein with substantial identity is not a known AAV capsid. In specific embodiments, a capsid protein with substantial identity is a capsid protein provided herein (e.g., in Table 9) with the “BCD_” prefix.

In some embodiments, a VP1 capsid protein has substantial identity if there is about 90% to 99% identity to a VP1 capsid protein in any one of Table 9 using a technique described herein (e.g., ClustalW) or known to one of skill in the art. In some embodiments, a VP1 capsid protein has substantial identity if there is about 90% identity to a VP1 capsid protein in any one of Table 9 using a technique described herein (e.g., ClustalW) or known to one of skill in the art. In some embodiments, a VP1 capsid protein has substantial identity if there is about 91% identity to a VP1 capsid protein in any one of Table 9 using a technique described herein (e.g., ClustalW) or known to one of skill in the art. In some embodiments, a VP1 capsid protein has substantial identity if there is about 92% identity to a VP1 capsid protein in any one of Table 9 using a technique described herein (e.g., ClustalW) or known to one of skill in the art. In some embodiments, a VP1 capsid protein has substantial identity if there is about 93% identity to a VP1 capsid protein in any one of Table 9 using a technique described herein (e.g., ClustalW) or known to one of skill in the art. In some embodiments, a VP1 capsid protein has substantial identity if there is about 94% identity to a VP1 capsid protein in any one of Table 9 using a technique described herein (e.g., ClustalW) or known to one of skill in the art. In some embodiments, a VP1 capsid protein has substantial identity if there is about 95% identity to a VP1 capsid protein in any one of Table 9 using a technique described herein (e.g., ClustalW) or known to one of skill in the art. In some embodiments, a VP1 capsid protein has substantial identity if there is about 96% identity to a VP1 capsid protein in any one of Table 9 using a technique described herein (e.g., ClustalW) or known to one of skill in the art. In some embodiments, a VP1 capsid protein has substantial identity if there is about 97% identity to a VP1 capsid protein in any one of Table 9 using a technique described herein (e.g., ClustalW) or known to one of skill in the art. In some embodiments, a VP1 capsid protein has substantial identity if there is about 98% identity to a VP1 capsid protein in any one of Table 9 using a technique described herein (e.g., ClustalW) or known to one of skill in the art. In some embodiments, a VP1 capsid protein has substantial identity if there is about 99% identity to a VP1 capsid protein in any one of Table 9 using a technique described herein (e.g., ClustalW) or known to one of skill in the art. In specific embodiments, the VP1 capsid protein is one provided in Table 9 with a “BCD_” prefix. In specific embodiments, a capsid protein with substantial identity is not a known AAV capsid.

In another specific embodiment, a VP1 capsid protein has substantial identity if there is about 90% to 99% identity to a VP1 capsid protein in any one of Table 2 using a technique described herein (e.g., ClustalW) or known to one of skill in the art. In another specific embodiment, a VP1 capsid protein has substantial identity if there is about 90% identity to a VP1 capsid protein in any one of Table 2 using a technique described herein (e.g., ClustalW) or known to one of skill in the art. In another specific embodiment, a VP1 capsid protein has substantial identity if there is about 95% identity to a VP1 capsid protein in any one of Table 2 using a technique described herein (e.g., ClustalW) or known to one of skill in the art. In another specific embodiment, a VP1 capsid protein has substantial identity if there is about 96% identity to a VP1 capsid protein in any one of Table 2 using a technique described herein (e.g., ClustalW) or known to one of skill in the art. In another specific embodiment, a VP1 capsid protein has substantial identity if there is about 97% identity to a VP1 capsid protein in any one of Table 2 using a technique described herein (e.g., ClustalW) or known to one of skill in the art. In another specific embodiment, a VP1 capsid protein has substantial identity if there is about 98% identity to a VP1 capsid protein in any one of Table 2 using a technique described herein (e.g., ClustalW) or known to one of skill in the art. In another specific embodiment, a VP1 capsid protein has substantial identity if there is about 99% identity to a VP1 capsid protein in any one of Table 2 using a technique described herein (e.g., ClustalW) or known to one of skill in the art. In specific embodiments, the VP1 capsid protein with substantial identity to a VP1 capsid protein to a VP1 capsid protein in any one of Table 2 is not a known AAV.

In another specific embodiment, a VP1 capsid protein has substantial identity if there is about 90% to 99% identity to VP1 capsid protein No. 0 in any one of Table 2 using a technique described herein (e.g., ClustalW) or known to one of skill in the art. In another specific embodiment, a VP1 capsid protein has substantial identity if there is about 90% identity to VP1 capsid protein No. 0 in any one of Table 2 using a technique described herein (e.g., ClustalW) or known to one of skill in the art. In another specific embodiment, a VP1 capsid protein has substantial identity if there is about 95% identity to VP1 capsid protein No. 0 in any one of Table 2 using a technique described herein (e.g., ClustalW) or known to one of skill in the art. In another specific embodiment, a VP1 capsid protein has substantial identity if there is about 96% identity to VP1 capsid protein No. 0 in any one of Table 2 using a technique described herein (e.g., ClustalW) or known to one of skill in the art. In another specific embodiment, a VP1 capsid protein has substantial identity if there is about 97% identity to VP1 capsid protein No. 0 in any one of Table 2 using a technique described herein (e.g., ClustalW) or known to one of skill in the art. In another specific embodiment, a VP1 capsid protein has substantial identity if there is about 98% identity to VP1 capsid protein No. 0 in any one of Table 2 using a technique described herein (e.g., ClustalW) or known to one of skill in the art. In another specific embodiment, a VP1 capsid protein has substantial identity if there is about 99% identity to VP1 capsid protein No. 0 in any one of Table 2 using a technique described herein (e.g., ClustalW) or known to one of skill in the art. In specific embodiments, the VP1 capsid protein with substantial identity to a VP1 capsid protein of No. 0 in any one of Table 2 is not a known AAV.

In some embodiments, a VP1 capsid protein has structural homology with a VP1 capsid protein in an AAV clade in any one of Table 2 if there is at least about 90% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 91% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 91% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 91% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 92% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 92% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 93% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 93% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 94% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 94% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 95% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 95% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 96% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 96% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 97% identity between the VP1 capsid amino acid sequences using a technique described herein (e.g., ClustalW) or known to one of skill in the art. In a specific embodiment, a VP1 capsid protein has structural homology with VP1 capsid protein No. 0 in an AAV clade in any one of Table 2 if there is at least about 97% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 98% identity between the VP1 capsid amino acid sequences using a technique described herein (e.g., ClustalW) or known to one of skill in the art. In a specific embodiment, a VP1 capsid protein has structural homology with VP1 capsid protein of No. 0 in an AAV clade in any one of Table 2 if there is at least about 98% identity between the VP1 capsid amino acid sequences using a technique described herein (e.g., ClustalW) or known to one of skill in the art. In some embodiments, a VP1 capsid protein has structural homology with a VP1 capsid protein in an AAV clade in any one of Table 2 if there is at least about 99% identity between the VP1 capsid amino acid sequences using a technique described herein (e.g., ClustalW) or known to one of skill in the art. In a specific embodiment, a VP1 capsid protein has structural homology with VP1 capsid protein of No. 0 in an AAV clade in any one of Table 2 if there is at least about 99% identity between the VP1 capsid amino acid sequences using a technique described herein (e.g., ClustalW) or known to one of skill in the art. In specific embodiments, the VP1 capsid protein with structural homology to a VP1 capsid protein of No. 0 in an AAV clade in any one of Table 2 is not a known AAV.

In specific embodiments, VP1 capsid proteins with about 90% to 99% identity to a VP1 capsid protein in any one of Table 2 using a technique described herein (e.g., ClustalW) or known to one of skill in the art are not encompassed by the present disclosure if the VP1 AAV capsid protein was known in the art, such as AAV VP1 sequences disclosed in any one of Table 2 with a prefix other than “BCD”, or VP2 and VP3 capsid proteins derived therefrom.

In specific embodiments, VP1 capsid proteins with about 90% to 99% identity to VP1 capsid protein No. 0 in any one of Table 2 using a technique described herein (e.g., ClustalW) or known to one of skill in the art are not encompassed by the present disclosure if the VP1 AAV capsid protein is one listed in Table 4 or Item A or Item B.

TABLE 2
AAV Clades, AAV Branches, and Representative Sequence in subtables 2.6-2.15
Table 2.6: Branch 2 - Clade 0

| | | | % identity to | |
|---|---|---|---|---|
| | AA | | representative | Varying amino acid residues relative |
| No. | Length | Name | sequence | to the representative sequence |
| 0 | 739aa | BCD_0160 | * | |
| | | (SEQ ID | | |
| | | NO: 28) | | |
| 1 | 739aa | BCD_0161 | 99.86% | G308E |
| 2 | 739aa | BCD_0165 | 98.11% | G42S, V118A, Q168P, N385D, V522A, |
| | | | | S527A, K549R, E559D, V563I, V569I, |
| | | | | G576A, N590G, T591S, T666A |
| 3 | 739aa | BCD_0174 | 98.51% | G42S, Q168P, V522A, S527A, K549R, |
| | | | | E559D, V563I, V569I, G576A, N590G, |
| | | | | T591S |
| 4 | 739aa | BCD_0175 | 98.24% | G42S, G49W, Q168P, F285S, V522A, |
| | | | | S527A, K549R, E559D, V563I, V569I, |
| | | | | G576A, N590G, T591S |
| 5 | 739aa | BCD_0190 | 96.89% | A24D, Q31K, H38K, N41D, E67A, K81Q, |
| | | | | E84K, Q85A, K92R, I125V, A135G, Q168P, |
| | | | | I191L, S267T, Q413S, M487T, V522T, |
| | | | | S527A, D565S, V569I, G576A, T598S, |
| | | | | I604V |
| 6 | 739aa | BCD_0192 | 96.75% | A24D, Q31K, H38K, N41D, E67A, K81Q, |
| | | | | E84K, Q85A, K92R, I125V, A135G, Q168P, |
| | | | | I191L, S267T, Q413S, T462A, M487T, |
| | | | | V522T, S527A, D565S, V569I, G576A, |
| | | | | T598S, I604V |
| 7 | 739aa | BCD_0274 | 97.56% | N41D, G42S, Q168P, H258R, F316S, |
| | | | | Y355C, S473P, V522A, S527A, K549Q, |
| | | | | E559D, V563I, D565S, V569I, G576A, |
| | | | | N590G, T591S, T598S |
| 8 | 739aa | BCD_0275 | 97.97% | Q31K, G42S, Q101R, Q168P, V522A, |
| | | | | S527A, K549Q, E559D, V563I, D565S, |
| | | | | V569I, G576A, N590G, T591S, T598S |
| 9 | 739aa | BCD_0276 | 97.83% | Q31K, G42S, Q168P, Q193P, V522A, |
| | | | | S527A, K549Q, E559D, V563I, D565S, |
| | | | | V569I, G576A, N590G, T591S, T598S, |
| | | | | S668P |
| 10 | 739aa | BCD_0277 | 98.11% | Q31K, G42S, Q168P, V522A, S527A, |
| | | | | K549Q, E559D, V563I, D565S, V569I, |
| | | | | G576A, N590G, T591S, T598S |
| 11 | 739aa | BCD_0278 | 97.16% | Q31K, G42S, P140L, Q168P, N288D, |
| | | | | V323A, D371G, V522A, S527A, K549Q, |
| | | | | E559D, V563I, D565S, V569I, G576A, |
| | | | | N590G, T591S, T598S, Q618R, P634T, |
| | | | | Y734C |
| 12 | 739aa | BCD_0279 | 97.70% | Q31K, G42S, S109F, L126P, Q168P |
| | | | | F401S, V522A, S527A, K549Q, E559D, |
| | | | | V563I, D565S, V569I, G576A, N590G, |
| | | | | T591S, T598S |
| 13 | 739aa | BCD_0280 | 97.97% | G42S, Q168P, Q169R, P253H, V522A, |
| | | | | S527A, K549Q, E559D, V563I, D565S, |
| | | | | V569I, G576A, S589A, N590S, T591N |
| 14 | 739aa | BCD_0281 | 97.70% | G42S, Q168P, T280A, V326A, S389P, |
| | | | | V522A, S527A, F547S, K549Q, E559D, |
| | | | | V563I, D565S, V569I, G576A, S589A, |
| | | | | N590S, T591N |
| 15 | 739aa | BCD_0282 | 97.97% | G42S, R103H, Q168P, V522A, S527A, |
| | | | | K549Q, E559D, V563I, D565S, V569I, |
| | | | | G576A, L587P, S589A, N590S, T591N |
| 16 | 739aa | BCD_0283 | 97.97% | G42S, K51E, Q168P, V522A, S527A, |
| | | | | K549Q, E559D, V563I, D565S, V569I, |
| | | | | G576A, S589A, N590S, T591N, K644E, |
| 17 | 739aa | BCD_0284 | 98.24% | G42S, Q168P, V522A, S527A, K549Q, |
| | | | | E559D, V563I, D565S, V569I, G576A, |
| | | | | S589A, N590S, T591N |
| 18 | 739aa | BCD_0285 | 97.70% | G42S, Q168P, L314H, L465P, V522A, |
| | | | | S527A, K549Q, T553A, E559D, V563I, |
| | | | | D565S, V569I, G576A, S589A, N590S, |
| | | | | T591N, F665S |
| 19 | 739aa | BCD_0286 | 98.51% | Q168P, V522A, S527A, K549Q, E559D, |
| | | | | V563I, D565S, V569I, G576A, N590S, |
| | | | | T598S |
| 20 | 739aa | BCD_0287 | 98.11% | Q168P, I259V, S265G, V522A, S527A, |
| | | | | K549Q, E559D, V563I, D565S, V569I, |
| | | | | G576A, N590S, T598S, V718A |
| 21 | 739aa | BCD_0288 | 98.24% | Q168P, V522A, S527A, K549Q, E559D, |
| | | | | V563I, D565S, V569I, G576A, N590S, |
| | | | | T598S, T655A, P700L |
| 22 | 739aa | BCD_0289 | 98.24% | T149A, Q168P, V522A, S527A, K549Q, |
| | | | | E559D, N560T, V563I, D565S, V569I, |
| | | | | G576A, N590S, T598S |
| 23 | 739aa | BCD_0290 | 98.11% | Q168P, D240G, N500S, V522A, S527A, |
| | | | | K549Q, E559D, V563I, D565S, V569I, |
| | | | | G576A, N590S, T598S, Q703R |
| 24 | 739aa | BCD_0291 | 97.97% | G18C, Q168P, K312E, I318V, S425G, |
| | | | | V522A, S527A, K549Q, E559D, V563I, |
| | | | | D565S, V569I, G576A, N590S, T598S |
| 25 | 739aa | BCD_0292 | 98.51% | Q168P, V522A, S527A, K549Q, E559D, |
| | | | | V563I, D565S, V569I, G576A, N590S, |
| | | | | T598S |
| 26 | 739aa | BCD_0293 | 98.11% | K61E, Q168P, L353P, V522A, S527A, |
| | | | | K549Q, E559D, V563I, D565S, V569I, |
| | | | | G576A, N590S, T598S, K692E |
| 27 | 739aa | BCD_0294 | 98.24% | F56L, Q168P, K312E, V522A, S527A, |
| | | | | K549Q, E559D, V563I, D565S, V569I, |
| | | | | G576A, N590S, T598S |
| 28 | 739aa | BCD_0295 | 98.11% | V118A, P150R, Q168P, V522A, S527A, |
| | | | | K549Q, E559D, V563I, D565S, V569I, |
| | | | | G576A, N590S, T598S, M638V |
| 29 | 739aa | BCD_0296 | 97.83% | Q31K, G42S, Q152R, Q168P, V522A, |
| | | | | S527A, G543E, K549Q, E559D, V563I, |
| | | | | D565S, V569I, G576A, N590G, T591S, |
| | | | | T598S |
| 30 | 739aa | BCD_0297 | 97.97% | Q31K, G42S, Q168P, N204S, V522A, |
| | | | | S527A, K549Q, E559D, V563I, D565S, |
| | | | | V569I, G576A, N590G, T591S, T598S |
| 31 | 739aa | BCD_0298 | 98.11% | Q168P, H258R, Q362R, V522A, S527A, |
| | | | | K549Q, E559D, V563I, D565S, V569I, |
| | | | | G576A, N590S, T598S, F716S |
| 32 | 739aa | BCD_0299 | 98.78% | K142R, R407G, K549Q, E559D, V563I, |
| | | | | G576A, N590G, T591S, F670S |
| 33 | 739aa | BCD_0300 | 98.78% | R20C, S527P, K549Q, E559D, V563I, |
| | | | | G576A, E578G, N590G, T591S |
| 34 | 739aa | BCD_0301 | 98.78% | Y52H, E67K, F538L, K549Q, E559D, |
| | | | | V563I, G576A, N590G, T591S |
| 35 | 739aa | BCD_0446 | 96.08% | A24D, Q31K, H38K, N41D, E67A, K81Q, |
| | | | | E84K, Q85A, K92R, I125V, A135G, Q168P, |
| | | | | I191L, S267T, Q413S, M487T, V522T, |
| | | | | S527A, N560H, D565S, V569I, G576A, |
| | | | | N590S, T591A, T592A, N593G, T595Q, |
| | | | | E597Q, I604V |
| 36 | 736aa | BCD_0454 | 91.30% | AA24D, Q31K, H38K, N41D, D60N, K61R, |
| | | | | E67A, K81Q, E84K, Q85A, K92R, I125V, |
| | | | | L129F, A135G, L146V, K148Q, T149S, |
| | | | | N151Q, Q152E, T154D, D155S, T156S, |
| | | | | Q157S, A158G, A159I, 161-162insKSG, |
| | | | | T162Q, Q168L, Q169N, G170F, E171G, |
| | | | | S173T, 177-179delARR, Q180E, L181S, |
| | | | | D182V, F183P, 187-189delPAA, I191L, |
| | | | | Q193E, A197T, S199A, P200A, S203P, |
| | | | | N204T, G212A, D217N, Q219E, E266D, |
| | | | | S267T, Q413S, R463K, M487T, V522T, |
| | | | | S527A, N551D, D565S, V569I, G576A, |
| | | | | T591A, T598S, I604V, D719N |
| 37 | 739aa | BCD_0455 | 97.02% | A24N, Q31K, H38K, E67A, K81Q, E84K, |
| | | | | Q85A, K92R, I125V, A135G, Q168P, I191L, |
| | | | | S267T, Q413S, M487T, V522T, S527A, |
| | | | | D565S, V569I, G576A, T598S, I604V |
| 38 | 739aa | BMN_0261 | 97.56% | D40G, G42S, P55L, N88D, Q168P, D184G, |
| | | | | S199P, I321V, V522A, S527A, K549Q, |
| | | | | E559D, D565S, V569I, G576A, S589A, |
| | | | | N590S, T591N |
| 39 | 739aa | BMN_0262 | 97.56% | G42S, F100L, Q168P, S199P, C233R, |
| | | | | V522A, S527A, K549Q, E559D, V563I, |
| | | | | D565S, V569I, G576A, S589A, N590S, |
| | | | | T591N, N601S, S695G |
| 40 | 739aa | BMN_0263 | 97.83% | G42S, K143R, Q168P, Y444C, V522A, |
| | | | | S527A, K549Q, E559D, V563I, D565S, |
| | | | | V569I, G576A, S589A, N590S, T591N, |
| | | | | N694D |
| 41 | 739aa | BMN_0264 | 97.83% | G42S, Q168P, E171G, Q352L, V522A, |
| | | | | S527A, K549Q, E559D, V563I, D565S, |
| | | | | V569I, G576A, S589A, N590S, T591N, |
| | | | | N694D |
| 42 | 739aa | BMN_0265 | 99.19% | G42S, N113I, Q168P, S527A, G576A, |
| | | | | V718A |
| 43 | 739aa | BMN_0266 | 97.43% | G42S, A135V, Q168P, V522A, S527A, |
| | | | | G543R, K549Q, E559D, V563I, D565S, |
| | | | | V569I, G576A, S589A, N590S, T591N, |
| | | | | N593D, A623V, I674T, E689G |
| 44 | 739aa | BMN_0267 | 97.83% | G42S, D69Y, L126P, Q168P, S393P, |
| | | | | V522A, S527A, K549Q, E559D, V563I, |
| | | | | D565S, V569I, G576A, S589A, N590S, |
| | | | | T591N |
| 45 | 739aa | BMN_0268 | 97.83% | G42S, K137E, Q168P, I329T, M437I, |
| | | | | V522A, S527A, K549Q, E559D, V563I, |
| | | | | D565S, V569I, G576A, S589A, N590S, |
| | | | | T591N |
| 46 | 739aa | BMN_0269 | 97.83% | E21G, G42S, Y90C, Q168P, V522A, S527A, |
| | | | | K549Q, E559D, V563I, D565S, V569I, |
| | | | | G576A, S589A, N590S, T591N, I621T |
| 47 | 739aa | BMN_0270 | 98.11% | G42S, Q168P, N411S, V522A, S527A, |
| | | | | K549Q, E559D, V563I, D565S, V569I, |
| | | | | G576A, S589A, N590S, T591N |
| 48 | 739aa | BMN_0271 | 99.19% | G42S, E67G, Q168P, S527A, G576A, P729L |
| 49 | 739aa | BMN_0272 | 99.46% | G42S, Q168P, S527A, G576A |
| 50 | 739aa | BMN_0273 | 99.19% | G42S, Q168P, N500D, S527A, G576A, |
| | | | | V609A |

TABLE 2.7
Branch 2 - Clade 1

| | | | % identity to | |
|---|---|---|---|---|
| | AA | | representative | Varying amino acid residues relative |
| No. | Length | Name | sequence | to the representative sequence |
| 0 | 738aa | AAV-10 | * | |
| | | (SEQ ID | | |
| | | NO: 10) | | |
| 1 | 738aa | AAV-8 | 93.36% | D24A, K84Q, A135G, K169R, E179D, |
| | | | | I189L, G195A, L199V, S201P, G202N, |
| | | | | S269A, E413Q, S415T, S453T, Q458A, |
| | | | | G459N, Q462T, L464G, A468G, A471N, |
| | | | | N472T, S474A, A475N, L495T, S496G, |
| | | | | G507A, A508G, D517N, V520A, V524I, |
| | | | | S540N, V542I, M544I, G549N, G551A, |
| | | | | V555A, S559D, Q578E, V581I, A589Q, |
| | | | | G592A, I594Q, V595I, N597T, S665N, |
| | | | | A667S, A670N, N712S, T722V |
| 2 | 736aa | BCD_0125 | 90.62% | D24N, D41H, A67E, A135G, P148Q, |
| | | | | 152delR, S153E, T158S, K163T, E179D, |
| | | | | G195A, G202N, A206S, S224N, R313K, |
| | | | | S315N, Q328T, I343V, V390L, E413Q, |
| | | | | S449V, 453delS, G455T, Q458G, Q462T, |
| | | | | L464A, A468G, A471S, N472T, S474A, |
| | | | | A475N, K478R, L481V, L495T, S496N, |
| | | | | T509A, Y511F, H512K, V520M, T528S, |
| | | | | E534D, M544I, G551N, R552K, N554G, |
| | | | | V555A, S559Q, M561L, L562I, S564D, |
| | | | | T570A, Q578E, V581S, D584I, L586N, |
| | | | | Q588N, N590S, G592Q, P593A, I594Q, |
| | | | | V595T, N597L, N599H, S600N, A603V, |
| | | | | L604I, T662L, S665N, Q666E, A670N, |
| | | | | T722V |
| 3 | 738aa | BCD_0126 | 97.29% | D24N, D41H, A67E, E134S, A135P, |
| | | | | A136V, E179D, S315N, T417Q, Q458A, |
| | | | | A471N, C485M, R552K, S559R, A589Q, |
| | | | | T591V, G592A, N597A, E720D, T722N |
| 4 | 736aa | BCD_0127 | 96.06% | D24N, D41N, K84Q, E134S, A135P, |
| | | | | A136V |
| | | | | E179D, 263delN, G264S, T265Q, 267delG, |
| | | | | S269A, T274H, Y275F, K312R, R313K, |
| | | | | S315R, T417Q, Q458T, Q462K, A471N, |
| | | | | L495T, S496N, R552K, Q578E, A589Q, |
| | | | | T591V, G592A, N597A, S665N |
| 5 | 736aa | BCD_0146 | 93.75% | D24N, D41T, K51T, K84Q, E134S, |
| | | | | A135P, A136V, E179D, 263delN, G264S, |
| | | | | T265Q, 267delG, S269A, T274H, Y275F, |
| | | | | K312R, |
| | | | | R313K, S315R, T417Q, Q458T, G459R, |
| | | | | Q462K, A471N, L495T, S496N, R552K, |
| | | | | Q578E, A589Q, T591V, G592A, N597A |
| | | | | N630H, D659N, T662A, T663E, Q666A, |
| | | | | A667T, L669F, I701V, Y708A, T711A, |
| | | | | A716T, N718D, T719N, E720N, T722L, |
| | | | | S724T |
| 6 | 738aa | BCD_0177 | 97.70% | K84Q, E134S, A135P, A136V, E179D, |
| | | | | S315N, T417Q, Q458T, A471N, L495T, |
| | | | | S496N, R552K, A589Q, T591A, G592A, |
| | | | | N597A, S665N |
| 7 | 736aa | BCD_0180 | 90.90% | D41N, A135G, P148Q, 152delR, S153E, |
| | | | | T158S, K163T, E179D, I189L, G195A, |
| | | | | S201P, G202N, A206S, S224N, S315N, |
| | | | | Q328T, I343V, I375V, V390L, E413Q, |
| | | | | S449V, 453delS, G455T, Q458G, Q462T |
| | | | | L464A, A471S, N472S, S474A, A475N, |
| | | | | K478R, L481V, C485S, L495T, S496N, |
| | | | | T509A, Y511F, H512K, V520M, T528S, |
| | | | | E533D, E534D, M544I, R552N, N554G, |
| | | | | S559Q, M561L, L562I, S564D, T570A, |
| | | | | Q578E, V581A, D584I, L586N, Q588A |
| | | | | G592Q, P593A, I594Q, V595T, N597L, |
| | | | | N599H, S600N, A603V, L604I, T662L, |
| | | | | S665N, A670N, N712S, T722V |
| 8 | 738aa | BCD_0181 | 98.10% | E134T, A135P, E179D, S315N, E413T, |
| | | | | I441V, Q458A, A471N, R552K, A589Q, |
| | | | | T591A, G592A, N597A, E720D |
| 9 | 736aa | BCD_0182 | 91.17% | E74Q, A135G, P148Q, 152delR, S153E, |
| | | | | T158S, K163T, E179D, I189L, G195A, |
| | | | | S201P, G202N, A206S, S224N, S315N, |
| | | | | Q328T, I343V, I375V, V390L, E413Q, |
| | | | | S449V, 453delS, G455T, Q458G, Q462T, |
| | | | | L464A, A471S, N472S, S474A, A475N, |
| | | | | K478R, L481V, L495T, S496N, T509A, |
| | | | | Y511F, H512K, V520M, T528S, E533D, |
| | | | | E534D, M544I, R552N, N554G, S559Q, |
| | | | | M561L, L562I, S564D, T570A, Q578E, |
| | | | | V581A, D584I, L586N, Q588A, G592Q, |
| | | | | P593A, I594Q, V595T, N597L, N599H, |
| | | | | S600N, A603V, L604I, T662L, S665N, |
| | | | | A670N, T722V |
| 10 | 736aa | BCD_0185 | 91.44% | A135G, P148Q, 152delR, S153E, T158S, |
| | | | | K163T, E179D, I189L, G195A, S201P, |
| | | | | G202N, A206S, S224N, S315N, Q328T, |
| | | | | I343V, I375V, V390L, E413Q, S449V, |
| | | | | 453delS, G455T, Q458G, Q462T, L464A, |
| | | | | A471S, N472S, S474A, A475N, K478R, |
| | | | | L481V, L495T, S496N, T509A, Y511F, |
| | | | | H512K, V520M, T528S, E534D, M544I, |
| | | | | R552N, N554G, S559Q, M561L, L562I, |
| | | | | S564D, T570A, Q578E, V581A, D584I, |
| | | | | L586N, Q588A, G592Q, P593A, I594Q, |
| | | | | V595T, N597L, N599H, S600N, A603V, |
| | | | | L604I, T662L, S665N, A670N, T722V |
| 11 | 738aa | BCD_0186 | 96.75% | A135G, K169R, T177I, E179D, I189L, |
| | | | | G195A, G198S, L199V, E413S, Q458A, |
| | | | | A471S, K478R, H529N, E534D, V542I, |
| | | | | R552K, S559N, A589Q, G592A, N597A, |
| | | | | T663A, S665N, A670N, T722V |
| 12 | 738aa | BCD_0187 | 96.88% | A135G, K169R, E179D, I189L, G195A, |
| | | | | G198S, L199V, E413S, Q458A, A471S, |
| | | | | K478R, H529N, E534D, V542I, R552K, |
| | | | | S559N, A589Q, G592A, N597A, T663A, |
| | | | | S665N, A670N, T722V |
| 13 | 734aa | BCD_0193 | 90.60% | D41H, A67E, V125I, A135G, P148Q, |
| | | | | 152delR, S153E, T158S, K163T, E179D, |
| | | | | G195A, G202N, A206S, S224N, R313K, |
| | | | | S315N, Q328T, I343V, I375V, V390L, |
| | | | | E413Q, S449V, 453delS, G455T, Q458G, |
| | | | | Q462T, L464A, A468G, A471S, N472T, |
| | | | | S474A, A475N, K478R, L481V, L495Q, |
| | | | | S496N, T509A, Y511F, H512K, V520M, |
| | | | | T528S, E534D, M544I, G551N, R552K, |
| | | | | N554G, V555A, S559Q, M561L, L562I, |
| | | | | S564D, T570A, Q578E, D584I, L586Q, |
| | | | | Q588N, G592Q, P593A, I594Q, V595T, |
| | | | | N597A, N599H, S600N, A603V, L604I, |
| | | | | T662L, S665T, Q666E, A670N, N712S, |
| | | | | T722V, 738delL |
| 14 | 736aa | BCD_0195 | 90.08% | D41H, A67E, Q105K, V125I, A135G, |
| | | | | P148Q, 152delR, S153E, T158S, K163T, |
| | | | | R171K, L172F, E179D, S180P, G195A, |
| | | | | G202N, A206S, S224N, R313K, S315N, |
| | | | | Q328T, I343V, I375V, V390L, E413Q, |
| | | | | S449V, 453delS, G455T, Q458G, Q462T, |
| | | | | L464A, A468G, A471S, N472T, S474A, |
| | | | | A475N, K478R, L481V, L495Q, S496N, |
| | | | | T509A, Y511F, H512K, V520M, T528S, |
| | | | | E534D, M544I, G551N, R552K, N554G, |
| | | | | V555A, S559Q, M561L, L562I, S564D, |
| | | | | T570A, Q578E, D584I, L586Q, Q588N, |
| | | | | G592Q, P593A, I594Q, V595T, N597A, |
| | | | | N599H, S600N, A603V, L604I, T662L, |
| | | | | S665T, Q666E, A670N, N712S, T722V |
| 15 | 738aa | BCD_0197 | 97.83% | E17K, D41N, E134S, A135P, A136V, |
| | | | | E179D, S315N, T417N, Q458A, A471N, |
| | | | | R552K, A589Q, T591A, G592A, N597A, |
| | | | | S665N |
| 16 | 731aa | BCD_0198 | 94.12% | D24A, K31Q, A67E, K77R, K84Q, |
| | | | | A135G, 148-153delPSPQRS, T158S, |
| | | | | K163T, E179D, S180A, I189L, G190S, |
| | | | | S224N, S225A, T270S, R313K, S315N, |
| | | | | I343V, E413T, 453delS, G455N, Q458N, |
| | | | | G459A, Q462T, S466A, A471Q, L495V, |
| | | | | A504T, R552K, D556E, S558Q, S559N, |
| | | | | A589T, T591S, G592A, N597A, T662V, |
| | | | | T663N, S665T, Q666D, T711A, T719A, |
| | | | | E720D, T722V |
| 17 | 736aa | BCD_0386 | 90.49% | A135G, P148Q, 152delR, S153E, T158S, |
| | | | | K163T, E179D, I189L, G195A, L199V, |
| | | | | G202N, A206S, S224N, G264S, |
| | | | | T270S, S315N, Q328D, E330N, T332V, |
| | | | | I343V, N385D, E413Q, Y447F, 453delS, |
| | | | | G455T, Q458A, Q462T, L464Q, A471S, |
| | | | | N472S, S474A, A475N, K478R, L481V, |
| | | | | L495V, S496T, N502E, T506P, V520M, |
| | | | | T528S, D531E, D532G, E534D, S539L, |
| | | | | M544I, A550T, Y557A, S558D, S559K, |
| | | | | L562I, S564N, Q578E, V581A, D584T, |
| | | | | L586H, Q588S, N590Q, T591A, G592Q, |
| | | | | P593A, I594Q, V595T, N597L, N599H, |
| | | | | S600N, A603V, S665N, A670N, T711N, |
| | | | | D714E, T722V, Y733F |
| 18 | 738aa | BCD_0448 | 91.19% | D41N, A135G, V146L, P148K, S149T, |
| | | | | 151delQ, R152N, S153Q, 155-157delDSS, |
| | | | | G159D, I160T, G161Q, K162A, K163A, |
| | | | | Q166T, L172P, N173Q, F174G, G175E, |
| | | | | T177S, E179D, E181A, S182R, V183R, |
| | | | | P184Q, 185-186insL, 186-187insFD, |
| | | | | I189A, 190-191insAPL, E191Q, G195A, |
| | | | | G198P, L199V, G202N, A206S, A210G, |
| | | | | N215D, E217Q, S224N, S225A, 263delN, |
| | | | | G264S, T265Q, 267delG, S269A, T274H, |
| | | | | Y275F, K312R, R313K, S315R, T417Q, |
| | | | | Q458T, Q462K, A471N, L495T, S496N, |
| | | | | R552K, Q578E, A589Q, T591V, G592A, |
| | | | | N597A, S665N |
| 19 | 738aa | BCD_0449 | 97.29% | D24N, D41H, A67E, E134S, A135P, |
| | | | | A136V, E179D, S315N, T417Q, Q458A, |
| | | | | A471N, C485M, R552K, S559R, A589Q, |
| | | | | T591V, G592A, N597A, E720D, T722N |
| 20 | 738aa | BCD_0450 | 97.29% | D24N, D41H, A67E, E134S, A135P, |
| | | | | A136V, E179D, S315N, T417Q, Q458A, |
| | | | | A471N, C485M, R552K, D556A, A589Q, |
| | | | | T591V, G592A, N597A, E720D, T722N |
| 21 | 738aa | BCD_0486 | 93.36% | D24A, D41N, K84Q, A135G, Q165P, |
| | | | | K169R, E179D, V183A, I189L, G195A, |
| | | | | L199V, G202N, S269A, S315N, E413Q, |
| | | | | S453T, Q458A, G459N, Q462T, L464G, |
| | | | | A468G, A471N, N472T, S474A, A475N, |
| | | | | L495T, S496G, G507A, A508G, D517N, |
| | | | | V520A, V524I, S540N, V542I, M544I, |
| | | | | G549N, G551A, V555A, S559D, |
| | | | | Q578E, V581I, A589Q, G592A, I594Q, |
| | | | | V595T, N597T, S665N, A670N, T722V |
| 22 | 738aa | BCD_0487 | 93.50% | D24A, K84Q, A135G, Q165P, K169R, |
| | | | | E179D, V183A, I189L, G195A, L199V, |
| | | | | G202N, S269A, S315N, E413Q, S453T, |
| | | | | Q458A, G459N, Q462T, L464G, A468G, |
| | | | | A471N, N472T, S474A, A475N, L495T, |
| | | | | S496G, G507A, A508G, D517N, V520A, |
| | | | | V524I, S540N, V542I, M544I, G549N, |
| | | | | G551A, V555A, S559D, Q578E, V581I, |
| | | | | A589Q, G592A, I594Q, V595T, N597T, |
| | | | | S665N, A670N, T722V |
| 23 | 738aa | BCD_0488 | 92.28% | D24A, D41N, K84Q, A135G, Q165P, |
| | | | | K169R, E179D, V183A, I189L, G195A, |
| | | | | L199V, G202N, S269A, S315N, E413Q, |
| | | | | S453T, Q458A, G459N, Q462T, L464G, |
| | | | | A468G, A471N, N472T, S474A, A475N, |
| | | | | L495T, S496G, G507A, A508G, D517N, |
| | | | | V520A, V524I, S540N, V542I, M544I, |
| | | | | G549N, G551A, V555A, S559D, Q578E, |
| | | | | V581I, A589Q, G592A, I594Q, V595T, |
| | | | | N597T, S665N, A670N, Y707F, Y708D, |
| | | | | T711N, N712S, A716S, N718D, T719A, |
| | | | | E720Q, T722V |
| 24 | 737aa | BMN_0208 | 95.52% | D24A, K84Q, A135G, P148Q, 152delR, |
| | | | | S153E, T158S, E179D, I189L, G195A, |
| | | | | G198S, S201L, G202N, W235R, R313K, |
| | | | | P354S, E413S, Q458A, A471N, C485M, |
| | | | | A508G, H529N, E534D, R552K, A589Q, |
| | | | | G592A, N597A, G602E, T663A, S665N, |
| | | | | A670N, N718D, T722V, P726H |
| 25 | 737aa | BMN_0209 | 95.93% | D24A, K84Q, A135G, P148Q, 152delR, |
| | | | | S153E, T158S, E179D, I189L, G195A, |
| | | | | G198S, S201L, G202N, R313K, L357P, |
| | | | | E413S, Q458A, A471N, C485M, A508G, |
| | | | | H529N, E534D, R552K, A589Q, G592A, |
| | | | | N597A, L616P, T663A, S665N, A670N, |
| | | | | T722V |
| 26 | 737aa | BMN_0210 | 95.79% | D24A, K84Q, L129P, A135G, P148Q, |
| | | | | 152delR, S153E, T158S, E179D, I189L, |
| | | | | G195A, G198S, S201L, G202N, R313K, |
| | | | | S394F, E413S, Q458A, A471N, C485M, |
| | | | | A508G, H529N, E534D, R552K, A589Q, |
| | | | | G592A, N597A, T663A, S665N, A670N, |
| | | | | W687R, T722V |
| 27 | 737aa | BMN_0211 | 95.66% | D24A, K84Q, A135G, K143E, P148Q, |
| | | | | 152delR, S153E, T158S, E179D, I189L, |
| | | | | G195A, G198S, S201L, G202N, R313K, |
| | | | | E413S, Q458A, A471N, C485M, A508G, |
| | | | | H529N, E534D, G541A, R552K, A575T, |
| | | | | A589Q, G592A, N597A, T663A, S665N, |
| | | | | A670N, G721S, T722V |
| 28 | 737aa | BMN_0212 | 95.79% | D24A, L59P, K84Q, A135G, P148Q, |
| | | | | 152delR, S153E, T158S, E179D, I189L, |
| | | | | G195A, G198S, S201L, G202N, F290S, |
| | | | | R313K, E413S, Q458A, A471N, C485M, |
| | | | | A508G, H529N, E534D, R552K, K569R, |
| | | | | A589Q, G592A, N597A, T663A, S665N, |
| | | | | A670N, T722V |
| 29 | 737aa | BMN_0213 | 95.93% | D24A, K84Q, A135G, P148Q, 152delR, |
| | | | | S153E, T158S, E179D, I189L, G195A, |
| | | | | G198S, S201L, G202N, R313K, E413S, |
| | | | | Q458A, A471N, C485M, A508G, H529N, |
| | | | | E534D, F536L, R552K, I568V, A589Q, |
| | | | | G592A, N597A, T663A, S665N, A670N, |
| | | | | T722V |
| 30 | 737aa | BMN_0214 | 95.79% | D24A, K84Q, K123R, A135G, P148Q, |
| | | | | 152delR, S153E, T158S, E179D, I189L, |
| | | | | G195A, G198S, S201L, G202N, R313K, |
| | | | | H361R, E413S, Q458A, A471N, C485M, |
| | | | | A508G, H529N, E534D, R552K, A589Q, |
| | | | | G592A, N597A, T663A, S665N, A670N, |
| | | | | N718D, T722V |
| 31 | 737aa | BMN_0215 | 96.07% | D24A, K84Q, A135G, P148Q, 152delR, |
| | | | | S153E, T158S, E179D, I189L, G195A, |
| | | | | G198S, S201L, G202N, R313K, N320D, |
| | | | | E413S, Q458A, A471N, C485M, A508G, |
| | | | | H529N, E534D, R552K, A589Q, G592A, |
| | | | | N597A, T663A, S665N, A670N, T722V |
| 32 | 737aa | BMN_0216 | 95.93% | D24A, K84Q, A135G, P148Q, 152delR, |
| | | | | S153E, T158S, E179D, I189L, G195A, |
| | | | | G198S, S201L, G202N, R313K, E413S, |
| | | | | Q458A, A471N, C485M, A508G, H529N, |
| | | | | E534D, G541A, R552K, A589Q, G592A, |
| | | | | N597A, T663A, S665N, A670N, G721S, |
| | | | | T722V |
| 33 | 737aa | BMN_0217 | 96.07% | D24A, K84Q, A135G, P148Q, 152delR, |
| | | | | S153E, T158S, E179D I189L, G195A, |
| | | | | G198S, S201L, G202N, R313K, E413S, |
| | | | | Q458A, A471N, C485M, A508G, N521Y, |
| | | | | H529N, E534D, R552K, A589Q, G592A, |
| | | | | N597A, T663A, S665N, A670N, T722V |
| 34 | 737aa | BMN_0218 | 95.12% | D24A, E63G, K84Q, Q101R, A135G, |
| | | | | P148Q, 152delR, S153E, T158S, E179D, |
| | | | | I189L, G195A, G198S, S201L, G202N, |
| | | | | N273S, F290S, R313K, S387G, E413S, |
| | | | | T417I, Q458A, A471N, C485M, A508G, |
| | | | | H529N, E534D, R552K, A589Q, G592A, |
| | | | | N597A, T663A, S665N, A670N, Q690L, |
| | | | | N693S, T722V |
| 35 | 736aa | BMN_0302 | 90.35% | L53P, L126F, A135G, P148Q, 152delR, |
| | | | | S153E, T158S, K163T, E179D, I189L, |
| | | | | G195A, S201P, G202N, A206S, S224N, |
| | | | | G264S, S315N, Q328T, I343V, I375V, |
| | | | | V390L, E413Q, S449V, 453delS, G455T, |
| | | | | Q458G, Q462T, L464A, A471S, N472S, |
| | | | | S474A, A475N, K478R, L481V, C485S, |
| | | | | L495K, S496N, T509A, Y511F, H512K, |
| | | | | D517N, V520M, T528S, E534D, M544I, |
| | | | | R552N, N554G, S559Q, M561L, L562I, |
| | | | | S564D, T570A, Q578E, V581E, D584I, |
| | | | | L586D, Q588A, G592Q, P593A, I594Q, |
| | | | | V595T, N597L, N599H, S600N, A603V, |
| | | | | L604I, K643M, T662L, S665N, A670N, |
| | | | | T719S, E720D, T722V |
| 36 | 736aa | BMN_0303 | 90.22% | E102G, A135G, P148Q, 152delR, S153E, |
| | | | | T158S, K163T, E179D, I189L, G195A, |
| | | | | S201P, G202N, A206S, S224N, G264S, |
| | | | | L314P, S315N, Q328T, I343V, V345A, |
| | | | | I375V, V390L, E413Q, S449V, 453delS, |
| | | | | G455T, Q458G, Q462T, L464A, A471S, |
| | | | | N472S, S474A, A475N, K478R, L481V, |
| | | | | C485S, L495K, S496N, T509A, Y511F, |
| | | | | H512K, D517N, V520M, T528S, E534D, |
| | | | | F537S, M544I, R552N, N554G, S559Q, |
| | | | | M561L, L562I, S564D, T570A, Q578E, |
| | | | | V581E, D584I, L586D, Q588A, G592Q, |
| | | | | P593A, I594Q, V595T, N597L, N599H, |
| | | | | S600N, A603V, L604I, T662L, S665N, |
| | | | | A670N, T719S, E720D, T722V |
| 37 | 736aa | BMN_0304 | 90.49% | E102G, A135G, P148Q, 152delR, S153E, |
| | | | | T158S, K163T, E179D, I189L, G195A, |
| | | | | S201P, G202N, A206S, S224N, G264S, |
| | | | | S315N, Q328T, I343V, I375V, V390L, |
| | | | | E413Q, S449V, 453delS, G455T, Q458G, |
| | | | | Q462I, L464A, A471S, N472S, S474A, |
| | | | | A475N, K478R, L481V, C485S, L495K, |
| | | | | S496N, T509A, Y511F, H512K, D517N, |
| | | | | V520M, T528S, E534D, M544I, R552N, |
| | | | | N554G, V555M, S559Q, M561L, L562I, |
| | | | | S564D, T570A, Q578E, V581E, D584I, |
| | | | | L586D, Q588A, G592Q, P593A, I594Q, |
| | | | | V595T, N597L, N599H, S600N, A603V, |
| | | | | L604I, T662L, S665N, A670N, T719S, |
| | | | | E720D, T722V |
| 38 | 736aa | BMN_0305 | 90.35% | A135G, P148Q, 152delR, S153E, T158S |
| | | | | K163T, E179D, I189L, G195A, S201P, |
| | | | | G202N, A206S, S224N, G264S, S269G, |
| | | | | S315N, Q328T, I343V, I375V, Y380H, |
| | | | | V390L, E413Q, S449V, 453delS, G455T |
| | | | | Q458G, Q462T, L464A, A471S, N472S, |
| | | | | S474A, A475N, K478R, L481V, C485S, |
| | | | | L495K, S496D, T509A, Y511F, H512K, |
| | | | | D517N, V520M, T528S, E534D, M544I, |
| | | | | R552N, N554G, S559Q, M561L, L562I, |
| | | | | S564D, T570A, Q578E, V581E, D584V, |
| | | | | L586D, Q588A, G592Q, P593A, I594Q, |
| | | | | V595T, N597L, N599H, S600N, A603V, |
| | | | | L604I, T662L, F664L, S665N, A670N, |
| | | | | T719S, E720D, T722V |
| 39 | 736aa | BMN_0306 | 90.76% | A135G, P148Q, 152delR, S153E, T158S, |
| | | | | K163T, E179D, I189L, G195A, S201P, |
| | | | | G202N, A206S, S224N, G264S, S269G, |
| | | | | S315N, Q328T, I343V, I375V, Y380H, |
| | | | | V390L, E413Q, S449V, 453delS, G455T, |
| | | | | Q458G, Q462T, L464A, A471S, N472S, |
| | | | | S474A, A475N, K478R, L481V, C485S, |
| | | | | L495K, S496D, T509A, Y511F, H512K, |
| | | | | D517N, V520M, T528S, E534D, M544I, |
| | | | | R552N, N554G, S559Q, M561L, L562I, |
| | | | | S564D, T570A, Q578E, V581E, D584V, |
| | | | | L586D, Q588A, G592Q, P593A, I594Q, |
| | | | | V595T, N597L, N599H, S600N, A603V, |
| | | | | L604I, T662L, F664L, S665N, A670N, |
| | | | | T719S, E720D, T722V |
| 40 | 736aa | BMN_0307 | 90.49% | V118I, A135G, P148Q, 152delR, S153E, |
| | | | | T158S, K163T, E179D, I189L, G195A, |
| | | | | S201P, G202N, A206S, S224N, S225P, |
| | | | | W235R, S315N, Q328T, I343V, I375V, |
| | | | | V390L, G409S, E413Q, S449V, 453delS, |
| | | | | G455T, Q458G, Q462T, L464A, A471S, |
| | | | | N472S, S474A, A475N, K478R, L481V, |
| | | | | L495T, S496N, T509A, Y511F, H512K, |
| | | | | V520M, T528S, E533D, E534D, M544I, |
| | | | | A550T, R552N, N554G, S559Q, M561L, |
| | | | | L562I, S564D, T570A, Q578E, V581A, |
| | | | | D584I, L586N, Q588A, G592Q, P593A, |
| | | | | I594Q, V595T, N597L, N599H, S600N, |
| | | | | A603V, L604I, H644L, T662L, S665N, |
| | | | | A670N, T722V |
| 41 | 736aa | BMN_0308 | 90.90% | V118I, A135G, P148Q, 152delR, S153E, |
| | | | | S157P, T158S, K163T, E179D, I189L, |
| | | | | G195A, S201P, G202N, A206S, S224N, |
| | | | | S315N, Q328T, I343V, I375V, V390L, |
| | | | | E413Q, S449V, 453delS, G455T, Q458G, |
| | | | | Q462T, L464A, A471S, N472S, S474A, |
| | | | | A475N, K478R, L481V, L495T, S496N, |
| | | | | T509A, Y511F, H512K, V520M, T528S, |
| | | | | E533D, E534D, M544I, A550T, R552N, |
| | | | | N554G, S559Q, M561L, L562I, S564D, |
| | | | | T570A, Q578E, V581A, D584I, L586N, |
| | | | | Q588A, G592Q, P593A, I594Q, V595T, |
| | | | | N597L, N599H, S600N, A603V, L604I, |
| | | | | T662L, S665N, A670N, T722V |
| 42 | 736aa | BMN_0309 | 90.49% | V118I, K123R, A135G, P148Q, 152delR |
| | | | | S153E, T158S, K163T, E179D, I189L, |
| | | | | G195A, S201P, G202N, A206S, S224N, |
| | | | | S315N, Q328T, I343V, I375V, V390L, |
| | | | | G409S, E413Q, Y427C, S449V, 453delS, |
| | | | | G455T, Q458G, Q462T, L464A, A471G, |
| | | | | N472S, S474A, A475N K478R, L481V, |
| | | | | L495T, S496N, T509A, Y511F, H512K, |
| | | | | V520M, T528S, E533D, E534D, M544I, |
| | | | | A550T, R552N, N554G, S559Q, M561L, |
| | | | | L562I, S564D, T570A, Q578E, V581A, |
| | | | | D584I, L586N, Q588A, G592Q, P593A, |
| | | | | I594Q, V595T, N597L, N599H, S600N, |
| | | | | A603V, L604I, S634P, T662L, S665N, |
| | | | | A670N, T722V |
| 43 | 736aa | BMN_0310 | 90.35% | L47F, V118I, A135G, P148Q, 152delR, |
| | | | | S153E, T158S, K163T, Q165R, E179D, |
| | | | | I189L, G195A, S201P, G202N, A206S, |
| | | | | S224N, S266P, T280A, S315N, Q328T, |
| | | | | I343V, I375V, V390L, E413Q, S449V, |
| | | | | 453delS, G455T, Q458G, Q462T, L464A, |
| | | | | A471S, N472S, S474A, A475N, K478R, |
| | | | | L481V, L495T, S496N, T509A, Y511F, |
| | | | | H512K, V520M, T528S, E533D, E534D, |
| | | | | M544I, A550T, R552N, N554G, S559Q, |
| | | | | M561L, L562I, S564D, T570A, Q578E, |
| | | | | V581A, D584I, L586N, Q588A, G592Q, |
| | | | | P593A, I594Q, V595T, N597L, N599H, |
| | | | | S600N, A603V, L604I, L616Q, T662L, |
| | | | | S665N, A670N, T722V |
| 44 | 736aa | BMN_0311 | 90.62% | V118I, A135G, P148Q, 152delR, S153E, |
| | | | | T158S, K163T, F174S, E179D, I189L, |
| | | | | G195A, S201P, G202N, A206S, S224N |
| | | | | S315N, Q328T, I343V, H361Y, I375V, |
| | | | | V390L, E413Q, F414S, S449V, 453delS, |
| | | | | G455T, Q458G, Q462T, L464A, A471S, |
| | | | | N472S, S474, A475N, K478R, L481V, |
| | | | | L495T, S496N, T509A, Y511F, H512K, |
| | | | | V520M, T528S, E533D, E534D, M544I, |
| | | | | A550T, R552N, N554G, S559Q, M561L, |
| | | | | L562I, S564D, T570A, Q578E, V581A, |
| | | | | D584I, L586N, Q588A, G592Q, P593A, |
| | | | | I594Q, V595T, N597L, N599H, S600N, |
| | | | | A603V, L604I, T662L, S665N, A670N, |
| | | | | T722V |
| 45 | 736aa | BMN_0312 | 90.76% | P30S, A135G, A139V, P148Q, 152delR, |
| | | | | S153E, T158S, K163T, E179D, I189L, |
| | | | | G195A, S201P, G202N, A206S, S224N, |
| | | | | S315N, Q328T, I343V, H361Y, I375V, |
| | | | | V390L, E413Q, S449V, 453delS, G455T, |
| | | | | Q458G, Q462T, L464A, A471S, N472S, |
| | | | | S474A, A475N, K478R, L481V, L495T, |
| | | | | S496N, T509A, Y511F, H512K, V520M, |
| | | | | T528S, E533D, E534D, M544I, A550T, |
| | | | | R552N, N554G, S559Q, M561L, L562I, |
| | | | | S564D, T570A, Q578E, V581A, D584I, |
| | | | | L586N, Q588A, G592Q, P593A, I594Q, |
| | | | | V595T, N597L, N599H S600N, A603V, |
| | | | | L604I, T662L, S665N, A670N, T722V |
| 46 | 736aa | BMN_0313 | 90.76% | P30S, A135G, A139V, P148Q, 152delR, |
| | | | | S153E, T158S, K163T, E179D, I189L, |
| | | | | G195A, S201P, G202N, A206S, S224N, |
| | | | | S315N, Q328T, I343V, H361Y, I375V, |
| | | | | V390L, E413Q, S449V, 453delS, G455T, |
| | | | | Q458G, Q462T, L464A, A471S, N472S, |
| | | | | S474A, A475N, K478R, L481V, L495T, |
| | | | | S496N, T509A, Y511F, H512K, V520M, |
| | | | | T528S, E533D, E534D, M544I, A550T, |
| | | | | R552N, N554G, S559Q, M561L, L562I, |
| | | | | S564D, T570A, Q578E, V581A, D584I, |
| | | | | L586N, Q588A, G592Q, P593A, I594Q, |
| | | | | V595T, N597L, N599H, S600N, A603V, |
| | | | | L604I, T662L, S665N, A670N, T722V |
| 47 | 736aa | BMN_0314 | 90.62% | A135G, P148Q, 152delR, S153E, T158S, |
| | | | | K163T, E179D, S180T, I189L, G190R, |
| | | | | G195A, S201P, G202N, A206S, S224N, |
| | | | | S315N, L318P, Q328T, L339P, I343V, |
| | | | | H361R, I375V, V390L, N410D, E413Q, |
| | | | | S449V, 453delS, G455T, Q458G, Q462T, |
| | | | | L464A, A471S, N472S, S474A, A475N, |
| | | | | K478R, L481V, L495T, S496N, T509A, |
| | | | | Y511F, H512K, V520M, T528S, E534D, |
| | | | | M544I, R552N, N554G, S559Q, M561L, |
| | | | | L562I, S564D, T570A, Q578E, V581A, |
| | | | | D584I, L586N, Q588A, G592L, P593A, |
| | | | | I594Q, V595T, N597L, N599H, S600N, |
| | | | | A603V, L604I, T662L, S665N, A670N, |
| | | | | T722V |
| 48 | 736aa | BMN_0315 | 91.03% | A135G, K137N, P148Q, 152delR, S153E, |
| | | | | T158S, K163T, E179D, I189L, G190R, |
| | | | | G195A, S201P, G202N, A206S, S224N, |
| | | | | N305S, S315N, Q328T, I343V, I375V, |
| | | | | V390L, E413Q, S449V, 453delS, G455T, |
| | | | | Q458G, Q462T, L464A, A471S, N472S, |
| | | | | S474A, A475N, K478R, L481V, L495T, |
| | | | | S496N, T509A, Y511F, H512K, V520M, |
| | | | | T528S, E534D, M544I, R552N, N554G, |
| | | | | S559Q, M561L, L562I, S564D, T570A, |
| | | | | Q578E, V581A, D584I, L586N, Q588A, |
| | | | | G592Q, P593A, I594Q, V595T, N597L, |
| | | | | N599H, S600N, A603V, L604I, T662L, |
| | | | | S665N, A670N, T722V |

TABLE 2.8
Branch 2 - Clade 4

| | | | % identity to | |
|---|---|---|---|---|
| | AA | | representative | Varying amino acid residues relative to the |
| No. | Length | Name | sequence | representative sequence |
| 0 | 728aa | BCD_0360 | * | |
| | | (SEQ ID | | |
| | | NO: 91) | | |
| 1 | 726aa | AAV_po.6 | 93.94% | 263-264delTG, G265S, E326Q, N327T, |
| | | | | E328D, I373V, P460H, S461N, S485P, |
| | | | | E487Q, N490T, E492D, S494A, A498T, |
| | | | | K500R, D507N, V509A, D524E, Q538E, |
| | | | | G539D, S540A, N541E, A542K, S543N, |
| | | | | V545A, Q546V, L551I, E567G, F571L, |
| | | | | S575N, L576F, 579delG, 584-585insA, |
| | | | | T586E, R587M, Y616H, G648A, P651A, |
| | | | | Q656P, Q657A, D704E, N708D, S709N, |
| | | | | D710N |
| 2 | 726aa | BCD_0106 | 95.45% | T178A, 263-264delTG, G265S, N268S, |
| | | | | Q386H, S461N, K483T, T484V, S485P, |
| | | | | E487Q, G491A, D507N, V509A, S518T, |
| | | | | D524E, S540A, N541D, A542K, S543T, |
| | | | | V545A, Q546V, D548E, L551I, I552V, |
| | | | | T560A, F571V, S575N, T582Q, D584T, |
| | | | | T586N, R587D, N655S, Q657N, S709G |
| 3 | 726aa | BCD_0351 | 95.32% | T178A, 263-264delTG, G265S, N268S, |
| | | | | E326Q, N327T, E328D, I373V, S461T, |
| | | | | S485P, E487Q, N490T, E492D, S494A, |
| | | | | A498T, K500R, S540A, N541E, A542K, |
| | | | | S543N, V545A, Q546V, L551I, F571L, |
| | | | | G579N, N580T, T586E, G648A, P651A, |
| | | | | Q656P, Q657A, N708D, S709T, D710N, |
| | | | | V712M |
| 4 | 726aa | BCD_0353 | 95.04% | S206G, 263-264delTG, G265S, N268S, |
| | | | | E326Q, N327T, E328D, I373V, S485P, |
| | | | | E487Q, N490T, E492D, S494A, A498T, |
| | | | | K500R, Q538K, G539D, S540A, N541E, |
| | | | | A542K, S543N, V545A, Q546V, L551I, |
| | | | | F571L, 579delG, 584-585insA, T586E, |
| | | | | G648A, P651A, Q656P, Q657A, D704E, |
| | | | | N708D, S709N, D710N |
| 5 | 727aa | BCD_0354 | 92.98% | T178A, 262delQ, T263S, G264S, G265F, |
| | | | | N268T, N271D, E326D, N327T, E328D, |
| | | | | Q450N, N451G, G454A, P460H, S461T, |
| | | | | S462N, D465N, S485P, G491Q, D524N, |
| | | | | K537T, Q538P, G539N, S540A, N541A, |
| | | | | S543T, V545A, D548G, K549D, F571I, |
| | | | | T574N, S575N, L576K, N578S, G579S, |
| | | | | N580S, T581I, T582Q, D584T, R587N, |
| | | | | Y616H, R634H, G648S, D649N, T652E, |
| | | | | N655K, Q657E, A660N, S661A, N708D, |
| | | | | S709T, D710N |
| 6 | 727aa | BCD_0359 | 93.54% | I117V, T178A, A182P, 262delQ, T263S, |
| | | | | G264S, G265F, N268T, E328D, I373V, |
| | | | | Q450A, N451G, G454A, S461T, S485P, |
| | | | | G491Q, D524N, K537T, Q538P, G539N, |
| | | | | S540A, S543T, V545A, D548N, K549D, |
| | | | | F571I, T574N, S575N, L576K, N578S, |
| | | | | G579S, N580S, T581I, T582Q, D584T, |
| | | | | R587N, Y616H, R634H, G648S, D649N, |
| | | | | T652E, N655K, Q657D, A660N, S661A, |
| | | | | N708D, S709T, D710N |
| 7 | 727aa | BCD_0361 | 92.71% | I117V, T178A, A182P, 262delQ, T263S, |
| | | | | G264N, G265Y, N327T, E328D, Q450A, |
| | | | | N451G, G454A, S461T, D465N, I481V, |
| | | | | K483E, T484R, E492D, A498T, K500R, |
| | | | | V509A, K537T, Q538P, G539N, S540A, |
| | | | | N541T, V545A, Q546P, K549N, F571I, |
| | | | | T574N, S575N, L576K, N578D, G579N, |
| | | | | N580S, T582Q, D584T, T586A, R587I, |
| | | | | Y616H, R634H, G648S, D649N, T652E, |
| | | | | T653K, N655T, Q657E, A660N, S661A, |
| | | | | N708D, S709T, D710N, V712M |
| 8 | 727aa | BCD_0362 | 94.50% | 262delQ, T263S, G264Q, G265A, N268S, |
| | | | | E326D, N327T, Q450N, N451G, K483T, |
| | | | | T484V, S485P, E487Q, E492D, D507N, |
| | | | | V509A, D524H, S540A, N541E, A542K, |
| | | | | S543N, V545A, Q546I, L551I, I558V, |
| | | | | N578S, G579S, N580T, T581E, T582A, |
| | | | | D584E, T586A, Y616H, G648S, T652L, |
| | | | | Q656P, Q657A, D704E, N708D, S709N, |
| | | | | D710N |
| 9 | 727aa | BCD_0389 | 93.12% | E62D, P134L, T178A, A182P, 262delQ, |
| | | | | T263S, G264N, G265Y, N327T, Q386H, |
| | | | | S461T, D465N, I481V, K483E, T484R, |
| | | | | E492D, D507N, V509A, K537T, Q538P, |
| | | | | G539N, S540A, N541T, V545T, Q546P, |
| | | | | D548E, K549N, F571I, T574N, S575N, |
| | | | | L576K, N578D, N580S, T582Q, D584T, |
| | | | | T586A, R587I, Y616H, R634H, G648S, |
| | | | | D649N, T652E, T653K, N655T, Q657E, |
| | | | | A660N, S661A, N708D, S709T, D710N, |
| | | | | V712M |
| 10 | 727aa | BCD_0390 | 93.26% | S206G, 262delQ, T263S, G264S, G265F, |
| | | | | N268T, N271D, E326D, N327T, E328D, |
| | | | | Q450N, N451G, G454A, P460H, S461T, |
| | | | | S462N, D465N, S485P, G491Q, D524N, |
| | | | | K537T, Q538P, G539N, S540A, S543T, |
| | | | | V545A, D548N, K549D, F571I, T574N, |
| | | | | S575N, L576K, N578S, G579S, N580S, |
| | | | | T582Q, D584T, R587N, Y616H, R634H, |
| | | | | G648S, D649N, T652E, N655K, Q657E, |
| | | | | A660N, S661A, N708D, S709T, D710N |
| 11 | 728aa | BCD_0391 | 95.33% | A150V, T178A, S206G, Q262S, T263S, |
| | | | | G265S, N268T, N327T, E328D, I373V, |
| | | | | G454A, D465N, D524H, S540A, N541D, |
| | | | | A542K, S543T, V545A, Q546I, L551I, |
| | | | | I552V, T560A, S575T, N580Q, T582A, |
| | | | | R634H, G648S, D649N, T652D, Q656P, |
| | | | | Q657E, A660N, N698D, S709G |
| 12 | 727aa | BCD_0392 | 93.26% | S206G, 262delQ, T263S, G264N, G265Y, |
| | | | | N327T, E328D, Q386H, S461T, D465N, |
| | | | | I481V, K483E, T484R, E492D, A498T, |
| | | | | V509A, K537T, Q538P, G539N, S540A, |
| | | | | S543T, V545A, Q546P, D548E, K549N, |
| | | | | F571I, T574N, S575N, L576K, N578D, |
| | | | | G579S, N580S, T582Q, D584T, T586A, |
| | | | | R587I, Y616H, R634H, G648S, D649N, |
| | | | | T652E, T653K, N655T, Q657E, A660N, |
| | | | | S661A, N708D, S709T, D710N, V712M |
| 13 | 727aa | BCD_0393 | 93.81% | 262delQ, T263S, G264Q, G265A, E326D, |
| | | | | N327T, E328D, I373V, Q450N, N451G, |
| | | | | S461N, K483T, T484V, S485P, E487Q, |
| | | | | E492D, T496A, D507N, V509A, D524H, |
| | | | | S540A, N541E, A542K, S543N, V545A, |
| | | | | Q546I, L551I, N578S, G579P, N580T, |
| | | | | T581E, T582A, D584E, T586A, Y616H, |
| | | | | R634H, G648S, D649N, T652E, N655T, |
| | | | | Q657E, A660N, S661A, N708D, S709T, |
| | | | | D710N |
| 14 | 727aa | BCD_0394 | 93.67% | S206G, 262delQ, T263S, G264Q, G265A, |
| | | | | E326D, N327T, E328D, I373V, Q450N, |
| | | | | N451G, S461N, K483T, T484V, S485P, |
| | | | | E487Q, E492D, T496A, D507N, V509A, |
| | | | | D524H, S540A, N541E, A542K, S543N, |
| | | | | V545A, Q546I, L551I, N578S, G579P, |
| | | | | N580T, T581E, T582A, D584E, T586A, |
| | | | | Y616H, R634H, G648S, D649N, T652E, |
| | | | | N655T, Q657E, A660N, S661A, N708D, |
| | | | | S709T, D710N |
| 15 | 727aa | BCD_0395 | 93.54% | T178A, 262delQ, T263S, G264Q, G265A, |
| | | | | E326D, N327T, E328D, I373V, Q450N, |
| | | | | N451G, S461N, K483T, T484V, S485P, |
| | | | | E487Q, E492D, T496A, D507N, V509A, |
| | | | | D524H, S540A, N541E, A542K, S543N, |
| | | | | V545A, Q546I, L551I, A573S, N578S, |
| | | | | G579P, N580T, T581E, T582A, D584E, |
| | | | | T586A, Y616H, R634H, G648S, D649N, |
| | | | | T652E, N655T, Q657E, A660N, S661A, |
| | | | | N708D, S709T, D710N |
| 16 | 727aa | BCD_0396 | 93.95% | S206G, 262delQ, T263S, G264S, G265F, |
| | | | | N268T, E328D, I373V, Q450A, N451G, |
| | | | | G454A, S461T, S485P, G491Q, D524N, |
| | | | | K537T, Q538P, G539N, S540A, S543T, |
| | | | | V545A, D548N, K549D, F571I, T574N, |
| | | | | S575N, L576K, N578S, G579S, N580S, |
| | | | | T582Q, D584T, R587N, Y616H, R634H, |
| | | | | G648S, D649N, T652E, N655K, Q657E, |
| | | | | A660N, S661A, N708D, S709T, D710N |
| 17 | 727aa | BCD_0413 | 93.54% | A181V, 262delQ, T263G, G264N, G265Y, |
| | | | | E326D, N327T, E328D, Q450N, N451G, |
| | | | | G454A, P460H, S461T, S462N, D465N, |
| | | | | S485P, G491Q, D524N, K537T, Q538S, |
| | | | | G539N, S540A, S543T, V545A, D548N, |
| | | | | K549D, F571I, T574N, S575N, L576K, |
| | | | | N578S, G579S, N580S, T582Q, D584T, |
| | | | | R587N, Y616H, R634H, G648S, D649N, |
| | | | | T652E, Q656P, Q657A, A660N, S661A, |
| | | | | N708D, S709T, D710N |
| 18 | 726aa | BCD_0414 | 95.18% | A133E, S206G, 263-264delTG, G265S, |
| | | | | E326Q, N327T, E328D, I373V, S485P, |
| | | | | E487Q, N490T, E492D, S494A, A498T, |
| | | | | K500R, Q538K, G539D, S540A, N541E, |
| | | | | A542K, S543N, V545A, Q546V, L551I, |
| | | | | F571L, G579N, N580T, T586E, G648A, |
| | | | | P651A, Q656P, Q657A, D704E, N708D, |
| | | | | S709N, D710N |
| 19 | 726aa | BCD_0415 | 95.18% | S206G, 263-264delTG, G265S, N268T, |
| | | | | N327T, G454A, S461N, N469K, E487Q, |
| | | | | G491N, S494A, D507N, V509A, S518T, |
| | | | | D524H, S540A, N541E, A542K, S543N, |
| | | | | V545A, Q546I, L551I, T560A, A565S, |
| | | | | F571V, S575N, T582Q, D584T, T586N, |
| | | | | R587D, Y616H, R634H, G648S, T652L, |
| | | | | N698D, S709G |
| 20 | 726aa | BCD_0443 | 94.35% | I145V, S153G, K156S, Q163H, T178A, |
| | | | | S206G, 263-264delTG, G265S, N268S, |
| | | | | E326Q, N327T, E328D, I373V, S462N, |
| | | | | S485P, E487Q, N490T, E492D, S494A, |
| | | | | A498T, K500R, G539D, S540A, N541E, |
| | | | | A542K, S543N, V545A, Q546V, L551I, |
| | | | | F571L, 579delG, 584-585insA, T586E, |
| | | | | G648A, P651A, Q656P, Q657A, N708D, |
| | | | | S709T, D710N, V712M |
| 21 | 726aa | BCD_0459 | 94.77% | A69E, S206G, 263-264delTG, G265S, |
| | | | | N268S, E326Q, N327T, E328D, I373V, |
| | | | | Q386H, S461T, S485P, E487Q, N490T, |
| | | | | E492D, S494A, A498T, K500R, G539D, |
| | | | | S540A, N541E, A542K, S543N, V545A, |
| | | | | Q546V, L551I, F571L, 579delG, 584- |
| | | | | 585insA, T586E, G648A, P651A, Q656P, |
| | | | | Q657A, N708D, S709T, D710N, V712M |
| 22 | 727aa | BCD_0461 | 92.71% | A69E, L113I, S206G, 262delQ, T263S, |
| | | | | G264N, G265Y, N327T, Q450A, N451G, |
| | | | | G454A, S461T, S462T, D465N, I481V, |
| | | | | K483E, T484R, E492D, A498T, K500R, |
| | | | | V509A, K537T, Q538P, G539N, S540A, |
| | | | | N541T, V545A, Q546P, K549N, F571I, |
| | | | | T574N, S575N, L576K, N578D, G579S, |
| | | | | N580S, T582Q, D584T, T586A, R587I, |
| | | | | Y616H, R634H, G648S, D649N, T652E, |
| | | | | T653K, N655T, Q657E, A660N, S661A, |
| | | | | N708D, S709T, D710N, V712M |
| 23 | 727aa | BCD_0462 | 93.12% | A69E, S206G, 262delQ, T263S, G264S, |
| | | | | G265F, N268T, N271D, E326D, N327T, |
| | | | | E328D, Q450N, N451G, G454A, P460H, |
| | | | | S461T, S462N, D465N, S485P, G491Q, |
| | | | | D524N, K537T, Q538P, G539N, S540A, |
| | | | | S543T, V545A, D548N, K549D, F571I, |
| | | | | T574N, S575N, L576K, N578S, G579S, |
| | | | | N580S, T582Q, D584T, R587N, Y616H, |
| | | | | R634H, G648S, D649N, T652E, N655K, |
| | | | | Q657E, A660N, S661A, N708D, S709T, |
| | | | | D710N |
| 24 | 726aa | BCD_0466 | 94.77% | A69E, S206G, 263-264delTG, G265S, |
| | | | | I373V, Q386H, G454A, S461N, E487Q, |
| | | | | G491N, S494A, D507N, V509A, D524H, |
| | | | | S540A, N541D, A542K, S543T, V545A, |
| | | | | Q546I, L551I, I552V, T560A, F571V, |
| | | | | S575N, T582Q, D584T, T586N, R587D, |
| | | | | Y616H, R634H, G648S, D649N, T652D, |
| | | | | Q656P, Q657E, A660N, N698D, S709G |
| 25 | 727aa | BCD_0467 | 93.12% | A69E, S206G, 262delQ, T263S, G264S, |
| | | | | G265F, N268T, N271D, I373V, Q386H, |
| | | | | Q450N, N451G, G454A, P460H, S461T, |
| | | | | S462N, D465N, S485P, G491Q, K537T, |
| | | | | G539N, S540A, N541A, S543T, V545A, |
| | | | | Q546L, D548N, K549D, F571I, T574N, |
| | | | | S575N, L576K, N578S, G579S, N580S, |
| | | | | T581I, T582Q, D584T, R587N, Y616H, |
| | | | | R634H, G648S, D649N, T652E, N655K, |
| | | | | Q657E, A660N, S661A, N708D, S709T, |
| | | | | D710N |
| 26 | 726aa | BCD_0468 | 94.49% | A69E, S206G, 263-264delTG, G265S, |
| | | | | I373V, Q386H, G454A, S461N, S485P, |
| | | | | G491Q, D524N, K537T, Q538P, G539N, |
| | | | | S540A, S543T, V545A, D548N, K549D, |
| | | | | F571I, T574N, S575N, L576K, N578S, |
| | | | | G579S, N580S, T582Q, D584T, R587N, |
| | | | | Y616H, R634H, G648S, D649N, T652E, |
| | | | | N655K, Q657E, A660N, S661A, N708D, |
| | | | | S709T, D710N |
| 27 | 727aa | BCD_0492 | 92.98% | K104A, A182P, S206G, 262delQ, |
| | | | | T263S, G264S, G265F, N268T, N271D, |
| | | | | E326D, N327T, E328D, Q450N, N451G, |
| | | | | G454A, P460H, S461T, S462N, D465N, |
| | | | | S485P, G491Q, D524N, K537T, Q538P, |
| | | | | G539N, S540A, S543T, V545T, D548N, |
| | | | | K549D, F571I, T574N, S575N, L576K, |
| | | | | N578S, G579S, N580S, T582Q, D584T, |
| | | | | R587N, Y616H, R634H, G648S, D649N, |
| | | | | T652E, N655K, Q657D, A660N, S661A, |
| | | | | N708D, S709T, D710N |
| 28 | 728aa | BCD_0505 | 95.60% | S206G, Q262S, T263S, G265S, N268T, |
| | | | | N327T, E328D, I373V, G454A, D465N, |
| | | | | D524H, S540A, N541D, A542K, S543T, |
| | | | | V545A, Q546I, L551I, I552V, T560A, |
| | | | | S575T, N580Q, T582A, R634H, G648S, |
| | | | | D649N, T652D, Q656P, Q657E, A660N, |
| | | | | N698D, S709G |
| 29 | 727aa | BCD_0506 | 94.36% | A182P, 262delQ, T263S, G264Q, G265A, |
| | | | | N268S, E326D, N327T, Q450N, N451G, |
| | | | | S461N, K483T, T484V, S485P, E487Q, |
| | | | | E492D, T496A, D507N, V509A, D524H, |
| | | | | S540A, N541E, A542K, S543N, V545A, |
| | | | | Q546I, L551I, N578S, G579P, N580T, |
| | | | | T581E, T582A, D584E, T586A, Y616H, |
| | | | | G648S, T652L, Q656P, Q657A, N708D, |
| | | | | S709N, D710N |

TABLE 2.9
Branch 2 - Clade 6

| | | | % identity to | |
|---|---|---|---|---|
| | AA | | representative | Varying amino acid residues relative to the |
| No. | Length | Name | sequence | representative sequence |
| 0 | 737aa | AAV-7 | * | |
| | | (SEQ ID | | |
| | | NO: 7) | | |
| 1 | 728aa | BCD_0120 | 91.76% | D24N, N41H, A67E, A141G, V146I, 148- |
| | | | | 152delPSPQR, R169K, R171K, S198G, |
| | | | | V199L, V204M, 264delE, T265Q, A266S, |
| | | | | S268A, T273H, Y274F, K311R, S416T, |
| | | | | D419E, N453T, P454T, G456S, 458- |
| | | | | 460delAGN, R461K, Y466H, G468A, |
| | | | | S471N, A477S, F486Y, V491L, T494N, |
| | | | | L495R, Q497S, K510T, V520T, H529N, |
| | | | | R535H, S539I, S540N, I544V, T551A, |
| | | | | N563S, R568K, P569T, I580V, 587delA, |
| | | | | A588S, N589S, A592G, 593-594insP, |
| | | | | V596T, N599S, F706Y, E707A, Q709S, |
| | | | | T710N, G711N, D713E, D717N, S718N, |
| | | | | S723T |
| 2 | 737aa | BCD_0176 | 96.47% | N41D, E134S, G135P, A136V, A141G, |
| | | | | R169K, L189I, A195G, S198G, V199L, |
| | | | | V204M, E264Q, T265S, T273V, R314N, |
| | | | | I342V, E412T, S416T, P454A, S471T, |
| | | | | Q489K, V524I, D534E, A588S, N589S, |
| | | | | E707D |
| 3 | 728aa | BCD_0178 | 91.76% | N41D, A141G, 148-152delPSPQR, R169K, |
| | | | | S198G, V199L, S201P, G202T, V204M, |
| | | | | A206S, 264delE, T265Q, A266S, S268A, |
| | | | | T273H, Y274F, K311R, S416T, D419E, |
| | | | | 453delN, P454T, G455T, T457S, 459- |
| | | | | 460delGN, Y466H, G468A, S471N, A477S, |
| | | | | F486Y, V491L, T494N, L495I, Q497S, |
| | | | | V520T, H529N, R535Q, S539I, S540N, |
| | | | | I544V, T551A, N563S, R568K, P569T, |
| | | | | I580V, 587delA, A588S, N589S, A592G, |
| | | | | 593-594insP, V596T, V597I, N599S, F706Y, |
| | | | | E707A, Q709S, T710N, G711N, D713E, |
| | | | | D717N, S718N, Q719E, S723T |
| 4 | 737aa | BCD_0179 | 96.47% | N41D, K84Q, E134S, G135P, A136V, |
| | | | | A141G, R169K, L189I, A195G, S198G, |
| | | | | V199L, V204M, E264Q, T265S, T273V, |
| | | | | R314N, I342V, E412T, S416T, P454A, |
| | | | | S471T, V524I, D534E, A588S, N589S, |
| | | | | E707D |
| 5 | 737aa | BCD_0188 | 99.73% | N41D, N460T |
| 6 | 729aa | BCD_0189 | 91.91% | D24A, N41D, A141G, 145-149delPVEPS, |
| | | | | Q151I, R152D, R169K, R171K, V204M, |
| | | | | E264S, T265S, A266S, S268A, T273H, |
| | | | | I342V, N453T, P454T, G456S, 458- |
| | | | | 460delAGN, Y466H, G468A, S471N, |
| | | | | A477S, F486Y, V491L, T494N, Q497F, |
| | | | | G507A, V520T, V524I, A525P, H529N, |
| | | | | R535Q, S539I, S540N, I544V, T551A, |
| | | | | N563S, R568K, P569T, I580V, 587delA, |
| | | | | A588S, N589S, A592G, 593-594insP, T594S, |
| | | | | V596T, V597I, N599S, F706Y, E707A, |
| | | | | Q709S, T710N, G711N, D713E, D717N, |
| | | | | S718P, Q719D, S723T |
| 7 | 730aa | BCD_0194 | 91.92% | D24A, K31Q, N41D, A67E, G135A, A141G, |
| | | | | 147-151delEPSPQ, R152D, S157T, T158S, |
| | | | | S180A, L189I, S198G, V199L, V204M, |
| | | | | D238N, E264S, T265S, A266S, S268A, |
| | | | | T273H, K312R, I342V, S416E, 453delN, |
| | | | | P454S, G455T, 458delA, G459S, N460T, |
| | | | | Y466H, G468A, S471A, A477S, V491I, |
| | | | | L495T, Q497N, V520T, A525P, D534S, |
| | | | | R535V, S539I, S540N, I544V, T551S, |
| | | | | N563D, R568K, P569A, I580V, L585I, |
| | | | | A587S, A588Q, T590S, A591N, A592P, |
| | | | | Q593T, V596T, V662T, F706Y, E707D, |
| | | | | S718T |
| 8 | 733aa | BCD_0196 | 90.86% | N41H, A67E, Q105K, V125I, A141G, |
| | | | | P148Q, 152delR, S153E, T158S, K163T, |
| | | | | R169K, R171K, S180P, L189I, A195G, |
| | | | | S198G, V199L, V204M, E264S, T265S, |
| | | | | A266S, S268A, T273H, R314N, I342V, |
| | | | | E412T, S416T, N453T, P454T, G456S, 458- |
| | | | | 460delAGN, Y466H, G468A, S471N, |
| | | | | A477S, V491L, T494N, Q497F, G507A, |
| | | | | V520T, H529N, R535Q, S539I, S540N, |
| | | | | I544V, T551S, N563S, R568K, P569T, |
| | | | | I580Y, 587delA, A588S, N589S, A592G, |
| | | | | 593-594insP, T594S, V596T, A666S, F706Y, |
| | | | | E707A, Q709S, T710N, G711N, D713E, |
| | | | | D717N, S718T, Q719E, S723T |
| 9 | 734aa | BCD_0485 | 92.78% | D24A, N41D, K84Q, A141G, Q165P, |
| | | | | V183A, S201P, G202V, V204M, E264S, |
| | | | | T265S, A266S, S268A, T273H, K312R, |
| | | | | R314N, I342V, E412Q, S416T, N453T, |
| | | | | P454T, G456S, 458-460delAGN, Y466H, |
| | | | | G468A, S471N, F486Y, T494V, N499P, |
| | | | | G507A, N517D, V520A, V524I, H529N, |
| | | | | S539I, S540N, V542T, L543I, T551S, |
| | | | | N563S, I580V, A588S, A591N, A592P, |
| | | | | Q593T, T594S, V596T, A666S, E707D, |
| | | | | Q709S, T710N, G711S, A715S, S718A |
| 10 | 729aa | BCD_0489 | 92.18% | D24A, N41D, K84Q, A141G, V146I, 148- |
| | | | | 152delPSPQR, T158S, R169K, R171K, |
| | | | | D179E, D185E, S201P, G202V, V204M, |
| | | | | E264S, T265S, A266S, S268A, T273H, |
| | | | | K312R, R314N, I342V, E412Q, S416T, |
| | | | | N453T, P454T, G456S, 458-460delAGN, |
| | | | | Y466H, G468A, S471N, F486Y, T494V, |
| | | | | N499P, G507A, N517D, V520A, V524I, |
| | | | | H529N, S539I, S540N, V542T, L543I, |
| | | | | T551S, N563S, I580V, A588S, A591N, |
| | | | | A592P, Q593T, T594S, V596T, A666S, |
| | | | | E707D, Q709S, T710N, G711S, A715S, |
| | | | | S718A |
| 11 | 730aa | BMN_0250 | 91.51% | D24A, K31Q, N41D, A67E, F119L, G135A, |
| | | | | A141G, 147-151delEPSPQ, R152D, S157T, |
| | | | | T158S, S180A, L189I, S198G, V199L, |
| | | | | V204M, D238N, E264S, T265S, A266S, |
| | | | | S268A, T273H, K312R, I342V, S416E, |
| | | | | 453delN, P454S, G455T, 458delA, G459S, |
| | | | | N460T, Y466H, G468A, S471A, A477S, |
| | | | | V491I, L495T, Q497N, N517S, V520T, |
| | | | | A525P, D534S, R535V, S539I, S540N, |
| | | | | I544V, T551S, N563D, R568K, P569A, |
| | | | | I580V, L585I, A587S, A588Q, T590S, |
| | | | | A591N, A592P, Q593T, V596T, V662T, |
| | | | | F706Y, E707D, D713G, S718T |
| 12 | 730aa | BMN 0251 | 91.92% | D24A, K31Q, N41D, A67E, G135A, A141G, |
| | | | | 147-151delEPSPQ, R152D, S157T, T158S, |
| | | | | S180A, L189I, S198G, V199L, V204M, |
| | | | | D238N, E264S, T265S, A266S, S268A, |
| | | | | T273H, K312R, I342V, S416E, 453delN, |
| | | | | P454S, G455T, 458delA, G459S, N460T, |
| | | | | Y466H, G468A, S471A, A477S, V491I, |
| | | | | L495T, Q497N, V520T, A525P, D534S, |
| | | | | R535V, S539I, S540N, I544V, T551S, |
| | | | | N563D, R568K, P569A, I580V, L585I, |
| | | | | A587S, A588Q, T590S, A591N, A592P, |
| | | | | Q593T, V596T, V662T, F706Y, E707D, |
| | | | | S718T |
| 13 | 730aa | BMN_0253 | 91.51% | D24A, K31Q, N41D, A67E, G135A, A141G, |
| | | | | 147-151delEPSPQ, R152D, S157T, T158S, |
| | | | | S180A, L189I, S198G, V199L, V204M, |
| | | | | S226P, D238N, E264S, T265S, A266S, |
| | | | | S268A, T273H, Q299P, K312R, I342V, |
| | | | | S416E, L444P, 453delN, P454S, G455T, |
| | | | | 458delA, G459S, N460T, Y466H, G468A, |
| | | | | S471A, A477S, V491I, L495T, Q497N, |
| | | | | V520T, A525P, D534S, R535V, S539I, |
| | | | | S540N, I544V, T551S, N563D, R568K, |
| | | | | P569A, I580V, L585I, A587S, A588Q, |
| | | | | T590S, A591N, A592P, Q593A, V596T, |
| | | | | V662T, F706Y, E707D, S718T |
| 14 | 730aa | BMN_0254 | 91.10% | D24A, K31Q, N41D, V46M, A67E, G135A, |
| | | | | A141G, 147-151delEPSPQ, R152D, S157T, |
| | | | | T158S, S180A, L189I, S198G, V199L, |
| | | | | V204M, N228S, D238N, E264S, T265S, |
| | | | | A266S, S268A, T273H, K312R, I342V, |
| | | | | S386R, S416E, L444P, 453delN, P454S, |
| | | | | G455T, 458delA, G459S, N460T, Y466H, |
| | | | | G468A, S471A, A477S, V491I, L495T, |
| | | | | Q497N, V520T, A525P, D534S, R535V, |
| | | | | S539I, S540N, I544V, T551S, L560P, |
| | | | | N563D, R568K, P569A, I580V, L585I, |
| | | | | A587S, A588Q, T590S, A591N, A592P, |
| | | | | Q593T, V596T, V662T, S693N, F706Y, |
| | | | | E707D, S718T |
| 15 | 730aa | BMN 0255 | 91.51% | D24A, K31Q, N41D, N57S, A67E, G135A, |
| | | | | A141G, 147-151delEPSPQ, R152D, S157T, |
| | | | | T158S, S180A, L189I, S198G, V199L, |
| | | | | V204M, D238N, N255D, E264S, T265S, |
| | | | | A266S, S268A, T273H, K312R, I342V, |
| | | | | S416E, 453delN, P454S, G455T, 458delA, |
| | | | | G459S, N460T, Y466H, G468A, S471A, |
| | | | | A477S, V491I, L495T, Q497N, V520T, |
| | | | | A525P, D534S, R535V, S539I, S540N, |
| | | | | I544V, T551S, N563D, R568K, P569A, |
| | | | | I580V, L585I, A587S, A588Q, T590S, |
| | | | | A591N, A592P, Q593T, V596T, Q609R, |
| | | | | V662T, F706Y, E707D, S718T |
| 16 | 730aa | BMN 0256 | 91.51% | D24A, K31Q, N41D, A67E, G135A, A141G, |
| | | | | 147-151delEPSPQ, R152D, S157T, T158S, |
| | | | | S180A, L189I, S198G, V199L, V204M, |
| | | | | D238N, E264S, T265S, A266S, S268A, |
| | | | | T273H, K312R, I342V, S416E, L447M, |
| | | | | 453delN, P454S, G455T, 458delA, G459S, |
| | | | | N460T, L463P, Y466H, G468A, S471A, |
| | | | | A477S, V491I, L495T, Q497N, V520T, |
| | | | | A525P, D534S, R535V, S539I, S540N, |
| | | | | I544V, T551S, N563D, R568K, P569A, |
| | | | | I580V, L585I, A587S, A588Q, T590S, |
| | | | | A591N, A592P, Q593T, V596T, N629D, |
| | | | | V662T, F706Y, E707D, S718T |
| 17 | 730aa | BMN 0257 | 91.78% | D24A, K31Q, N41D, A67E, G135A, A141G, |
| | | | | 147-151delEPSPQ, R152D, S157T, T158S, |
| | | | | S180A, L189I, S198G, V199L, V204M, |
| | | | | D238N, E264G, T265S, A266S, S268A, |
| | | | | T273H, K312R, I342V, M404V, S416E, |
| | | | | 453delN, P454S, G455T, 458delA, G459S, |
| | | | | N460T, Y466H, G468A, S471A, A477S, |
| | | | | V491I, L495S, Q497N, V520T, A525P, |
| | | | | D534S, R535V, S539I, S540N, I544V, |
| | | | | T551S, N563D, R568K, P569A, I580V, |
| | | | | L585I, A587S, A588Q, T590S, A591N, |
| | | | | A592P, Q593T, V596T, V662T, F706Y, |
| | | | | E707D, S718T |
| 18 | 730aa | BMN 0258 | 91.23% | D24A, K31Q, N41D, K51R, A67E, V125I, |
| | | | | G135A, A141G, K143E, 147-151delEPSPQ, |
| | | | | R152D, S157T, T158S, S180A, L189I, |
| | | | | S198G, V199L, V204M, D238N, N254D, |
| | | | | E264S, T265S, A266S, S268A, T273H, |
| | | | | K312R, I342V, S416E, 453delN, P454S, |
| | | | | G455T, 458delA, G459S, N460T, Y466H, |
| | | | | G468A, S471A, A477S, V491I, L495T, |
| | | | | Q497N, V520T, A525P, D534S, R535V, |
| | | | | S539I, S540N, I544V, F545L, T551S, |
| | | | | N563D, R568K, P569A, I580V, L585I, |
| | | | | A587S, A588Q, T590S, A591N, A592P, |
| | | | | Q593T, V596T, V662T, F706Y, E707D, |
| | | | | S718T |
| 19 | 730aa | BMN 0260 | 91.64% | D24A, K31R, N41D, A67E, G135A, A141G, |
| | | | | 147-151delEPSPQ, R152D, S157T, T158S, |
| | | | | S180A, L189I, S198G, V199L, V204M, |
| | | | | D238N, E264S, T265S, A266S, S268A, |
| | | | | T273H, K312R, I342V, C396R, S416E, |
| | | | | 453delN, P454S, G455T, 458delA, G459S, |
| | | | | N460T, Y466H, G468A, S471A, A477S, |
| | | | | V491I, L495T, Q497N, V520T, A525P, |
| | | | | K530R, D534S, R535V, S539I, S540N, |
| | | | | I544V, T551S, N563D, R568K, P569A, |
| | | | | I580V, L585I, A587S, A588Q, T590S, |
| | | | | A591N, A592P, Q593T, V596T, V662T, |
| | | | | F706Y, E707D, S718T |

TABLE 2.10
Branch 2 - Clade 11

| | | | % identity to | |
|---|---|---|---|---|
| | AA | | representative | Varying amino acid residues relative to the |
| No. | Length | Name | sequence | representative sequence |
| 0 | 736aa | AAV-9 | * | |
| | | (SEQ ID | | |
| | | NO: 9) | | |
| 1 | 736aa | BCD_0112 | 99.59% | V146I, A157V, Q412T |
| 2 | 736aa | BMN 0316 | 98.37% | H38K, N41D, A42G, G56F, A67E, K92R, |
| | | | | K105Q, L125V, Q412T, T460A, M635V, |
| | | | | S680R |
| 3 | 736aa | BMN 0317 | 98.51% | H38K, N41D, A42G, G56F, A67E, K92R, |
| | | | | K105Q, L125V, L405P, Q412T, E682A |
| 4 | 736aa | BMN 0318 | 98.64% | H38K, N41D, A42G, G56F, A67E, K92R, |
| | | | | K105Q, L125V, Q412T, L435P |
| 5 | 736aa | BMN 0319 | 98.37% | H38K, N41D, A42G, G56F, A67E, K92R, |
| | | | | K105Q, L125V, K168I, T244I, N287S, |
| | | | | Q412T |
| 6 | 736aa | BMN 0320 | 98.78% | H38K, N41D, A42G, G56F, A67E, |
| | | | | K92R, K105Q, L125V, Q412T |
| 7 | 736aa | BMN 0321 | 98.51% | H38K, N41D, A42G, G56F, A67E, |
| | | | | K92R, K105Q, L125V, Q233R, V331A, |
| | | | | Q412T |

TABLE 2.11
Branch 2 - Clade 12

| | | | % identity to | |
|---|---|---|---|---|
| | AA | | representative | Varying amino acid residues relative to the |
| No. | Length | Name | sequence | representative sequence |
| 0 | 735aa | AAV-2 | * | |
| | | (SEQ ID | | |
| | | NO: 4) | | |
| 1 | 735aa | BCD_0105 | 96.05% | A35N, E36Q, R37Q, K39R, R81H, D84K, |
| | | | | S85Q, K105Q, P135A, V151A, A162S, |
| | | | | A179T, D180E, L188I, Q190E, L198V, |
| | | | | T200S, T205S, S207G, Q263E, S267T, |
| | | | | R310K, R447K, S492A, S578A, R585S, |
| | | | | R588T, A593S, D711E |
| 2 | 735aa | BCD_0137 | 97.69% | V125I, V151A, A162S, T205S, P293Y, |
| | | | | Q297E, R298T, N312S, Q457M, S492A, |
| | | | | E499D, F533Y, G546D, E548G, R585S, |
| | | | | R588T, A593S |
| 3 | 735aa | BCD_0447 | 97.96% | V151A, A162S, N312S, R447K, T450A, |
| | | | | Q457M, S492A, E499D, F533Y, G546D, |
| | | | | E548G, S578A, R585S, R588T, A593S |

TABLE 2.12
Branch 2 - Clade 13

| | | | % identity to | |
|---|---|---|---|---|
| | AA | | representative | Varying amino acid residues relative to the |
| No. | Length | Name | sequence | representative sequence |
| 0 | 739aa | BCD_0159 | * | |
| | | (SEQ ID | | |
| | | NO: 27) | | |
| 1 | 739aa | BCD_0167 | 99.59% | N719D, A720T, D721N |
| 2 | 739aa | BCD_0168 | 98.65% | P48L, K61R, V65A, H75Y, S519P, V609A, |
| | | | | N699S, N719D, A720T, D721N |
| 3 | 739aa | BCD_0169 | 99.19% | T138A, I244T, Q323L, K463R, S467F, |
| | | | | E566V |
| 4 | 739aa | BCD_0170 | 99.19% | H75F, N151S, F670S, N719D, A720T, |
| | | | | D721N |
| 5 | 739aa | BCD_0171 | 99.32% | N151I, S236P, Y256H, L319P, D435G |
| 6 | 739aa | BCD_0172 | 99.05% | H75Y, F291L, L643P, L690P, N719D, |
| | | | | A720T, D721N |

TABLE 2.13
Branch 2 - Clade 16

| | | | % identity to | |
|---|---|---|---|---|
| | AA | | representative | Varying amino acid residues relative to the |
| No. | Length | Name | sequence | representative sequence |
| 0 | 736aa | AAV-1 | * | |
| | | (SEQ ID | | |
| | | NO: 3) | | |
| 1 | 736aa | AAV-6 | 99.18% | L129F, E418D, E531K, F584L, A598V, |
| | | | | N642H |
| 2 | 736aa | BCD_0441 | 97.83% | A67E, L188I, T194A, A196S, A197G, |
| | | | | V198L, P200S, T201N, A224S, E418D, |
| | | | | G505S, F584L, A592V, A598V, A601V, |
| | | | | N642H |
| 3 | 736aa | BCD_0442 | 98.51% | D24N, D41N, K84Q, L129F, T162S, |
| | | | | E418D, G505S, F584L, A592V, A598V, |
| | | | | N642H |

TABLE 2.14
Branch 2 - Clade 21

| | | | % identity to | |
|---|---|---|---|---|
| | AA | | representative | Varying amino acid residues relative to the |
| No. | Length | Name | sequence | representative sequence |
| 0 | 736aa | AAV LK03 | * | |
| | | (SEQ ID | | |
| | | NO: 1) | | |
| 1 | 733aa | AAV-13 | 94.54% | 2delA, A3T, I19V, K105Q, L125I, D147E, |
| | | | | Q151A, V159I, K164Q, S179T, E190Q, |
| | | | | T196S, S197G, L198V, N201T, S267T, |
| | | | | K310R, S312N, 450delG, T452A, 455delT, |
| | | | | N457Q, Q469T, R476K, T492Q, A505G, |
| | | | | S507T, E531K, N540T, T549N, S551N, |
| | | | | E554D, D556E, A581S, S586N, T589A, |
| | | | | A590G, R594G, D598H, T661N, P664A, |
| | | | | P735N |
| 2 | 736aa | AAV-3b | 98.91% | Q26K, A29V, K31Q, A42R, A67E, K105Q, |
| | | | | L125I, P735N |
| 3 | 735aa | BCD_0113 | 91.56% | N14T, E21Q, A24K, Q26K, A29P, |
| | | | | K31P, A34P, N35A, Q36E, Q37R, Q39K, |
| | | | | N41D,A42S, G56F, A67E, Q81R, K84D, |
| | | | | A85S, L125V, A135P, A136V, D147E, |
| | | | | Q148H, Q151A, V159I, S162A, K164Q, |
| | | | | S179A, E180D, E190Q, T196S, S197G, |
| | | | | N201T, S205T, G207S, M211V, K310R, |
| | | | | S312N, R447K, 450delG, T451S, T452N, |
| | | | | T456L, N457Q, Q469T, R476K, T492Q, |
| | | | | S507T, N540T, E546Q, T549N, S551N, |
| | | | | N552D, E554D, T579Y, A581S, S586N, |
| | | | | A590G, R594G, D598H, T661N, P664S, |
| | | | | P735N |
| 4 | 735aa | BCD_0114 | 91.56% | N14T, E21Q, A24K, Q26K, A29P, |
| | | | | K31P, A34P, N35A, Q36E, Q37R, Q39K, |
| | | | | N41D, A42S, G56F, A67E, Q81R, K84D, |
| | | | | A85S, L125V, A135P, A136V, D147E, |
| | | | | Q148H, Q151A, V159T, S162A, K164Q, |
| | | | | S179A, E180D, E190Q, T196S, S197G, |
| | | | | N201T, S205T, G207S, M211V, K310R, |
| | | | | S312N, R447K, 450delG, T451S, T452N, |
| | | | | T456L, N457Q, Q469T, R476K, T492Q, |
| | | | | S507T, N540T, E546Q, T549N, S551N, |
| | | | | N552D, E554D, T579Y, A581S, S586N, |
| | | | | A590G, R594G, D598H, T661N, P664S, |
| | | | | P735N |
| 5 | 735aa | BCD_0115 | 91.43% | N14T, E21Q, A24K, Q26K, A29P, K31P, |
| | | | | A34P, N35A, Q36E, Q37R, Q39K, N41D, |
| | | | | A42S, G56F, A67E, Q81R, K84D, A85S, |
| | | | | L125V, A135P, A136V, D147E, Q148H, |
| | | | | Q151A, V159T, S162A, K164Q, S179A, |
| | | | | E180D, E190Q, T196S, S197G, N201T, |
| | | | | S205T, G207S, K310R, S312N, Q410T, |
| | | | | R447K, 450delG, T451S, T452N, T456L, |
| | | | | N457Q, Q469N, R476K, T492Q, S507T, |
| | | | | N540T, E546Q, T548A, T549N, S551N, |
| | | | | N552D, E554D, T579Y, A581S, S586N, |
| | | | | A590G, R594G, D598H, T661N, P664A, |
| | | | | P735N |
| 6 | 735aa | BCD_0117 | 91.56% | N14T, E21Q, A24K, Q26K, A29P, K31P, |
| | | | | A34P, N35A, Q36E, Q37R, Q39K, N41D, |
| | | | | A42S, G56F, A67E, Q81R, K84D, A85S, |
| | | | | L125V, A135P, A136V, D147E, Q148H, |
| | | | | Q151A, V159I, S162A, K164Q, S179A, |
| | | | | E180D, E190Q, T196S, S197G, N201T, |
| | | | | S205T, G207S, M211V, K310R, S312N, |
| | | | | R447K, 450delG, T451S, T452N, T456L, |
| | | | | N457Q, Q469T, R476K, T492Q, S507T, |
| | | | | N540T, E546Q, T549N, S551N, N552D, |
| | | | | E554D, T579Y, A581S, S586N, A590G, |
| | | | | R594G, D598H, T661N, P664S, P735N |

TABLE 2.15
Branch 2 - Clade 22

| | | | % identity to | |
|---|---|---|---|---|
| | AA | | representative | Varying amino acid residues relative to the |
| No. | Length | Name | sequence | representative sequence |
| 0 | 741aa | BCD_0135 | * | |
| | | (SEQ ID NO: | | |
| | | 23) | | |
| 1 | 741aa | BCD_0157 | 99.73% | P168Q, N175D |

In some embodiments, an AAV clade member is a capsid protein with an amino acid identity of at least 90% identity, at least 91% identity, at least 92% identity, at least 93% identity, or at least 94% identity to a VP1, VP2, or VP3 of a novel reference capsid (e.g., a VP1 capsid protein of AAV VP1 capsid protein No. 0 in any one of Table 2, or VP2 or VP3 capsid proteins derived therefrom). In certain embodiments, an AAV clade member is a capsid protein with an amino acid identity of at least 95% identity, at least 96% identity, at least 97% identity, at least 98% identity, at least 99% identity, or 100% identity to a VP1, VP2, or VP3 of a novel reference capsid (e.g., a VP1 capsid protein of AAV VP1 capsid protein No. 0 in any one of Table 2, or VP2 or VP3 capsid proteins derived therefrom). In accordance with these embodiments, in specific embodiments, AAV clade members that are known in the art are not encompassed by the present disclosure (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure).

In some embodiments, an AAV clade member is a capsid protein with an amino acid identity of at least 90% identity, at least 91% identity, at least 92% identity, at least 93% identity, or at least 94% identity to a VP1 of a novel reference capsid (e.g., a VP1 capsid protein of AAV VP1 capsid protein No. 0 in any one of Table 2). In certain embodiments, an AAV clade member is a capsid protein with an amino acid identity of at least 95% identity, at least 96% identity, at least 97% identity, at least 98% identity, at least 99% identity, or 100% identity to a VP1 of a novel reference capsid (e.g., a VP1 capsid protein of AAV VP1 capsid protein No. 0 in any one of Table 2). In accordance with these embodiments, in specific embodiments, AAV clade members that are known in the art are not encompassed by the present disclosure (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure).

In some embodiments, an AAV clade member is a capsid protein with an amino acid identity of at least 90% identity, at least 91% identity, at least 92% identity, at least 93% identity, or at least 94% identity to a VP2 of a novel reference capsid (e.g., a VP2 capsid protein of AAV VP1 capsid protein No. 0 in any one of Table 2). In certain embodiments, an AAV clade member is a capsid protein with an amino acid identity of at least 95% identity, at least 96% identity, at least 97% identity, at least 98% identity, at least 99% identity, or 100% identity to a VP2 of a novel reference capsid (e.g., a VP2 capsid protein of AAV VP1 capsid protein No. 0 in any one of Table 2). In accordance with these embodiments, in specific embodiments, AAV clade members that are known in the art are not encompassed by the present disclosure (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure).

In some embodiments, an AAV clade member is a capsid protein with an amino acid identity of at least 90% identity, at least 91% identity, at least 92% identity, at least 93% identity, or at least 94% identity to a VP3 of a novel reference capsid (e.g., a VP3 capsid protein of AAV VP1 capsid protein No. 0 in any one of Table 2). In certain embodiments, an AAV clade member is a capsid protein with an amino acid identity of at least 95% identity, at least 96% identity, at least 97% identity, at least 98% identity, at least 99% identity, or 100% identity to a VP3 of a novel reference capsid (e.g., a VP3 capsid protein of AAV VP1 capsid protein No. 0 in any one of Table 2). In accordance with these embodiments, in specific embodiments, AAV clade members that are known in the art are not encompassed by the present disclosure (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure).

In some embodiments, the structural homology of an AAV capsid protein (e.g., VP1, VP2 or VP3) can be determined by structural alignment with the SSM (Secondary Structure Matching) program. See Krissinel E, Henrick K. Secondary-structure matching (SSM), a new tool for fast protein structure alignment in three dimensions. Acta Crystallogr D Biol Crystallogr. 2004 December; 60 (Pt 12 Pt 1): 2256-68. doi: 10.1107/S0907444904026460. For example, the crystal structure of a AAV capsid viral protein (VP) can be determined and compared to the crystal structure of a VP of a representative member of an AAV clade. The crystal structure of an AAV capsid protein can be determined using cryo-EM (X-ray crystallography) or cryo-reconstruction.

### 6.3.2.2 Representative Sequence

The present disclosure also provides AAV clades grouped by their VP1 sequence being substantially related to a representative sequence. Representative sequences of such AAV clades are described in Table 2 and are designated No. “0”.

A representative sequence of a novel AAV clade can be determined using algorithms such as, ClustalW (e.g., ClustalW with cost matrix BLOSUM, a gap open cost of 10, and a gap extend cost of 0.1), and a clustering algorithm such as CD-HIT or USEARCH., as described in the following papers, Weizhong Li, Lukasz Jaroszewski & Adam Godzik. Bioinformatics (2001) 17:282-283, Weizhong Li, Lukasz Jaroszewski & Adam Godzik. Bioinformatics (2002) 18:77-82, PDF, Pubmed; Weizhong Li & Adam Godzik. Bioinformatics (2006) 22:1658-165, which are incorporated by reference. Briefly, a clustering algorithm will sort a set of amino acid sequences by length. Typically, the longest sequence will become the representative sequence of a cluster. Then each remaining sequence in the set is compared to the representative sequence. If the sequence similarity or identity of each remaining sequence is within the threshold of interest as compared to the representative sequence, then it is included as a member of that cluster.

Using the representative sequence novel clades were identified. The alignment of the VP1 capsid proteins for all the AAV clades with two or more members are provided in FIGS. 4-13. For AAV clades with only one member identified thus far, alignments are not provided but are shown in Table 2.

In certain embodiments, a clade member can include one or more of the AAV clade members listed in any one of Tables 2.6 to 2.15. In specific embodiments, novel AAV capsid proteins of clade 1 include any one of or all of Nos. 2 to 48. In specific embodiments, novel AAV capsid proteins of clade 4 include any one of or all of Nos. 2 to 59. In specific embodiments, novel AAV capsid proteins of clade 6 include any one of or all of Nos. 1 to 19. In specific embodiments, novel AAV capsid proteins of clade 11 include any one of or all of Nos. 1 to 7. In specific embodiments, novel AAV capsid proteins of clade 12 include any one of or all of Nos. 1 to 3. In specific embodiments, novel AAV capsid proteins of clade 13 include any one of or all of Nos. 0 to 6. In specific embodiments, novel AAV capsid proteins of clade 16 include any one of or all of Nos. 2 to 3. In specific embodiments, novel AAV capsid proteins of clade 21 include any one of or all of Nos. 3 to 6. In specific embodiments, novel AAV capsid proteins of clade 22 include any one of or all of Nos. 0 to 1.

In specific embodiments, a member of clade 0 comprises an amino acid sequence of an AAV capsid of any one of Nos. 0-50 in Table 2.6 with one or more mutations, such as one or more amino acid substitutions (e.g., 1, 2, 3, 4, 5, 6, 7, 8, 9 or more), recited in Table 2.6, provided that the mutation(s) (e.g., amino acid substitution(s)) does not result in a known AAV capsid. In specific embodiments, a member of clade 1 comprises an amino acid sequence of an AAV capsid of any one of Nos. 0-48 in Table 2.7 with one or more mutations, such as one or more amino acid substitutions (e.g., 1, 2, 3, 4, 5, 6, 7, 8, 9 or more), recited in Table 2.7, provided that the mutation(s) (e.g., amino acid substitution(s)) does not result in a known AAV capsid. In specific embodiments, a member of clade 4 comprises an amino acid sequence of an AAV capsid of any one of Nos. 0-29 in Table 2.8 with one more mutations, such as one or more amino acid substitutions (e.g., 1, 2, 3, 4, 5, 6, 7, 8, 9 or more), recited in Table 2.8, provided that the mutation(s) (e.g., amino acid substitution(s)) does not result in a known AAV capsid. In specific embodiments, a member of clade 6 comprises an amino acid sequence of an AAV capsid of any one of Nos. 0-19 in Table 2.9 with one or more mutations, such as one or more amino acid substitutions (e.g., 1, 2, 3, 4, 5, 6, 7, 8, 9 or more), recited in Table 2.9, provided that the mutation(s) (e.g., amino acid substitution(s) does not result in a known AAV capsid. In specific embodiments, a member of clade 11 comprises an amino acid sequence of an AAV capsid of any one of Nos. 0-7 in Table 2.10 with one or more mutations, such as one or more amino acid substitutions (e.g., 1, 2, 3, 4, 5, 6, 7, 8, 9 or more), recited in Table 2.10, provided that the mutation(s) (e.g., amino acid substitution(s) does not result in a known AAV capsid. In specific embodiments, a member of clade 12 comprises an amino acid sequence of an AAV capsid of any one of Nos. 0-3 in Table 2.11 with one or more mutations, such as one or more amino acid substitutions (e.g., 1, 2, 3, 4, 5, 6, 7, 8, 9 or more), recited in Table 2.11, provided that the mutation(s) (e.g., amino acid substitution(s) does not result in a known AAV capsid. In specific embodiments, a member of clade 13 comprises an amino acid sequence of an AAV capsid of any one of Nos. 0-6 in Table 2.12 with one or more mutations, such as one or more amino acid substitutions (e.g., 1, 2, 3, 4, 5, 6, 7, 8, 9 or more), recited in Table 2.12, provided that the mutation(s) (e.g., amino acid substitution(s) does not result in a known AAV capsid. In specific embodiments, a member of clade 16 comprises an amino acid sequence of an AAV capsid of any one of Nos. 0-3 in Table 2.13 with one or more mutations, such as one or more amino acid substitutions (e.g., 1, 2, 3, 4, 5, 6, 7, 8, 9 or more), recited in Table 2.13, provided that the mutation(s) (e.g., amino acid substitution(s) does not result in a known AAV capsid. In specific embodiments, a member of clade 21 comprises an amino acid sequence of an AAV capsid of any one of Nos. 0-6 in Table 2.14 with one or more mutations, such as one or more amino acid substitutions (e.g., 1, 2, 3, 4, 5, 6, 7, 8, 9 or more), recited in Table 2.14, provided that the mutation(s) (e.g., amino acid substitution(s) does not result in a known AAV capsid. In specific embodiments, a member of clade 22 comprises an amino acid sequence of an AAV capsid of any one of Nos. 0-1 in Table 2.15 with one or more mutations, such as one or more amino acid substitutions (e.g., 1, 2, 3, 4, 5, 6, 7, 8, 9 or more), recited in Table 2.15, provided that the mutation(s) (e.g., amino acid substitution(s) does not result in a known AAV capsid.

In a specific embodiment, an AAV capsid member of a novel AAV clade of the disclosure has an VP1 amino acid sequence that is substantially related to a representative amino acid sequence of a novel AAV clade. In another specific embodiment, an AAV clade member encompassed by the disclosure comprises a VP1 capsid protein with at least about 90% identity to VP1 of AAV clade member No. 0 in any one of Table 2. In another specific embodiment, an AAV clade member encompassed by the disclosure comprises a VP1 capsid protein with at least about 91% identity to VP1 of AAV clade member No. 0 in any one of Table 2. In another specific embodiment, an AAV clade member encompassed by the disclosure comprises a VP1 capsid protein with at least about 92% identity to VP1 of AAV clade member No. 0 in any one of Table 2. In a specific embodiment, an AAV clade member encompassed by the disclosure comprises a VP1 capsid protein with at least about 93% identity to VP1 of AAV clade member No. 0 in any one of Table 2. In another specific embodiment, an AAV clade member encompassed by the disclosure comprises a VP1 capsid protein with at least about 94% identity to VP1 of AAV clade member No. 0 in any one of Table 2. In another specific embodiment, an AAV clade member encompassed by the disclosure comprises a VP1 capsid protein with at least about 95% identity to VP1 of AAV clade member No. 0 in any one of Table 2. In another specific embodiment, an AAV clade member encompassed by the disclosure comprises a VP1 capsid protein with at least about 96% identity to VP1 of AAV clade member No. 0 in any one of Table 2. In another specific embodiment, an AAV clade member encompassed by the disclosure comprises a VP1 capsid protein with at least about 97% identity to VP1 of AAV clade member No. 0 in any one of Table 2. In another specific embodiment, an AAV clade member encompassed by the disclosure comprises a VP1 capsid protein with at least about 98% identity to VP1 of AAV clade member No. 0 in any one of Table 2. In another specific embodiment, an AAV clade member encompassed by the disclosure comprises a VP1 capsid protein with at least about 99% identity to VP1 of AAV clade member No. 0 in any one of Table 2. In some embodiments, the AAV clade member is a member of clade 0. In some embodiments, the AAV clade member is a member of clade 1. In some embodiments, the AAV clade member is a member of clade 4. In some embodiments, the AAV clade member is a member of clade 6. In some embodiments, the AAV clade member is a member of clade 11. In some embodiments, the AAV clade member is a member of clade 12. In some embodiments, the AAV clade member is a member of clade 13. In some embodiments, the AAV clade member is a member of clade 16. In some embodiments, the AAV clade member is a member of clade 21. In some embodiments, the AAV clade member is a member of clade 22.

In some embodiments, an AAV clade member encompassed by the disclosure comprises a VP1 capsid protein with at least about 90% identity to VP1 of AAV clade member No. 0 in Table 2.6. In some embodiments, an AAV clade member encompassed by the disclosure comprises a VP1 capsid protein with at least about 90% identity to VP1 of AAV clade member No. 0 in Table 2.7. In some embodiments, an AAV clade member encompassed by the disclosure comprises a VP1 capsid protein with at least about 90% identity to VP1 of AAV clade member No. 0 in Table 2.8. In some embodiments, an AAV clade member encompassed by the disclosure comprises a VP1 capsid protein with at least about 90% identity to VP1 of AAV clade member No. 0 in Table 2.9. In some embodiments, an AAV clade member encompassed by the disclosure comprises a VP1 capsid protein with at least about 90% identity to VP1 of AAV clade member No. 0 in Table 2.10. In some embodiments, an AAV clade member encompassed by the disclosure comprises a VP1 capsid protein with at least about 90% identity to VP1 of AAV clade member No. 0 in Table 2.11. In some embodiments, an AAV clade member encompassed by the disclosure comprises a VP1 capsid protein with at least about 90% identity to VP1 of AAV clade member No. 0 in Table 2.12. In some embodiments, an AAV clade member encompassed by the disclosure comprises a VP1 capsid protein with at least about 90% identity to VP1 of AAV clade member No. 0 in Table 2.13. In some embodiments, an AAV clade member encompassed by the disclosure comprises a VP1 capsid protein with at least about 90% identity to VP1 of AAV clade member No. 0 in Table 2.14. In some embodiments, an AAV clade member encompassed by the disclosure comprises a VP1 capsid protein with at least about 90% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 91% identity to VP1 of AAV clade member No. 0 in Table 2.6. In some embodiments, an AAV clade member encompassed by the disclosure comprises a VP1 capsid protein with at least about 91% identity to VP1 of AAV clade member No. 0 in Table 2.7. In some embodiments, an AAV clade member encompassed by the disclosure comprises a VP1 capsid protein with at least about 91% identity to VP1 of AAV clade member No. 0 in Table 2.8. In some embodiments, an AAV clade member encompassed by the disclosure comprises a VP1 capsid protein with at least about 91% identity to VP1 of AAV clade member No. 0 in Table 2.9. In some embodiments, an AAV clade member encompassed by the disclosure comprises a VP1 capsid protein with at least about 91% identity to VP1 of AAV clade member No. 0 in Table 2.10. In some embodiments, an AAV clade member encompassed by the disclosure comprises a VP1 capsid protein with at least about 91% identity to VP1 of AAV clade member No. 0 in Table 2.11. In some embodiments, an AAV clade member encompassed by the disclosure comprises a VP1 capsid protein with at least about 91% identity to VP1 of AAV clade member No. 0 in Table 2.12. In some embodiments, an AAV clade member encompassed by the disclosure comprises a VP1 capsid protein with at least about 91% identity to VP1 of AAV clade member No. 0 in Table 2.13. In some embodiments, an AAV clade member encompassed by the disclosure comprises a VP1 capsid protein with at least about 91% identity to VP1 of AAV clade member No. 0 in Table 2.14. In some embodiments, an AAV clade member encompassed by the disclosure comprises a VP1 capsid protein with at least about 91% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 92% identity to VP1 of AAV clade member No. 0 in Table 2.6. In some embodiments, an AAV clade member encompassed by the disclosure comprises a VP1 capsid protein with at least about 92% identity to VP1 of AAV clade member No. 0 in Table 2.7. In some embodiments, an AAV clade member encompassed by the disclosure comprises a VP1 capsid protein with at least about 92% identity to VP1 of AAV clade member No. 0 in Table 2.8. In some embodiments, an AAV clade member encompassed by the disclosure comprises a VP1 capsid protein with at least about 92% identity to VP1 of AAV clade member No. 0 in Table 2.9. In some embodiments, an AAV clade member encompassed by the disclosure comprises a VP1 capsid protein with at least about 92% identity to VP1 of AAV clade member No. 0 in Table 2.10. In some embodiments, an AAV clade member encompassed by the disclosure comprises a VP1 capsid protein with at least about 92% identity to VP1 of AAV clade member No. 0 in Table 2.11. In some embodiments, an AAV clade member encompassed by the disclosure comprises a VP1 capsid protein with at least about 92% identity to VP1 of AAV clade member No. 0 in Table 2.12. In some embodiments, an AAV clade member encompassed by the disclosure comprises a VP1 capsid protein with at least about 92% identity to VP1 of AAV clade member No. 0 in Table 2.13. In some embodiments, an AAV clade member encompassed by the disclosure comprises a VP1 capsid protein with at least about 92% identity to VP1 of AAV clade member No. 0 in Table 2.14. In some embodiments, an AAV clade member encompassed by the disclosure comprises a VP1 capsid protein with at least about 92% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 93% identity to VP1 of AAV clade member No. 0 in Table 2.6. In some embodiments, an AAV clade member encompassed by the disclosure comprises a VP1 capsid protein with at least about 93% identity to VP1 of AAV clade member No. 0 in Table 2.7. In some embodiments, an AAV clade member encompassed by the disclosure comprises a VP1 capsid protein with at least about 93% identity to VP1 of AAV clade member No. 0 in Table 2.8. In some embodiments, an AAV clade member encompassed by the disclosure comprises a VP1 capsid protein with at least about 93% identity to VP1 of AAV clade member No. 0 in Table 2.9. In some embodiments, an AAV clade member encompassed by the disclosure comprises a VP1 capsid protein with at least about 93% identity to VP1 of AAV clade member No. 0 in Table 2.10. In some embodiments, an AAV clade member encompassed by the disclosure comprises a VP1 capsid protein with at least about 93% identity to VP1 of AAV clade member No. 0 in Table 2.11. In some embodiments, an AAV clade member encompassed by the disclosure comprises a VP1 capsid protein with at least about 93% identity to VP1 of AAV clade member No. 0 in Table 2.12. In some embodiments, an AAV clade member encompassed by the disclosure comprises a VP1 capsid protein with at least about 93% identity to VP1 of AAV clade member No. 0 in Table 2.13. In some embodiments, an AAV clade member encompassed by the disclosure comprises a VP1 capsid protein with at least about 93% identity to VP1 of AAV clade member No. 0 in Table 2.14. In some embodiments, an AAV clade member encompassed by the disclosure comprises a VP1 capsid protein with at least about 93% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 94% identity to VP1 of AAV clade member No. 0 in Table 2.6. In some embodiments, an AAV clade member encompassed by the disclosure comprises a VP1 capsid protein with at least about 94% identity to VP1 of AAV clade member No. 0 in Table 2.7. In some embodiments, an AAV clade member encompassed by the disclosure comprises a VP1 capsid protein with at least about 94% identity to VP1 of AAV clade member No. 0 in Table 2.8. In some embodiments, an AAV clade member encompassed by the disclosure comprises a VP1 capsid protein with at least about 94% identity to VP1 of AAV clade member No. 0 in Table 2.9. In some embodiments, an AAV clade member encompassed by the disclosure comprises a VP1 capsid protein with at least about 94% identity to VP1 of AAV clade member No. 0 in Table 2.10. In some embodiments, an AAV clade member encompassed by the disclosure comprises a VP1 capsid protein with at least about 94% identity to VP1 of AAV clade member No. 0 in Table 2.11. In some embodiments, an AAV clade member encompassed by the disclosure comprises a VP1 capsid protein with at least about 94% identity to VP1 of AAV clade member No. 0 in Table 2.12. In some embodiments, an AAV clade member encompassed by the disclosure comprises a VP1 capsid protein with at least about 94% identity to VP1 of AAV clade member No. 0 in Table 2.13. In some embodiments, an AAV clade member encompassed by the disclosure comprises a VP1 capsid protein with at least about 94% identity to VP1 of AAV clade member No. 0 in Table 2.14. In some embodiments, an AAV clade member encompassed by the disclosure comprises a VP1 capsid protein with at least about 94% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 95% identity to VP1 of AAV clade member No. 0 in Table 2.6. In some embodiments, an AAV clade member encompassed by the disclosure comprises a VP1 capsid protein with at least about 95% identity to VP1 of AAV clade member No. 0 in Table 2.7. In some embodiments, an AAV clade member encompassed by the disclosure comprises a VP1 capsid protein with at least about 95% identity to VP1 of AAV clade member No. 0 in Table 2.8. In some embodiments, an AAV clade member encompassed by the disclosure comprises a VP1 capsid protein with at least about 95% identity to VP1 of AAV clade member No. 0 in Table 2.9. In some embodiments, an AAV clade member encompassed by the disclosure comprises a VP1 capsid protein with at least about 95% identity to VP1 of AAV clade member No. 0 in Table 2.10. In some embodiments, an AAV clade member encompassed by the disclosure comprises a VP1 capsid protein with at least about 95% identity to VP1 of AAV clade member No. 0 in Table 2.11. In some embodiments, an AAV clade member encompassed by the disclosure comprises a VP1 capsid protein with at least about 95% identity to VP1 of AAV clade member No. 0 in Table 2.12. In some embodiments, an AAV clade member encompassed by the disclosure comprises a VP1 capsid protein with at least about 95% identity to VP1 of AAV clade member No. 0 in Table 2.13. In some embodiments, an AAV clade member encompassed by the disclosure comprises a VP1 capsid protein with at least about 95% identity to VP1 of AAV clade member No. 0 in Table 2.14. In some embodiments, an AAV clade member encompassed by the disclosure comprises a VP1 capsid protein with at least about 95% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 96% identity to VP1 of AAV clade member No. 0 in Table 2.6. In some embodiments, an AAV clade member encompassed by the disclosure comprises a VP1 capsid protein with at least about 96% identity to VP1 of AAV clade member No. 0 in Table 2.7. In some embodiments, an AAV clade member encompassed by the disclosure comprises a VP1 capsid protein with at least about 96% identity to VP1 of AAV clade member No. 0 in Table 2.8. In some embodiments, an AAV clade member encompassed by the disclosure comprises a VP1 capsid protein with at least about 96% identity to VP1 of AAV clade member No. 0 in Table 2.9. In some embodiments, an AAV clade member encompassed by the disclosure comprises a VP1 capsid protein with at least about 96% identity to VP1 of AAV clade member No. 0 in Table 2.10. In some embodiments, an AAV clade member encompassed by the disclosure comprises a VP1 capsid protein with at least about 96% identity to VP1 of AAV clade member No. 0 in Table 2.11. In some embodiments, an AAV clade member encompassed by the disclosure comprises a VP1 capsid protein with at least about 96% identity to VP1 of AAV clade member No. 0 in Table 2.12. In some embodiments, an AAV clade member encompassed by the disclosure comprises a VP1 capsid protein with at least about 96% identity to VP1 of AAV clade member No. 0 in Table 2.13. In some embodiments, an AAV clade member encompassed by the disclosure comprises a VP1 capsid protein with at least about 96% identity to VP1 of AAV clade member No. 0 in Table 2.14. In some embodiments, an AAV clade member encompassed by the disclosure comprises a VP1 capsid protein with at least about 96% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 97% identity to VP1 of AAV clade member No. 0 in Table 2.6. In some embodiments, an AAV clade member encompassed by the disclosure comprises a VP1 capsid protein with at least about 97% identity to VP1 of AAV clade member No. 0 in Table 2.7. In some embodiments, an AAV clade member encompassed by the disclosure comprises a VP1 capsid protein with at least about 97% identity to VP1 of AAV clade member No. 0 in Table 2.8. In some embodiments, an AAV clade member encompassed by the disclosure comprises a VP1 capsid protein with at least about 97% identity to VP1 of AAV clade member No. 0 in Table 2.9. In some embodiments, an AAV clade member encompassed by the disclosure comprises a VP1 capsid protein with at least about 97% identity to VP1 of AAV clade member No. 0 in Table 2.10. In some embodiments, an AAV clade member encompassed by the disclosure comprises a VP1 capsid protein with at least about 97% identity to VP1 of AAV clade member No. 0 in Table 2.11. In some embodiments, an AAV clade member encompassed by the disclosure comprises a VP1 capsid protein with at least about 97% identity to VP1 of AAV clade member No. 0 in Table 2.12. In some embodiments, an AAV clade member encompassed by the disclosure comprises a VP1 capsid protein with at least about 97% identity to VP1 of AAV clade member No. 0 in Table 2.13. In some embodiments, an AAV clade member encompassed by the disclosure comprises a VP1 capsid protein with at least about 97% identity to VP1 of AAV clade member No. 0 in Table 2.14. In some embodiments, an AAV clade member encompassed by the disclosure comprises a VP1 capsid protein with at least about 97% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 98% identity to VP1 of AAV clade member No. 0 in Table 2.6. In some embodiments, an AAV clade member encompassed by the disclosure comprises a VP1 capsid protein with at least about 98% identity to VP1 of AAV clade member No. 0 in Table 2.7. In some embodiments, an AAV clade member encompassed by the disclosure comprises a VP1 capsid protein with at least about 98% identity to VP1 of AAV clade member No. 0 in Table 2.8. In some embodiments, an AAV clade member encompassed by the disclosure comprises a VP1 capsid protein with at least about 98% identity to VP1 of AAV clade member No. 0 in Table 2.9. In some embodiments, an AAV clade member encompassed by the disclosure comprises a VP1 capsid protein with at least about 98% identity to VP1 of AAV clade member No. 0 in Table 2.10. In some embodiments, an AAV clade member encompassed by the disclosure comprises a VP1 capsid protein with at least about 98% identity to VP1 of AAV clade member No. 0 in Table 2.11. In some embodiments, an AAV clade member encompassed by the disclosure comprises a VP1 capsid protein with at least about 98% identity to VP1 of AAV clade member No. 0 in Table 2.12. In some embodiments, an AAV clade member encompassed by the disclosure comprises a VP1 capsid protein with at least about 98% identity to VP1 of AAV clade member No. 0 in Table 2.13. In some embodiments, an AAV clade member encompassed by the disclosure comprises a VP1 capsid protein with at least about 98% identity to VP1 of AAV clade member No. 0 in Table 2.14. In some embodiments, an AAV clade member encompassed by the disclosure comprises a VP1 capsid protein with at least about 98% identity to VP1 of AAV clade member No. 0 in Table 2.15. In specific embodiments, an AAV clade member encompassed by the disclosure is not a known AAV capsid.

In some embodiments, an AAV clade member encompassed by the disclosure comprises a VP1 capsid protein with at least about 99% identity to VP1 of AAV clade member No. 0 in Table 2.6. In some embodiments, an AAV clade member encompassed by the disclosure comprises a VP1 capsid protein with at least about 99% identity to VP1 of AAV clade member No. 0 in Table 2.7. In some embodiments, an AAV clade member encompassed by the disclosure comprises a VP1 capsid protein with at least about 99% identity to VP1 of AAV clade member No. 0 in Table 2.8. In some embodiments, an AAV clade member encompassed by the disclosure comprises a VP1 capsid protein with at least about 99% identity to VP1 of AAV clade member No. 0 in Table 2.9. In some embodiments, an AAV clade member encompassed by the disclosure comprises a VP1 capsid protein with at least about 99% identity to VP1 of AAV clade member No. 0 in Table 2.10. In some embodiments, an AAV clade member encompassed by the disclosure comprises a VP1 capsid protein with at least about 99% identity to VP1 of AAV clade member No. 0 in Table 2.11. In some embodiments, an AAV clade member encompassed by the disclosure comprises a VP1 capsid protein with at least about 99% identity to VP1 of AAV clade member No. 0 in Table 2.12. In some embodiments, an AAV clade member encompassed by the disclosure comprises a VP1 capsid protein with at least about 99% identity to VP1 of AAV clade member No. 0 in Table 2.13. In some embodiments, an AAV clade member encompassed by the disclosure comprises a VP1 capsid protein with at least about 99% identity to VP1 of AAV clade member No. 0 in Table 2.14. In some embodiments, an AAV clade member encompassed by the disclosure comprises a VP1 capsid protein with at least about 99% identity to VP1 of AAV clade member No. 0 in Table 2.15. In specific embodiments, an AAV clade member encompassed by the disclosure is not a known AAV capsid.

In a specific embodiment, an AAV capsid member of a novel AAV clade of the disclosure has a VP1 amino acid sequence that is substantially related to a representative amino acid sequence of a novel AAV clade and one or more (e.g., 1, 2, 3, 4, 5, 6, 7, 8 or all) of the variable regions of the VP1 amino acid sequence are identical to the corresponding one or more variable regions of a representative amino acid sequence of the novel AAV clade. In another specific embodiment, an AAV clade member encompassed by the disclosure comprises a VP1 capsid protein with at least about 90% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 91% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 92% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In a specific embodiment, an AAV clade member encompassed by the disclosure comprises a VP1 capsid protein with at least about 93% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 94% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 95% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 96% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 97% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0. In another specific embodiment, an AAV clade member encompassed by the disclosure comprises a VP1 capsid protein with at least about 98% identity to VP1 of AAV clade member No. 0 in any one of Table 2 and one or more (e.g., 1, 2, 3, 4, 5, 6, 7, 8 or all) of the corresponding one or more variable regions of the VP1 capsid protein are identical to the corresponding one or more variable regions of clade member 0. In another specific embodiment, an AAV clade member encompassed by the disclosure comprises a VP1 capsid protein with at least about 99% identity to VP1 of AAV clade member No. 0 in any one of Table 2 and one or more (e.g., 1, 2, 3, 4, 5, 6, 7, 8 or all) of the variable regions of the VP1 capsid protein are identical to the corresponding one or more variable regions of clade member 0.

In a specific embodiment, an AAV capsid member of a novel AAV clade of the disclosure has an VP1 amino acid sequence that is substantially related to a representative amino acid sequence of a novel AAV clade and one or more (e.g., 1, 2, 3, 4, 5, 6, 7, 8 or all) of VRI-VRIX, and either the GBS, the GH loop or both the GBS and GH loop of the VP1 amino acid sequence are identical to the corresponding one or more (e.g., 1, 2, 3, 4, 5, 6, 7, 8 or all) of VRI-VRIX, and either the GBS, the GH loop or both the GBS and GH loop of a representative amino acid sequence of the novel AAV clade. In another specific embodiment, an AAV clade member encompassed by the disclosure comprises a VP1 capsid protein with at least about 90% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 91% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 92% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In a specific embodiment, an AAV clade member encompassed by the disclosure comprises a VP1 capsid protein with at least about 93% identity to VP1 of AAV clade member No. 0 in any one of Table 2 and one or more (e.g., 1, 2, 3, 4, 5, 6, 7, 8 or all) of the corresponding one or more variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 94% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 95% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 96% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 97% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 98% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0. In another specific embodiment, an AAV clade member encompassed by the disclosure comprises a VP1 capsid protein with at least about 99% identity to VP1 of AAV clade member No. 0 in any one of Table 2 and one or more (e.g., 1, 2, 3, 4, 5, 6, 7, 8 or all) of the variable regions, and either the GBS, the GH loop or both the GBS and GH loop of the VP1 capsid protein are identical to the corresponding one or more variable regions, and either the GBS, the GH loop or both the GBS and GH loop of clade member 0.

In certain embodiments, an AAV capsid member of a novel AAV clade of the disclosure has an VP1 amino acid sequence that is substantially related to a representative amino acid sequence of a novel AAV clade, provided that the AAV capsid member is not known in the art. In other words, the present disclosure does not encompass AAV capsid members that are known in the art. For example, AAV VP1 capsid sequences disclosed in any one of Table 2 with a prefix other than “BCD” and VP1 capsid sequences of any of the AAVs listed in Table 4 or Item A or Item B are not encompassed by the present disclosure.

In certain embodiments, an AAV clade member encompassed by the disclosure comprises a VP1 capsid protein with at least about 90% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In some embodiments, an AAV clade member encompassed by the disclosure comprises a VP1 capsid protein with at least about 91% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In certain embodiments, an AAV clade member encompassed by the disclosure comprises a VP1 capsid protein with at least about 92% identity to VP1 of AAV clade member No. 0 of in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In some embodiments, an AAV clade member encompassed by the disclosure comprises a VP1 capsid protein with at least about 93% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In certain embodiments, an AAV clade member encompassed by the disclosure comprises a VP1 capsid protein with at least about 94% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In some embodiments, an AAV clade member encompassed by the disclosure comprises a VP1 capsid protein with at least about 95% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In certain embodiments, an AAV clade member encompassed by the disclosure comprises a VP1 capsid protein with at least about 96% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In some embodiments, an AAV clade member encompassed by the disclosure comprises a VP1 capsid protein with at least about 97% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure). In certain embodiments, an AAV clade member encompassed by the disclosure comprises a VP1 capsid protein with at least about 98% identity to VP1 of AAV clade member No. 0 in any one of Table 2, provided that the AAV capsid member is not known in the art (e.g., AAV VP1 capsid sequences disclosed in any one of Table 2 with a prefix other than “BCD” and VP1 capsid sequences of any of the AA Vs listed in Table 4 or Item A or Item B are not encompassed by the present disclosure). In some embodiments, an AAV clade member encompassed by the disclosure comprises a VP1 capsid protein with at least about 99% identity to VP1 of AAV clade member No. 0 in Table 1, provided that the AAV capsid member is not known in the art (e.g., AAV VP1 capsid sequences disclosed in any one of Table 2 with a prefix other than “BCD” and VP1 capsid sequences of any of the AAVs listed in Table 4 or Item A or Item B are not encompassed by the present disclosure).

### 6.3.2.3 Common Variable Regions

The present disclosure also provides AAV clades grouped based on a common variable region (e.g., VRI-VRIX, GBS, or GH Loop). In some embodiments, an AAV member of a novel AAV clade of the disclosure has one or more common variable regions. That is, the amino acid sequences of one or more common variable regions across the viral capsid protein(s) (e.g., VP1, VP2, or VP3), such that the variable regions have substantial sequence similarity or identity between the AAV clade members.

In some embodiments, a member of a novel AAV clade of the disclosure has a common variable region(s) (e.g., VRI-VRIX, GBS, or GH Loop) if there is about 90% to 99% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 90% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 91% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 92% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 93% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 94% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 95% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 96% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 97% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 98% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 99% similarity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art.

In some embodiments, a member of a novel AAV clade of the disclosure has a common variable region(s) (e.g., VRI-VRIX, GBS, or GH Loop) if there is about 90% to 99% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 90% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 91% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 92% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 93% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 94% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 95% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 96% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 97% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 98% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art. In some embodiments, a member of a novel AAV clade of the disclosure has a common variable region(s) (e.g., VRI-VRIX, GBS, or GH Loop) if there is about 99% identity between the variable region(s) of the viral capsid protein(s) (e.g., VP1, VP2, or VP3) of the AAV clade member and the variable region(s) of the viral capsid protein(s) (e.g., VP1, VP2, or VP3) of another AAV clade member (e.g., AAV clade member No. 0 in any one of Table 2) using a technique described herein (e.g., ClustalW) or known to one of skill in the art.

In some embodiments, the variable regions of a capsid protein (e.g., VP1, VP2, or VP3) can be determined by a multiple sequence alignment of the amino acid sequence with a capsid viral protein (e.g., VP1, VP2, or VP3) of unrelated or related AAV capsid. For example, the variable regions spanning the VP1 capsid protein of AAV-9 can be identified by comparing it to the VP1 capsid proteins of AAV-2 or AAV-4; such a multiple sequence alignment will determine the variable regions (amino acid residues that vary) in the AAV9 VP1 capsid viral protein relative to AAV-2 and AAV-4.

In other embodiments, the variable regions of a capsid protein (e.g., VP1, VP2, or VP3) can be determined by structural alignment with the SSM (Secondary Structure Matching) program. See Krissinel E, Henrick K. Secondary-structure matching (SSM), a tool for fast protein structure alignment in three dimensions. Acta Crystallogr D Biol Crystallogr. 2004 December; 60 (Pt 12 Pt 1): 2256-68. doi: 10.1107/S0907444904026460, which is incorporated herein by reference in its entirety. For example, the crystal structure of a novel AAV capsid viral protein (VP) can be determined and compared to the VPs of AAVs such as AAV-2, AAV-3b, AAV-4, AAV-6, or AAV-8, for which high-resolution crystal structures are available. The crystal structure of an AAV capsid protein can be determined using cryo-EM (X-ray crystallography) or cryo-reconstruction. In some embodiments, clade-specific loop conformations are used as determined an AAV clade, as disclosed in Mietzsch M, et al. Viruses. 2021; 13 (1): 101. doi.org/10.3390/v13010101, which is incorporated herein by reference in its entirety.

In a specific embodiment, the location of the variable regions of a novel AAV capsid VP1 protein can be identified as described in Table 8, Example 9. In certain embodiments, the location of the N-terminal and/or C-terminal ends of the variable regions may vary by from up to 1 amino acid, 2 amino acids, 3 amino acids, 4 amino acids or 5 amino acids from the amino acid locations of the determined variable regions.

### 6.3.2.4 Phylogeny

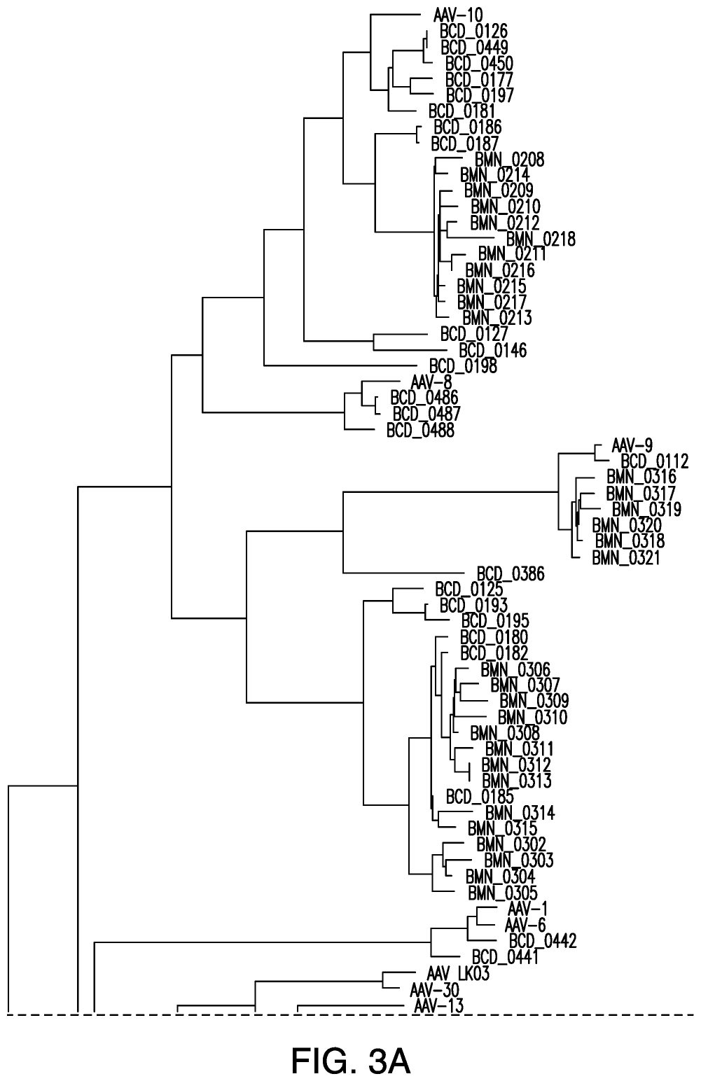

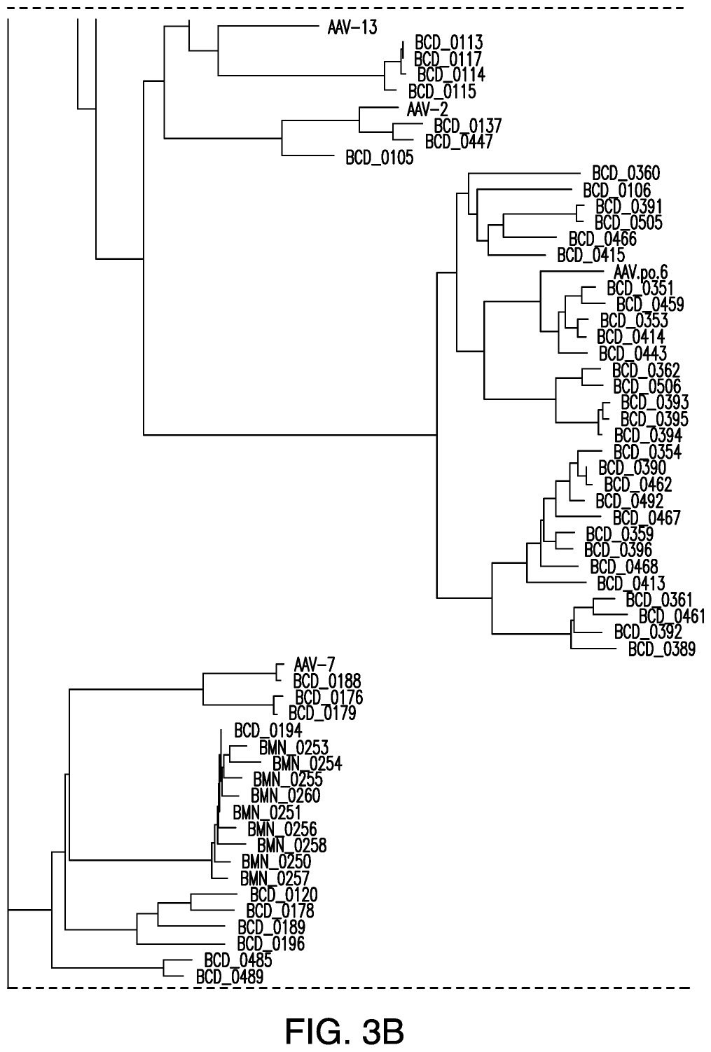

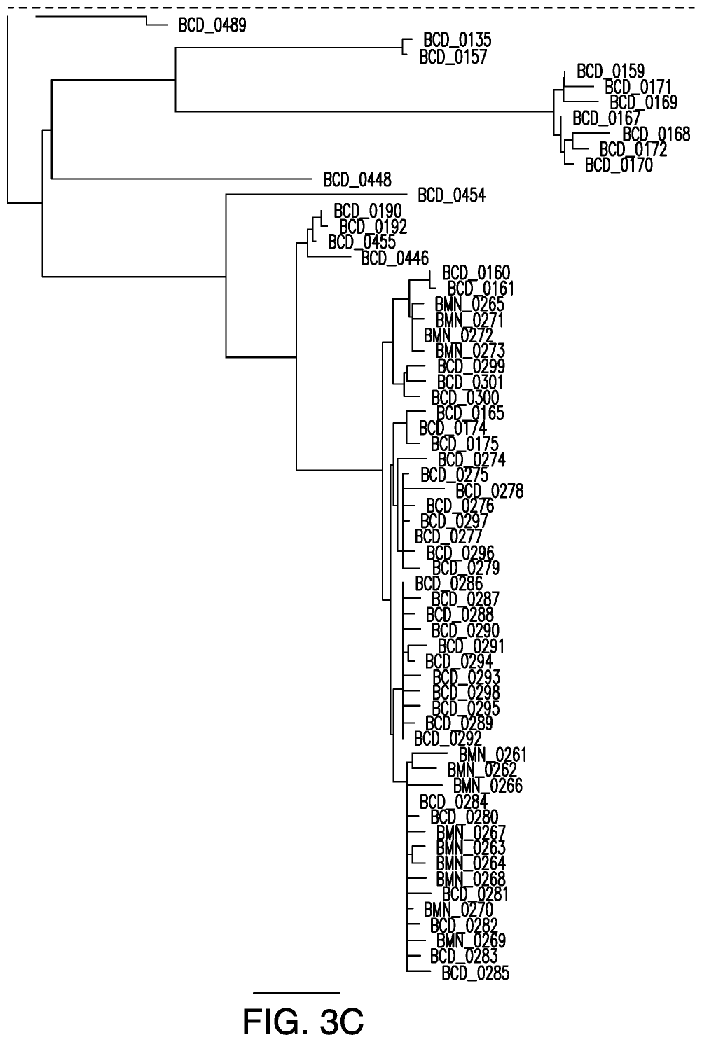

The present disclosure also provides AAV clades of AAV isolates (i.e., AAV members) grouped by their VP1 phylogenetic similarity. Also, provided are distinct AAV clades comprising AAV members with VP1 capsid sequences that are phylogenetically unrelated. In some embodiments, an AAV clade comprises one AAV member. In other embodiments, an AAV clade comprises at least two AAV members. Examples of phylogenetically related and distinct AAV clades of the disclosure are provided in FIGS. 3-13.

The phylogeny of an AAV VP1 amino acid sequence can be determined by a multiple sequence alignment with an VP1 capsid protein using an alignment program such as Clustal (e.g., Clustal W) or the like. Other nonlimiting multiple sequence alignment programs for amino acid sequences that can be used are, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one can use any other algorithm or computer program which provides at least the percent identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999).

After the multiple sequence alignment is performed, the genetic distance for the phylogenetic trees are generated using Neighbor-Joining or UPGMA method using bioinformatics software such Geneious Prime or the like, and selecting an appropriate genetic distance model such as, Jukes-Cantor. The phylogeny of an AAV VP1 amino acid sequence, if it is phylogenetically related to a novel AAV clade of the disclosure, is determined by such phylogenetic output. Examples of phylogenetically AAV clades and AAV branches of the disclosure are provided in FIGS. 3-14.

The genetic distances of AAV clades of the disclosure are provided in Table 3. The mean, min, and max genetic distance as compared to AAV members within the same AAV clade, with other AAV clades within the same AAV branch, or with other AAV clades in unrelated AAV branches are provided in Table 3 below.

TABLE 3
Genetic distances of Novel AAV Clades and AAV Branches
Genetic distances of AAV Clades and AAV Branches (mean, min and max)

| | | | | | Mean | | | | | |
|---|---|---|---|---|---|---|---|---|---|---|
| | | | | | to | Min to | Max to | | | |
| | | | | | Other | Other | Other | | | |
| | | Mean | Min | Max | Clades | Clades | Clades | Mean to | Min to | Max to |
| | | within | Within | Within | within | within | within | Other | Other | Other |
| AAV | AAV | the | the | the | the | the | the | Clades in | Clades in | Clades in |
| Clade | Branch | same | same | same | Same | Same | Same | Other | Other | Other |
| No. | No. | Clade | Clade | Clade | Branch | Branch | Branch | Branches | Branches | Branches |
| 0 | 2 | 0.019 | 0.000 | 0.093 | 0.186 | 0.110 | 0.238 | 0.349 | 0.287 | 0.493 |
| 1 | 2 | 0.066 | 0.000 | 0.126 | 0.167 | 0.072 | 0.221 | 0.350 | 0.276 | 0.503 |
| 4 | 2 | 0.051 | 0.001 | 0.081 | 0.203 | 0.156 | 0.238 | 0.340 | 0.234 | 0.500 |
| 6 | 2 | 0.060 | 0.000 | 0.094 | 0.156 | 0.092 | 0.206 | 0.346 | 0.273 | 0.497 |
| 11 | 2 | 0.009 | 0.001 | 0.018 | 0.171 | 0.072 | 0.220 | 0.356 | 0.302 | 0.503 |
| 12 | 2 | 0.029 | 0.011 | 0.044 | 0.161 | 0.069 | 0.192 | 0.349 | 0.288 | 0.493 |
| 13 | 2 | 0.011 | 0.003 | 0.020 | 0.182 | 0.121 | 0.214 | 0.347 | 0.301 | 0.499 |
| 16 | 2 | 0.016 | 0.008 | 0.022 | 0.162 | 0.113 | 0.197 | 0.347 | 0.276 | 0.496 |
| 21 | 2 | 0.050 | 0.000 | 0.083 | 0.156 | 0.069 | 0.189 | 0.345 | 0.284 | 0.490 |
| 22 | 2 | 0.003 | 0.003 | 0.003 | 0.163 | 0.108 | 0.221 | 0.345 | 0.294 | 0.490 |

In some embodiments, an AAV clade comprises at least two AAV members, wherein each AAV member is phylogenetically related as determined by comparing its VP1 amino acid sequence using the Neighbor-joining method, wherein the VP1 amino acid sequence of an AAV member and has a genetic distance (i.e., mean, min, or max genetic distance within a clade) provided in Table 3 to the VP1 amino acid sequence of each other AAV member. In certain embodiments, at least one AAV member of the AAV clade comprises a VP1 amino acid sequence of any one of SEQ ID NOs: 1-180 and 361-362.

### Novel AAV Branches

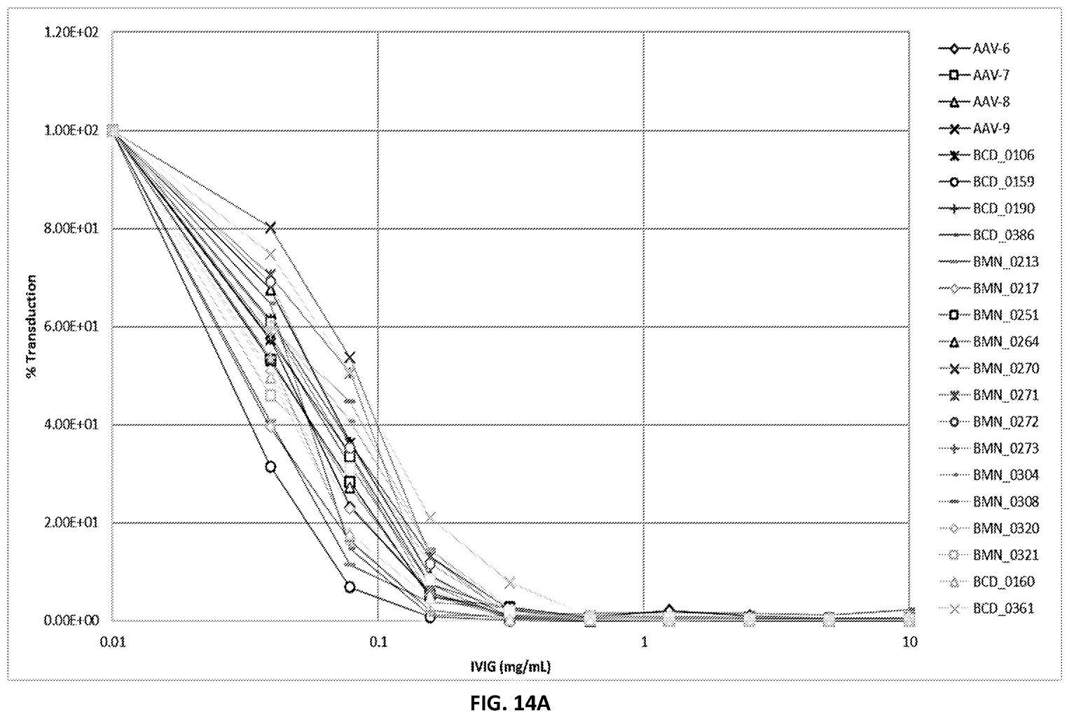

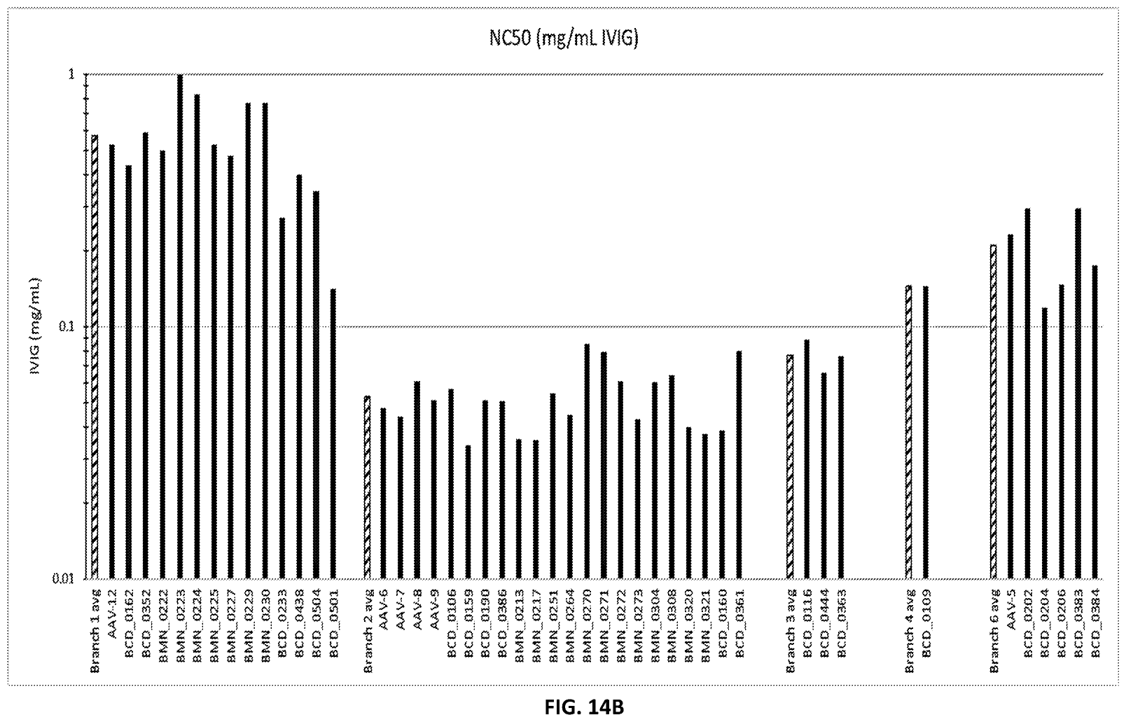

In addition to novel AAV clades, the present disclosure also provides novel AAV branches (i.e., a group of different AAV clades) based on common phylogeny (e.g., genetic distance or capsid sequence identity) and/or common function. In some embodiments, the AAV branch is defined by min, max, and average genetic distance as described in Table 3. In some embodiments, the AAV branch is defined by the AAV clades profile to evade neutralization by human serum (i.e., evading AAV humoral immunity). See, e.g., Example 4 and FIGS. 14A-B. In some embodiments, the AAV branch is defined by the genetic distance and the profile of evading AAV humoral immunity.

In some embodiments, provided herein are branches of AAV capsid sequences that are phylogenetically related. In some embodiments, an AAV branch comprises at least two AAV members, wherein each AAV member is phylogenetically related as determined by comparing its VP1 amino acid sequence using the Neighbor joining method, wherein the VP1 amino acid sequence of an AAV member and has a genetic distance (i.e., mean or a range min or max genetic distance as other clades in the same branch) provided in Table 3. In some embodiments, at least one AAV member of the AAV branch comprises a VP1 amino acid sequence of any one of SEQ ID NOs: 1-180 and 361-362.

In specific embodiments, an adeno-associated virus (AAV) branch, comprises at least two AAV members, wherein each AAV member is phylogenetically related as determined by comparing its VP1 amino acid sequence using the Neighbor joining method, wherein the VP1 amino acid sequence of an AAV member has at least 40% identity to the VP1 amino acid sequence of each other AAV member, and wherein at least one AAV member of AAV branch comprises a VP1 amino acid sequence of No. 0 in Table 2. In certain embodiments, each AAV member of an AAV branch comprises one or more variable region(s) (e.g., 1, 2, 3, 4, 5, 6, 7, 8 or 9) of the VP1 amino acid sequence that is identical to the corresponding one or more variable region(s) of the VP1 amino acid sequence of No. 0 in Table 2. In some embodiments, each AAV member of an AAV branch comprises a GBS, GH loop, or both a GBS and GH loop of the VP1 amino acid sequence that is identical to the GBS, the GH loop, or both the GBS and GH loop of the VP1 amino acid sequence of No. 0 in Table 2. In certain embodiments, each AAV member of an AAV branch comprises one or more (e.g., 1, 2, 3, 4, 5, 6, 7, 8 or all) of VRI-VRIX and either the GBS, the GH loop, or both the GBS and GH loop of the VP1 amino acid sequence that is identical to the corresponding one or more (e.g., 1, 2, 3, 4, 5, 6, 7, 8 or all) of VRI-VRIX and either the GBS, the GH loop, or both the GBS and GH loop of the VP1 amino acid sequence of No. 0 in Table 2.

### 6.3.2.5 Tropism

Upon reviewing the present disclosure, it will be readily apparent to one of skilled in the art that certain novel AAV clades, AAV branches, and/or individual AAV capsid sequences of the disclosure are particularly useful as rAAV-based vectors and/or rAAV viral particles for certain biomedical applications based on their cell/tissue specificity, e.g., tropism.

For example, viral particles made using the novel AAV sequence(s) of a clade are useful for delivering a biomolecule (e.g., a therapeutic biomolecule) or agent to muscle tissue, including the heart muscle. While viral particles made using AAV capsid sequence(s) of a different clade are useful for delivering a biomolecule (e.g., a therapeutic biomolecule) or agent to the liver, brain or CNS. Uses of such of novel AAV sequences comprising the clade are not limited and one of skill in the art may utilize these for delivery to other cell types, tissues, or organs.

In some embodiments, a novel rAAV viral particle of the disclosure has similar or comparable tropism for a cell type or tissue as compared to a reference AAV. In some embodiments, a novel rAAV viral particle of the disclosure has enhanced/increased tropism for a cell type or tissue as compared to a reference AAV. The reference AAV may be a naturally occurring AAV serotype (e.g., AAV-1, AAV-2, AAV-3, AAV-4, AAV-5, AAV-6, AAV-7, AAV-8, AAV-9, AAV-10, AAV-rh10, AAV-11, AAV-12, or AAV-13). Alternatively, the reference AAV may be a known AAV comprising a chimeric, engineered, or hybrid capsid. In some embodiments, the reference AAV is one described in the Examples, infra. In some embodiments, the reference AAV is AAVrh.8, AAVrh.10, AAVrh.39, AAVrh.43, or AAV-9. In instances where a novel rAAV viral particle comprises a modified AAV capsid sequence (such as described in Section 6.3.1.4, supra), a novel rAAV viral particle with the corresponding unmodified AAV capsid sequence may be used as a reference AAV. In addition, a reference AAV may be one novel rAAV viral particle of the disclosure compared to another novel viral particle of the disclosure.

In some embodiments, a novel rAAV viral particle of the disclosure has tropism for a cell or tissue from the CNS, heart, lung, trachea, esophagus, muscle, bone, cartilage, stomach, pancreas, intestine, liver, bladder, kidney, ureter, urethra, uterus, fallopian tube, ovary, testes, prostate, eye, blood, lymph, or oral mucosa. In certain embodiments, a novel rAAV viral particle of the disclosure has tropism for a muscle cell (e.g., skeletal muscle cell, smooth muscle cell, diaphragm muscle cell, and/or cardiac muscle cell) or muscle tissue (e.g., skeletal muscle, smooth muscle, diaphragm muscle, and/or cardiac muscle). In some embodiments, a novel rAAV viral particle of the disclosure has tropism for a liver cell or liver tissue. In some embodiments, a novel rAAV viral particle of the disclosure has tropism for an ear cell or ear tissue. In certain embodiments, a novel rAAV viral particle of the disclosure has tropism for a spinal cord cell or spinal cord tissue. In some embodiments, a novel rAAV viral particle of the disclosure has tropism for a CNS cell or CNS tissue (e.g., including brain tissues). In certain embodiments, a novel rAAV viral particle of the disclosure has tropism for one, two or more of the following cells: neurons, glial cells, astrocytes, oligodendroglia, microglia, Schwann cells, ependymal cells, stellate fat storing cells, Kupffer cells, hepatocytes, liver endothelial cells, ocular cells, epithelial cells, cardiomyocytes, smooth muscle cells, pancreatic cells, lung cells, T-cells, B cells, hematopoietic stem cells, and embryonic stem cells. In a specific embodiment, biodistribution of a novel rAAV viral particle of the disclosure is assessed using a technique known to one of skill in the art or described herein (e.g., in the Examples (e.g., Example 6 or 7), infra). In another specific embodiment, the distribution in brain tissue of a novel rAAV viral particle of the disclosure is assessed using a technique known to one of skill in the art or described herein (e.g., in the Examples (e.g., Example 8 or 11), infra).

In some embodiments, a novel rAAV viral particle of the disclosure has enhanced muscle tropism (e.g., human skeletal muscle tropism or smooth muscle tropism) as compared to a reference AAV (e.g., AAV-1, AAV-6, AAV-7, AAV-8, AAV-9, or AAV-rh.10). In other embodiments, a novel rAAV viral particle of the disclosure has similar or comparable muscle tropism (e.g., human skeletal muscle tropism or smooth muscle tropism) as compared to a reference AAV (e.g., AAV-1, AAV-6, AAV-7, AAV-8, AAV-9 or AAV-rh.10). In certain embodiments, a novel rAAV viral particle of the disclosure has enhanced central nervous system (CNS) tropism as compared to a reference AAV (e.g., AAV-1, AAV-4, AAV-5, AAV-6, AAV-9, AAV-9-PHP.eB, or AAV-rh. 10). In other embodiments, a novel rAAV viral particle of the disclosure has similar or comparable central nervous system (CNS) tropism as compared to a reference AAV (e.g., AAV-1, AAV-4, AAV-5, AAV-6, AAV-9, AAV-9-PHP.eB, or AAV-rh10). In some embodiments, a novel rAAV viral particle of the disclosure has enhanced brain tropism as compared to a reference AAV (e.g., AAVrh.8, AAVrh. 10, AAVrh.39, AAVrh.43, AAV-9, or AAV-9-PHP.eB). In other embodiments, a novel AAV of the disclosure has similar or comparable brain tropism as compared to a reference AAV (e.g., AAVrh.8, AAVrh. 10, AAVrh.39, AAVrh.43, AAV-9, or AAV-9-PHP.eB). In certain embodiments, a novel AAV of the disclosure has enhanced heart tropism as compared to a reference AAV (e.g., AAV-2, AAV-3, AAV-6, AAV-7, AAV-8, AAV-9, or AAV-rh. 10). In other embodiments, a novel AAV of the disclosure has similar or comparable heart tropism as compared to a reference AAV (e.g., AAV-2, AAV-3, AAV-6, AAV-7, AAV-8, AAV-9, or AAV-rh.10). In certain embodiments, a novel AAV of the disclosure has enhanced liver tropism as compared to a reference AAV (e.g., AAV-2, AAV-3, AAV-5, AAV-6, AAV-7, AAV-8, AAV-9, or AAV-rh.10). In other embodiments, a novel AAV of the disclosure has similar or comparable liver tropism as compared to a reference AAV (e.g., AAV-2, AAV-3, AAV-5, AAV-6, AAV-7, AAV-8, AAV-9, or AAV-rh. 10). In other embodiments, a novel AAV of the disclosure has lower liver tropism as compared to a reference AAV (e.g., AAV-2, AAV-3, AAV-6, AAV-5, AAV-7, AAV-8, AAV-9, or AAV-rh10). In some embodiments, novel AAV of the disclosure has similar or comparable ear tropism as compared to a reference AAV (e.g., AAV-2, AAV-3, AAV-6, AAV-7, AAV-8, AAV-9, AAV-9-PHP.B or AAV-rh10). In other embodiments, a novel AAV of the disclosure has similar or comparable ear tropism as compared to a reference AAV (e.g., AAV-2, AAV-3, AAV-6, AAV-7, AAV-8, AAV-9, AAV-9-PHP.B or AAV-rh10). The tropism may be assessed by a technique described herein (e.g., in the Examples, infra) or one known to one of skill in the art. In certain embodiments, a novel AAV of the disclosure has enhanced cancer tropism as compared to a reference AAV (e.g., AAV-2, AAV-3, AAV-6, AAV-7, AAV-8, AAV-9, or AAV-rh10). In other embodiments, a novel AAV of the disclosure has similar or comparable cancer tropism as compared to a reference AAV (e.g., AAV-2, AAV-3, AAV-6, AAV-7, AAV-8, AAV-9, or AAV-rh10). In some embodiments, the capsid is one referenced in the Examples, infra. In some embodiments, the capsid protein is a BCD_0126, BCD_0282, BCD_0176, BCD_0446, BCD_0160, BCD_0195, BCD_0180, BCD_0192, BCD_0185, BCD_0454, BCD_0277, BCD_0174, BCD_0167, BCD_0126, BCD_0125, BCD_0193, BCD_0286, BCD_0182, BCD_0283, BCD_0106, or BCD_0361 capsid protein.

In some embodiments, a novel rAAV viral particle of the disclosure has about 5%, 10%, 15%, 20%, 25%, 30%, 35%, 40%, 45%, or 50% greater tropism for a particular cell type or tissue as compared to a reference AAV. In some embodiments, a novel rAAV viral particle of the disclosure has at least about 5%, 10%, 15%, 20%, 25%, 30%, 35%, 40%, 45%, or 50% greater tropism for a particular cell type or tissue as compared to a reference AAV. In some embodiments, a novel rAAV viral particle of the disclosure has about 55%, 60%, 65%, 70%, 75%, 80%, 85%, 90%, 95%, 99%, or 100% greater tropism for a particular cell type or tissue as compared to a reference AAV. In some embodiments, a novel rAAV viral particle of the disclosure has at least about 55%, 60%, 65%, 70%, 75%, 80%, 85%, 90%, 95%, 99%, or 100% greater tropism for a particular cell type or tissue as compared to a reference AAV. In some embodiments, a novel rAAV viral particle of the disclosure has about 125%, 150%, 175%, 200%, 250%, 300%, 350%, 400%, or 500% greater tropism for a particular cell type or tissue as compared to a reference AAV. In some embodiments, a novel rAAV viral particle of the disclosure has at least about 125%, 150%, 175%, 200%, 250%, 300%, 350%, 400%, or 500% greater tropism for a particular cell type or tissue as compared to a reference AAV.

In some embodiments, a novel rAAV viral particle of the disclosure has 5% to 25%, 15% to 30%, 25% to 50%, or 40%, to 50% greater tropism for a particular cell type or tissue as compared to a reference AAV. In certain embodiments, a novel rAAV viral particle of the disclosure has 55% to 75%, 70% to 85%, 75% to 95%, 90% to 99%, or 75%, to 100% greater tropism for a particular cell type or tissue as compared to a reference AAV. In some embodiments, a novel rAAV viral particle of the disclosure has about 125% to 200%, 200% to 250%, 150% to 300%, 200% to 400%, 250% to 500%, or 400% to 500% greater tropism for a particular cell type or tissue as compared to a reference AAV. In a specific embodiment, the tropism for a particular cell type or tissue is assessed using a technique known to one of skill in the art or described herein. In some embodiments, the cell type or tissue is muscle. In some embodiments, the cell type or tissue is muscle and the reference AAV is AAV-1, AAV-6, AAV-7, AAV-8, AAV-9, or AAV-rh.10. In some embodiments, the cell type or tissue is heart. In some embodiments, the cell type or tissue is heart and the reference AAV is AAV-1, AAV-6, AAV-7, AAV-8, AAV-9, or AAV-rh.10. In some embodiments, the cell type or tissue is brain. In some embodiments, the cell type or tissue is brain and the reference AAV is AAVrh.8, AAVrh. 10, AAVrh.39, AAVrh.43, or AAV-9. In some embodiments, the cell type is a neuron. In some embodiments, the cell type is a neuron and the reference AAV is AAVrh.8, AAVrh.10, AAVrh.39, AAVrh.43, AAV-9, or AAV-9-PHP.eB. In some embodiments, the cell type or tissue is ear. In some embodiments, the cell type or tissue is ear and the reference AAV is AAV-2, AAV-3, AAV-6, AAV-7, AAV-8, AAV-9, AAV-9-PHP.B or AAV-rh10. In some embodiments, the cell type or tissue is plasma. In some embodiments, the cell type or tissue is plasma and the reference AAV is AAV-2, AAV-3, AAV-6, AAV-7, AAV-8, AAV-9, or AAV-rh10. In some embodiments, the cell type or tissue is kidney. In some embodiments, the cell type or tissue is kidney and the reference AAV is AAV-2, AAV-3, AAV-6, AAV-7, AAV-8, AAV-9, or AAV-rh10. In some embodiments, the cell type or tissue is liver. In some embodiments, the cell type or tissue is liver and the reference AAV is AAV-2, AAV-3, AAV-5. AAV-6, AAV-7, AAV-8, AAV-9, or AAV-rh.10. In some embodiments, the cell type or tissue is ear. In some embodiments, the cell type or tissue is ear and the reference AAV is AAV-2, AAV-3, AAV-6, AAV-7, AAV-8, AAV-9, AAV-9-PHP.B, or AAV-rh.10.

In some embodiments, a novel rAAV viral particle of the disclosure with increased tropism for a particular cell type or tissue as compared to a reference AAV has increased expression of a gene product encoded by a transgene incorporated into the novel rAAV viral particle as compared to the expression of the same gene product encoded by the same transgene incorporated into the reference AAV. In certain embodiments, the expression of the gene product encoded by the transgene incorporated into the novel rAAV viral particle is 5% to 25%, 15% to 30%, 25% to 50%, or 40%, to 50% greater than the expression of the same gene product encoded by the same transgene incorporated into the reference AAV. In some embodiments, the expression of the gene product encoded by the transgene incorporated into the novel rAAV viral particle is 55% to 75%, 70% to 85%, 75% to 95%, 90% to 99%, or 75%, to 100% greater than the expression of the same gene product encoded by the same transgene incorporated into the reference AAV. In certain embodiments, the expression of the gene product encoded by the transgene incorporated into the novel rAAV viral particle is 125% to 200%, 200% to 250%, 150% to 300%, 200% to 400%, 250% to 500%, or 400% to 500% greater than the expression of the same gene product encoded by the same transgene incorporated into the reference AAV. In some embodiments, the expression of a gene product is measured at the RNA level by a technique known to one of skill in the art (e.g., Northern blot, RT-PCR, etc.) or described herein. In certain embodiments, the expression of a gene product is measured at the protein level by a technique known to one of skill in the art (e.g., Western blot, ELISA, or another immunoassay) or described herein. In some embodiments, the cell type or tissue is muscle. In some embodiments, the cell type or tissue is muscle and the reference AAV is AAV-1, AAV-6, AAV-7, AAV-8, AAV-9, or AAV-rh10. In some embodiments, the cell type or tissue is heart. In some embodiments, the cell type or tissue is heart and the reference AAV is AAV-1, AAV-6, AAV-7, AAV-8, AAV-9, or AAV-rh10. In some embodiments, the cell type or tissue is brain. In some embodiments, the cell type or tissue is brain and the reference AAV is AAVrh.8, AAVrh. 10, AAVrh.39, AAVrh.43, or AAV9. In some embodiments, the cell type is a neuron. In some embodiments, the cell type is a neuron and the reference AAV is AAVrh.8, AAVrh.10, AAVrh.39, AAVrh.43, or AAV9. In some embodiments, the cell type or tissue is plasma. In some embodiments, the cell type or tissue is ear. In some embodiments, the cell type or tissue is plasma and the reference AAV is AAV-2, AAV-3, AAV-6, AAV-7, AAV-8, AAV-9, AAV-9-PHP.B, or AAV-rh. 10. In some embodiments, the cell type or tissue is kidney. In some embodiments, the cell type or tissue is kidney and the reference AAV is AAV-2, AAV-3, AAV-6, AAV-7, AAV-8, AAV-9, or AAV-rh10. In some embodiments, the cell type or tissue is liver. In some embodiments, the cell type or tissue is liver and the reference AAV is AAV-2, AAV-3, AAV-5, AAV-6, AAV-7, AAV-8, AAV-9, or AAV-rh10. In some embodiments, the cell type or tissue is ear. In some embodiments, the cell type or tissue is ear and the reference AAV is AAV-2, AAV-3, AAV-6, AAV-7, AAV-8, AAV-9, AAV-9-PHP.B, or AAV-rh.10.

In some embodiments, a novel rAAV viral particle of the disclosure has increased transduction efficiency as compared to a reference AAV. In certain embodiments, the transduction efficiency of a novel rAAV viral particle is increased by about 1-fold, 2-fold, 3-fold, 4-fold, 5-fold, 6-fold, 7-fold, 8-fold, 9-fold, 10-fold, 11-fold, 12-fold, 15-fold, 20-fold, 25-fold, 30-fold, 40-fold, 50-fold, or more than 50-fold compared to a reference AAV. In some embodiments, transduction efficiency of a novel rAAV viral particle is increased by at least about 1-fold, 2-fold, 3-fold, 4-fold, 5-fold, 6-fold, 7-fold, 8-fold, 9-fold, 10-fold, 11-fold, 12-fold, 15-fold, 20-fold, 25-fold, 30-fold, 40-fold, 50-fold, or more than 50-fold compared to a reference AAV. In certain embodiments, the transduction efficiency of a novel rAAV viral particle is increased by about 1-fold to 3-fold, 2-fold to 5-fold, 5-fold to 10-fold, or 10-fold to 20-fold compared to a reference AAV. In specific embodiments, transduction efficiency is determined by a technique known to one of skill in the art or described herein (e.g., in the Examples (e.g., Example 5), infra).

In some embodiments, a novel rAAV viral particle of the disclosure with increased tropism for a particular cell or tissue relative to a reference AAV, while de-targeting another cell or tissue, as assessed by a technique known to one of skill in the art or described herein (e.g., as described in Examples 6 and 7). In specific embodiments, a novel rAAV viral particle of the disclosure with increased tropism for the heart relative to a reference AAV, while de-targeting the liver, as assessed by a technique known to one of skill in the art or described herein (e.g., as described in Example 6).

In some embodiments, a novel rAAV viral particle of the disclosure has decreased tropism for a particular cell or tissue (e.g., a liver cell or the liver) as compared to a reference AAV. In some embodiments, the decrease is about 5%, 10%, 15%, 20%, 25%, 30%, 35%, 40%, 45%, or 50% decrease in tropism for a particular cell type or tissue (e.g., a liver cell or the liver) as compared to a reference AAV.

In some embodiments, a novel rAAV viral particle of the disclosure has increased transduction efficiency as compared to a reference AAV. In certain embodiments, the transduction efficiency of a novel rAAV viral particle is increased by about 0.5 log, 1 log, 1.5 logs, 2 logs, 2.5 logs, 3 logs, or more compared to a reference AAV. In some embodiments, transduction efficiency of a novel rAAV viral particle is increased by at least about 0.5 log, at least about 1 log, at least about 1.5 logs, at least about 2 logs, at least about 2.5 logs, at least about 3 logs, or more compared to a reference AAV. In certain embodiments, the transduction efficiency of a novel rAAV viral particle is increased by about 0.5 log to 3 log, 0.5 log to 2.5 logs, 0.5 log to 2 logs, 0.5 log to 1.5 logs, or 0.5 log to 1 log compared to a reference AAV. In certain embodiments, the transduction efficiency of a novel rAAV viral particle is increased by about 1 log to 3 log, 1 log to 2.5 logs, 1 log to 2 logs, or 1 log to 1.5 logs compared to a reference AAV. In certain embodiments, the transduction efficiency of a novel rAAV viral particle is increased by about 2 log to 2.5 log or 2 log to 3 logs compared to a reference AAV. In specific embodiments, transduction efficiency is determined by a technique known to one of skill in the art or described herein (e.g., in the Examples (e.g., Example 5), infra).

In some embodiments, a novel rAAV viral particle of the disclosure has increased tropism for a particular cell or tissue relative to a reference AAV (e.g., AAV-1, AAV-6, AAV-7, AAV-8, AAV-9, or AAV-rh10), while de-targeting another cell or tissue, as assessed by a technique known to one of skill in the art or described herein (e.g., as described in Example 6, 7, 8, 10, or 11). In some embodiments, the increase is an increase that is at least 10%, at least 20%, at least 30%, at least 40%, at least 50%, at least 60%, or at least 70% greater than the AAV reference. In some embodiments, the increase is an increase that is at least 0.5 log, at least 1 log, at least 1.5 logs, at least 2 logs, at least 2.5 logs, or at least 3 logs greater than the AAV reference. In some embodiments, the increase is an increase that is 0.5 log, 1 log, 1.5 logs, 2 logs, 2.5 logs, or 3 logs greater than the AAV reference. In some embodiments, the increase is an increase that is at least 0.5 log to 3 logs greater than the AAV reference. In some embodiments, the increase is an increase that is 0.5 log to 2 logs, 0.5 log to 2.5 logs, 0.5 log to 3 logs, 1 log to 3 logs, or 1 log to 2 logs greater than the AAV reference. In specific embodiments, a novel rAAV viral particle of the disclosure has increased tropism for the heart relative to a reference AAV, while de-targeting the liver, as assessed by a technique known to one of skill in the art or described herein (e.g., as described in Example 6). In a specific embodiment, the novel rAAV viral particle comprises a capsid protein with a “BCD_” prefix in Example 6, infra. In a specific embodiment, the novel rAAV particle comprises BCD_0126 capsid protein. In another specific embodiment, the novel rAAV viral particle comprises a BCD_0282 capsid protein. In another specific embodiment, the novel rAAV viral particle comprises a BCD_0176 capsid protein. In another specific embodiment, the novel rAAV viral particle comprises a BCD_0446 capsid protein. In another specific embodiment, the novel rAAV viral particle comprises a BCD_0160 capsid protein. In another specific embodiment, the novel rAAV viral particle comprises a BCD_0195 capsid protein. In another specific embodiment, the novel rAAV viral particle comprises a BCD_0180 capsid protein. In another specific embodiment, the novel rAAV viral particle comprises a BCD_0192 capsid protein. In another specific embodiment, the novel rAAV viral particle comprises a BCD_0185 capsid protein. In another specific embodiment, the novel rAAV viral particle comprises a BCD_0454 capsid protein. In another specific embodiment, the novel rAAV viral particle comprises a BCD_0277 capsid protein. In another specific embodiment, the novel rAAV viral particle comprises a BCD_0174 capsid protein. In another specific embodiment, the novel rAAV viral particle comprises a BCD_0167 capsid protein. In another specific embodiment, the novel rAAV viral particle comprises a BCD_0126 capsid protein. In another specific embodiment, the novel rAAV viral particle comprises a BCD_0125 capsid protein. In another specific embodiment, the novel rAAV viral particle comprises a BCD_0193 capsid protein. In another specific embodiment, the novel rAAV viral particle comprises a BCD_0286 capsid protein. In another specific embodiment, the novel rAAV viral particle comprises a BCD_0182 capsid protein. In another specific embodiment, the novel rAAV viral particle comprises a BCD_0283 capsid protein. In another specific embodiment, the novel rAAV viral particle comprises a BCD_0361 capsid protein. In another specific embodiment, the novel rAAV viral particle comprises a BCD_0106 capsid protein.

In some embodiments, a novel rAAV viral particle has increased tropism for a brain tissue relative to a reference AAV (e.g., AAVrh.8, AAVrh.10, AAVrh.39, AAVrh.43, or AAV-9, AAV-9-PHP.eb). In some embodiments, a novel rAAV viral particle has increased tropism for a brain neuron relative to a reference AAV (e.g., AAVrh.8, AAVrh.10, AAVrh.39, AAVrh.43, or AAV-9, AAV-9-PHP.eb). In some embodiments, the increase is an increase that is at least 30%, at least 40%, at least 50%, at least 60%, or at least 70% greater than the AAV reference. In some embodiments, the increase is an increase that is 30% to 50% or 50% to 75% greater than the AAV reference. In some embodiments, the increase is an increase that is at least 0.5 log, at least 1 log, at least 1.5 logs, at least 2 logs, at least 2.5 logs, or at least 3 logs greater than the AAV reference. In some embodiments, the increase is an increase that is 0.5 log, 1 log, 1.5 logs, 2 logs, 2.5 logs, or 3 logs greater than the AAV reference. In some embodiments, the increase is an increase that is at least 0.5 log to 3 logs greater than the AAV reference. In some embodiments, the increase is an increase that is 0.5 log to 2 logs, 0.5 log to 2.5 logs, 0.5 log to 3 logs, 1 log to 3 logs, or 1 log to 2 logs greater than the AAV reference. In a specific embodiment, the novel rAAV viral particle comprises a capsid protein with a “BCD_” prefix in Example 6, 8, or 11, infra. In a specific embodiment, the novel rAAV particle comprises BCD_0126 capsid protein. In another specific embodiment, the novel rAAV viral particle comprises a BCD_0282 capsid protein. In another specific embodiment, the novel rAAV viral particle comprises a BCD_0176 capsid protein. In another specific embodiment, the novel rAAV viral particle comprises a BCD_0446 capsid protein. In another specific embodiment, the novel rAAV viral particle comprises a BCD_0160 capsid protein. In another specific embodiment, the novel rAAV viral particle comprises a BCD_0195 capsid protein. In another specific embodiment, the novel rAAV viral particle comprises a BCD_0180 capsid protein. In another specific embodiment, the novel rAAV viral particle comprises a BCD_0192 capsid protein. In another specific embodiment, the novel rAAV viral particle comprises a BCD_0185 capsid protein. In another specific embodiment, the novel rAAV viral particle comprises a BCD_0454 capsid protein. In another specific embodiment, the novel rAAV viral particle comprises a BCD_0277 capsid protein. In another specific embodiment, the novel rAAV viral particle comprises a BCD_0174 capsid protein. In another specific embodiment, the novel rAAV viral particle comprises a BCD_0167 capsid protein. In another specific embodiment, the novel rAAV viral particle comprises a BCD_0126 capsid protein. In another specific embodiment, the novel rAAV viral particle comprises a BCD_0125 capsid protein. In another specific embodiment, the novel rAAV viral particle comprises a BCD_0193 capsid protein. BCD_0286 capsid protein. In another specific embodiment, the novel rAAV viral particle comprises a BCD_0182 capsid protein. In another specific embodiment, the novel rAAV viral particle comprises a BCD_0283 capsid protein. In another specific embodiment, the novel rAAV viral particle comprises a BCD_0106 capsid protein.

In some embodiments, a novel rAAV viral particle has increased tropism for the ear relative to a reference AAV (e.g., AAV-2, AAV-3, AAV-6, AAV-7, AAV-8, AAV-9, or AAV-rh10). In a specific embodiment, the novel rAAV viral particle comprises a capsid protein with a “BCD_” prefix in Example 10, infra. In a specific embodiment, the novel rAAV viral particle comprises a BCD_0361 capsid protein. In a specific embodiment, the novel rAAV viral particle comprises a BCD_0106 capsid protein.

In some embodiments, a novel rAAV viral particle has increased tropism for the heart or liver relative to a reference AAV (e.g., AAV-2, AAV-3, AAV-6, AAV-7, AAV-8, AAV-9, or AAV-rh10). In a specific embodiment, the novel rAAV viral particle comprises a BCD_0126 capsid protein.

In some embodiments, a novel rAAV viral particle of the disclosure has increased activity (e.g., expression of a transgene) in a particular cell or tissue relative to a reference AAV (e.g., AAV-1, AAV-6, AAV-7, AAV-8, AAV-9, or AAV-rh10), as assessed by a technique known to one of skill in the art or described herein. In some embodiments, a novel rAAV viral particle of the disclosure has increased activity (e.g., expression of a transgene) in heart cells or heart tissue relative to a reference AAV (e.g., AAV-1, AAV-6, AAV-7, AAV-8, AAV-9, or AAV-rh10), as assessed by a technique known to one of skill in the art or described herein. In some embodiments, the increase is an increase that is at least 10%, at least 20%, at least 30%, at least 40%, at least 50%, at least 60%, or at least 70% greater than the AAV reference. In some embodiments, the increase is an increase that is at least 0.5 log, at least 1 log, at least 1.5 logs, at least 2 logs, at least 2.5 logs, or at least 3 logs greater than the AAV reference. In some embodiments, the increase is an increase that is 0.5 log, 1 log, 1.5 logs, 2 logs, 2.5 logs, or 3 logs greater than the AAV reference. In some embodiments, the increase is an increase that is at least 0.5 log to 3 logs greater than the AAV reference. In some embodiments, the increase is an increase that is 0.5 log to 2 logs, 0.5 log to 2.5 logs, 0.5 log to 3 logs, 1 log to 3 logs, or 1 log to 2 logs greater than the AAV reference. In a specific embodiment, the novel rAAV viral particle comprises a capsid protein with a “BCD_” prefix set forth in any one of Example 4-8, and 10-11 infra.

In some embodiments, a reference AAV is AAV-1, AAV-2, AAV-4, AAV-5, AAV-6, AAV-7, AAV-8, AAV-9, AAV-10, AAV-11, AAV-12, and/or AAV-13. In certain embodiments, a reference AAV is AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu. 10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu. 1, AAVhu.2, AAVhu.3, AAVhu.4, AAVhu.5, AAVhu.6, AAVhu. 7, AAVhu.9, AAVhu. 10, AAVhu.11, AAVhu.13, AAVhu.15, AAVhu. 16, AAVhu. 17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu. 48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 14/9, AAVhu.t 19, AAVrh.2, AAVrh.2R, AAVrh.8, AAVrh.8R, AAVrh.12, AAVrh.13, AAVrh. 13R, AAVrh.14, AAVrh.17, AAVrh. 18, AAVrh. 19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1. 16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu. 19, AAVhu. 11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo. 1, AAV_avian_DA-1, or AAV_mouse_NY1. This paragraph is sometimes referred to herein as “Item A”.

Techniques known to one of skill in the art may be used to assess the tropism of a novel rAAV for a particular cell type or tissue type. For example, IVIS assays, immunoassays (e.g., immunostaining or immunohistochemistry), in cells, tissues, or a subject, may be used to determine tropism. Also see, Examples 6-8, and 10-11.

### 6.3.2.6 Evading Pre-Existing AAV Humoral Immunity

The present disclosure provides rAAV viral particle that are particularly useful as AAV-based vectors and/or rAAV viral particles for certain biomedical applications based on their ability (i.e., profile) to evade the recognition, binding, and/or neutralization by pre-existing antibodies (NAbs) in polyclonal plasma or sera to AAVs. NAbs function primarily by binding to the exposed surface of the AAV capsid and blocking processes essential for cellular transduction. As such, the ability to evade pre-existing AAV humoral immunity (also referred to as AAV humoral immunity) can be determined for a novel AAV capsid using one or more of the in vitro assays, such as binding, IVIg neutralization, or cell transduction. See, e.g., Giles, A. R. et al. (2018). Journal of virology, 92 (20), 1011-18.

The ability of the rAAV viral particle to evade pre-existing AAV humoral immunity can be assessed by the determining the percentage of cellular transduction (% transduction) in a given cell line in pooled plasma or serum (i.e., IgG pooled from normal subjects in the appropriate media for the cell line). See, e.g., Example 4.

In some embodiments, a novel rAAV viral particle has a range from about 2% to about 500% greater transduction as compared to a reference AAV. In some embodiments, a novel rAAV viral particle has at least about 2%, 3%, 5%, 10%, 15%, 20%, 25%, 30%, 35%, 40%, 45%, or 50% greater transduction in cells as compared to a reference AAV. In some embodiments, a novel rAAV viral particle has at least about 20%, 25%, 30%, 35%, 40%, 45%, or 50% greater transduction in cells compared to a reference AAV. In some embodiments, a novel rAAV viral particle has at least about 55%, 60%, 65%, 70%, 75%, 80%, 85%, 90%, 95%, 99%, or 100% greater transduction in cells compared to a reference AAV. In some embodiments, a novel rAAV viral particle has at least about 110%, 125%, 150%, 175%, 200%, 250%, 300%, 350%, 400%, or 500% greater transduction in cells as compared to a reference AAV. In some embodiments, a novel rAAV viral particle has at least about 250%, 300%, 350%, 400%, or 500% greater transduction in cells as compared to a reference AAV.

In some other embodiments, the ability of rAAV viral particle to evade AAV humoral immunity can be assessed by determining the effective IgG neutralizing titer of a novel AAV capsid that results in neutralizing antibody (NAb) titer reduction as compared to a reference AAV. In some embodiments, a novel rAAV viral particle has a range from about a 1-fold to about 4,000-fold NAb titer reduction as compared to a reference AAV, e.g., a novel rAAV viral particle has from about 10-fold to about 25-fold, from about 25-fold to about 50-fold, from about 50-fold to about 75-fold, from about 75-fold to about 100-fold, from about 100-fold to about 150-fold, from about 150-fold to about 200-fold, from about 200-fold to about 250-fold, from about 250-fold to about 300-fold, at least about 350-fold, at least about 400-fold, from about 400-fold to about 450-fold, from about 450-fold to about 500-fold, from about 500-fold to about 550-fold, from about 550-fold to about 600-fold, from about 600-fold to about 700-fold, from about 700-fold to about 800-fold, from about 800-fold to about 900-fold, from about 900-fold to about 1000-fold, from about 1,000-fold to about 2,000-fold, from about 2,000-fold to about 3,000-fold, from about 3,000-fold to about 4,000-fold NAb titer reduction as compared to a reference AAV.

In some embodiments, the ability of a novel AAV capsid, AAV clade, or AAV branch member to evade AAV humoral immunity can be assessed by determining the best fit line of NC₅₀ for a novel AAV capsid by plotted the data on a log scale against a reference AAV on a linear scale (semi-log plot) and then determining the best fit line using scientific graphing program, such as, for example, GraphPad Prism. In some embodiments, a novel rAAV viral particle has a range from about 1-fold to about 600-fold increase in NC₅₀ as compared to a reference AAV, e.g., a novel rAAV particle has from about 10-fold to about 25-fold, from about 25-fold to about 50-fold, from about 50-fold to about 75-fold, from about 75-fold to about 100-fold, from about 100-fold to about 150-fold, from about 150-fold to about 200-fold, from about 200-fold to about 250-fold, from about 250-fold to about 300-fold, at least about 350-fold, at least about 400-fold, from about 400-fold to about 450-fold, from about 450-fold to about 500-fold, from about 500-fold to about 550-fold, from about 550-fold to about 600-fold increase in NC₅₀ as compared to a reference AAV. In some embodiments, the ability of a novel AAV capsid, AAV clade, or AAV branch member to evade AAV humoral immunity can be assessed by comparing the NC₅₀ of a novel AAV capsid, AAV clade, or AAV branch member. For example, the capsid protein, AAV-6 had an NC₅₀ from an IVIg assay, such as described in Example 4, of about 0.0476 mg/mL, while average NC₅₀ of a capsid protein in Branch 1 is about 0.5722 mg/mL. The same NC₅₀ analysis can be conducted at the clade and/or branch level. The difference in the NC₅₀ value indicates the enhanced ability of a novel capsid protein as provided herein to evade AAV humoral immunity in a population (e.g., a human population). In other embodiments, the novel capsid is compared to an AAV capsid protein such as, but not limited to, AAV-12 which has NC₅₀ from an IVIg assay, such as described in Example 4, of about 0.5263 mg/mL, AAV-6 which has an NC₅₀ from an IVIg assay, such as described in Example 4, of about 0.0476 mg/mL, AAV-7 which has NC₅₀ from an IVIg assay, such as described in Example 4, of about 0.0441 mg/mL, AAV-8 which has NC₅₀ from an IVIg assay, such as described in Example 4, of about 0.0610 mg/mL, AAV-9 which has NC₅₀ from an IVIg assay, such as described in Example 4, of about 0.0513 mg/mL, AAV-5 which has NC₅₀ from an IVIg assay, such as described in Example 4, of about 0.2326 mg/mL, AAV-2 which has NC₅₀ from an IVIg assay, such as described in Example 4, of about <0.0305 mg/mL, or a combination of one more AAVs, wherein the AAV-2 less than (“<”) NC₅₀ is estimated owing to the fact that the IVIg is lower than the test limit in the assay.

### 6.3.3 Novel Recombinant AAV Viral Particles

The disclosure provides recombinant AAV (rAAV) viral particles and pseudotyped novel rAAV viral particles comprising a novel AAV capsid sequences of the disclosure. Such particles can be made from a recombinant AAV vector genome and/or one or more vectors (e.g., plasmid, bacmid, cosmid or the like), and appropriate host cell as described herein.

The disclosure also provides novel rAAV viral particles and pseudotype viral particles comprising a modified novel AAV capsid amino acid sequence, see Section 6.3.1.4. Production of a novel rAAV viral particles is provided below. Also, see Example 3.

6.3.3.1 Vector and rAAV Vector Genome Constructs

The disclosure also provides a vector (e.g., plasmid, bacmid, cosmid or the like) and a rAAV vector genome comprising the novel AAV capsid sequences of the disclosure.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest.

A novel rAAV viral particle of the disclosure may be generated by a method comprising providing to a suitable host cell with an rAAV vector genome, together with Rep and Cap (e.g., any one of SEQ ID NOs: 192-307 and 363-364) genes and/or a transgene either in one, two, three, or four separate vectors, thereby delivery a complete rAAV vector genome to the host cell. The vector configuration used and number of vectors used depends on the type of production system used.

The elements used to make an rAAV vector genome of the disclosure are described in more detail below. A person skilled in the art will select the appropriate elements depending on the application. In some embodiments, the rAAV vector genome used to make a novel rAAV viral particle comprises, (a) one or both of (i) an AAV inverted terminal repeat (ITR) sequence and (ii) an AAV 3′ ITR, (b) a heterologous regulatory element for expression in a specific cell type, and (c) a nucleic acid sequence comprising a nucleotide sequence encoding a transgene (e.g., a therapeutic transgene or biomolecule). For example, it may comprise one or both 5′ and 3′ ITRs of AAV-2, a tissue-specific promoter (e.g., a liver-specific promoter or muscle-specific promoter), and a transgene. See Section 6.3.3.1A for exemplary ITRs, section 6.3.3.1C for transgenes, and Section 6.3.3.1D for regulatory elements. Depending on the application the appropriate promoter can be used.

In some embodiments, the rAAV vector genome comprises a therapeutic transgene comprising a nucleic acid sequence encoding a functional version of a protein (e.g., endogenous protein) operably linked to a heterologous expression control element, e.g., a promoter or enhancer; optionally an intron; and optionally a polyadenylation (polyA) signal that allows for expression in the host cell (i.e., delivery to a target cell).

Generation of a vector and/or an rAAV vector genome of the disclosure may be made using any suitable genetic engineering techniques well known in the art, including, without limitation, the standard techniques of restriction endonuclease digestion, ligation, transformation, plasmid purification, and DNA sequencing, for example as described in Green, M. and Sambrook, J., Molecular Cloning: A Laboratory Manual, 4th Ed., Cold Spring Harbor Laboratory (Cold Spring Harbor, N.Y. 2012).

Polynucleotides comprising a transgene (e.g., a therapeutic transgene) of the rAAV vector genome, such as codon-optimized, mini-genes, etc. can be made using various standard cloning, recombinant DNA technology, via cell expression or in vitro translation and chemical synthesis techniques known to those of skill in the art. See Green, M. and Sambrook, J., Molecular Cloning: A Laboratory Manual, 4th Ed., Cold Spring Harbor Laboratory (Cold Spring Harbor, N.Y. 2012).

### A. Inverted Terminal Repeats (ITRs)

An rAAV vector genome of the disclosure will often comprise an AAV inverted terminal repeat element (ITR). In some embodiments, an rAAV vector genome comprises one ITR or a fragment thereof. In some embodiments, an rAAV vector genome comprises a 5′ or 3′ ITR. In specific embodiments, an rAAV vector genome comprises two ITRs or a fragment thereof. In some embodiments, an rAAV vector genome comprises a 5′ ITR and a 3′ ITR. The AAV ITRs, together with the Rep coding region, provide for efficient excision and rescue from, and integration of a nucleotide sequence, such as a therapeutic transgene, interposed between two flanking ITRs into a host cell genome. Sequences of certain AAV-associated ITRs are disclosed by Yan et al., J. Virol. 79 (1): 364-379 (2005). In a specific embodiment, the AAV ITR sequences are from a different AAV serotype or different AAV clade than the AAV cap sequences. In some embodiments, the AAV ITR sequences are from the same AAV serotype or AAV clade as the AAV cap sequences. In some embodiments, the AAV ITR sequences are from a different AAV serotype or AAV clade than the AAV rep sequences. In some embodiments, the AAV ITR sequences are from the same AAV serotype or AAV clade as the AAV rep sequences. In another specific embodiment, the AAV ITR sequences are from a different AAV serotype or different AAV clade than the AAV cap sequences and rep sequences. In some embodiments, the AAV ITR sequences are from the same AAV serotype or AAV clade as the AAV cap sequences and rep sequences. Genomic sequences of various serotypes of AAV, as well as sequences of native terminal repeats (TRs), Rep proteins and capsid subunits are known in the art (e.g., such sequences can be found in the literature or in public databases such as GenBank). For example, GenBank accession numbers that provide the genomic sequences of various serotypes of AAV include NC_002077.1 (AAV1), AF063497.1 (AAV1), NC_001401.2 (AAV2), AF043303.1 (AAV2), J01901.1 (AAV2), U48704.1 (AAV3A), NC_001729.1 (AAV3A), AF028705.1 (AAV3B), NC_001829.1 (AAV4), U89790.1 (AAV4), NC_006152.1 (AA5), AF085716.1 (AAV-5), AF028704. 1 (AAV6), NC_006260.1 (AAV7), AF513851.1 (AAV7), AF513852.1 (AAV8), NC_006261.1 (AAV-8), AY530579.1 (AAV9), AAT46337 (AAV10) AAO88208 (AAVrh10), AY631966.1 (AAV11), DQ813647.1 (AAV12), and EU28SS62.1 (AAV13), the entirety of each of which is incorporated herein by reference, disclose nucleic acid and amino acid sequences. The ITR sequences of those GenBank sequences can be used in a rAAV vector genome described herein. In certain embodiments, the ITR sequences of an AAV disclosed in Table 4 may be used in an rAAV vector genome described herein. In a specific embodiment, an rAAV vector genome comprises the ITRs of AAV-2 or fragments thereof. This paragraph is sometimes referred to herein as “Item B”.

TABLE 4
AAV Reference Sequences and ITR Sequences
Table 4: AAV Reference Sequences

| | NCBI Reference Sequence No./ |
|---|---|
| | Genbank Accession No. |
| AAV Serotype | (each herein incorporated by reference) |
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704-1 |
| AAV-7 | NC_006260.1 |
| AAV-8 | NC_006261.1 |
| AAV-9 | AX7S3250.1 |
| AAV-10 | AY631965.1 |
| AAV-11 | AY631966.1 |
| AAV-12 | DQ813647.1 |
| AAV-13 | EU28SS62.1 |

### B. AAV Rep and Cap Gene Sequences

A novel rAAV vector genome of the disclosure may comprise an AAV “rep” and “cap” nucleotide sequences encoding replication and encapsidation proteins, respectively. The AAV cap nucleotide sequences include the nucleotide sequences of the novel AAV capsids described herein.

Often, for gene therapy and/or delivery applications, the rep and cap genes will be provided in a separate vectors along with the rAAV vector genome (e.g. novel rAAV vector genome of the disclosure). In a specific embodiment, the AAV rep sequences are from a different AAV serotype or different AAV clade than the AAV cap sequences. In a specific embodiment, the AAV rep sequences are from the same AAV serotype or different AAV clade than the AAV cap sequences. In another specific embodiment, the AAV rep sequences are from a different AAV serotype or different AAV clade than the AAV cap sequences and ITR sequence(s). In another specific embodiment, the AAV rep sequences are from the same AAV serotype or different AAV clade than the AAV cap sequences and ITR sequence(s). Examples of AAV rep sequences include the AAV rep sequence of an AAV serotype in Table 4, supra. Examples of the same and different AAV clades or serotypes are provided herein (see FIGS. 3-14, Table 4, and Table 2).

The AAV cap gene encodes a cap protein (see Section 6.3.1) which is capable of packaging AAV vector genomes in the presence of rep and a helper function (e.g., adeno helper function) and is capable of binding a target cell. In some embodiments, the helper function can be provided by the host cell. The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes. In various embodiments, a vector providing AAV helper functions includes a nucleotide sequence(s) that encode Cap proteins or Rep proteins.

### C. Transgenes

A recombinant novel rAAV viral particle of the disclosure will often comprise a heterologous transgene (e.g., a therapeutic transgene). A transgene incorporated into the novel rAAV viral particle is not limited and may be any heterologous nucleotide sequence of interest (e.g., a heterologous gene of interest). The transgene is a nucleic acid sequence, heterologous to the vector genome sequences flanking the transgene, which encodes a polypeptide, protein, or other product, of interest. The nucleic acid coding sequence is operatively linked to one or more regulatory components (e.g., promoter, enhancer, poly-A, microRNA binding elements that either restrict and or enhance transgene expression, 3′UTR, Woodchuck Hepatitis Virus Posttranscriptional Regulatory Element (WPRE)) in a manner which permits transgene transcription, translation, and/or expression in a host cell. The composition of the heterologous transgene sequence will depend upon the application (e.g., the therapeutic application or indication to be treated). In some embodiments, a novel rAAV viral particle comprises two or more heterologous transgenes, for example, two, three, four or five heterologous transgenes. In other embodiments, a novel rAAV viral particle comprises one heterologous transgene incorporated into the rAAV viral particle.

The size of the nucleotide sequence of a transgene can vary. For example, the nucleotide sequence of a transgene encoding a therapeutic protein can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide sequence of a transgene encoding a therapeutic protein is at least about 1.4 kb in length. In certain embodiments, the nucleotide sequence of a transgene encoding a therapeutic protein is about 1.4 kb to 5 kb in length. In some embodiments, the nucleotide sequences of a transgene encoding a therapeutic protein is 1.4 kb to 5 kb or 5 kb to 10 kb. Alternatively, the nucleotide sequence of a transgene is at least about 30 nucleotides, at least about 40 nucleotides, at least about 50 nucleotides, at least about 75 nucleotides in length, at least about 100 nucleotides in length, at least about 150 nucleotides in length, at least about 200 nucleotides in length, at least about 250 nucleotides in length, at least about 300 nucleotides in length, at least about 350 nucleotides in length, at least about 400 nucleotides in length, at least about 500 nucleotides in length, at least about 600 nucleotides in length, at least about 700 nucleotides in length, at least about 800 nucleotides in length, at least about 900 nucleotides in length, at least about 1000 nucleotides in length, or at least about 1200 nucleotides in length. In some embodiments, the nucleotide sequence of a transgene is about 30 to 150 nucleotides in length or about 150 to 500 nucleotides in length. In certain embodiments, the nucleotide sequence of a transgene is about 100 to 500 nucleotides in length or 500 to 1000 nucleotides in length. In some embodiments, the nucleotide sequence of a transgene is 500 nucleotides to 1200 nucleotides in length.

In specific embodiments, a novel rAAV viral particle of the disclosure comprises a therapeutic transgene. A therapeutic transgene of the disclosure is typically a sequence that encodes a biomolecule (e.g., a therapeutic biomolecule) which is useful in biology and treatment of a disease, such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., CRISPR gene editing system, antisense oligonucleotides (AONs), antisense oligonucleotide (AON)-mediated exon skipping, a poison exon(s) that triggers nonsense mediated decay (NMD), or a dominant negative mutant.

In certain embodiments, a transgene comprises a nucleic acid sequence encoding a sequence useful for gene therapy applications. For example, certain diseases come about when one or more loss-of-function mutations (e.g., null mutation and/or haploinsufficiency) within a gene reduce or abolish the amount or activity of the protein encoded by the gene. In certain embodiments, a transgene utilized herein encodes a functional version of the protein. In some embodiments, a functional version of the protein retains one, two, or more activities of an endogenous protein (e.g., a protein found in a human or non-human animal).

In other embodiments, a novel rAAV viral particle comprises a transgene comprising a nucleic acid sequence encoding a sequence useful for gene therapy applications that benefit from gene silencing. For example, certain diseases come about when gain-of-function mutations within a gene result in an aberrant amount or activity of the protein encoded by the gene. In certain embodiments, a transgene utilized herein encodes an inhibitory polynucleotide, e.g., an inhibitory RNA such as an miRNA or siRNA, or one or more components of gene editing system, e.g., a CRISPR gene editing system. In some embodiments, a transgene comprises a nucleic acid encoding a CRISPR-Cas system for targeted gene disruption or correction.

In other embodiments, a transgene comprising a nucleic acid sequence encodes a sequence useful for gene therapy applications that benefit from gene addition. In certain embodiments, a transgene utilized herein encodes a gene product, e.g., a protein, not present in a recipient, e.g., a human subject, of the gene therapy.

In some embodiments, a transgene comprises a nucleic acid sequence encoding an RNA sequence useful in biology and medicine, such as, e.g., tRNA, dsRNA, ribosomal RNA, catalytic RNA, siRNA, miRNA, pre-miRNA, lncRNA, snoRNA, small hairpin RNA, trans-splicing RNA, and antisense RNA. One example of a useful RNA sequence is a sequence which inhibits or extinguishes expression of a targeted nucleic acid sequence in a treated subject. Suitable target nucleic acid sequences may include oncologic sequences and viral sequences. In some embodiments, a transgene comprises a nucleic acid sequence encoding a small nuclear RNA (snRNA) construct which induces exon skipping. In certain embodiments, an RNAi agent targets a gene of interest at a location of a single-nucleotide polymorphism (SNP) or a variant within the nucleotide sequence.

In some embodiments, an RNAi agent is an siRNA duplex, wherein the siRNA duplex contains an antisense strand (guide strand) and a sense strand (passenger strand) hybridized together forming a duplex structure, wherein the antisense strand is at least partially complementary to the nucleic acid sequence of the targeted gene, and wherein the sense strand is at least partially homologous to the nucleic acid sequence of the targeted gene. In some embodiments, the 5′end of the antisense strand has a 5′phosphate group and the 3′end of the sense strand contains a 3′hydroxyl group. In some embodiments, there are none, one or 2 nucleotide overhangs at the 3′end of one or both strands. In some embodiments, one or more than one nucleotide of an antisense strand and/or a sense strand is modified. Non-limiting examples of nucleotide modifications include 2′deoxy, 2′-fluoro, 2′ O-methyl, 2′deoxy-2′fluoro, a phosphorothioate, 5′-morpholinno, a universal base modified nucleotide, a terminal cap molecule at the 3′-end, the 5′-end, or both 3′ and 5′-ends, an inverted abasic, or an inverted abasic locked nucleic acid modification at the 5′-end and/or 3′ end.

In some embodiments, each strand of an siRNA duplex targeting a gene of interest is about 19 to 25, 19 to 24 or 19 to 21 nucleotides in length. In some embodiments, an siRNA or dsRNA includes at least two sequences that are complementary to each other. The dsRNA includes a sense strand having a first sequence and an antisense strand having a second sequence. In some embodiments, the antisense strand includes a nucleotide sequence that is substantially complementary to at least part of an mRNA encoding the target gene, and the region of complementarity is 30 nucleotides or less, and at least 15 nucleotides in length. In some embodiments, the dsRNA is 19 to 25, 19 to 24 or 19 to 21 nucleotides in length. In some embodiments, the dsRNA is from about 15 to about 25 nucleotides in length. In some embodiments, the dsRNA is from about 25 to about 30 nucleotides in length. In some embodiments, the dsRNA is about, at least about, or at most about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, a novel rAAV viral particle of the disclosure comprises a transgene comprising a nucleic acid sequence encoding a protein, peptide or other product that corrects or ameliorates a genetic deficiency or other abnormality in a subject. Such genetic deficiencies may include deficiencies in which gene products are expressed at less than levels considered normal for a particular subject (e.g., a human subject) or deficiencies in which a functional gene product is not expressed. In some embodiments, a novel rAAV viral particle of the disclosure comprises multiple transgenes to, e.g., correct or ameliorate a genetic defect caused by a multi-subunit protein. In some instances, a different transgene may be used to encode each subunit of a protein, or to encode different peptides or proteins. This may be desirable when the size of the nucleic acid sequence encoding the protein subunit is large, non-limiting examples include e.g., for an immunoglobulin, the platelet-derived growth factor, or a dystrophin protein. A host cell may be infected with a novel rAAV viral particle of the disclosure containing transgenes, wherein each transgene comprises a nucleic acid sequence encoding a different subunit of a multi-subunit protein, in order to produce the multi-subunit protein. Alternatively, a novel rAAV viral particle of the disclosure may comprise a single transgene comprising nucleic acid sequences encoding different subunits of a multi-subunit protein. In this case, a single transgene comprises nucleic acid sequences encoding each of the subunits and the nucleic acid sequence encoding each subunit may be separated by an internal ribozyme entry site (IRES). This may be desirable when the size of the nucleic acid sequence encoding each of the subunits is small, e.g., the total size of the nucleic acid sequences encoding the subunits and the IRES is less than five kilobases. As an alternative to an IRES, the nucleic acid sequence may be separated by sequences encoding a peptide, such as, e.g., 2A peptide, which self-cleaves in a post-translational event. See, e.g., Donnelly et al, J. Gen. Virol., 78 (Pt 1): 13-21 (January 1997); Furler, et al, Gene Ther., 8(11): 864-873 (June 2001); Klump et al., Gene Ther., 8 (10): 811-817 (May 2001). A 2A peptide is significantly smaller than an IRES, making it well suited for use when space is a limiting factor. More often, when a transgene is large, consists of multi-subunits, or both, two or more AAV viral particles (including a novel rAAV viral particle of the disclosure) each carrying a desired transgene may be co-administered to allow them to concatamerize in vitro or in vivo to form a single vector genome. See, e.g., Yang et al., J Virol. 1999 November; 73 (11): 9468-9477 for information regarding the concatamerization of AAV. For example, a first AAV viral particle may comprise a single transgene and a second AAV viral particle may comprise a different transgene for co-expression in a host cell.

In some embodiments, a transgene comprises a nucleic acid sequence encoding a protein heterologous to AAV (e.g., a therapeutic protein). In some embodiments, a transgene comprises a nucleic acid sequence encoding a therapeutic protein that is endogenously expressed in one or more of a muscle, heart, brain, plasma, kidney, ear, or liver cell/tissue of a subject. In certain embodiments, a transgene comprises a nucleic acid sequence encoding a therapeutic protein that is endogenously expressed in one or more of a muscle, heart, brain, plasma, kidney, or liver cell/tissue of a subject.

In some embodiments, a transgene comprises a nucleic acid sequence, which upon expression produces a detectable signal. In some instances, such a nucleic acid sequence encodes an enzyme (such as, e.g., β-lactamase, β-galactosidase (LacZ), alkaline phosphatase, thymidine kinase, chloramphenicol acetyltransferase (CAT), and luciferase), a fluorescent protein (such as, e.g., green fluorescent protein (GFP), yellow fluorescent protein, and red fluorescent protein), a membrane bound protein (such as, e.g., CD2, CD4, CD8, the influenza hemagglutinin protein, and others well known in the art, to which high affinity antibodies directed thereto exist or can be produced by conventional means) or a fusion protein comprising a membrane bound protein appropriately fused to an antigen tag domain from, among others, hemagglutinin or Myc. These nucleic acid sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry (IHC). For example, where the nucleic acid sequence encodes LacZ, the presence of an AAV vector genome expressing LacZ may be detected by assays for beta-galactosidase activity. In another example, where the transgene comprises a nucleic acid sequence encoding green fluorescent protein or luciferase, an AAV vector genome expressing the green fluorescent protein or luciferase may be detected visually by color or light production in a luminometer. An AAV viral particle comprising a transgene that comprises a nucleotide sequence encoding a product with a detectable signal may be used a selectable marker as discussed below or may be used to trace the virus.

### D. Regulatory Control Elements

Depending on the application, an AAV vector genome of the disclosure can include one or more regulatory control elements (e.g., transcription initiation sequence, termination sequence, promoter, enhancer, regulatory binding sites, poly-A, microRNA binding elements, 3′UTR, Woodchuck Hepatitis Virus Posttranscriptional Regulatory Element (WPRE)).

Often, a regulatory control element is heterologous and is operably linked to the transgene (e.g., therapeutic transgene) in a manner which permits its transcription, translation and/or expression in a host cell transfected with the novel rAAV vector genome of the disclosure. As used herein, “operably linked” includes both expression control sequences that are contiguous with the gene of interest and expression control sequences that act in trans or at a distance (e.g., an enhancer) to control the gene of interest.

Exemplary of regulatory control elements that can be used, include but are not limited to, transcription initiation, termination, promoter and/or enhancer sequences; efficient RNA processing signals such as splicing and polyadenylation (poly A) signals; sequences that stabilize cytoplasmic mRNA; sequences that enhance translation efficiency (i.e., Kozak consensus sequence); sequences that enhance protein stability; and when desired, sequences that enhance secretion of the encoded product. Where an AAV vector genome includes a coding sequence, a therapeutic transgene to be translated into a therapeutic protein or peptide, a promoter is included in the AAV vector genome.

Depending on the application and level of expression needed, a skilled artisan can use a promoter which is native to the cell type or subject to which the AAV vector genome is to be delivered. In some embodiments, a promoter is a constitutive promoter, inducible promoter and/or tissue-specific promoter. Further, the combination of regulatory control elements can be used in an AAV vector genome depends on the vector and its application.

In certain embodiments, a regulatory control element comprises a regulatory control element that modulates gene expression specifically in muscle tissue. In certain embodiments, a regulatory control element comprises a regulatory element that modulates gene expression specifically in the heart.

In certain embodiments, a regulatory control element comprises a regulatory element that modulates gene expression specifically in the brain. In some embodiments, a regulatory control element comprises a regulatory element that modulates gene expression specifically in the central nervous system. For example, in certain embodiments, a regulatory control element comprises a human synapsin 1 gene (hSyn1), human elongation factor 1α (hEF1α), or rat Calcium/calmodulin-dependent protein kinase type II alpha (CaMKIIα) promoter may be used.

In certain embodiments, a regulatory control element comprises a regulatory element that modulates gene expression specifically in the plasma. In certain embodiments, a regulatory control element comprises a regulatory element that modulates gene expression specifically in the kidney. In certain embodiments, a regulatory control element comprises a regulatory element that modulates gene expression specifically in the ear. In certain embodiments, a regulatory control element comprises a regulatory element that modulates gene expression specifically in a tissue or cell identified in an Example, infra.

In some embodiments, a regulatory control element comprises a regulatory element that modulates gene expression specifically in liver tissue. Examples of liver-specific regulatory elements include, but are not limited to, the mouse thyretin promoter (mTTR), the endogenous human factor VIII promoter (F8), human alpha-1-antitrypsin promoter (hAAT) and active fragments thereof, human albumin minimal promoter, and mouse albumin promoter. Enhancers derived from liver specific transcription factor binding sites are also contemplated, such as EBP, DBP, HNF1, HNF3, HNF4, HNF6, with Enh1.

### E. Selectable Markers or Reporter Genes

In some embodiments, the rAAV vector genome (e.g., novel rAAV vector genome) further comprises a selectable marker or reporter gene. Examples of selectable marker or reporter gene may include sequences encoding geneticin, hygromicin or purimycin resistance, among others. Such selectable reporters or marker genes (preferably located outside the viral genome to be rescued by the method of the invention) can be used to signal the presence of the plasmids in bacterial cells, such as ampicillin resistance. Other components of the plasmid may include an origin of replication. Selection of these and other promoters and vector elements are conventional and many such sequences are available. See, for example, Green, M. and Sambrook, J., Molecular Cloning and other references cited herein.

### 6.3.3.2 Novel Pseudotyped AAV Viral Particles

The disclosure also provides various novel pseudotyped AAV viral particles using the novel AAV capsid sequences described herein or modified novel AAV capsid sequences described herein and the genome elements (i.e., Rep or ITR sequences) of a different AAV (e.g., different AAV serotype and/or clade).

In one embodiment, the novel pseudotyped AAV viral particles of the disclosure comprise one or more the novel AAV capsid sequences described herein or modified novel AAV capsid sequences described herein, Rep or ITR sequences or fragments thereof of a different AAV, and a transgene (e.g., a transgene that comprises a nucleotide sequence encoding a therapeutic protein). In another embodiment, the novel pseudotyped AAV viral particles of the disclosure comprise one or more the novel AAV capsid sequences described herein or modified novel AAV capsid sequences described herein, Rep and ITR sequences of a different AAV, and a transgene (e.g., a transgene that comprises a nucleotide sequence encoding a therapeutic protein). Examples of different AAVs that can be used to make a novel pseudotyped AAV viral particles, include any one of the reference AAVs provided herein (e.g., in Table 4 or Item A or Item B).

### 6.3.3.3 Other AAV Capsid Based Vectors

The novel AAV capsid sequences of the disclosure can be adapted for use in other viral vector systems for in vitro, ex vivo or in vivo gene delivery. For instance, the novel AAV capsid sequences may be used to construct a hybrid vector comprising an expression cassette for a parvovirus other than AAV. For example, a hybrid vector may comprise a parovirus-derived (e.g., an autonomous parvovirus H1-derived or parovirus B19-derived) expression cassette, a promoter (e.g., p4 promoter), a gene encoding a protein of interest, and another promoter (e.g., p38 promoter) flanked by AAV ITRs and packaged into the novel capsids of the disclosure.

See, e.g., Krüger et al., Cancer Gene Therapy volume 15, pages 252-267 (2008) for a discussion of methods for producing a hybrid vector comprising AAV capsids. In addition, the novel AAV capsid sequences of the disclosure can be used to generate AAV virus-like particles (VLPs). See, e.g., Le et al. Sci Rep 9, 18631 (2019) for methods for producing AAV VLPs.

### 6.3.4 Host Cells

The disclosure also provides a host cell (e.g., an in vivo or an in vitro host cell) comprising a novel AAV capsid sequence, a modified AAV capsid sequence, or a novel rAAV viral particle of the disclosure and a recombinant nucleic acid molecule that further comprises a heterologous sequence (e.g., a therapeutic biomolecule or transgene and/or regulatory element). In some embodiments, the disclosure provides a host cell (e.g., an in vivo or an in vitro host cell) comprising a novel AAV capsid sequence or a modified AAV capsid sequence. In some embodiments, the disclosure provides a host cell (e.g., an in vivo or an in vitro host cell) comprising a novel rAAV viral particle of the disclosure. In some embodiments, the disclosure provides a host cell (e.g., an in vivo or an in vitro host cell) comprising a recombinant nucleic acid molecule that further comprises a heterologous sequence (e.g., a therapeutic biomolecule or transgene and/or regulatory element).

As used herein, the term “host” refers to organisms (e.g., insects, animals (including humans and non-human animals), yeast, bacteria, etc.) and/or cells which harbor a nucleic acid molecule or an AAV viral particle of the present disclosure, as well as organisms (e.g., humans and non-human animals) and/or cells that are suitable for use in expressing a recombinant gene or protein. It is not intended that the present disclosure be limited to any particular type of cell or organism. Indeed, it is contemplated that any suitable organism and/or cell will find use herein as a host. A host cell may be in the form of a single cell, a population of similar or different cells, for example in the form of a culture (such as a liquid culture or a culture on a solid substrate), an organism or part thereof. The host cell includes progeny of the cells infected by a novel rAAV viral particle described herein. Any host cell which allows for replication of an AAV and/or production of the therapeutic transgene in an AAV, and which can be maintained in culture is a part of the present disclosure.

Thus, the host cell of the disclosure can be, for example, a bacterial, a yeast, an insect, or a mammalian cell, or a human cell. Examples of preferred insect cells are High Five, Sf9, Se301, SeIZD2109, SeUCR1, Sf900+, Sf21, BTI-TN-5B1-4, MG-1, Tn368, HzAm1, BM-N, Ha2302, Hz2E5, or A38. Examples of preferred mammalian cells are HEK293, HeLa, CHO, NSO, SP2/0, PER.C6, Vero, RD, BHK, HT 1080, A549, Cos-7, ARPE-19, or MRC-5 cells. The host cell may be one described herein (e.g., in Section 6.4.3, or the Examples). In specific embodiments, the host cell is a non-human mammalian cell. In other embodiments, the host cell is a bacterial, yeast or insect cell. In certain embodiments, the host cell is a human cell. In specific embodiments, the human cell is a primary cell isolated from a human subject (e.g., the subject to be treated with gene therapy). In some embodiments, the host cell is from a cell line. In some embodiments, the host cell is in vitro or in cell culture (i.e., a cultured host cell). In other embodiments, the host cell is in vivo. In specific embodiments, a host cell(s) is isolated from a tissue.

In specific embodiments, provided herein is a host cell(s) comprising a novel AAV capsid sequence of the disclosure (e.g., VP1, VP2, or VP3). In specific embodiments, provided herein is a host cell(s) expressing a novel AAV capsid sequence of the disclosure (e.g., VP1, VP2, or VP3). In some embodiments, provided herein is a host cell comprising a novel AAV capsid sequence of the disclosure (e.g., VP1, VP2, or VP3) and a vector. In certain embodiments, provided herein is a host cell comprising a novel AAV capsid sequence of the disclosure (e.g., VP1, VP2, or VP3), a Rep gene and a vector. In certain embodiments, provided herein is a host cell comprising a novel AAV capsid sequence of the disclosure (e.g., VP1, VP2, or VP3), a Rep gene and a rAAV vector genome. In specific embodiments, provided herein is a host cell producing a novel rAAV viral particle of the disclosure.

In specific embodiments, provided herein is a host cell comprising a modified novel AAV capsid sequence of the disclosure (e.g., VP1, VP2, or VP3). In specific embodiments, provided herein is a host cell(s) expressing a modified novel AAV capsid sequence of the disclosure (e.g., VP1, VP2, or VP3). In some embodiments, provided herein is a host cell comprising a modified novel AAV capsid sequence of the disclosure (e.g., VP1, VP2, or VP3) and a vector. In certain embodiments, provided herein is a host cell comprising a modified novel AAV capsid sequence of the disclosure (e.g., VP1, VP2, or VP3), a Rep gene and a vector. In certain embodiments, provided herein is a host cell comprising a modified novel AAV capsid sequence of the disclosure (e.g., VP1, VP2, or VP3), a Rep gene and a rAAV vector genome.

The rAAV viral particles, host cells, and methods/use of the present disclosure are useful in a method of delivering a transgene (e.g., a therapeutic biomolecule) into a host cell. A host cell of the disclosure is often used for the manufacture of the novel rAAV viral particles. In a specific embodiment, a host cell described herein (e.g., in the Examples; in particular Example 3) is used in the production of a novel rAAV viral particle.

### 6.3.5 Pharmaceutical Compositions

The disclosure provides for compositions (e.g., pharmaceutical compositions) comprising a novel AAV capsid sequence or rAAV viral particle. In one embodiment, a composition comprises a novel AAV capsid sequence described herein or a vector comprising such a sequence as described in Section 6.3.1. In certain embodiments, a composition comprises a novel AAV capsid sequence described in the Examples herein. In some embodiments, a composition comprises a modified novel AAV capsid sequence or a vector comprising such a sequence. In a specific embodiment, provided herein is a composition (e.g., pharmaceutical compositions) comprising a novel rAAV viral particle. A pharmaceutical composition may comprise any novel rAAV viral particle(s) described herein. In some embodiments, a composition (e.g., pharmaceutical compositions) comprises two or more novel rAAV viral particles described herein.

In specific embodiments, provided herein are pharmaceutical compositions comprising a novel rAAV viral particle comprising a biomolecule or transgene described in Section 6.3.3.1C. In certain embodiments, provided herein are pharmaceutical compositions comprising a novel rAAV viral particle comprising a transgene(s) that comprises a nucleic acid sequence encoding a therapeutic protein useful for administration to subjects suffering from a genetic disorder. In some embodiments, provided herein are pharmaceutical compositions comprising a novel rAAV viral particle comprising a transgene that comprises a nucleic acid sequence of an RNA (e.g., siRNA, antisense RNA, miRNA, etc.). In certain embodiments, provided herein are pharmaceutical compositions comprising a novel rAAV viral particle comprising a transgene that comprises a component of the CRISPR system.

In certain embodiments, provided herein is a pharmaceutical composition comprising a plurality of rAAV viral particles (e.g., novel AAV capsid protein of the disclosure) comprising transgenes that comprise nucleic acid sequences encoding various elements of a CRISPR system. In some embodiments, a first AAV viral particle comprise a transgene comprising a nucleic acid sequence encoding a CRISPR-Cas (CRISPR-Cas9 enzyme); and a second AAV viral particle comprise a transgene comprising a nucleic acid sequence encoding a guide RNA sequence for a target gene to allow disruption of the target gene. In some embodiments, a first AAV viral particle comprise a transgene comprising a nucleic acid sequence encoding a CRISPR-Cas (CRISPR-Cas9 enzyme); a second AAV viral particle comprise a transgene comprising a nucleic acid sequence encoding a guide RNA sequence for a target gene; and a third AAV viral particle comprise a transgene comprising a nucleic acid sequence encoding a donor nucleic acid sequence for correction or replacement of a target gene. In some embodiments, a first AAV viral particle comprise a transgene comprising a nucleic acid sequence encoding a CRISPR-Cas (CRISPR-Cas9 enzyme) and a guide RNA sequence for a target gene; and a second AAV viral particle comprise a transgene comprising a nucleic acid sequence encoding a donor nucleic acid sequence for correction or replacement of a target gene. In some embodiments, a single AAV viral particle comprises a transgene comprising a nucleic acid sequence encoding a CRISPR-Cas (CRISPR-Cas9 enzyme) and a guide RNA sequence for a target gene to allow disruption of the target gene.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In certain embodiments, a pharmaceutical composition provided herein is a liquid composition that comprises a novel rAAV viral particle. In other embodiments, a pharmaceutical composition provided herein that comprises a novel rAAV viral particle is a lyophilized composition. In certain embodiments, the concentration of a novel recombinant AAV virion in the composition may range from 1×10¹² vg/ml to 2×10¹⁶ vg/ml. See Section 6.4.2 for other doses.

The compositions described herein may comprises an excipient or carrier, e.g., a buffer. In a specific embodiment, provided herein are various pharmaceutical compositions comprising a novel AAV capsid sequence or viral particle as well as a pharmaceutically acceptable carrier and/or other medicinal agent, pharmaceutical agent or adjuvant, etc. In certain embodiments, a pharmaceutical composition described herein comprises a novel rAAV viral particle of the disclosure and one or more other agents, such as described in Section 6.3.5.

In some embodiments, the terms “pharmaceutically acceptable” and “physiologically acceptable” are used interchangeably. Typically, an agent (e.g., an excipient or carrier) is pharmaceutically acceptable when it is safe, non-toxic, and is not biologically or otherwise undesirable, and is acceptable for veterinary use as well as human pharmaceutical use.

In certain embodiments, a pharmaceutical composition provided herein comprises one or more pharmaceutically acceptable excipients to provide the composition with advantageous properties for storage and/or administration to subjects for the treatment of the genetic disorder. In some embodiments, the pharmaceutical compositions provided herein are capable of being stored at −65° C. for a period of at least 2 weeks, in one embodiment at least 4 weeks, in another embodiment at least 6 weeks and yet another embodiment at least about 8 weeks, without detectable change in stability. In this regard, the term “stable” means that the recombinant AAV virus present in the composition essentially retains its physical stability, chemical stability and/or biological activity during storage. For example, in certain embodiments, the recombinant AAV virus present in the pharmaceutical composition retains at least about 80% of its biological activity in a human patient during storage for a determined period of time (e.g., 1 to 6 months, 3 to 6 months, 3 to 9 months, or 6 to 12 months) at −65° C.; in other embodiments at least about 85%, 90%, 95%, 98% or 99% of the recombinant AAV virus' biological activity is retained in a human subject. In one embodiment, the subjects are juvenile human subjects (e.g., human subjects less than 18 years old). In some embodiments, the recombinant AAV virus present in the pharmaceutical composition retains at least about 80% of its biological activity assessed in in vitro assay in a host cell during storage for a determined period of time (e.g., 1 to 6 months, 3 to 6 months, 3 to 9 months, or 6 to 12 months) at −65° C.; in other embodiments at least about 85%, 90%, 95%, 98% or 99% of the recombinant AAV virus' biological activity is retained as assessed in an in vitro assay in a host cell.

In certain aspects, a pharmaceutical composition comprising a novel rAAV viral particle further comprises one or more buffering agents. For example, in various embodiments, a pharmaceutical composition provided herein comprises sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In one embodiment, a pharmaceutical composition provided herein comprises about 1.42 mg/ml of sodium phosphate, dibasic (dried). Another buffering agent that may find use in a pharmaceutical compositions provided herein is sodium phosphate, monobasic monohydrate which, in some embodiments, finds use at a concentration of from about 0.1 mg/ml to about 3 mg/ml, about 0.5 mg/ml to about 2.5 mg/ml, about 1 mg/ml to about 2 mg/ml, or about 1.3 mg/ml to about 1.5 mg/ml. In one embodiment, a pharmaceutical composition of the present embodiment comprises about 1.38 mg/ml of sodium phosphate, monobasic monohydrate. In another embodiment, a pharmaceutical composition provided herein comprises about 1.42 mg/ml of sodium phosphate, dibasic and about 1.38 mg/ml of sodium phosphate, monobasic monohydrate.

In another embodiment, a pharmaceutical composition provided herein may comprise one or more isotonicity agents, such as sodium chloride, in one embodiment at a concentration of about 1 mg/ml to about 20 mg/ml, for example, about 1 mg/ml to about 10 mg/ml, about 5 mg/ml to about 15 mg/ml, or about 8 mg/ml to about 20 mg/ml. In another embodiment, a pharmaceutical composition provided herein comprises about 8.18 mg/ml sodium chloride. Other buffering agents and isotonicity agents known in the art are suitable and may be routinely employed for use in the compositions provided herein.

In another embodiment, a pharmaceutical composition provided herein may comprises one or more bulking agents. Exemplary bulking agents include without limitation mannitol, sucrose, dextran, lactose, trehalose, and povidone (PVP K24). In certain embodiments, a pharmaceutical composition provided herein comprises mannitol, which may be present in an amount from about 5 mg/ml to about 40 mg/ml, or from about 10 mg/ml to about 30 mg/ml, or from about 15 mg/ml to about 25 mg/ml. In another embodiment, mannitol is present at a concentration of about 20 mg/ml.

In yet another embodiment, a pharmaceutical composition provided herein may comprise one or more surfactants, which may be non-ionic surfactants. Exemplary surfactants include ionic surfactants, non-ionic surfactants, and combinations thereof. For example, the surfactant can be, without limitation, TWEEN 80 (also known as polysorbate 80, or its chemical name polyoxyethylene sorbitan monooleate), TWEEN 20 (also known as polysorbate 20), sodium dodecyl sulfate, sodium stearate, ammonium lauryl sulfate, TRITON AG 98 (Rhone-Poulenc), poloxamer 407, poloxamer 188 and the like, and combinations thereof. In one embodiment, a pharmaceutical composition of the present embodiment comprises poloxamer 188, which may be present at a concentration of from about 0.1 mg/ml to about 4 mg/ml, or from about 0.5 mg/ml to about 3 mg/ml, from about 1 mg/ml to about 3 mg/ml, about 1.5 mg/ml to about 2.5 mg/ml, or from about 1.8 mg/ml to about 2.2 mg/ml. In another embodiment, poloxamer 188 is present at a concentration of about 2.0 mg/ml.

The pharmaceutical compositions provided herein are stable and can be stored for extended periods of time without an unacceptable change in quality, potency, or purity. In one aspect, the composition is stable at a temperature of about 5° C. (e.g., 2° C. to 8° C.) for at least 1 month, for example, at least 1 month, at least 3 months, at least 6 months, at least 12 months, at least 18 months, at least 24 months, or more. In another embodiment, the composition is stable at a temperature of less than or equal to about −20° C. for at least 6 months, for example, at least 6 months, at least 12 months, at least 18 months, at least 24 months, at least 36 months, or more. In another embodiment, the composition is stable at a temperature of less than or equal to about −40° C. for at least 6 months, for example, at least 6 months, at least 12 months, at least 18 months, at least 24 months, at least 36 months, or more. In another embodiment, the composition is stable at a temperature of less than or equal to about −60° C. for at least 6 months, for example, at least 6 months, at least 12 months, at least 18 months, at least 24 months, at least 36 months, or more.

Pharmaceutical compositions are typically sterile and stable under the conditions of manufacture and storage. Pharmaceutical compositions may be formulated as a solution, microemulsion, liposome, or other ordered structure suitable to accommodate high drug concentration. The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In some embodiments, isotonic agents, for example, sugars, polyalcohols such as mannitol, sorbitol, or sodium chloride are included in the composition. Prolonged absorption of the injectable compositions can be brought about by including in the composition an agent which delays absorption, for example, monostearate salts and gelatin. In certain embodiments, a novel rAAV viral particle provided herein may be administered in a time or controlled release composition, for example in a composition which includes a slow release polymer or other carriers that will protect the compound against rapid release, including implants and microencapsulated delivery systems. Biodegradable, biocompatible polymers may for example be used, such as ethylene vinyl acetate, polyanhydrides, polyglycolic acid, collagen, polyorthoesters, polylactic acid and polylactic, polyglycolic copolymers (PLG).

In some embodiments, a novel rAAV viral particle of the disclosure may be administered in via a delivery vehicle, such as a nanocapsule, microparticle, microsphere, lipid particle, exosome, exosome-like particle, or nanoparticle. The novel rAAV viral particle may be encapsulated within such a delivery vehicle. The delivery vehicle with the novel rAAV viral particle encapsulated may be in a pharmaceutical composition comprising an excipient, such as a buffer or other carrier.

In some embodiments, the pharmaceutical composition comprising a novel rAAV viral particle is used for transferring a transgene (e.g., therapeutic biomolecule) to a host cell. Depending on the application and disease to be treated, the transfer can be in vitro, ex vivo, in vivo, or a combination thereof.

### 6.4 Methods

The disclosure also provides various methods of use and treatment comprising a novel AAV capsid sequence of the disclosure (e.g., a novel rAAV viral particle or a composition thereof).

### 6.4.1 Methods of Delivery

Also provided are methods of using a novel rAAV viral particle or a composition of the disclosure for delivery of a biomolecule, e.g., a therapeutic biomolecule, to a cell, tissue, and/or organ. The method of delivery can be in vivo, in vitro, or ex vivo delivery.

The method comprises contacting a cell with an AAV viral particle as provided in Section 6.3.3. In some embodiments, the method is used to deliver a biomolecule (e.g., a therapeutic biomolecule) and/or composition to a particular cell, tissue, or organ type. For example, the method can be used to deliver a biomolecule (e.g., a therapeutic biomolecule) and/or composition to a muscle, heart, liver, plasma, kidney, brain, or cancer cell, or a combination thereof. Alternatively, the method of delivery can compromise one or more cell/tissue specificity, e.g., tropism as provided in Section 6.3.2.5.

In some embodiments, the method is used to deliver a therapeutic to a broad range of in vivo cells, including dividing or non-dividing cells. In some embodiments, the method is used to deliver a therapeutic gene to an in vitro cell, e.g., to produce a polypeptide encoded by such a therapeutic transgene for ex vivo gene therapy. It is contemplated that the methods of delivery provided by the disclosure can be for in vivo, in vitro, and/or ex vivo gene therapy approaches.

The method of delivery can be used to treat a disease or disorder. The structural and/or functional features of the novel AAV capsid sequences presented herein allow for an AAV-capsid platform approach for multiple disease indications that have one or more common defects or therapeutic needs as discussed in more detail below.

### 6.4.2 Methods of Treatment

The present disclosure provides methods of treatment of a disease or disorder that can be treated by delivery of a biomolecule to a particular tissue or cell type (e.g., muscle, heart, brain/CNS, plasma, kidney, liver, or cancer cell), comprising administering a novel rAAV viral particle, AAV vector construct, host cell or pharmaceutical composition of the disclosure comprising a therapeutic transgene or secreted therapeutic protein to a subject (e.g., a mammal or a human subject) in need thereof. In a specific embodiment, provided herein is a method for treating a disease or disorder, comprising administering to a subject (e.g., a human subject) in need thereof a therapeutically effective amount of a novel rAAV viral particle or a pharmaceutical composition thereof. In some embodiments, the disease or disorder treated can be a muscle, a heart, a brain, a CNS, a plasma, a kidney, a liver, an ear, or a cell proliferation (e.g., cancer or begin tumor) related disease or disorder. In some embodiments, the disease or disorder is one associated with one or more loss-of-function mutations within a gene, which reduces or abolishes the amount or activity of the protein encoded by the gene. In some embodiments, the disease or disorder is one associated with one or more haploinsufficiency mutations within a gene. In some embodiments, the disease or disorder is one associated with one or more gain-of-function mutations within a gene, which results in an aberrant amount or activity of the protein encoded by the gene.

In a specific embodiment, provided herein are methods of readministering or redosing subjects with an AAV viral particle, comprising administering two or more different AAV viral particles, wherein the AAV viral particle administered for the second or subsequent dosing comprises a different AAV capsid than the first AAV viral particle, and wherein at least one of the AAV viral particles is a novel rAAV viral particle described herein. In some embodiments, the first and second AAV viral particles each comprise a capsid protein(s) (e.g., VP1, VP2, or VP3) and those capsid proteins have less than or equal to 77%, 75%, 70%, 65%, 60%, 55%, or 50% sequence identity to each other. In some embodiments, the first and second AAV viral particles each comprise a capsid protein(s) (e.g., VP1, VP2, or VP3) and those capsid proteins have 50%, 55%, 60%, 65%, 70%, 75%, or 80% sequence identity to each other. In some embodiments, the first and second AAV viral particles each comprise a capsid protein(s) (e.g., VP1, VP2, or VP3) and those capsid proteins have 50% to 80%, 50% to 75%, 50% to 70%, 50% to 65%, or 50% to 60% sequence identity to each other. The AAV viral particles may comprise the same or a different transgene. Without wishing to be bound by theory, it is hypothesized that use of first and second AAV viral particles that have low capsid sequence identity will result in a lower immune response to the second AAV viral particle that is reduced in, e.g., an assay described herein (e.g., an assay described in Example 5) or known to one of skill in the art compared to readministration of the same first AAV viral particle, thereby permitting better transduction efficiency and transgene expression in the subject.

In various embodiments, the first and second capsids are phylogenetically distinct. In various embodiments, the phylogenetic difference is based on a threshold level of sequence homology. In various embodiments, the sequence homology of the capsids, or capsid proteins, may be less than or equal to 90%, 89%, 88%, 87%, 86%, 85%, 84%, 83%, 82%, 81%, 80%, 79%, 78%, 77%, 76%, 75% or lower sequence homology. In various embodiments, the sequence homology of the capsids, or capsid proteins, may be from about 30% to 90% homologous, from about 45% to 87% homologous, from about 40% to 86% homologous, from about 50% to 85% homologous, or from about 60% to 80% homologous, or from about 65% to 75% homologous. See, for example phylogenetically distinct capsids provided in FIGS. 3-13.

In some embodiments, there is low pre-existing immunity in the subject (e.g., a mammal or a human subject) to the first capsid and/or second capsid. In a specific embodiment, a subject exhibits low pre-existing immunity to either the first capsid, the second capsid or both the first and second capsids. In some embodiments, an in vitro assay to measure neutralizing antibody to AAV capsid is used to determine if a subject exhibits pre-existing immunity to the first capsid, the second capsid, or both the first and second capsids. In a specific embodiment, a technique known to one of skill in the art or described herein (e.g., in the Examples, infra) is used to assess pre-existing immunity in a subject.

In various embodiments, the first capsid may be from an AAV capsid in one clade in any one of Table 2 and the second AAV capsid is selected from a different clade, wherein there is sufficient phylogenetic distance between the viruses and/or amino acid sequence identity of the VP1 protein between the first and second AAV capsid. For example, the first capsid may be selected from an AAV capsid in clade 0 and the second capsid may be selected from an AAV capsid in any one of clades 0, 1, 4, 6, 11, 12, 13, 16, 21, and 22 of the disclosure, or vice versa.

Dosages of an AAV viral particle administered to a subject will depend on a variety of factors such as the disease or disorder being treated, the severity of the disease or disorder being treated, the age of the subject being treated, weight of the subject to be treated, and the age of the subject being treated. In specific embodiments, a novel AAV particle or a composition thereof is administered to a subject at a dose of from about 1×10⁹ vg/kg to about 6×10¹⁶ vg/kg of body weight. In some embodiments, a novel rAAV viral particle of the disclosure or a composition thereof is administered at a dose that is lower than a dose of a reference AAV (see, e.g., Table 4 or Item A or Item B, supra, for examples of reference AAV). For example, a lower dose of a novel rAAV viral particle of the disclosure or a composition thereof is required or necessary to obtain the same or better therapeutic effect as compared to the dose of a reference (see, e.g., Table 4 or Item A or Item B, supra, for examples of reference AAV).

Examples of routes of administration that can be used include but are not limited to, direct delivery to the selected organ, oral, inhalation, intravenous, intramuscular, subcutaneous, intradermal, intranasal, intrathecal, intrapancreatic, intraperitoneal, and other parental routes of administration.

### 6.4.3 Methods of Manufacture

The disclosure provides methods of manufacture using the novel AAV capsid sequences of the disclosure to produce a novel rAAV viral particle or a biomolecule (e.g., a therapeutic protein). Depending on the application, the biomolecule (e.g., the therapeutic protein) can be produced in vitro or in vivo.

6.4.3.1 Production of Novel rAAV Viral Particles

Any method known in the art may be used for the preparation of a novel rAAV viral particle of the disclosure. In some embodiments, a novel rAAV viral particle is produced in mammalian cells (e.g., HEK293). In some embodiments, a novel rAAV viral particle is produced in insect cells (e.g., Sf9). In some embodiments, an AAV viral particle is prepared by providing to a host cell with an AAV vector genome comprising a transgene together with a Rep and Cap gene. In some embodiments, an AAV vector genome comprises a transgene, an AAV Rep gene and an AAV Cap gene. In some embodiments, an rAAV viral particle is prepared by providing to a host cell with two or more vectors. For example, in some embodiments, an AAV vector genome comprising a transgene is introduced (e.g., transfected or transduced) into a cell with a vector (e.g., a plasmid or baculovirus) comprising an AAV Rep gene and a AAV Cap gene. In some embodiments, a cell is transfected or transduced with an AAV vector genome comprising a transgene, a vector (e.g., a plasmid or baculovirus) comprising an AAV Rep gene, and a vector (e.g., a plasmid or baculovirus) comprising an AAV Cap gene. In some embodiments, a method for producing a rAAV viral particle comprises culturing a host cell comprising a rAAV vector genome, a Rep protein, and a Cap gene, wherein the Cap gene encodes a capsid protein described herein. In some embodiments, the method further comprises isolating the rAAV viral particle from the host cell.

Methods of making AAV viral particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety.

In some embodiments, a novel rAAV viral particle is produced in host cell that allows for the production and replication of the novel rAAV particle. For example, the host cell may be a bacterial cell or eukaryotic cell, such as, e.g., an insect cell, yeast cell and mammalian cell (e.g., human cell or non-human mammalian cell). In specific embodiments, the host cell is from a cell line. Host cells commonly used for production of rAAV viral particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. Patent Application Publication No. 2002/0081721, and International Patent Publication Nos. WO 2000047757, WO 2000024916, and WO 1996017947, the contents of each of which are herein incorporated by reference in their entirety. In some embodiments, the HEK293 cells may be HEK-293T cells. Other examples of mammalian cells that may be used for the production of AAV viral particles include A549, WEH1, 3T3, 10T1/2, BHK, MDCK, COS 1, COS 7, BSC 1, BSC 40, BMT 10, VERO, W138, Saos, C2C12, L cells, HT1080, HepG2 and primary fibroblast, hepatocyte and myoblast cells derived from mammals. In some embodiments, host cells used for the production of AAV viral particles are cells derived from mammalian species including, but not limited to, human, monkey, mouse, rat, rabbit, and hamster. In some embodiments, host cells used for the production of AAV viral particles are cells derived from a cell type, including but not limited to fibroblast, hepatocyte, tumor cell, cell line transformed cell, etc.

In some embodiments, a novel rAAV viral particle is produced in an insect cell. Growing conditions for insect cells in culture, and production of heterologous products in insect cells in culture are well-known in the art, see U.S. Pat. No. 6,204,059, the contents of which are herein incorporated by reference in its entirety.

Any insect cell which allows for replication of parvovirus and which can be maintained in culture can be used in accordance with the disclosure. Cell lines may be used from Spodoptera frugiperda, including, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which is herein incorporated by reference in its entirety.

Baculovirus expression vectors for producing viral particles in insect cells, including but not limited to Spodoptera frugiperda (Sf9) cells, provide high titers of viral particle product. Infectious baculovirus particles released from a primary infection secondarily infect additional cells in the culture, exponentially infecting the entire cell culture population in a number of infection cycles that is a function of the initial multiplicity of infection, see Urabe, M. et al., J. Virol. 2006 February; 80 (4): 1874-85, the contents of which are herein incorporated by reference in their entirety.

Production of novel rAAV viral particles with baculovirus in an insect cell system may address known baculovirus genetic and physical instability. In some embodiments, the production system addresses baculovirus instability over multiple passages by utilizing a titerless infected-cells preservation and scale-up system. In some embodiments, small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al., Protein Expr Purif. 2009 June; 65 (2): 122-32, the contents of which are herein incorporated by reference in their entirety.

In some embodiments, a genetically stable baculovirus is used as the source of one or more of the components for producing AAV viral particles in invertebrate cells. In some embodiments, defective baculovirus expression vectors are maintained episomally in insect cells. In some embodiments, the bacmid vector is engineered with replication control elements, including but not limited to promoters, enhancers, and/or cell-cycle regulated replication elements.

In some embodiments, stable host cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, a novel rAAV viral particle of the disclosure is produced by triple transfection or baculovirus mediated virus production, or any other method known in the art. Any suitable permissive or packaging cell known in the art may be employed to produce the particles. In some embodiments, trans-complementing packaging cell lines that provide functions deleted from a replication-defective helper virus is used, e.g., 293 cells or other Ela trans-complementing cells. A packaging cell line may be used that is stably transformed to express cap and/or rep genes. Alternatively, a packaging cell line may be used that is stably transformed to express helper constructs necessary for AAV viral particle assembly.

In some embodiments, a novel rAAV viral particle production is modified to increase the scale of production. Large scale viral production methods according to the disclosure is any of those disclosed in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. In some embodiments, methods of increasing viral particle production scale include increasing the number of host cells. In some embodiments, host cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE (Corning Corp., Corning, NY) and NUNC™ CELL FACTORY® (Thermo Scientific, Waltham, MA). In some embodiments, large-scale adherent cell surfaces include from about 1,000 cm² to about 100,000 cm². In some embodiments, large-scale adherent cell cultures include from about 10⁷ to about 10⁹ cells, from about 10⁸ to about 10¹⁰ cells, from about 10⁹ to about 10¹² cells or at least 10¹² cells. In some embodiments, large-scale adherent cultures produce from about 10⁹ to about 10¹², from about 10¹⁰ to about 10¹³, from about 10¹¹ to about 10¹⁴, from about 10¹² to about 10¹⁵ or at least 10¹⁵ AAV viral particles.

In some embodiments, large-scale viral production methods of the disclosure include the use of suspension cell cultures. Suspension cell culture allows for significantly increased numbers of cells. Typically, the number of adherent cells that can be grown on about 10-50 cm² of surface area can be grown in about 1 cm³ volume in suspension.

In some embodiments, transfection of host cells in large-scale culture formats is carried out according to any methods known in the art. In some embodiments, for large-scale adherent cell cultures, transfection methods include, but are not limited to the use of inorganic compounds (e.g., calcium phosphate), organic compounds (e.g., polyethyleneimine (PEI)) or the use of non-chemical methods (e.g., electroporation.) With cells grown in suspension, transfection methods can include, but are not limited to the use of calcium phosphate and the use of PEI. In some embodiments, transfection of large scale suspension cultures is carried out according to the section entitled “Transfection Procedure” described in Feng, L. et al., 2008. Biotechnol Appl. Biochem. 50:121-32, the contents of which are herein incorporated by reference in its entirety. In some embodiments, PEI-DNA complexes is formed for introduction of plasmids to be transfected. In some embodiments, cells being transfected with PEI-DNA complexes are ‘shocked’ prior to transfection. This comprises lowering cell culture temperatures to 4° C. for a period of about 1 hour. In some embodiments, cell cultures are shocked for a period of from about 10 minutes to about 5 hours. In some embodiments, cell cultures are shocked at a temperature of from about 0° C. to about 20° C.

In specific embodiments, a novel rAAV viral particle is isolated from the host cells it is produced in. In a specific embodiment, a novel rAAV viral particle is produced by the methods disclosed in the Examples (e.g., Example 3).

### 6.4.3.2 In Vitro Production of a Biomolecule

As a non-limiting example, a novel rAAV viral particle disclosed herein can be used to produce a biomolecule of interest (e.g., a protein of interest) in vitro, for example, in a cell culture. As one non-limiting example, in some embodiments, a method for producing a protein of interest in vitro, where the method includes contacting a novel rAAV viral particle comprising a nucleotide sequence encoding a biomolecule (e.g., a heterologous protein) with a cell in a cell culture, whereby the AAV viral particle expresses the biomolecule (e.g., protein of interest) in the cell.

### 6.4.3.3 In Vivo Production of a Biomolecule

A novel rAAV viral particle disclosed herein can be used to produce a biomolecule of interest (e.g., protein of interest) in vivo, for example in an animal such as a mammal. Some embodiments provide a method for producing a biomolecule of interest (e.g., protein of interest) in vivo, where the method includes administering a novel rAAV viral particle comprising a nucleotide sequence that comprises a transgene encoding the biomolecule (e.g., protein of interest) to the subject, whereby the AAV viral particle expresses the biomolecule of interest (e.g., protein of interest) in the subject. The subject can be, in some embodiments, a non-human mammal, for example, a monkey, a dog, a cat, a mouse, or a cow. In specific embodiments, the subject is a human. See Section 6.4.2 for routes of administration and for doses.

Other aspects and advantages of the present disclosure will be understood upon consideration of the following illustrative examples.

### 6.5 Examples

### 6.5.1 Example 1: Identification of Novel AAV Capsid Proteins from Animal Tissue Sources

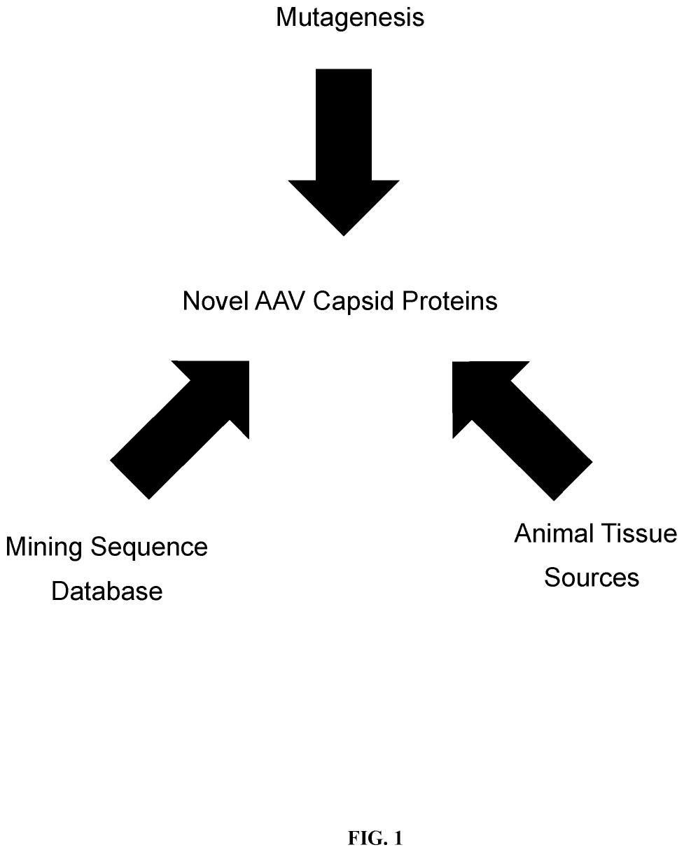

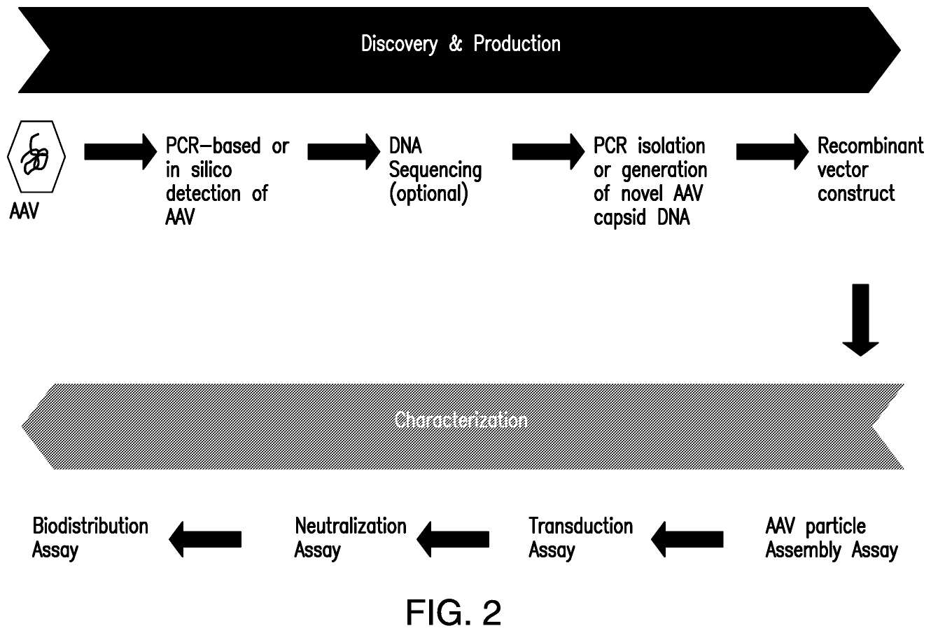

To identify novel AAV capsid proteins, genomic DNA was collected from various animal tissue sources and publicly available NGS sequence databases were evaluated, as shown in FIGS. 1-2 and described in more detail below.

### Genomic DNA Preparation and Isolation:

Genomic DNA was prepared from the collected sample using the DNeasy Blood & Tissue kit (Qiagen catalog #69504). Polymerase chain reaction (PCR) was carried out on the genomic DNA using a forward primer complimentary to the helicase domain in rep and a reverse primer complementary to one of the several DNA binding domains in cap protein. The expected size of the PCR fragments is 1.5 kb under the following conditions: initial incubation: 97° C., 120 sec, denaturation step: 97° C., 15 sec, annealing step: 58° C., 60° C., or 62° C., 15 sec, extension step: 72° C., 120 sec. The denaturation, annealing, and extension steps were performed for 42 cycles. Then the reaction was incubated at 72° C., 7 min and stored at 4° C. until analyzed.

The PCR products were separated by electrophoresis on the FlashGel System (Lonza catalog #57034), PCR product is purified by Select-a-Size DNA Clean and Concentrator Kit (Zymo catalog #D4080) and cloned into pCR4-TOPO-TA (Invitrogen catalog #450030) according to the manufacturer's instructions. After transformation of E. coli, NEB5α cells, DNA was prepared from ampicillin resistant colonies and sequenced from both ends to determine if the insert encoded an AAV-related sequence.

If the inserts in pCR4-TOPO TA were related to AAV sequences various methods were used for isolation of capsid sequence. PCR based isolation methods included best guessed 3 prime UTR primer design, genome walking or around the episome PCR. In one method, sequence-specific primers were designed to the rep portion of the sequence to perform “around the episome PCR” (hereinafter “ATE PCR”) to obtain a complete capsid gene. ATE PCR is based on the notion that persistent AAV genomes forms circular episomes in animal tissues. Accordingly, one can use a “divergent” set of primers corresponding to a sequence in the rep gene to perform polymerase chain reactions to isolate most or all of any AAV sequence that may exist in that episome, and in particular one could isolate a complete contiguous capsid gene. Multimers of episomes can form, for example by homologous recombination, and in that case, it is possible to isolate more than one capsid gene (which usually are not the same) from a single ATE PCR reaction.

### Computational Analysis of PCR Amplified DNA Sequences:

Once complete sequences were determined they were identified as being AAV capsid genes using the BLAST algorithm (available at the NCBI website). Their relationship to previously published AAVs was determined using various alignment programs such as Geneious Prime, Clustal Omega (available at the EBI web site) or Vector NTI (Invitrogen, Inc.).

Production of Vectors and rAAV Viral Particles:

To produce the AAV, AAV capsid genes were subcloned into an expression plasmid (pAAV-RC; Agilent, Inc.), then transfected into HEK293 cells along with a vector (pAAV luciferase) and adenovirus helper plasmid (pHELPER; Agilent, Inc.). AAV production was allowed to occur for 3 days and then crude lysates were made by freeze-thawing the cells three times. Debris was pelleted and the supernatant (crude AAV) was titered by Q-PCR to determine a genomic titer (which confirms the capsid is capable of assembly and DNA packaging) and then used to assess transduction by the AAVs on various cells.

### Identification of AAV in NGS Databases:

Using Short Read Archive (SRA) from publicly available NGS databases, Magic-BLAST was used to align and compare the SRAs to a diverse AAV reference sequence that was constructed and assembled from published AAV from various animal sources.

The Magic-BLAST output was then parsed and compared to the diverse AAV reference sequence to identify sequences that matched the diverse AAV reference. Next, Megablast analysis was conducted to determine if the matches do not align with any published AAV sequence. SRA matches that aligned and met the criteria above are used to assemble a full length capsid VP1 protein.

### 6.5.2 Example 2: Generation of Vectors and rAAV Vector Genomes

Depending on the assay and production system used, vectors and/or rAAV vector genomes were generated as described below.

Briefly, constructs were prepared with the nucleic acid sequences for cap genes SEQ ID NOs. 1-180 and 361-362, rep gene, at least one ITR, such as AAV-2 (see Table 4), a promoter, one or more regulatory control elements and a transgene or a reporter gene using conventional cloning techniques. See Green, M. and Sambrook, J., Molecular Cloning: A Laboratory Manual, 4th Ed., Cold Spring Harbor Laboratory (Cold Spring Harbor, N.Y. 2012).

Depending on the production system used, the HEK293 cells and/or baculovirus, the vectors and/or rAAV vector genome was configured into either a vector genome or, for example there can be either: two, three, or four separate vectors comprising the needed genetic elements for AAV and transgene expression (e.g., ITRs, rep, cap, regulatory elements, transgene and/or adenovirus helper functions) for a rAAV vector genome/

The rAAV viral particles comprising the novel AAV capsid sequences were produced as provided in Example 3.

### 6.5.3 Example 3: Production of Novel rAAV Viral Particles in HEK293 and Baculovirus Infected Insect Cells

The vectors and/or rAAV vector genomes described in Example 2 were used to produce rAAV viral particles using the HEK293 cells and/or baculovirus infected insect cell production system as described below.

### Generation of Recombinant Bacmid:

DH10 Bac competent cells were thawed on ice. Recombinant shuttle plasmid (e.g., pFB-GFP) was added and gently mixed with the competent cells and incubated on ice for 30 minutes. The competent cells were then subjected to heat at a temperature of approximately 42° C. for 30 seconds and then chilled on ice for 2 minutes. The competent cells were shocked with heat for 30 seconds at 42° C. and chilled on ice for 2 min. SOC was added to the cells and the cells were allowed to incubate at 37° C. with agitation for 4 hours to allow recombination to take place. During the incubation period, X-gal was spread onto two LB-plates (additionally containing various antibiotics (e.g., kanamycin, gentamycin and tetracycline) for transformation, followed by IPTG.

An amount of the incubation mixture was obtained, diluted, and then spread onto the two LB-plates and incubated at 37° C. for approximately 30-48 hours. Several white colonies were selected from each plate and cultured overnight in LB medium containing the same combination of antibiotics provided in the LB-plates. Next, Bacmid DNA and a glycerol stock was prepared and stored at −80° C.

### Purification of Recombinant Bacmid DNA:

An amount of the Bacmid glycerol stock was removed and inoculated in LB medium containing the a combination of antibiotics for selection. Cultures were grown overnight at 37° C. with shaking. Next, an amount of the culture was spun in a microfuge at full speed for approximately 30 seconds.

The pellets were resuspended in a resuspension buffer using a pipette followed by a lysis buffer, and the tube was inverted several times to mix the buffer and then incubated at room temperature for approximately 5 minutes. An exemplary resuspension buffer comprises 50 mM Tris-CL, pH 8.0, 10 mM EDTA and 100 μg/mL RNase A. An exemplary lysis buffer comprises 200 mM NaOH and 1% SDS. An amount of precipitate buffer (e.g., a buffer comprising 3.0 M potassium acetate, pH 5.5) was slowly added and the tube was inverted several times to mix the buffer and then incubated on ice for approximately 10 minutes. The tube was centrifuged for approximately 10 minutes at full speed and the supernatant is poured into a tube containing isopropanol. The tube was inverted several times to mix the solution.

Next, the solution was centrifuged at full speed for approximately 15 minutes at room temperature and the supernatant was removed immediately after centrifuge with pipette.

An amount of 70% ethanol was added to rinse the pellet and spun again at full speed for 1 minute. The ethanol was then removed, and the solution was spun again to remove trace of the ethanol. An amount of TE/EB Buffer was added to each tube and the pellet was carefully dissolved by pipette. The solution was stored at −20° C. if not used immediately.

### Production of PO Stock of Recombinant Baculovirus:

Sf9 cells were seeded at approximately 1×10⁶ cells/well in a 6-well plate (or 6×10⁶ cells in a 10-cm plate or 1.7×10⁷ cells in a 15-cm dish) and the cells were allowed to attach for at least 1 hour before transfection.

Transfection solutions A and B were prepared as follows: Solution A: an amount of the Bacmid was diluted into an amount of serum free media without antibiotics in a 15-mL tube. Solution B: an amount of CellFectin was diluted into an amount of serum free media without antibiotics in a 15-mL tube. Solution B was added to Solution A and gently mixed by pipette approximately 3 times by pipette and incubated at room temperature for 30-45 minutes. Next, medium from the plate was aspirated and an amount of serum free media without antibiotics was added to wash the cells. An amount of SF900II without antibiotics was added to each tube containing lipid-DNA mixtures.

The medium from the cells was aspirated, the transfection solution was added to the cells and the cells were incubated for approximately 5 hours at 28° C. The transfection solution was removed and an amount of serum free media with antibiotics was added, and incubated for approximately 4 days at 28° C. Media that contains the recombinant baculovirus was collected and spun for approximately 5 minutes at 1000 rpm to remove cell debris. The baculovirus was stored at 4° C. under dark.

### Amplification of Baculovirus (P1):

Sf9 cells were grown to approximately 4×10⁶ cells/mL and diluted to approximately 2×10⁶ cells/mL with fresh medium in shaking flasks. An amount of the Sf9 cells was infected with an amount of the PO stock baculovirus. The multiplicity of infection (MOI) was approximately 0.1.

The Sf9 cells were incubated for approximately 3 days and the baculovirus was harvested. The cells were spun at 2,000 rpm for 5 minutes to pellet the cells and the supernatant was collected and stored at 4° C. under dark. The titer of the baculovirus was determined according to Clontech's Rapid Titer Kit protocol.

### Production of AAV Using P1 Recombinant Baculovirus:

Sf9 cells were grown to about 1×10⁷ cells/mL and diluted to about 5×10⁶ cells/mL. An amount of the diluted Sf9 cells were infected with Bac-vector (5 Moi) and Bac-helper (15 Moi) for 3 days. Cell viability was assessed on the third day (approximately 50%-70% dead cells were observed).

Cell pellets were harvested by centrifugation at 3000 rpm for 10 minutes. Media was removed and the cells lysed (or the cell pellets were stored at −20° C. if not used immediately).

### Lysis and Banding Protocol:

An amount of Sf9 lysis buffer plus Benzonase was added to each cell pellet and vortexed thoroughly to resuspend the cells. The resuspended Sf9 cells were incubated on ice for approximately 10 min. to cool lysate. The lysate was sonicated for approximately 20 seconds to lyse the cells thoroughly and then incubated at 37° C. for approximately 30 minutes.

An amount of 5 M NaCl was added and the mixture was vortexed and then incubated for another 30 minutes at 37° C. An amount of NaCl was added to bring the salt concentration to about 500 mM, vortexed and centrifuged at 8,000 rpm for 20 minutes at 15° C. to produce a cleared lysate.

The cleared lysate proceeds to ultracentrifugation steps. A CsCl-gradient was prepared by adding the cleared lysate first, then an amount of 1.32 g/cc and an amount of 1.55 g/cc CsCl solutions through a syringe with long needle. The interface between the CsCl solutions was marked. PBS was added up to the top of the centrifuge tubes and the tubes were carefully balanced and sealed.

The tubes were centrifuged at 55,000 rpm for approximately 20 hours at 15° C. A hole was puncture on the top of each tube and the AAV band located slightly above the interface mark of the two CsCl solutions was marked.

A second CsCl centrifugation was conducted by transferring the AAV solution to centrifuge tube for 70.1 Ti rotor and an amount of CsCl solution to near top of the tube was added. The tubes were balanced and sealed. The tubes were centrifuged at 65,000 rpm for approximately 20 hours and the AAV band (lower band, the higher band is empty capsids) was collected.

Three separate vectors and/or rAAV vector genomes were selected and tested for their ability to produce rAAV viral particle in HEK293 and/or baculovirus infected insect cells. The majority of the selected clones produced viral particles in either HEK293 cells and/or baculovirus infected insect cells (data not shown). Selected rAAV viral particles were chosen for further analysis as provided in the Examples below.

### 6.5.4 Example 4: Evaluation of Human Serum Neutralization of Novel rAAV Viral Particles

The ability of antibodies in human serum to neutralize selected rAAVs and novel rAAV viral particles was evaluated as described below.

### Intravenous Immunoglobulin (IVIg) Neutralization Assay:

HEK293T cells were seeded in density 4E4 cells/well in a 96 well plate and incubated overnight. Purified rAAVs were diluted to final titer of 2E6 vg/uL after mixing 1:1 with serial dilutions (0-20 mg/mL) of IVIg for 1 hour. Recombinant AAVs were added onto HEK293T cells at an MOI of 1000 with 10 μM Etoposide and incubated in 37° C. Seventy-two hours following viral addition, percent transduction was assessed by luciferase activity measured in Relative Luminescence Units (RLU) relative to control transductions with vector+BSA only.

Table 5 and FIGS. 14A-B show data for neutralization assays in HEK293T cells for each individual rAAV in the presence of increasing concentrations of purified human immunoglobulin (IVIg) from pooled healthy human serum. Luciferase expression was quantified and the data expressed as percent “%” transduction from the avg of duplicates. Luciferase expression was quantified and the data expressed as mean RLU.

FIG. 14B shows NC₅₀ data. The NC₅₀ was determined by calculating the concentration of IVIg (x-value of the point on a line) where transduction equals 50%, where the line is made by connecting two points above and below 50% transduction.

TABLE 5
IVIg neutralization data presented in (%) transduction.
Branch 2—IVIG neutralization data presented in (%) transduction

| Name/ | | | | | | | | | | |
|---|---|---|---|---|---|---|---|---|---|---|
| IVIg in | 10 | 5 | 2.5 | 1.25 | 0.625 | 0.312 | 0.156 | 0.078 | 0.039 | 0.00 |
| mg/ml | mg/ml | mg/ml | mg/ml | mg/ml | mg/ml | mg/ml | mg/ml | mg/ml | mg/ml | mg/ml |
| AAV-6 | 4.7E−01 | 3.9E−01 | 1.0E+00 | 2.1E+00 | 8.1E−01 | 1.0E+00 | 5.8E+00 | 2.3E+01 | 5.7E+01 | 100 |
| AAV-7 | 3.7E−01 | 4.3E−01 | 2.5E−01 | 4.3E−01 | 5.7E−01 | 2.7E+00 | 4.9E+00 | 2.8E+01 | 5.3E+01 | 100 |
| AAV-8 | 4.4E−02 | 4.0E−02 | 9.3E−02 | 3.8E−02 | 6.5E−02 | 7.3E−01 | 9.2E+00 | 3.6E+01 | 6.8E+01 | 100 |
| AAV-9 | 1.4E−01 | 6.0E−02 | 1.1E−01 | 1.0E−01 | 1.3E−01 | 2.2E+00 | 7.4E+00 | 3.4E+01 | 5.7E+01 | 100 |
| BCD_ | 4.8E−02 | 1.8E−02 | 2.9E−02 | 9.0E−02 | 1.8E−01 | 2.3E+00 | 1.3E+01 | 3.6E+01 | 6.1E+01 | 100 |
| 0106 | | | | | | | | | | |
| BCD_ | 1.8E−02 | 1.5E−02 | 5.4E−02 | 7.4E−02 | 2.7E−02 | 9.1E−02 | 7.7E−01 | 6.8E+00 | 3.1E+01 | 100 |
| 0159 | | | | | | | | | | |
| BCD_ | 1.0E−02 | 9.9E−03 | 1.3E−02 | 1.1E−01 | 1.9E−02 | 2.4E−01 | 7.5E+00 | 3.2E+01 | 5.8E+01 | 100 |
| 0190 | | | | | | | | | | |
| BMN_ | 2.8E−01 | 2.9E−01 | 2.1E−01 | 2.1E−01 | 4.2E−01 | 2.5E+00 | 3.8E+00 | 1.1E+01 | 4.1E+01 | 100 |
| 0213 | | | | | | | | | | |
| BMN_ | 1.4E−01 | 1.9E−01 | 9.6E−02 | 1.2E−01 | 1.2E−01 | 2.2E−01 | 2.1E+00 | 1.6E+01 | 3.9E+01 | 100 |
| 0217 | | | | | | | | | | |
| BMN_ | 4.9E−02 | 6.3E−02 | 2.8E−02 | 1.2E−01 | 9.9E−02 | 5.3E−01 | 5.3E+00 | 3.4E+01 | 6.1E+01 | 100 |
| 0251 | | | | | | | | | | |
| BMN_ | 5.2E−01 | 5.5E−01 | 5.5E−01 | 9.2E−01 | 7.7E−01 | 7.3E−01 | 5.6E+00 | 2.7E+01 | 5.4E+01 | 100 |
| 0264 | | | | | | | | | | |
| BMN_ | 4.8E−01 | 4.6E−01 | 6.6E−01 | 5.7E−01 | 5.4E−01 | 6.9E−01 | 1.3E+01 | 5.4E+01 | 8.0E+01 | 100 |
| 0270 | | | | | | | | | | |
| BMN_ | 6.4E−01 | 4.0E−01 | 7.6E−01 | 7.4E−01 | 1.1E+00 | 1.0E+00 | 6.2E+00 | 5.1E+01 | 7.1E+01 | 100 |
| 0271 | | | | | | | | | | |
| BMN_ | 2.4E−01 | 1.9E−01 | 2.3E−01 | 2.0E−01 | 1.5E−01 | 1.6E+00 | 1.2E+01 | 3.5E+01 | 6.9E+01 | 100 |
| 0272 | | | | | | | | | | |
| BMN_ | 2.2E−01 | 1.8E−01 | 1.9E−01 | 1.8E−01 | 2.2E−01 | 5.8E−01 | 1.9E+00 | 1.6E+01 | 5.4E+01 | 100 |
| 0273 | | | | | | | | | | |
| BMN_ | 1.4E−01 | 1.3E−01 | 1.9E−01 | 1.8E−01 | 2.3E−01 | 2.6E+00 | 1.5E+01 | 4.1E+01 | 6.1E+01 | 100 |
| 0304 | | | | | | | | | | |
| BMN_ | 6.1E−01 | 6.1E−01 | 6.0E−01 | 5.2E−01 | 5.7E−01 | 1.3E+00 | 1.4E+01 | 4.5E+01 | 6.0E+01 | 100 |
| 0308 | | | | | | | | | | |
| BMN_ | 7.1E−01 | 5.6E−01 | 5.6E−01 | 9.3E−01 | 1.1E+00 | 1.3E+00 | 8.5E+00 | 2.3E+01 | 5.1E+01 | 100 |
| 0320 | | | | | | | | | | |
| BMN_ | 1.2E−01 | 1.2E−01 | 1.2E−01 | 5.7E−01 | 1.8E−01 | 2.0E+00 | 8.4E+00 | 3.1E+01 | 4.6E+01 | 100 |
| 0321 | | | | | | | | | | |
| BCD_ | 2.2E+00 | 1.2E+00 | 1.6E+00 | 1.7E+00 | 1.5E+00 | 9.8E−01 | 1.2E+00 | 1.5E+01 | 6.5E+01 | 100 |
| 0386 | | | | | | | | | | |
| BCD_ | 6.85E−02 | 5.44E−02 | 1.03E−01 | 6.04E−02 | 1.22E−01 | 1.21E−01 | 4.52E+00 | 1.77E+01 | 4.97E+01 | 100 |
| 0160 | | | | | | | | | | |
| BCD_ | 9.85E−02 | 1.22E−01 | 7.27E−02 | 6.00E−02 | 8.61E−01 | 7.77E+00 | 2.10E+01 | 5.07E+01 | 7.48E+01 | 100 |
| 0361 | | | | | | | | | | |

The ability of antibodies in human serum to neutralize the novel rAAV viral particles compared to other AAVs are provided in the Table 5 and in FIGS. 14A-B. FIG. 14B provides the NC₅₀ calculated from the IVIg assay and as provided herein, for various capsids including some rAAV viral particles comprising an AAV capsid protein. The fold difference from a novel rAAV viral particle's NC₅₀ from a AAV capsid's NC₅₀ indicates the enhanced ability of the disclosed novel capsid to evade AAV humoral immunity. For example, in the IVIg assay the AAV capsid, AAV-12 had NC₅₀ of about 0.5263 mg/mL, AAV-6 had an NC₅₀ of about 0.0476 mg/mL, AAV-7 had an NC₅₀ of about 0.0441 mg/mL, AAV-8 had an NC₅₀ of about 0.0610 mg/mL, AAV-9 had an NC₅₀ of about 0.0513 mg/mL, and AAV-5 had an NC₅₀ of about 0.2326 mg/mL, AAV-2 had an estimated NC₅₀ of about less than <0.0305 mg/mL. The rAAV capsids in Branch 2 had an average NC₅₀ of 0.05259 mg/mL, while the novel rAAVs capsid (“BCDs) had an average of 0.05188 mg/ml and a range of 0.0337 mg/mL to 0.08 mg/mL. These data show that several novel rAAV viral particles comprising the novel capsid protein can evade the recognition, binding, and/or neutralization by antibodies in human plasma/sera and therefore have enhanced ability to evade AAV humoral immunity.

### 6.5.5 Example 5: Evaluation of In Vitro Cell Transduction of Novel rAAV Viral Particles

Selected novel rAAV viral particles were examined for their ability to transduce human cell lines.

Production and Purification of rAAV Viral Particles:

rAAV viral particles are produced by triple transfection of HEK293 cells using a rAAV vector genome plasmid, a rep and cap plasmid, and an AAV helper plasmid with Calcium Phosphate. AAV viral particles were purified by double Cesium Chloride gradient ultracentrifugation. AAV viral tier was determined by qPCR.

### HEK293 and HepG2 Cell Assays:

HEK293 and HepG2 cells were seeded at 4.5×10⁴ cells/well on a 96-well plate and incubated overnight. Etoposide was added on the day of infection to a final concentration of 4 μM and 20 μM for HEK293 and HepG2 cells, respectively. Next, purified AAV viral particles were added at a MOI of 2000 and transduction data was measured in relative luciferase units (RLU) 72 hours post-infection.

### U87MG Cell Assay:

Human glioblastoma U87MG cells were seeded at 4.5×10⁴ cells/well on a 96-well plate and incubated overnight. Etoposide was added on the day of infection to a final concentration of 4 μM. Purified AAV particles were added at a MOI of 2000 and transduction data was measured in RLU units 72 hours post-infection.

In Table 6, in vitro cell line transduction data for HEK293, HepG2, and U87MG are provided for each novel rAAV viral particle tested. The average cell transduction efficiency is shown as averaged RLU units (AVG), and the standard deviation (SD) is indicated. Blank cells indicate the assay has not yet been performed.

TABLE 6
In vitro cell transduction data presented in averaged
RLU units (AVG) and standard deviation (SD).
Branch 2—In vitro cell transduction data presented in RLU units

| | HepG2 | HepG2 | HEK293 | HEK293 | U87MG | U87MG |
|---|---|---|---|---|---|---|
| Name | (AVG) | (SD) | (AVG) | (SD) | (AVG) | (SD) |
| AAV-7 | 5.11E+04 | 1.26E+03 | 1.02E+04 | 7.75E+02 | 2.19E+04 | 1.76E+03 |
| AAV-8 | 7.70E+04 | 5.35E+02 | 1.14E+05 | 3.92E+03 | 6.64E+05 | 1.46E+04 |
| AAV-9 | 3.26E+04 | 1.64E+03 | 5.90E+03 | 6.22E+02 | 4.25E+04 | 2.72E+03 |
| BCD_0106 | 5.34E+04 | 6.98E+03 | 3.95E+04 | 5.50E+03 | 1.65E+04 | 2.79E+03 |
| BCD_0113 | 3.18E+04 | 4.00E+02 | 1.78E+04 | 1.24E+03 | 1.72E+05 | 1.23E+04 |
| BCD_0126 | 6.80E+04 | 6.50E+03 | 2.40E+04 | 4.10E+03 | 4.30E+05 | 1.20E+04 |
| BCD_0127 | 1.00E+05 | 5.90E+03 | 4.00E+04 | 1.70E+03 | 4.80E+05 | 1.60E+04 |
| BCD_0146 | 6.50E+04 | 1.20E+04 | 1.40E+04 | 1.40E+03 | 8.20E+04 | 4.40E+03 |
| BCD_0157 | 1.84E+05 | 1.84E+04 | 4.71E+04 | 2.89E+03 | 2.70E+05 | 1.26E+04 |
| BCD_0159 | 8.10E+03 | 5.95E+02 | 2.45E+04 | 5.33E+03 | 9.27E+04 | 9.10E+03 |
| BCD_0160 | 9.72E+04 | 6.87E+03 | 2.19E+04 | 4.40E+03 | 3.98E+05 | 2.48E+04 |
| BCD_0177 | 4.50E+04 | 4.00E+03 | 2.10E+04 | 7.30E+02 | 2.50E+05 | 1.20E+04 |
| BCD_0181 | 3.80E+04 | 3.30E+03 | 2.00E+04 | 2.80E+03 | 2.50E+05 | 1.10E+04 |
| BCD_0186 | 4.20E+04 | 4.40E+03 | 1.90E+04 | 2.40E+03 | 2.70E+05 | 1.80E+04 |
| BCD_0187 | 5.50E+04 | 5.30E+03 | 2.40E+04 | 1.60E+03 | 3.50E+05 | 1.60E+04 |
| BCD_0190 | 4.55E+04 | 6.29E+03 | 2.50E+04 | 5.51E+03 | 1.08E+05 | 3.42E+03 |
| BCD_0197 | 4.80E+04 | 4.50E+03 | 2.40E+04 | 2.50E+03 | 2.90E+05 | 1.70E+04 |
| BMN_0213 | 1.15E+04 | 2.69E+02 | 1.40E+04 | 3.50E+03 | 3.59E+04 | 4.12E+03 |
| BMN_0217 | 2.01E+04 | 6.73E+02 | 3.56E+04 | 5.30E+03 | 6.09E+04 | 1.57E+04 |
| BMN_0250 | | | | | 5.60E+01 | 4.00E+00 |
| BMN_0251 | 1.03E+05 | 9.73E+02 | 6.47E+04 | 1.37E+04 | 5.86E+04 | 4.03E+03 |
| BMN_0258 | 5.00E+01 | 4.00E+00 | 4.96E+03 | 1.68E+03 | 1.00E+02 | 2.40E+01 |
| BMN_0261 | 1.38E+03 | 2.68E+02 | 4.02E+02 | 5.00E+01 | 4.42E+02 | |
| BMN_0264 | 2.15E+04 | 1.88E+03 | 6.20E+03 | 2.80E+02 | 1.20E+04 | 4.47E+03 |
| BMN_0270 | 4.67E+04 | 2.37E+03 | 2.26E+04 | 6.14E+03 | 1.27E+05 | 4.17E+03 |
| BMN_0271 | 3.72E+04 | 7.33E+01 | 4.20E+03 | 5.55E+02 | 1.45E+04 | 4.55E+02 |
| BMN_0272 | 4.37E+04 | 2.40E+03 | 1.83E+04 | 2.28E+02 | 2.01E+04 | 2.96E+02 |
| BMN_0273 | 2.08E+04 | 3.36E+02 | 2.54E+04 | 9.12E+02 | 4.95E+03 | 1.60E+02 |
| BCD_0276 | 1.60E+04 | 2.87E+02 | 8.13E+03 | 8.00E+02 | 8.59E+04 | 7.12E+03 |
| BCD_0284 | 1.58E+04 | 2.62E+03 | 6.91E+03 | 5.95E+02 | 8.90E+04 | 1.01E+04 |
| BCD_0286 | 1.72E+04 | 1.25E+03 | 8.68E+03 | 1.35E+03 | 9.86E+04 | 1.10E+04 |
| BMN_0302 | | | | | 2.80E+01 | 6.00E+00 |
| BMN_0304 | 7.29E+04 | 4.31E+03 | 2.50E+04 | 3.48E+02 | 5.01E+04 | 2.68E+03 |
| BMN_0306 | 2.76E+02 | 1.00E+01 | 9.15E+02 | 1.49E+02 | 1.56E+03 | 2.88E+02 |
| BMN_0308 | 1.18E+04 | 6.79E+02 | 6.53E+03 | 1.03E+03 | 3.51E+04 | 1.82E+03 |
| BMN_0316 | 7.00E+01 | 2.00E+00 | 1.67E+03 | 3.53E+02 | 2.15E+03 | 1.88E+02 |
| BMN_0320 | 2.22E+04 | 1.47E+03 | 4.39E+04 | 9.70E+02 | 8.35E+03 | 5.50E+02 |
| BMN_0321 | 1.25E+04 | 1.87E+03 | 3.02E+04 | 1.97E+03 | 1.22E+05 | 1.31E+03 |
| BCD_0361 | 1.55E+04 | 2.71E+03 | 2.20E+04 | 2.91E+03 | 2.52E+04 | 1.93E+03 |

Blank cells indicate that the assay has not yet been performed. Where possible, transduction efficiency was compared to another AAV capsid. These results demonstrate that most of the novel rAAV viral particles are functional in that they are capable of transducing either HEK293, HepG2, and/or U87MG cells with varying efficiencies.

### 6.5.6 Example 6: Evaluation of In Vivo Tropism of Novel rAAV Viral Particles in Mice

An in vivo imaging system (IVIS) assay was conducted to investigate the tropism of selected novel rAAV viral particles in mice.

Production of rAAV Viral Particle with Reporter Gene:

rAAV comprising the novel VP1, VP2, and VP3 capsids sequences and expressing the luciferase transgene were generated (AAV-RSV-efp-T2A-Fluc2).

### In Vivo Tissue Expression Assay:

Male Balb/C or C57BL mice were purchased from Charles River Breeding Laboratories. A dose of 2×10¹³ vg/kg of AAV-RSV-egfp-T2A-Fluc2 vector was injected into the tail vein of 8 week old mice.

### IVIS Lumina LT Imaging:

At 3- and 5-weeks post injection, in vivo bioluminescent imaging was performed using an in vivo imagining device (IVIS Lumina LT obtained from PerkinElmer Inc., Waltham, MA). In brief, the mice were anesthetized with 2% isofluorane and oxygen. 150 μl of 30 mg/ml of RediJect D-Luciferin Bioluminescent Substrate was injected intraperitoneally. Ten minutes after substrate injection, the animals were imaged with IVIS Lumina LT system, equipped with a cooled charge-coupled device (CCD) camera. Images were taken in the dorsal positions of the animals. Anesthesia was maintained throughout the entire imaging session by isofluorane-oxygen delivery in the light-tight imaging chamber. The mice were sacrificed after the imaging sessions at 5 weeks post AAV injection. Organs from the animals were immediately harvested and imaged using IVIS Lumina LT system. The measurement conditions were the same as those used for in vivo imaging.

For imaging, a gray-scale photograph of the animals was acquired, followed by bioluminescence image acquisition. Image data was processed and analyzed using Living Image Software® version 4.5.2 (PerkinsElmer Waltham, MA). Regions of interest were traced surrounding each animal as well as individual organs to quantify the total flux (TF) (photons/second) being released by luciferase activity. Results are shown in Table 7 below. Blank cells indicate that the assay has not yet been performed.

TABLE 7
IVIS biodistribution data in presented as total flux in tissue
(photons/sec/cm2/radian): presented as average AVG with standard deviation (SD)
Table 7: Branch 2 Total Flux in Tissue (photons/sec/cm2/radian): presented as AVG/(SD)

| Name | Biceps Brachii | Brain | Diaphragm | Eyes | Gastrocnemius |
|---|---|---|---|---|---|
| AAV-8 | 1.91E+05 | 2.31E+06 | | | 6.87E+06 |
| | (1.55E+04) | (7.88E+05) | | | (4.71E+06) |
| AAV-6 | 1.72E+03 | 5.72E+03 | 9.13E+04 | 4.56E+03 | 1.68E+04 |
| | (2.34E+03) | (3.77E+03) | (6.95E+04) | (2.91E+03) | (2.82E+04) |
| AAV-7 | 4.42E+04 | 1.47E+05 | 9.98E+05 | 6.27E+04 | 7.04E+05 |
| | (4.69E+04) | (1.01E+05) | (1.05E+06) | (3.23E+04) | (6.56E+05) |
| BCD_0386 | 1.93E+06 | 2.92E+05 | 4.78E+07 | 5.97E+05 | 1.01E+08 |
| | (1.01E+06) | (1.93E+05) | (3.12E+07) | (6.74E+05) | (8.42E+07) |
| BCD_0159 | 2.78E+06 | 5.48E+04 | 2.24E+07 | 7.88E+04 | 1.03E+07 |
| | (1.06E+06) | (1.69E+04) | (1.13E+07) | (5.23E+04) | (7.89E+06) |
| BCD_0190 | 7.87E+07 | 2.95E+05 | 1.02E+08 | 2.86E+05 | 1.68E+08 |
| | (5.46E+07) | (1.71E+05) | (6.43E+07) | (2.76E+05) | (7.53E+07) |
| BCD_0106 | 9.18E+03 | 1.24E+05 | 5.11E+06 | 8.55E+03 | 1.86E+06 |
| | (2.07E+03) | (4.22E+04) | (5.26E+06) | (6.88E+03) | (1.26E+06) |
| AAV-9 | 1.34E+07 | 4.94E+05 | 9.75E+07 | 1.31E+05 | 1.32E+08 |
| | (2.59E+07) | (2.31E+05) | (4.58E+07) | (6.75E+04) | (1.37E+08) |
| BCD_0126 | 5.56E+05 | 5.17E+05 | 3.10E+07 | 7.21E+04 | 3.57E+07 |
| | (2.26E+05) | (1.96E+05) | (2.42E+07) | (2.00E+04) | (2.24E+07) |
| BCD_0127 | 6.65E+04 | 2.25E+05 | 5.59E+06 | 1.73E+04 | 1.97E+07 |
| | (2.91E+04) | (6.03E+04) | (3.41E+06) | (1.59E+04) | (2.00E+07) |
| BCD_0146 | 5.43E+04 | 1.10E+05 | 2.90E+06 | 1.58E+04 | 1.58E+07 |
| | (3.57E+04) | (4.10E+04) | (1.22E+06) | (5.68E+03) | (2.04E+07) |
| BCD_0177 | 1.12E+05 | 1.15E+05 | 7.16E+06 | 2.70E+04 | 6.91E+06 |
| | (4.82E+04) | (3.69E+04) | (1.93E+06) | (1.38E+04) | (5.19E+06) |
| BCD_0181 | 2.11E+05 | 1.68E+05 | 1.02E+07 | 2.28E+04 | 2.65E+07 |
| | (1.34E+05) | (7.47E+04) | (4.72E+06) | (3.10E+04) | (2.14E+07) |
| BCD_0187 | 2.24E+05 | 3.01E+05 | 1.99E+07 | 3.78E+04 | 6.21E+07 |
| | (1.17E+05) | (7.50E+04) | (1.64E+07) | (3.30E+04) | (6.14E+07) |
| BCD_0160 | 2.35E+06 | 4.61E+05 | 7.64E+07 | 1.55E+05 | 1.24E+08 |
| | (1.72E+06) | (1.26E+05) | (5.23E+07) | (7.66E+04) | (8.20E+07) |
| BCD_0361 | 1.36E+04 | 8.85E+04 | 9.85E+05 | 1.75E+04 | 1.51E+06 |
| | (5.21E+03) | (6.33E+04) | (2.90E+05) | (2.19E+04) | (2.35E+06) |

| | | | | | Lymph |
|---|---|---|---|---|---|
| Name | Heart | Kidneys | Liver | Lung | Nodes |
| AAV-8 | 6.57E+06 | 2.63E+06 | 4.58E+08 | 4.83E+06 | |
| | (1.71E+06) | (5.58E+05) | (1.82E+08) | (2.34E+06) | |
| AAV-6 | 7.61E+04 | 1.87E+04 | 9.23E+06 | 6.60E+04 | 1.32E+04 |
| | (3.87E+04) | (1.14E+04) | (5.37E+06) | (4.18E+04) | (7.09E+03) |
| AAV-7 | 8.04E+05 | 1.13E+06 | 1.47E+08 | 1.03E+06 | 3.38E+06 |
| | (4.69E+05) | (1.01E+06) | (5.58E+07) | (6.64E+05) | (1.33E+06) |
| BCD_0386 | 9.97E+06 | 1.81E+05 | 5.14E+06 | 4.22E+05 | 1.54E+06 |
| | (6.03E+06) | (6.19E+04) | (3.25E+06) | (9.40E+04) | (9.64E+05) |
| BCD_0159 | 3.51E+06 | 8.50E+05 | 3.49E+06 | 2.72E+05 | |
| | (2.77E+06) | (9.48E+05) | (2.30E+06) | (1.88E+05) | |
| BCD_0190 | 7.96E+06 | 6.89E+05 | 3.52E+07 | 5.80E+05 | |
| | (4.42E+06) | (3.77E+05) | (1.85E+07) | (1.20E+05) | |
| BCD_0106 | 4.51E+05 | 7.21E+05 | 4.14E+08 | 2.60E+06 | |
| | (1.46E+05) | (3.19E+05) | (5.24E+07) | (8.09E+05) | |
| AAV-9 | 2.62E+07 | 3.95E+05 | 5.33E+07 | 1.47E+06 | |
| | (1.52E+07) | (1.15E+05) | (3.16E+07) | (8.65E+05) | |
| BCD_0126 | 2.36E+07 | 1.32E+06 | 2.99E+08 | 2.56E+06 | |
| | (9.68E+06) | (4.39E+05) | (2.92E+07) | (3.39E+05) | |
| BCD_0127 | 3.38E+06 | 3.77E+05 | 2.50E+08 | 9.17E+05 | |
| | (1.86E+06) | (1.68E+05) | (9.05E+07) | (1.78E+05) | |
| BCD_0146 | 2.72E+06 | 1.44E+05 | 7.56E+07 | 3.11E+05 | |
| | (2.23E+06) | (1.14E+05) | (1.70E+07) | (1.11E+05) | |
| BCD_0177 | 2.75E+06 | 3.39E+05 | 8.99E+07 | 8.86E+05 | |
| | (1.10E+06) | (7.10E+04) | (3.93E+07) | (4.67E+05) | |
| BCD_0181 | 5.67E+06 | 7.71E+05 | 1.32E+08 | 1.04E+06 | |
| | (1.13E+06) | (2.92E+05) | (5.94E+07) | (2.78E+05) | |
| BCD_0187 | 1.10E+07 | 4.75E+05 | 1.74E+08 | 1.10E+06 | |
| | (3.74E+06) | (1.85E+05) | (6.08E+07) | (3.36E+05) | |
| BCD_0160 | 2.15E+07 | 1.73E+06 | 2.90E+07 | 1.97E+06 | |
| | (7.33E+06) | (1.05E+06) | (9.98E+06) | (1.08E+06) | |
| BCD_0361 | 5.51E+05 | 1.73E+05 | 1.29E+08 | 6.07E+05 | |
| | (9.95E+04) | (6.13E+04) | (2.70E+07) | (1.79E+05) | |
| | Masseter | Mesenteric | | | |
| Name | muscle | Fat | Pancreas | Quadriceps | Skin |
| AAV-8 | | | | 9.25E+06 | |
| | | | | (4.83E+06) | |
| AAV-6 | 8.87E+02 | | 2.18E+04 | 3.19E+04 | 1.80E+04 |
| | (3.36E+03) | | (1.23E+04) | (4.26E+04) | (5.85E+03) |
| AAV-7 | 1.25E+05 | | 4.60E+05 | 6.52E+05 | 5.78E+04 |
| | (7.30E+04) | | (1.57E+05) | (5.11E+05) | (1.94E+04) |
| BCD_0386 | 7.03E+06 | | 8.68E+04 | 9.11E+07 | 2.59E+06 |
| | (4.03E+06) | | (3.13E+04) | (5.22E+07) | (2.01E+06) |
| BCD_0159 | 3.24E+06 | 2.48E+06 | 3.74E+04 | | 3.70E+05 |
| | (1.06E+06) | (2.56E+06) | (3.12E+04) | | (1.26E+05) |
| BCD_0190 | 2.75E+07 | 8.22E+06 | 1.65E+05 | | 2.77E+06 |
| | (2.30E+07) | (6.17E+06) | (1.85E+05) | | (1.76E+06) |
| BCD_0106 | 6.77E+04 | 3.81E+05 | 1.07E+06 | | 1.91E+04 |
| | (3.44E+04) | (4.75E+04) | (1.74E+05) | | (5.15E+03) |
| AAV-9 | 9.05E+06 | 1.51E+06 | 4.91E+05 | | 1.66E+06 |
| | (3.97E+06) | (8.73E+05) | (5.19E+05) | | (1.11E+06) |
| BCD_0126 | 8.85E+06 | 4.64E+06 | 1.06E+06 | | 6.58E+05 |
| | (4.68E+06) | (2.27E+06) | (6.55E+05) | | (2.75E+05) |
| BCD_0127 | 1.41E+06 | 3.04E+06 | 6.87E+05 | | 3.88E+05 |
| | (4.17E+05) | (1.66E+06) | (3.01E+05) | | (4.28E+05) |
| BCD_0146 | 4.80E+05 | 2.16E+05 | 2.10E+05 | | 2.93E+05 |
| | (3.91E+05) | (4.22E+04) | (9.79E+04) | | (2.71E+05) |
| BCD_0177 | 1.22E+06 | 2.21E+06 | 2.79E+05 | | 3.39E+05 |
| | (8.29E+05) | (9.55E+05) | (1.12E+05) | | (1.36E+05) |
| BCD_0181 | 5.41E+06 | 7.85E+05 | 4.01E+05 | | 3.89E+05 |
| | (4.45E+06) | (5.20E+05) | (1.72E+05) | | (1.42E+05) |
| BCD_0187 | 6.15E+06 | 3.24E+06 | 6.56E+05 | | 6.53E+05 |
| | (3.01E+06) | (8.81E+05) | (1.26E+05) | | (4.65E+05) |
| BCD_0160 | 1.00E+07 | 3.05E+06 | 2.07E+05 | | 9.08E+05 |
| | (9.58E+06) | (3.35E+06) | (9.27E+04) | | (4.61E+05) |
| BCD_0361 | 6.14E+04 | 1.45E+06 | 5.79E+05 | | 4.25E+04 |
| | (3.40E+04) | (5.21E+05) | (6.35E+05) | | (2.05E+04) |

| Name | Soleus | Spleen | Tibialis Anterior | Tongue |
|---|---|---|---|---|
| AAV-8 | | 7.34E+05 | | |
| | | (2.84E+05) | | |
| AAV-6 | | 4.98E+03 | 2.09E+04 | 7.27E+02 |
| | | (4.23E+03) | (1.44E+04) | (1.57E+03) |
| AAV-7 | | 1.47E+05 | 1.21E+05 | 4.45E+04 |
| | | (4.00E+04) | (9.65E+04) | (4.56E+04) |
| BCD_0386 | 1.90E+06 | 3.71E+04 | 1.90E+07 | 4.16E+06 |
| | (1.46E+06) | (2.05E+04) | (1.03E+07) | (1.98E+06) |
| BCD_0159 | | 3.73E+04 | 2.19E+06 | |
| | | (3.34E+04) | (1.52E+06) | |
| BCD_0190 | | 7.22E+04 | 3.45E+07 | |
| | | (7.59E+04) | (1.94E+07) | |
| BCD_0106 | | 7.77E+05 | 7.48E+04 | |
| | | (1.40E+05) | (4.82E+04) | |
| AAV-9 | | 1.82E+05 | 1.08E+07 | |
| | | (1.04E+05) | (1.14E+07) | |
| BCD_0126 | | 4.04E+05 | 2.65E+06 | |
| | | (1.04E+05) | (1.00E+06) | |
| BCD_0127 | | 2.23E+05 | 5.70E+05 | |
| | | (7.12E+04) | (3.42E+05) | |
| BCD_0146 | | 5.50E+04 | 1.85E+06 | |
| | | (3.17E+04) | (1.81E+06) | |
| BCD_0177 | | 1.73E+05 | 5.78E+05 | |
| | | (3.08E+04) | (3.22E+05) | |
| BCD_0181 | | 1.10E+05 | 1.52E+06 | |
| | | (7.07E+04) | (1.51E+06) | |
| BCD_0187 | | 1.96E+05 | 2.04E+06 | |
| | | (8.84E+04) | (5.57E+05) | |
| BCD_0160 | | 6.99E+04 | 8.37E+06 | |
| | | (4.71E+04) | (4.61E+06) | |
| BCD_0361 | | 1.33E+05 | 5.62E+04 | |
| | | (2.52E+04) | (5.68E+04) | |

Total flux activity observed in Table 7 is a proxy for AAV viral tropism (i.e., tissue/organ infectivity). A log or more difference in the average (photons/see/cm²/radian) for a specific tissue type/organ, compared to another AAV capsid indicates a significant increase or decrease in tropism/infectivity. These data show that AAV harboring specific novel capsid proteins demonstrate different tissue specificities/tropism profiles.

### 6.5.7 Example 7: Evaluation of Novel rAAV Viral Particles in Non-Human Primates

Non-human primate studies are conducted with cynomolgus monkeys (Macaca fascicularis) to evaluate the ability of a novel rAAV viral particle to transduce and express in various organs and tissue types.

Novel rAAV Viral Particles:

rAAV viral particles comprising a novel capsid protein sequence, see Table 9, and a βCG transgene are produced as provided in Example 3 above.

### Study Protocol:

Study groups (n=3) include vehicle and various doses, high dose “HD” and low dose “LD” of AAV virions containing a coding sequence for a gene. Efficacy endpoints include a run in of 3-4 weeks of weekly bleeds (plasma) for each animal baseline reads then weekly bleeds for a 8-13 weeks study. Efficacy is evaluated by plasma and tissue gene of interest activity and protein levels.

Clinical pathology and hematology readouts are monitored. Safety endpoints include weekly physical, and body weight measurements, as well as monitoring for anti-AAV antibody and anti-gene responses (e.g., therapeutic transgene or target thereof) and liver enzyme levels such as, ALT. The primates are monitored for adverse clinical signs, and if seen additional analyses are performed. At the time of study termination gross necropsy is performed and all major organs are assessed for gene of interest activity, protein, and pathology by quantitative polymerase chain reaction (qPCR) and immunohistochemistry (IHC).

Both qPCR and IHC assays show that rAAV viral particles comprising the novel capsid protein yield high BCG-positive expression in the organ which the novel AAV capsid shows enhanced tropism/delivery for, and low BCG-positive expression in the de-targeted cells.

6.5.8 Example 8A: Ex Vivo Evaluation of Novel rAAV Viral Particles Tropism in Brain Tissue

Studies using selected rAAVs and/or novel rAAV viral particles of the disclosure are conducted with ex vivo brain slices to evaluate their tropism for the brain.

Animals and Novel AAV Vector or rAAV Vector Genome:

Either non-human primate (Macaca nemestrina), C57BL/6 mice are used for this study. Novel AAV vectors or rAAV vector genomes comprising the novel AAV capsid sequences in Table 9 and/or other AAVs, a hSyn1 promoter, are tagged with either GFP or YFP are prepared as described in the examples above.

### Media and Reagents:

NMDG-HEPES aCSF (in mM): 92 NMDG, 2.5 KCl, 1.25 NaH2PO4, 30 NaHCO3, 20 HEPES, 25 glucose, 2 thiourea, 5 Na-ascorbate, 3 Na-pyruvate, 0.5 CaCl2·2H2O, and 10 MgSO4·7H2O. Titrate pH to 7.3-7.4 with 17 mL+/−0.5 mL of 5 M hydrochloric acid.

HEPES holding aCSF (in mM): 92 NaCl, 2.5 KCl, 1.25 NaH2PO4, 30 NaHCO3, 20 HEPES, 25 glucose, 2 thiourea, 5 Na-ascorbate, 3 Na-pyruvate, 2 CaCl2·2H2O, and 2 MgSO4·7H2O. Titrate pH to 7.3-7.4 with concentrated 10 N NaOH.

Recording aCSF (in mM): 124 NaCl, 2.5 KCl, 1.25 NaH2PO4, 24 NaHCO₃, 12.5 glucose, 5 HEPES, 2 CaCl2·2H2O, and 2 MgSO4·7H2O. Titrate pH to 7.3-7.4 concentrated 10 N NaOH.

Na+ spike-in solution (2 M): 580 mg of NaCl is dissolved in 5 mL of freshly prepared NMDG-HEPES aCSF.

2% agarose for tissue embedding: 2 g of agarose type 1B is dissolved in 100 mL of 1×PBS and microwaved until just boiling and swirled to mix. The mixture is poured into a sterile 10 cm Petri dishes and allowed to solidify. The agarose plate is stored in a sealed plastic bag at 4° C. until use.

Injectable anesthetic working stock solution: 2.5 g of 2,2,2-Tribromoethanol is mixed with 5 mL of 2-methyl-2-butanol. Next, the mixture is gradually dissolved into 200 mL PBS, pH 7.0-7.3 and filtered with a 0.22 μm filter before use and stored at 4° C., protected from light.

### Slicing Station:

A 250 ml beaker is filled with 200 mL of NMDG-HEPES aCSF and pre-chilled on ice with constant carbogenation (applied via a gas diffuser stone immersed in the media) for >10 min. The initial brain slice recovery chamber is filled with 150 mL of NMDG-HEPES aCSF (maintain constant carbogenation) and the chamber is placed into a heated water bath maintained at 32-34° C. A slice chamber after the design of Edwards and Konnerth (1992) Methods Enzymol. 207:208-22 is used. The netting is submerged approximately 1 cm under the liquid surface. The reservoir is filled with 450 mL of HEPES aCSF and warmed to room temperature under constant carbogenation until use.

Molten agarose is prepared for tissue embedding. The open end of a 50 ml conical vial is used to cut out a block of 2% agarose from the previously prepared dish. The conical vial is microwaved until the agarose is just melted. The molten agarose is poured into 1.5 mL tubes. The agarose is maintained in the molten state using a thermomixer set to 42° C. with vigorous shaking.

### Transcardial Perfusion:

Deeply anesthetize mice by intraperitoneal injection of anesthetic working stock solution (250 mg/kg: 0.2 mL of 1.25% anesthetic working stock solution per 10 g body weight). After ˜2-3 min, sufficient depth of anesthesia is verified by toe pinch reflex test.

A 30 ml syringe is loaded with 25 mL of carbogenated NMDG-HEPES aCSF from the pre-chilled (2-4° C.) 250 ml beaker. A 25 5/8 gauge needle is attached. The needle of the 30 mL syringe is inserted into the left ventricle and the right atrium is cut with fine scissors to allow blood to exit the heart. The syringe plunger is depressed using constant pressure and perfuse the animal with the chilled NMDG-HEPES aCSF at a rate of ˜10 mL/min.

### Brain Dissection and Slicing:

Animals are decapitated. Next, a scalpel is used to open the skin on the head and to expose the skull cap. Fine super-cut scissors are used to cut away the skin over the skull cap and to make small incisions laterally on either side at the caudal/ventral base of the skull. Additional shallow cuts are made starting at the caudal/dorsal aspect of the skull moving in the rostral direction up the dorsal midline. Finally, a ‘T’ cut perpendicular to the midline at the level of the olfactory bulbs is made.

Round-tip forceps are used to grasp the skull starting at the rostral-medial aspect and peel back towards the caudal-lateral direction. This step is repeated for both sides to crack open and remove the dorsal halves of the skull cap to expose the brain. The intact brain is scooped out and placed into the beaker of pre-chilled NMDG-HEPES aCSF and allowed to cool for approximately 1 min.

A large spatula is used to lift the brain out of the beaker and onto a petri dish covered with filter paper. The brain is trimmed and blocked to the preferred angle of slicing and desired brain region of interest.

The brain block is affixed to the specimen holder using adhesive glue. Next, the inner piece of the specimen holder is retracted to withdraw the brain block fully inside. Molten agarose is poured directly into the holder until the brain block is fully covered in agarose. A pre-cooled accessory chilling block is clamped around the specimen holder for ˜10 secs until the agarose is solidified.

The specimen holder is inserted into the receptacle on the slicer machine and proper alignment is verified. The reservoir is filled with remaining pre-chilled, oxygenated NMDG-HEPES aCSF with a bubble stone placed inside for the duration of slicing to ensure adequate oxygenation.

The micrometer is adjusted to slice the embedded brain specimen. The slicer is empirically adjusted for the advance speed and oscillation frequency and tissue is sliced in 300 μm increments until the region of interest is fully sectioned.

### Optimized NMDG Protective Recovery Procedure:

The slices are collected using a cut-off plastic Pasteur pipet and transferred into a pre-warmed (34° C.) initial recovery chamber filled with 150 mL of NMDG-HEPES aCSF. Transfer all slices in short succession and start a timer as soon as all slices are moved into the recovery chamber.

Next, a stepwise Na+ spike-in procedure is conducted by adding the indicated volumes of Na+ spike-in solution at the indicated times directly into the bubbler chimney of the initial recovery chamber.

The slices are transferred to the HEPES aCSF long-term holding chamber and then maintained at room temperature. Slices are allowed to recover for an additional 1 hour in the HEPES holding chamber prior to initiating experiments. Visualization of YFP or GFP expression in the brain slice is conducted by epifluorescence microscopy and/or IHC detection of the YFP or GFP protein.

Positive YFP or GFP expressing neuronal cells by either epifluorescence microscopy and/or IHC detection, as compared to a positive and negative controls, would indicate that the selected novel rAAV viral particles have neuronal tropism in brain tissue.

### 6.5.9 Example 8B: Ex Vivo Evaluation of Novel rAAV Viral Particles Tropism in Brain Tissue

Studies were conducted using selected rAAVs, including novel rAAV viral particles of the disclosure, with ex vivo brain slices to further evaluate their putative tropism for the brain.

Animals and Novel AAV Vector or rAAV Vector Genome:

Either non-human primate (Macaca nemestrina), C57BL/6 mice, or human brain tissue were used for this study. rAAV vectors and vector genomes comprising the novel AAV capsid sequences in Table 10 or selected rAAVs with the vector the CN1839-rAAV-hSyn1-SYFP2-10aa-H2B-WPRE3-BGHpA (Addgene plasmid #163509; http://n2t.net/addgene: 163509; RRID: Addgene_163509) and were prepared as described in the examples above.

### Media and Reagents:

NMDG-HEPES aCSF (in mM): 92 NMDG, 2.5 KCl, 1.25 NaH2PO4, 30 NaHCO3, 20 HEPES, 25 glucose, 2 thiourea, 5 Na-ascorbate, 3 Na-pyruvate, 0.5 CaCl2·2H2O, and 10 MgSO4·7H2O. Titrate pH to 7.3-7.4 with 17 mL+/−0.5 mL of 5 M hydrochloric acid.

HEPES holding aCSF (in mM): 92 NaCl, 2.5 KCl, 1.25 NaH2PO4, 30 NaHCO₃, 20 HEPES, 25 glucose, 2 thiourea, 5 Na-ascorbate, 3 Na-pyruvate, 2 CaCl2·2H2O, and 2 MgSO4·7H2O. Titrate pH to 7.3-7.4 with concentrated 10 N NaOH.

Recording aCSF (in mM): 124 NaCl, 2.5 KCl, 1.25 NaH2PO4, 24 NaHCO3, 12.5 glucose, 5 HEPES, 2 CaCl2·2H2O, and 2 MgSO4.7H2O. Titrate pH to 7.3-7.4 concentrated 10 N NaOH.

Na+ spike-in solution (2 M): 580 mg of NaCl was dissolved in 5 mL of freshly prepared NMDG-HEPES aCSF.

2% agarose for tissue embedding: 2 g of agarose type 1B was dissolved in 100 mL of 1×PBS and microwaved until just boiling and swirled to mix. The mixture was poured into a sterile 10 cm Petri dishes and allowed to solidify. The agarose plate was stored in a sealed plastic bag at 4° C. until use.

Injectable anesthetic working stock solution: 2.5 g of 2,2,2-Tribromoethanol was mixed with 5 mL of 2-methyl-2-butanol. Next, the mixture was gradually dissolved into 200 mL PBS, pH 7.0-7.3 and filtered with a 0.22 μm filter before use and stored at 4° C., protected from light.

### Slicing Station:

A 250 ml beaker was filled with 200 mL of NMDG-HEPES aCSF and pre-chilled on ice with constant carbogenation (applied via a gas diffuser stone immersed in the media) for >10 min. The initial brain slice recovery chamber was filled with 150 mL of NMDG-HEPES aCSF (maintain constant carbogenation) and the chamber was placed into a heated water bath maintained at 32-34° C. A slice chamber after the design of Edwards and Konnerth (1992) Methods Enzymol. 207:208-22 was used. The netting was submerged approximately 1 cm under the liquid surface. The reservoir was filled with 450 mL of HEPES aCSF and warmed to room temperature under constant carbogenation until use.

Molten agarose was prepared for tissue embedding. The open end of a 50 ml conical vial was used to cut out a block of 2% agarose from the previously prepared dish. The conical vial was microwaved until the agarose was just melted. The molten agarose was poured into 1.5 mL tubes. The agarose was maintained in the molten state using a thermomixer set to 42° C. with vigorous shaking.

### Transcardial Perfusion:

Deeply anesthetize mice by intraperitoneal injection of anesthetic working stock solution (250 mg/kg: 0.2 mL of 1.25% anesthetic working stock solution per 10 g body weight). After ˜2-3 min, sufficient depth of anesthesia was verified by toe pinch reflex test.

A 30 ml syringe was loaded with 25 mL of carbogenated NMDG-HEPES aCSF from the pre-chilled (2-4° C.) 250 ml beaker. A 25 5/8 gauge needle was attached. The needle of the 30 ml syringe was inserted into the left ventricle and the right atrium was cut with fine scissors to allow blood to exit the heart. The syringe plunger was depressed using constant pressure and perfuse the animal with the chilled NMDG-HEPES aCSF at a rate of ˜10 mL/min.

### Brain Dissection and Slicing:

Animals were decapitated. Next, a scalpel was used to open the skin on the head and to expose the skull cap. Fine super-cut scissors were used to cut away the skin over the skull cap and to make small incisions laterally on either side at the caudal/ventral base of the skull. Additional shallow cuts were made starting at the caudal/dorsal aspect of the skull moving in the rostral direction up the dorsal midline. Finally, a ‘T’ cut perpendicular to the midline at the level of the olfactory bulbs was made.

Round-tip forceps were used to grasp the skull starting at the rostral-medial aspect and peel back towards the caudal-lateral direction. This step was repeated for both sides to crack open and remove the dorsal halves of the skull cap to expose the brain. The intact brain was scooped out and placed into the beaker of pre-chilled NMDG-HEPES aCSF and allowed to cool for approximately 1 min.

A large spatula was used to lift the brain out of the beaker and onto a petri dish covered with filter paper. The brain was trimmed and blocked to the preferred angle of slicing and desired brain region of interest.

The brain block was affixed to the specimen holder using adhesive glue. Next, the inner piece of the specimen holder was retracted to withdraw the brain block fully inside. Molten agarose was poured directly into the holder until the brain block was fully covered in agarose. A pre-cooled accessory chilling block was clamped around the specimen holder for ˜10 secs until the agarose was solidified.

The specimen holder was inserted into the receptacle on the slicer machine and proper alignment was verified. The reservoir was filled with remaining pre-chilled, oxygenated NMDG-HEPES aCSF with a bubble stone placed inside for the duration of slicing to ensure adequate oxygenation.

The micrometer was adjusted to slice the embedded brain specimen. The slicer was empirically adjusted for the advance speed and oscillation frequency and tissue was sliced in 300 μm increments until the region of interest was fully sectioned.

### Optimized NMDG Protective Recovery Procedure:

The slices were collected using a cut-off plastic Pasteur pipet and transferred into a pre-warmed (34° C.) initial recovery chamber filled with 150 mL of NMDG-HEPES aCSF. Transfer all slices in short succession and start a timer as soon as all slices were moved into the recovery chamber.

Next, a stepwise Na+ spike-in procedure was conducted by adding the indicated volumes of Na+ spike-in solution at the indicated times directly into the bubbler chimney of the initial recovery chamber.

The slices were transferred to the HEPES aCSF long-term holding chamber and then maintained at room temperature. Slices were allowed to recover for an additional 1 hour in the HEPES holding chamber prior to initiating experiments. Visualization of YFP expression in the brain slice is conducted by epifluorescence microscopy and/or IHC detection of the YFP protein.

Positive YFP expressing neuronal cells by either epifluorescence microscopy and/or IHC detection, as compared to a AAV positive and negative controls, would indicate that the selected novel rAAV viral particles have neuronal tropism in brain tissue.

Shown in Table 10 is each rAAV's transduction profile for the indicated rAAV virial particle comprising the specified AAV capsid protein (e.g., AAV_ID) ran at least in duplicate and/or triplicate. The expression of the rAAV viral particle was normalized to AAV-9.PHP.eb, where AAV-9.PHP.eb was set to 100% for the given ex vivo model brain tissue slice tested (e.g., mouse, NHP, human). Blank cells have not yet been tested.

TABLE 10
Averaged Normalized rAAV Brain Expression

| AAV_ID | MOUSE | NHP | HUMAN |
|---|---|---|---|
| AAV-1 | 69.74% | 13.40% | |
| AAV-2 | 44.23% | 16.99% | |
| AAV-3b | 7.55% | 3.01% | |
| AAV-7 | 99.70% | 51.96% | |
| AAV-8 | 91.45% | 21.05% | |
| AAV-9 | 75.71% | 66.55% | 53.20% |
| AAV_LK03 | 36.40% | 69.52% | |
| BCD_0106 | 30.12% | 0.65% | |
| BCD_0125 | 54.23% | 38.24% | |
| BCD_0126 | 97.22% | 43.96% | 18.91% |
| BCD_0157 | 39.43% | 14.04% | |
| BCD_0160 | 81.35% | 83.28% | 69.91% |
| BCD_0167 | 28.15% | 48.04% | 46.45% |
| BCD_0174 | | 50.00% | |
| BCD_0176 | 57.62% | 90.36% | 101.72% |
| BCD_0178 | | 22.72% | |
| BCD_0180 | 100.15% | 65.10% | 60.49% |
| BCD_0182 | 75.24% | 32.39% | 64.06% |
| BCD_0185 | 28.67% | 60.34% | 47.38% |
| BCD_0190 | 35.25% | 0.60% | |
| BCD_0192 | | 62.76% | |
| BCD_0193 | 16.45% | 35.62% | |
| BCD_0195 | 69.22% | 80.40% | 50.15% |
| BCD_0196 | | 25.78% | |
| BCD_0198 | | 28.41% | |
| BCD_0270 | 63.90% | 31.28% | |
| BCD_0275 | 70.58% | 16.67% | |
| BCD_0276 | | 29.46% | |
| BCD_0277 | | 50.90% | |
| BCD_0282 | 61.07% | 96.44% | 136.21% |
| BCD_0283 | | 32.35% | |
| BCD_0284 | | 29.48% | |
| BCD_0286 | | 33.36% | |
| BCD_0289 | | 21.87% | |
| BCD_0295 | | 22.76% | |
| BCD_0297 | | 24.39% | |
| BCD_0300 | | 0.49% | |
| BCD_0353 | 54.74% | 6.62% | 3.09% |
| BCD_0360 | 25.81% | 6.38% | |
| BCD_0361 | 24.25% | 5.23% | |
| BCD_0391 | 16.21% | 2.18% | |
| BCD_0395 | 21.33% | 12.29% | |
| BCD_0396 | 10.97% | 18.33% | 6.72% |
| BCD_0446 | | 84.55% | 105.17% |
| BCD_0448 | | 19.51% | |
| BCD_0454 | | 60.16% | 93.10% |
| BCD_0455 | | 27.64% | |
| BMN_0251 | | 5.71% | |
| BMN_0258 | | 0.52% | |
| BMN_0271 | | 49.69% | |
| BMN_0272 | 55.51% | 50.40% | |
| BMN_0273 | | 10.00% | |
| BMN_0304 | | 16.01% | |
| BMN_0308 | 6.82% | 46.08% | |

The expression observed in Table 10 is a proxy for AAV viral tropism (i.e., tissue/organ infectivity in the brain). rAAVs with about 30% to about 50% or higher averaged normalized expression in the ex vivo brain slice assay are of particular interest. Alternatively, rAAVs with about 50% or higher averaged normalized expression in the ex vivo brain slice assay are of particular interest.

### 6.5.10 Example 9: Determining Variable Regions of a Novel AAV VP1 Capsid Protein

The associated variable regions, VR1-VRIX, GBS, and GH loop of the novel AAV VP1 capsid protein sequences are determined.

Briefly, a multiple sequence alignment is conducted using the VP1 amino acid sequences of a novel AAV capsid protein of the disclosure with different reference AAV serotypes, see for example, Table 4, using sequence alignment software provided herein.

Table 8 below, provides an example of previously described AAV capsid variable regions as published in International Application No. WO 2018/022608. The numbers indicated in the table refer to the amino acid residues representing each variable region (“VR”), GBS, and GH loop regions of the indicated AAV capsid sequence spanning its VP1 amino acid sequence.

In regards to the amino acid described locations of the VR, GBS and GH loop regions, it is noted that the location of the N-terminal and/or C-terminal ends of those regions may vary by from up to 1 amino acid, 2 amino acids, 3 amino acids, 4 amino acids, or 5 amino acids from the amino acid locations of those regions as they are explicitly described herein (particularly in Table 8).

TABLE 8
Associated AAV Capsid Variable Regions

| | VR- | VR- | VR- | VR- | | VR- | VR- | VR- | VR- | VR- | GH |
|---|---|---|---|---|---|---|---|---|---|---|---|
| AAV | I | II | III | IV | GBS | V | VI | VII | VIII | IX | loop |
| AAV-2 | 262- | 325- | 380- | 450- | 464- | 487- | 526- | 544- | 580- | 703- | 446- |
| | 268 | 330 | 384 | 459 | 475 | 504 | 538 | 557 | 592 | 711 | 600 |
| AAV-4 | 256- | 316- | 372- | 444- | 459- | 481- | 526- | 542- | 579- | 702- | 440- |
| | 260 | 321 | 378 | 454 | 470 | 504 | 536 | 556 | 591 | 710 | 599 |
| AAV-5 | 252- | 316- | 372- | 443- | 452- | 474- | 513- | 531- | 570- | 692- | 439- |
| | 258 | 321 | 377 | 446 | 462 | 491 | 525 | 547 | 582 | 700 | 590 |
| AAV-6 | 262- | 326- | 381- | 451- | 465- | 488- | 527- | 545- | 581- | 704- | 447- |
| | 269 | 331 | 385 | 460 | 476 | 505 | 539 | 558 | 593 | 712 | 601 |
| AAV-6.2 | 262- | 326- | 381- | 451- | 465- | 488- | 527- | 545- | 581- | 704- | 447- |
| | 269 | 331 | 385 | 460 | 476 | 505 | 539 | 558 | 593 | 712 | 601 |
| AAV-7 | 263- | 327- | 382- | 452- | 467- | 490- | 529- | 547- | 582- | 705- | 448- |
| | 270 | 332 | 386 | 462 | 478 | 507 | 541 | 559 | 594 | 713 | 602 |
| AAV-8 | 263- | 328- | 383- | 453- | 467- | 490- | 529- | 548- | 583- | 706- | 449- |
| | 271 | 333 | 387 | 462 | 478 | 507 | 541 | 560 | 595 | 714 | 603 |
| AAV-9 | 262- | 327- | 382- | 452- | 465- | 488- | 527- | 545- | 581- | 704- | 448- |
| | 270 | 332 | 386 | 460 | 476 | 505 | 539 | 558 | 593 | 712 | 602 |
| AAVrh8 | 262- | 327- | 382- | 452- | 465- | 488- | 527- | 545- | 581- | 704- | 448- |
| | 270 | 332 | 386 | 460 | 476 | 505 | 539 | 558 | 593 | 712 | 602 |
| AAVrh10 | 263- | 328- | 383- | 453- | 467- | 490- | 529- | 548- | 583- | 706- | 449- |
| | 271 | 333 | 387 | 462 | 478 | 507 | 541 | 560 | 595 | 714 | 603 |
| AAVbo | 258- | 318- | 374- | 446- | 462- | 484- | 528- | 544- | 581- | 704- | 442- |
| | 262 | 323 | 380 | 456 | 472 | 506 | 538 | 558 | 593 | 712 | 601 |
| BMN_0219 | 265- | 325- | 381- | 452- | 468- | 490- | 534- | 550- | 587- | 710- | 448- |
| (Bba. 21) | 269 | 330 | 386 | 462 | 478 | 512 | 544 | 564 | 599 | 718 | 607 |
| BMN_0220 | 265- | 325- | 381- | 452- | 468- | 490- | 534- | 550- | 587- | 710- | 448- |
| (Bba. 31) | 269 | 330 | 386 | 462 | 478 | 512 | 544 | 564 | 599 | 718 | 607 |
| BMN_0221 | 265- | 325- | 381- | 452- | 468- | 490- | 534- | 550- | 587- | 710- | 448- |
| (Bba. 32) | 269 | 330 | 386 | 462 | 478 | 512 | 544 | 564 | 599 | 718 | 607 |
| BMN_0222 | 265- | 325- | 381- | 452- | 468- | 490- | 534- | 550- | 587- | 710- | 448- |
| (Bba. 33) | 269 | 330 | 386 | 462 | 478 | 512 | 544 | 564 | 599 | 718 | 607 |
| BMN_0322 | 243- | 307- | 363- | 434- | 443- | 465- | 505- | 523- | 562- | 684- | 430- |
| (Bpo. 1) | 249 | 312 | 368 | 437 | 453 | 483 | 517 | 539 | 574 | 692 | 582 |
| BMN_0323 | 243- | 307- | 363- | 434- | 443- | 465- | 505- | 523- | 562- | 684- | 430- |
| (Bpo. 2) | 249 | 312 | 368 | 437 | 453 | 483 | 517 | 539 | 574 | 692 | 582 |
| BMN_0324 | 243- | 307- | 363- | 434- | 443- | 465- | 505- | 523- | 562- | 684- | 430- |
| (Bpo. 3) | 249 | 312 | 368 | 437 | 453 | 483 | 517 | 539 | 574 | 692 | 582 |
| BMN_0325 | 243- | 307- | 363- | 434- | 443- | 465- | 505- | 523- | 562- | 684- | 430- |
| (Bpo. 4) | 249 | 312 | 368 | 437 | 453 | 483 | 517 | 539 | 574 | 692 | 582 |
| BMN_0326 | 243- | 307- | 363- | 434- | 443- | 465- | 505- | 523- | 562- | 684- | 430- |
| (Bpo. 6) | 249 | 312 | 368 | 437 | 453 | 483 | 517 | 539 | 574 | 692 | 582 |
| BMN_0327 | 243- | 307- | 363- | 434- | 443- | 465- | 505- | 523- | 562- | 684- | 430- |
| (Bpo. 8) | 249 | 312 | 368 | 437 | 453 | 483 | 517 | 539 | 574 | 692 | 582 |
| BMN_0328 | 243- | 307- | 363- | 434- | 443- | 465- | 505- | 523- | 562- | 684- | 430- |
| (Bpo. 13) | 249 | 312 | 368 | 437 | 453 | 483 | 517 | 539 | 574 | 692 | 582 |
| BMN_0329 | 243- | 307- | 363- | 434- | 443- | 465- | 505- | 523- | 562- | 684- | 430- |
| (Bpo. 18) | 249 | 312 | 368 | 437 | 453 | 483 | 517 | 539 | 574 | 692 | 582 |
| BMN_0330 | 243- | 307- | 363- | 434- | 443- | 465- | 505- | 523- | 562- | 684- | 430- |
| (Bpo. 20) | 249 | 312 | 368 | 437 | 453 | 483 | 517 | 539 | 574 | 692 | 582 |
| BMN_0331 | 243- | 307- | 363- | 434- | 443- | 465- | 505- | 523- | 562- | 684- | 430- |
| (Bpo. 23) | 249 | 312 | 368 | 437 | 453 | 483 | 517 | 539 | 574 | 692 | 582 |
| BMN_0332 | 243- | 307- | 363- | 434- | 443- | 465- | 505- | 523- | 562- | 684- | 430- |
| (Bpo. 24) | 249 | 312 | 368 | 437 | 453 | 483 | 517 | 539 | 574 | 692 | 582 |
| BMN_0333 | 243- | 307- | 363- | 434- | 443- | 465- | 505- | 523- | 562- | 684- | 430- |
| (Bpo. 27) | 249 | 312 | 368 | 437 | 453 | 483 | 517 | 539 | 574 | 692 | 582 |
| BMN_0334 | 243- | 307- | 363- | 434- | 443- | 465- | 505- | 523- | 562- | 684- | 430- |
| (Bpo. 28) | 249 | 312 | 368 | 437 | 453 | 483 | 517 | 539 | 574 | 692 | 582 |
| BMN_0335 | 243- | 307- | 363- | 434- | 443- | 465- | 505- | 523- | 562- | 684- | 430- |
| (Bpo. 29) | 249 | 312 | 368 | 437 | 453 | 483 | 517 | 539 | 574 | 692 | 582 |
| BMN_0336 | 243- | 307- | 363- | 434- | 443- | 465- | 505- | 523- | 562- | 684- | 430- |
| (Bpo. 33) | 249 | 312 | 368 | 437 | 453 | 483 | 517 | 539 | 574 | 692 | 582 |
| BMN_0337 | 243- | 307- | 363- | 434- | 443- | 465- | 505- | 523- | 562- | 684- | 430- |
| (Bpo. 35) | 249 | 312 | 368 | 437 | 453 | 483 | 517 | 539 | 574 | 692 | 582 |
| BMN_0338 | 243- | 307- | 363- | 434- | 443- | 465- | 505- | 523- | 562- | 684- | 430- |
| (Bpo. 36) | 249 | 312 | 368 | 437 | 453 | 483 | 517 | 539 | 574 | 692 | 582 |
| BMN_0339 | 243- | 307- | 363- | 434- | 443- | 465- | 505- | 523- | 562- | 684- | 430- |
| (Bpo. 37) | 249 | 312 | 368 | 437 | 453 | 483 | 517 | 539 | 574 | 692 | 582 |
| AAVmo | 249- | 310- | 366- | 437- | 443- | 467- | 507- | 525- | 556- | 679- | 433- |
| | 252 | 315 | 371 | 439 | 455 | 484 | 519 | 533 | 568 | 689 | 576 |
| BMN_0316 | 262- | 327- | 382- | 452- | 465- | 488- | 527- | 545- | 581- | 704- | 448- |
| (Bce. 14) | 270 | 332 | 386 | 460 | 476 | 505 | 539 | 558 | 593 | 712 | 602 |
| BMN_0317 | 262- | 327- | 382- | 452- | 465- | 488- | 527- | 545- | 581- | 704- | 448- |
| (Bce. 15) | 270 | 332 | 386 | 460 | 476 | 505 | 539 | 558 | 593 | 712 | 602 |
| BMN_0318 | 262- | 327- | 382- | 452- | 465- | 488- | 527- | 545- | 581- | 704- | 448- |
| (Bce. 16) | 270 | 332 | 386 | 460 | 476 | 505 | 539 | 558 | 593 | 712 | 602 |
| BMN_0319 | 262- | 327- | 382- | 452- | 465- | 488- | 527- | 545- | 581- | 704- | 448- |
| (Bce. 17) | 270 | 332 | 386 | 460 | 476 | 505 | 539 | 558 | 593 | 712 | 602 |
| BMN_0320 | 262- | 327- | 382- | 452- | 465- | 488- | 527- | 545- | 581- | 704- | 448- |
| (Bce. 18) | 270 | 332 | 386 | 460 | 476 | 505 | 539 | 558 | 593 | 712 | 602 |
| BMN_0321 | 262- | 327- | 382- | 452- | 465- | 488- | 527- | 545- | 581- | 704- | 448- |
| (Bce. 20) | 270 | 332 | 386 | 460 | 476 | 505 | 539 | 558 | 593 | 712 | 602 |
| BMN_0314 | 262- | 327- | 382- | 452- | 465- | 488- | 527- | 545- | 581- | 704- | 448- |
| (Bma. 42) | 270 | 332 | 386 | 460 | 476 | 505 | 539 | 558 | 593 | 712 | 602 |
| BMN_0315 | 262- | 327- | 382- | 452- | 465- | 488- | 527- | 545- | 581- | 704- | 448- |
| (Bma. 43) | 270 | 332 | 386 | 460 | 476 | 505 | 539 | 558 | 593 | 712 | 602 |
| BMN_0302 | 262- | 327- | 382- | 452- | 465- | 488- | 527- | 545- | 581- | 704- | 448- |
| (Bce. 39) | 270 | 332 | 386 | 460 | 476 | 505 | 539 | 558 | 593 | 712 | 602 |
| BMN_0303 | 262- | 327- | 382- | 452- | 465- | 488- | 527- | 545- | 581- | 704- | 448- |
| (Bce. 40) | 270 | 332 | 386 | 460 | 476 | 505 | 539 | 558 | 593 | 712 | 602 |
| BMN_0304 | 262- | 327- | 382- | 452- | 465- | 488- | 527- | 545- | 581- | 704- | 448- |
| (Bce. 41) | 270 | 332 | 386 | 460 | 476 | 505 | 539 | 558 | 593 | 712 | 602 |
| BMN_0305 | 262- | 327- | 382- | 452- | 465- | 488- | 527- | 545- | 581- | 704- | 448- |
| (Bce. 42) | 270 | 332 | 386 | 460 | 476 | 505 | 539 | 558 | 593 | 712 | 602 |
| AAVanc110 | 262- | 327- | 382- | 452- | 465- | 488- | 527- | 545- | 581- | 704- | 448- |
| | 270 | 332 | 386 | 460 | 476 | 505 | 539 | 558 | 593 | 712 | 602 |
| AAVanc80 | 262- | 326- | 381- | 451- | 465- | 488- | 527- | 545- | 581- | 704- | 447- |
| | 269 | 331 | 385 | 460 | 476 | 505 | 539 | 558 | 593 | 712 | 601 |
| BMN_0337 | 258- | 322- | 377- | 447- | 460- | 483- | 522- | 540- | 575- | 698- | 443- |
| (Bce. 35) | 265 | 327 | 381 | 455 | 471 | 500 | 534 | 552 | 587 | 706 | 595 |
| BMN_0338 | 258- | 322- | 377- | 447- | 460- | 483- | 522- | 540- | 575- | 698- | 443- |
| (Bce. 36) | 265 | 327 | 381 | 455 | 471 | 500 | 534 | 552 | 587 | 706 | 595 |
| BMN_0253 | 258- | 322- | 377- | 447- | 460- | 483- | 522- | 540- | 575- | 698- | 443- |
| (Bce. 43) | 265 | 327 | 381 | 455 | 471 | 500 | 534 | 552 | 587 | 706 | 595 |
| BMN_0254 | 258- | 322- | 377- | 447- | 460- | 483- | 522- | 540- | 575- | 698- | 443- |
| (Bce. 44) | 265 | 327 | 381 | 455 | 471 | 500 | 534 | 552 | 587 | 706 | 595 |
| BMN_0255 | 258- | 322- | 377- | 447- | 460- | 483- | 522- | 540- | 575- | 698- | 443- |
| (Bce. 45) | 265 | 327 | 381 | 455 | 471 | 500 | 534 | 552 | 587 | 706 | 595 |
| BMN_0256 | 258- | 322- | 377- | 447- | 460- | 483- | 522- | 540- | 575- | 698- | 443- |
| (Bce. 46) | 265 | 327 | 381 | 455 | 471 | 500 | 534 | 552 | 587 | 706 | 595 |
| BMN_0257 | 258- | 322- | 377- | 447- | 460- | 483- | 522- | 540- | 575- | 698- | 443- |
| (Bcy. 20) | 265 | 327 | 381 | 455 | 471 | 500 | 534 | 552 | 587 | 706 | 595 |
| BMN_0258 | 258- | 322- | 377- | 447- | 460- | 483- | 522- | 540- | 575- | 698- | 443- |
| (Bcy. 22) | 265 | 327 | 381 | 455 | 471 | 500 | 534 | 552 | 587 | 706 | 595 |
| BMN_0260 | 258- | 322- | 377- | 447- | 460- | 483- | 522- | 540- | 575- | 698- | 443- |
| (Bcy. 23) | 265 | 327 | 381 | 455 | 471 | 500 | 534 | 552 | 587 | 706 | 595 |
| BMN_0261 | 265- | 328- | 383- | 453- | 469- | 492- | 531- | 549- | 584- | 707- | 449- |
| (Bba. 26) | 271 | 333 | 387 | 464 | 480 | 508 | 543 | 561 | 596 | 715 | 604 |
| BMN_0262 | 265- | 328- | 383- | 453- | 469- | 492- | 531- | 549- | 584- | 707- | 449- |
| (Bba. 27) | 271 | 333 | 387 | 464 | 480 | 508 | 543 | 561 | 596 | 715 | 604 |
| BMN_0263 | 265- | 328- | 383- | 453- | 469- | 492- | 531- | 549- | 584- | 707- | 449- |
| (Bba. 29) | 271 | 333 | 387 | 464 | 480 | 508 | 543 | 561 | 596 | 715 | 604 |
| BMN_0264 | 265- | 328- | 383- | 453- | 469- | 492- | 531- | 549- | 584- | 707- | 449- |
| (Bba. 30) | 271 | 333 | 387 | 464 | 480 | 508 | 543 | 561 | 596 | 715 | 604 |
| BMN_0265 | 265- | 328- | 383- | 453- | 469- | 492- | 531- | 549- | 584- | 707- | 449- |
| (Bba. 34) | 271 | 333 | 387 | 464 | 480 | 508 | 543 | 561 | 596 | 715 | 604 |
| BMN_0266 | 265- | 328- | 383- | 453- | 469- | 492- | 531- | 549- | 584- | 707- | 449- |
| (Bba. 35) | 271 | 333 | 387 | 464 | 480 | 508 | 543 | 561 | 596 | 715 | 604 |
| BMN_0267 | 265- | 328- | 383- | 453- | 469- | 492- | 531- | 549- | 584- | 707- | 449- |
| (Bba. 36) | 271 | 333 | 387 | 464 | 480 | 508 | 543 | 561 | 596 | 715 | 604 |
| BMN_0268 | 265- | 328- | 383- | 453- | 469- | 492- | 531- | 549- | 584- | 707- | 449- |
| (Bba. 37) | 271 | 333 | 387 | 464 | 480 | 508 | 543 | 561 | 596 | 715 | 604 |
| BMN_0269 | 265- | 328- | 383- | 453- | 469- | 492- | 531- | 549- | 584- | 707- | 449- |
| (Bba. 38) | 271 | 333 | 387 | 464 | 480 | 508 | 543 | 561 | 596 | 715 | 604 |
| BMN_0270 | 265- | 328- | 383- | 453- | 469- | 492- | 531- | 549- | 584- | 707- | 449- |
| (Bba. 41) | 271 | 333 | 387 | 464 | 480 | 508 | 543 | 561 | 596 | 715 | 604 |
| BMN_0271 | 265- | 328- | 383- | 453- | 469- | 492- | 531- | 549- | 584- | 707- | 449- |
| (Bba. 42) | 271 | 333 | 387 | 464 | 480 | 508 | 543 | 561 | 596 | 715 | 604 |
| BMN_0272 | 265- | 328- | 383- | 453- | 469- | 492- | 531- | 549- | 584- | 707- | 449- |
| (Bba. 43) | 271 | 333 | 387 | 464 | 480 | 508 | 543 | 561 | 596 | 715 | 604 |
| BMN_0273 | 265- | 328- | 383- | 453- | 469- | 492- | 531- | 549- | 584- | 707- | 449- |
| (Bba. 44) | 271 | 333 | 387 | 464 | 480 | 508 | 543 | 561 | 596 | 715 | 604 |
| BMN_0306 | 262- | 327- | 382- | 452- | 465- | 488- | 527- | 545- | 581- | 704- | 448- |
| (Brh. 26) | 270 | 332 | 386 | 460 | 476 | 505 | 539 | 558 | 593 | 712 | 601 |
| BMN_0307 | 262- | 327- | 382- | 452- | 465- | 488- | 527- | 545- | 581- | 704- | 448- |
| (Brh. 27) | 270 | 332 | 386 | 460 | 476 | 505 | 539 | 558 | 593 | 712 | 601 |
| BMN_0308 | 262- | 327- | 382- | 452- | 465- | 488- | 527- | 545- | 581- | 704- | 448- |
| (Brh. 28) | 270 | 332 | 386 | 460 | 476 | 505 | 539 | 558 | 593 | 712 | 601 |
| BMN_0309 | 262- | 327- | 382- | 452- | 465- | 488- | 527- | 545- | 581- | 704- | 448- |
| (Brh. 29) | 270 | 332 | 386 | 460 | 476 | 505 | 539 | 558 | 593 | 712 | 601 |
| BMN_0310 | 262- | 327- | 382- | 452- | 465- | 488- | 527- | 545- | 581- | 704- | 448- |
| (Brh. 30) | 270 | 332 | 386 | 460 | 476 | 505 | 539 | 558 | 593 | 712 | 601 |
| BMN_0311 | 262- | 327- | 382- | 452- | 465- | 488- | 527- | 545- | 581- | 704- | 448- |
| (Brh. 31) | 270 | 332 | 386 | 460 | 476 | 505 | 539 | 558 | 593 | 712 | 601 |
| BMN_0312 | 262- | 327- | 382- | 452- | 465- | 488- | 527- | 545- | 581- | 704- | 448- |
| (Brh. 32) | 270 | 332 | 386 | 460 | 476 | 505 | 539 | 558 | 593 | 712 | 601 |
| BMN_0313 | 262- | 327- | 382- | 452- | 465- | 488- | 527- | 545- | 581- | 704- | 448- |
| (Brh. 33) | 270 | 332 | 386 | 460 | 476 | 505 | 539 | 558 | 593 | 712 | 601 |
| BMN_0208 | 262- | 327- | 382- | 452- | 466- | 489- | 528- | 546- | 582- | 705- | 448- |
| (Bfm. 17) | 270 | 332 | 386 | 461 | 477 | 506 | 540 | 559 | 594 | 713 | 602 |
| BMN_0209 | 262- | 327- | 382- | 452- | 466- | 489- | 528- | 546- | 582- | 705- | 448- |
| (Bfm. 18) | 270 | 332 | 386 | 461 | 477 | 506 | 540 | 559 | 594 | 713 | 602 |
| BMN_0210 | 262- | 327- | 382- | 452- | 466- | 489- | 528- | 546- | 582- | 705- | 448- |
| (Bfm. 20) | 270 | 332 | 386 | 461 | 477 | 506 | 540 | 559 | 594 | 713 | 602 |
| BMN_0211 | 262- | 327- | 382- | 452- | 466- | 489- | 528- | 546- | 582- | 705- | 448- |
| (Bfm. 21) | 270 | 332 | 386 | 461 | 477 | 506 | 540 | 559 | 594 | 713 | 602 |
| BMN_0212 | 262- | 327- | 382- | 452- | 466- | 489- | 528- | 546- | 582- | 705- | 448- |
| (Bfm. 24) | 270 | 332 | 386 | 461 | 477 | 506 | 540 | 559 | 594 | 713 | 602 |
| BMN_0213 | 262- | 327- | 382- | 452- | 466- | 489- | 528- | 546- | 582- | 705- | 448- |
| (Bfm. 25) | 270 | 332 | 386 | 461 | 477 | 506 | 540 | 559 | 594 | 713 | 602 |
| BMN_0214 | 262- | 327- | 382- | 452- | 466- | 489- | 528- | 546- | 582- | 705- | 448- |
| (Bfm. 27) | 270 | 332 | 386 | 461 | 477 | 506 | 540 | 559 | 594 | 713 | 602 |
| BMN_0215 | 262- | 327- | 382- | 452- | 466- | 489- | 528- | 546- | 582- | 705- | 448- |
| (Bfm. 32) | 270 | 332 | 386 | 461 | 477 | 506 | 540 | 559 | 594 | 713 | 602 |
| BMN_0216 | 262- | 327- | 382- | 452- | 466- | 489- | 528- | 546- | 582- | 705- | 448- |
| (Bfm. 33) | 270 | 332 | 386 | 461 | 477 | 506 | 540 | 559 | 594 | 713 | 602 |
| BMN_0217 | 262- | 327- | 382- | 452- | 466- | 489- | 528- | 546- | 582- | 705- | 448- |
| (Bfm. 34) | 270 | 332 | 386 | 461 | 477 | 506 | 540 | 559 | 594 | 713 | 602 |
| BMN_0218 | 262- | 327- | 382- | 452- | 466- | 489- | 528- | 546- | 582- | 705- | 448- |
| (Bfm. 35) | 270 | 332 | 386 | 461 | 477 | 506 | 540 | 559 | 594 | 713 | 602 |

### 6.5.11 Example 10: In Vivo Evaluation of Novel rAAV Viral Particles Tropism in the Ear

Studies were conducted to determine if the novel rAAV viral particles could be delivered and expressed in the ear.

Viral vector production: rAAV comprising the novel capsids and an eGFP transgene were produced as described herein. Virus aliquots were stored at −80° C. and thawed just prior to use for in vivo injections.

Mice: Wild type C57BL/6J (Jackson Laboratories) mice were used for this study. Mice with ages P1-P2 were used for in vivo delivery of viral vectors according to approved protocols.

### In Vivo Viral Inner Ear Injections:

In brief, P1-2 mice were anesthetized with hypothermia through 3 min of exposure to ice water. During the surgery (10-15 min), mice were kept on an ice pad. Using a stereo microscope (Stemi 2000, Zeiss, Oberkochen, Germany) for visualization, a small postauricular incision was made to expose the cochlea bulla and semicircular canals surrounding the utricle. After puncturing the temporal bone, a glass micropipette was inserted into the puncture to manually inject 1-1.2 uL (1−2×10¹⁴ gc/mL) of AAV at a constant rate. Following the procedure, mice were placed on a 37° C. heating pad until fully recovered, and standard postoperative care was applied. All animal procedures were approved by Animal Care and Use Committee and are in accordance with the NIH Guide for Care and Use of Laboratory Animals.

Euthanization of P10 mice were conducted via CO₂ inhalation. Temporal bones were harvested, punctured at the round and oval windows and helicotrema, and fixed in 4% paraformaldehyde for 1 h at room temperature. Tissues were then decalcified in 120 mM EDTA for 16-24 h. Cochleas were sectioned into apical, middle, and basal portions. The organ of Corti was isolated and prepared for whole-mount processing by removal of the lateral wall, spiral limbus, and tectorial membrane.

### Tissue Processing and Image Analysis:

Tissues were permeabilized for 1 h in 0.25% Triton X-100, blocked for 1 h in 2.5% normal donkey serum, and stained at 4 C overnight with rabbit anti-myosin 7a (hair cell marker) primary antibody (1:500 Proteus Biosciences, Ramona, CA, USA #25-6790). After washing with PBS, samples were incubated for 3-4 h with fluorophore-conjugated donkey anti-rabbit secondary antibody (1:400 Alexa Fluor 647: Thermo Fisher #A31573) and fluorophore-conjugated phalloidin (1:400 Alexa Fluor Plus 405: Thermo Fisher #A30104). Tissues were then mounted on a glass coverslip with Vectashield mounting medium (Vector Laboratories, Burlingame, CA, USA). Confocal imaging was performed using an LSM 800 (Carl Zeiss) microscope. Maximum intensity projection images were generated in ImageJ.

The eGFP-positive cells in the ear were evaluated manually and scored by intensity as follows: “-” means no expression, “*”=low, “**”=medium, “***”=high, and/or “****”=extremely high expression. Uninjected control samples were used to exclude autofluorescence. The results are shown below in Table 11.

TABLE 11
Intensity of eGFP-positive cells in the Ear

| | Cochlear | | | | | |
|---|---|---|---|---|---|---|
| AAV_ID | Apex | Mid | Base | Saccule | Utricle | Ampulla |
| BCD_0106 | * | * | — | *** | **** | **** |
| BCD_0361 | ** | *** | ** | **** | **** | *** |

The intensity observed in Table 11 is a proxy for AAV viral tropism (i.e., tissue/organ infectivity in the ear). The results indicate that BCD_0106 and BCD_0361 has high infectivity/tropism in the ear.

### 6.5.12 Example 11: In Vivo Evaluation of Novel rAAV Viral Particles Tropism in the Mouse Brain

In vivo studies were conducted in mice to evaluate if a selected rAAV and a novel rAAV particle comprising a BCD_0160 capsid protein could expressed in the brain of mice.

Mice: Adult wild type C57BL/6 mice (6-8 weeks old) were used for this study.

### Viral Vector Production:

rAAVs comprising BCD_0160 capsid protein or selected rAAV AAV-9-PHP.eb, comprising the vector CN1839-rAAV-hSyn1-SYFP2-10aa-H2B-WPRE3-BGHpA (Addgene plasmid #163509; http://n2t.net/addgene: 163509; RRID: Addgene_163509) were made as described in the examples above

### Retro-Orbital (RO) Injections:

RO injections were performed in C57BL/6 adult mice. Briefly, mice were anesthetized with 1-5% isoflurane in oxygen. A 31-gauge needle syringe loaded with rAAV was used to inject in the retro-orbital sinus (2.5e11 vg/mouse) in a volume that does not exceed 10% of the mouse blood volume. After, the mice were recovered from anesthesia and placed back in a clean cage. All animal procedures were approved by Animal Care and Use Committee and are in accordance with the NIH Guide for Care and Use of Laboratory Animals.

### Tissue Processing and Image Analysis:

Four weeks after injection, the mice underwent euthanization by deep anesthesia with sodium pentobarbital. After, transcardial perfusion with phosphate buffered saline (PBS) was conducted and the brain was removed and bisected along the midline. The brain was drop-fixed in 10% neutral buffered formalin (Fisher Scientific, Waltham, MA) overnight at 4° C., fixed in 10% formalin, embedded in paraffin wax, sectioned in a sagittal at 5 μm thickness and then mounted on glass slides. The tissue sections were deparaffinized in xylene and rehydrated in a graded series of alcohols. Antigen retrieval was performed by steaming in distilled water for 30 min, and endogenous peroxidase activity was blocked by incubation in 0.03% hydrogen peroxide. Sections were then immunostained and mounted with Vectashield (Vector laboratories). Images were taken with a 40× Plan-Apochromat objective using a Zeiss AxioObserver equipped with an ApoTome Imaging System (Zeiss).

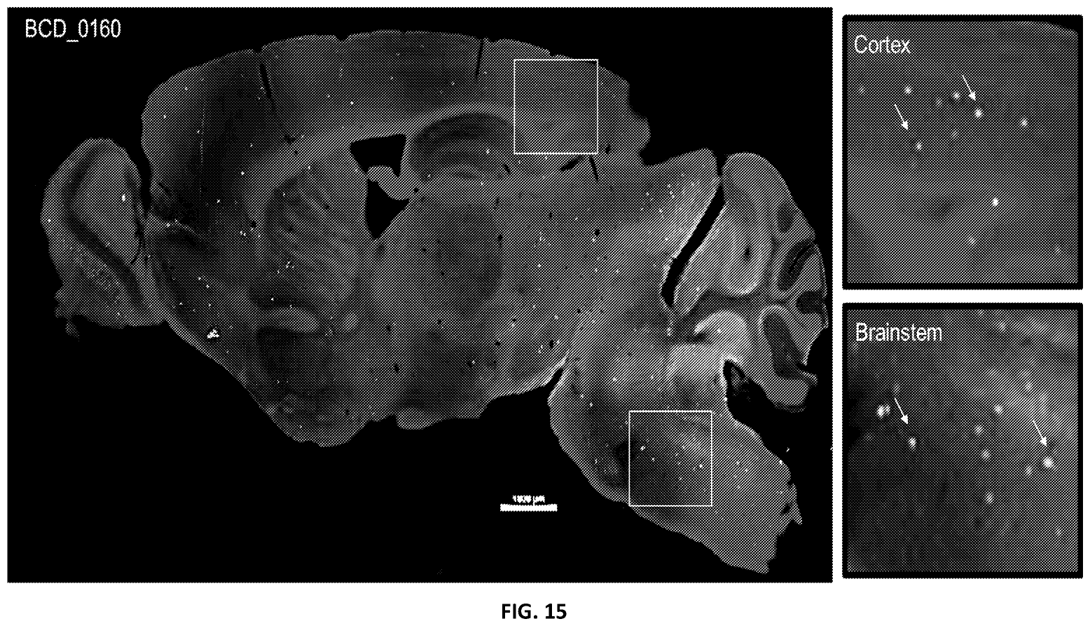

FIG. 15 shows a representative image of in vivo BCD_0160 expression in the brain. Bright spots in FIG. 15 show the expression of BCD_0160 vector-mediated fluorescent protein expression in the nuclei of neurons in a sagittal section of C57BL/6 adult mice. Zoomed-in images of areas within the Cortex and Brainstem of FIG. 15 of the sagittal section are shown. White arrows point to the colocalization of the BCD_0160 vector-mediated expression with a nuclear stain.

The embodiments described herein are intended to be merely exemplary, and those skilled in the art will recognize, or will be able to ascertain using no more than routine experimentation, numerous equivalents of specific compounds, materials, and procedures. All such equivalents are considered to be within the scope of the disclosure. The full scope of the disclosure is better understood with reference to the appended claims.

### 6.6 Sequences

Lengthy table referenced here
US20240376496A1-20241114-T00001
Please refer to the end of the specification for access instructions.

### 7. EMBODIMENTS

1. A member of an adeno-associated virus (AAV) clade, comprising: (a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362.
2. The AAV clade member of embodiment 1, wherein (a) the VP1 amino acid sequence has at least 95% identity to the VP1 amino acid sequence of any one of SEQ ID NOs: 12-127 and 361-362, (b) the VP2 amino acid sequence has at least 95% identity to the VP2 amino acid sequence of any one of SEQ ID NOs: 12-127 and 361-362, or (c) the VP3 amino acid sequence has at least 95% identity to the VP3 amino acid sequence of any one of SEQ ID NOs: 12-127 and 361-362.
3. The AAV clade member of embodiment 1, wherein the VP1 amino acid sequence has at least 98% identity to the VP1 amino acid sequence of any one of SEQ ID NOs: 12-127 and 361-362.
4. The AAV clade member of embodiment 1, 2, or 3, wherein the VP1, VP2, or VP3 amino acid sequence comprises a variable region sequence, wherein the variable region sequence is selected from the variable region of at least one of SEQ ID NOs: 12-127 and 361-362.
5. The AAV clade member any one of embodiments 1 to 4, wherein the VP1 amino acid sequence further comprises a GBS region sequence, wherein the GBS region sequence is selected from the GBS region sequence of at least one of: SEQ ID NOs: 12-127 and 361-362.
6. The AAV clade member any one of embodiments 1 to 5, wherein the VP1 amino acid sequence further comprises a GH loop sequence, wherein the GH loop sequence is selected from the GH loop of at least one of: SEQ ID NOs: 12-127 and 361-362.
7. The AAV clade member any one of embodiments 1 to 6, wherein the VP2 amino acid sequence is the VP2 amino acid sequence of any one of SEQ ID NOs: 12-127 and 361-362. 8. The AAV clade member any one of embodiments 1 to 6, wherein the VP3 amino acid sequence is the VP3 amino acid sequence of any one of SEQ ID NOs: 12-127 and 361-362.
9. The AAV clade member any one of embodiments 1 to 6, wherein the VP1 amino acid sequence is the VP1 amino acid sequence of any one of SEQ ID NOs: 12-127 and 361-362.
10. The AAV clade member of any one of embodiments 1 to 8, wherein the VP1, VP2, or VP3 amino acid sequence comprises one or more of the amino acid modifications listed in Table 2.
11. The AAV clade member of embodiment 10, wherein the one or more of the amino acid modifications of the VP1, VP2, or VP3 amino acid sequence are limited to the ones listed in Table 2.
12. A member of an adeno-associated virus (AAV) clade, comprising: a VP1 amino acid sequence that has a least 90% sequence identity to a representative VP1 amino acid sequence of a AAV clade, and wherein the representative sequence is selected from any one of: SEQ ID NOs: 28, 10, 91, 7, 9, 4, 27, 3, 1, and 23.
13. The AAV clade member of embodiment 12, wherein the VP1 amino acid sequence has at least 95% identity to any one of: SEQ ID NOs: 28, 10, 91, 7, 9, 4, 27, 3, 1, and 23.
14. The AAV clade member of embodiment 12, wherein the VP1 amino acid sequence has at least 98% identity to any one of: SEQ ID NOs: 28, 10, 91, 7, 9, 4, 27, 3, 1, and 23.
15. The AAV clade member of embodiment 12, wherein the VP1 amino acid sequence has at least 99% identity to any one of: SEQ ID NOs: 28, 10, 91, 7, 9, 4, 27, 3, 1, and 23.
16. The AAV clade member of any one of embodiments 12 to 15, wherein the VP1 amino acid sequence comprises one or more of the amino acid modifications listed in Table 2.
17. The AAV clade member of embodiment 16, wherein the VP1 amino acid sequence modifications are limited to the ones listed in Table 2.
18. A member of an adeno-associated virus (AAV) clade, comprising: a VP1 amino acid sequence that has a variable region amino acid sequence, wherein the variable region amino acid sequence has substantial sequence similarity or identity to a variable region amino acid sequence in any one of: SEQ ID NOs: 12-127 and 361-362.
19. The AAV clade member of embodiment 18, wherein the variable region amino acid sequence is selected from any one of VRI-VRIX, a GBS region, or a GH loop, or a combination thereof.
20. The AAV clade member of embodiment 18, wherein the any one of VRI-VRIX sequence has at least 90% sequence similarity or identity to any one of VRI-VRIX of any one of SEQ ID NOs: 12-127 and 361-362.
21. The AAV clade member of embodiment 18, wherein the GBS region sequence has at least 90% sequence similarity or identity to the GBS region of any one of SEQ ID NOs: 12-127 and 361-362.
22. The AAV clade member of embodiment 18, wherein the GH loop sequence has at least 90% sequence similarity or identity to the GH loop of any one of SEQ ID NOs: 12-127 and 361-362.
23. A member of an adeno-associated virus (AAV) clade, comprising: a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3.
24. The AAV clade member of embodiment 23, wherein the genetic distance is the mean genetic distance within the same AAV clade, as provided in Table 3.
25. The AAV clade member of embodiment 23, wherein the genetic distance is a range from about the min genetic distance within the same clade to about the max genetic distance within the same clade, as provided in Table 3.
26. The AAV clade member of any one of embodiments 23 to 25, wherein the second VP1 amino acid sequence comprises a VP1 amino acid sequence of any one of: SEQ ID NOs: 1-180 and 361-362.
27. A member of an adeno-associated virus (AAV) branch, comprising: a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3.
28. The AAV branch member of embodiment 27, wherein the genetic distance is the mean genetic distance within the same branch as provided in Table 3.
29. The AAV branch member of embodiment 27, wherein the genetic distance is a range from about the min genetic distance within the same branch to about the max genetic distance within the same branch as provided in Table 3.
30. The AAV branch member of embodiment 27, wherein the second VP1 amino acid sequence comprises a VP1 amino acid sequence of any one of SEQ ID NOs: 1-180 and 361-362.
31. The AAV clade or AAV branch member of any of the preceding embodiments, further comprising the ability to evade AAV humoral immunity as determined by an in vitro assay.
32. The AAV clade or AAV branch member of embodiment 31, wherein the in vitro assay is an IVIg assay that determines a percent (%) transduction, and wherein the % transduction is about 2% to about 500% greater transduction as compared to a reference AAV at a given IVIg concentration.
33. The AAV clade or AAV branch member of embodiment 31, wherein the in vitro assay is an IVIg assay that determines a neutralizing antibody (Nab) titer, and wherein the NAb titer is reduced to about 1-fold to about 4,000-fold as compared to a reference AAV.
34. The AAV clade or AAV branch member of embodiment 31, wherein the in vitro assay is an IVIg assay that determines a NC₅₀, and wherein the NC₅₀ increases from about 1-fold to about 600-fold as compared to a reference AAV.
35. An AAV capsid protein, comprising: (a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of the VP2 sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362.
36. The AAV capsid protein of embodiment 35, wherein (a) the VP1 amino acid sequence has at least 95% identity to the VP1 amino acid sequence of any one of SEQ ID NOs: 12-127 and 361-362, (b) the VP2 amino acid sequence has at least 95% identity to the VP2 amino acid sequence of ay one of SEQ ID NOs: 12-127 and 361-362, or (c) the VP3 amino acid sequence has at least 95% identity to the VP3 amino acid sequence of any one of SEQ ID NOs: 12-127 and 361-362.
37. The AAV capsid protein of embodiment 35, wherein (a) the VP1 amino acid sequence has at least 98% identity to the VP1 amino acid sequence of any one of SEQ ID NOs: 12-127 and 361-362, (b) the VP2 amino acid sequence has at least 98% identity to the VP2 amino acid sequence of any one of SEQ ID NOs: 12-127 and 361-362, or (c) the VP3 amino acid sequence has at least 98% identity to the VP3 amino acid sequence of any one of SEQ ID NOs: 12-127 and 361-362.
38. The AAV capsid protein of embodiment 35, wherein the VP1, VP2, or VP3 amino acid sequence is a VP1, VP2 or VP3 amino acid sequence of any one of SEQ ID NOs: 12-127 and 361-362.
39. The AAV capsid protein of any one of embodiments 35 to 38, wherein the VP1, VP2, or VP3 amino acid sequence comprises a variable region amino acid sequence, and wherein the variable region amino acid sequence is a VRI-VRIX of any one of: SEQ ID NOs: 12-127 and 361-362.
40. The AAV capsid protein of any one of embodiments 35 to 39, wherein the VP1, VP2, or VP3 amino acid sequence comprises a GBS region amino acid sequence, and wherein the GBS region amino acid sequence is a GBS region of any one of: SEQ ID NOs: 12-127 and 361-362.
41. The AAV capsid protein of any one of embodiments 35 to 40, wherein the VP1, VP2, or VP3 amino acid sequence comprises a GH loop amino acid sequence, and wherein the GH loop amino acid sequence is a GH loop selected from any one of: SEQ ID NOs: 12-127 and 361-362.
42. The AAV capsid protein of any one of embodiments 35 to 41, further comprising the ability to evade AAV humoral immunity as determined by an in vitro assay.
43. The AAV capsid protein of any one of embodiments 35 to 42, wherein the in vitro assay is an IVIg assay that determines a percent (%) transduction, and wherein the % transduction is about 2% to about 500% greater transduction as compared to a reference AAV at a given IVIg concentration.
44. The AAV capsid protein of any one of embodiments 35 to 42, wherein the in vitro assay is an IVIg assay that determines a NC₅₀, and wherein the NAb titer is reduced to about 1-fold to about 4,000-fold as compared to a reference AAV.
45. The AAV capsid protein of any one of embodiments 35 to 42, wherein the in vitro assay is an IVIg assay that determines a NC₅₀, and wherein the NC₅₀ increases from about 1-fold to about 600-fold as compared to a reference AAV.
46. A vector, comprising: (a) a nucleotide sequence encoding a VP1, VP2, or VP3 capsid protein that has at least 90% identity to the VP1, VP2, or VP3 of any one of SEQ ID NOs: 12-127 and 361-362; and (b) a heterologous regulatory sequence that controls expression of the capsid protein in a host cell.
47. The vector of embodiment 46, wherein the nucleotide sequence encoding a VP1, VP2, or VP3 capsid protein has at least 95% identity to the VP1, VP2, or VP3 of any one of SEQ ID NOs: 12-127 and 361-362.
48. The vector of embodiment 46, wherein the nucleotide sequence encoding a VP1, VP2, or VP3 capsid protein has at least 98% identity to the VP1, VP2, or VP3 of any one of SEQ ID NOs: 12-127 and 361-362.
49. A vector, comprising: (a) a nucleotide sequence encoding a VP1 amino acid sequence of the AAV clade member of any one of embodiments 1-26 or 31-34; and (b) a heterologous regulatory sequence that controls expression of the capsid protein in a host cell.
50. A vector, comprising: (a) a nucleotide sequence encoding a VP1 amino acid sequence of the AAV branch member of any one of embodiments 27-34; and (b) a heterologous regulatory sequence that controls expression of the capsid protein in a host cell.
51. The vector of any one of embodiments 46 to 50, wherein the vector further comprises a transgene comprising a nucleotide sequence encoding a biomolecule operably linked to a heterologous regulatory sequence that controls expression of the capsid protein in a host cell. 52. An in vitro host cell, comprising the nucleotide sequence encoding a VP1, VP2, or VP3 capsid protein of any one of embodiments 35 to 45.
53. A novel recombinant AAV viral particle, comprising: (a) a capsid, wherein the capsid comprises a VP1 amino acid sequence of a AAV clade member of any one of embodiments 1 to 26 or 31 to 34; and (b) an rAAV vector genome comprising a nucleotide sequence encoding a biomolecule operably linked to a heterologous regulatory sequence that controls expression of the nucleotide sequence in a host cell.
54. A novel recombinant AAV viral particle, comprising: (a) a capsid, wherein the capsid comprises a VP1 amino acid sequence of a AAV branch member of any one of embodiments 27 to 34; and (b) an rAAV vector genome comprising a nucleotide sequence encoding a biomolecule operably linked to a heterologous regulatory sequence that controls expression of the nucleotide sequence in a host cell.
55. A novel recombinant AAV viral particle, comprising: (a) the AAV capsid protein of any one of embodiments 35 to 45; and (b) an rAAV vector genome comprising a nucleotide sequence encoding a biomolecule operably linked to a heterologous regulatory sequence that controls expression of the nucleotide sequence in a host cell.
56. The novel recombinant AAV viral particle of any one of embodiments 53 to 55, wherein the rAAV vector genome comprises an AAV inverted terminal repeat or a fragment thereof.
57. The novel recombinant AAV viral particle of embodiment 56, wherein the AAV inverted terminal repeat is a 5′ AAV inverted terminal repeat selected from Table 4.
58. The novel recombinant AAV viral particle of embodiment 56, wherein the AAV inverted terminal repeat is a 3′ AAV inverted terminal repeat selected from Table 4.
59. The recombinant AAV viral particle of any one of embodiments 53 to 55, wherein the rAAV vector genome comprises a 5′ AAV inverted terminal repeat and a 3′ AAV inverted terminal repeat.
60. The recombinant AAV viral particle of embodiment 59, wherein the 5′ AAV inverted terminal repeat and 3′ AAV inverted terminal repeat are selected from Table 4.
61. The novel recombinant AAV viral particle of any one of embodiments 53 to 60, wherein the biomolecule is selected from a therapeutic protein, an enzyme, a peptide, an RNA, a component of CRISPR gene editing system, an antisense oligonucleotides (AONs), an AON-mediated exon skipping, a poison exon, or a dominant negative mutant protein.
62. The novel recombinant AAV viral particle of embodiment 61, wherein the therapeutic protein is endogenously expressed in one or more of a muscle, heart, brain, plasma, kidney, liver or cancer cell of a subject.
63. The novel recombinant AAV viral particle of embodiment 62, wherein therapeutic protein is a functional version of the endogenously expressed protein.
64. The novel recombinant AAV viral particle of any one of embodiments 53 to 63, wherein the recombinant AAV viral particle has enhanced tropism to the muscle cell as compared to a reference AAV.
65. The novel recombinant AAV viral particle of any one of embodiments 53 to 63, wherein the recombinant AAV viral particle has enhanced tropism to the heart cell as compared to a reference AAV.
66. The novel recombinant AAV viral particle of any one of embodiments 53 to 63, wherein the recombinant AAV viral particle has enhanced tropism to the brain cell as compared to a reference AAV.
67. The novel recombinant AAV viral particle of any one of embodiments 53 to 63, wherein the recombinant AAV viral particle has enhanced tropism to the plasma cell as compared to a reference AAV.
68. The novel recombinant AAV viral particle of any one of embodiments 53 to 63, wherein the recombinant AAV viral particle has enhanced tropism to the kidney cell as compared to a reference AAV.
69. The novel recombinant AAV viral particle of any one of embodiments 53 to 63, wherein the recombinant AAV viral particle has enhanced tropism to the liver cell as compared to a reference AAV.
70 The novel recombinant AAV viral particle of any one of embodiments 53 to 69, wherein the novel recombinant AAV viral particle de-targets cells in a subject other than the cell for which the novel rAAV has enhanced tropism.
71. The novel recombinant AAV viral particle of embodiment 70, wherein the de-targeted cell is selected from one or more of a muscle, heart, brain, plasma, kidney, or liver cell.
72. The novel recombinant AAV viral particle of any one of embodiments 53 to 71, wherein the recombinant AAV viral particle has the ability to evade AAV humoral immunity as determined by an in vitro assay.
73. The novel recombinant AAV viral particle of embodiment 72, wherein the in vitro assay is an IVIg assay that determines a percent (%) transduction, and wherein the % transduction is about 2% to about 500% greater transduction as compared to a reference AAV at a given IVIg concentration.
74. The novel recombinant AAV viral particle of embodiment 72, wherein the in vitro assay is an IVIg assay that determines a NC₅₀, and wherein the NAb titer is reduced to about 1-fold to about 4,000-fold as compared to a reference AAV.
75. The novel recombinant AAV viral particle of embodiment 72, wherein the in vitro assay is an IVIg assay that determines a NC₅₀, and wherein the NC₅₀ increases from about 1-fold to about 600-fold as compared to a reference AAV.
76. An in vitro cell or tissue comprising: the novel recombinant AAV viral particle of any one of embodiments 53 to 75.
77. An ex vivo cell or tissue comprising: the novel recombinant AAV viral particle of any one of embodiments 53 to 75.
78. A cultured host cell, comprising: a recombinant nucleic acid molecule encoding an AAV VP1 capsid protein comprising a sequence comprising the full length VP1 capsid protein of any one of SEQ ID NOs: 12-127 and 361-362; or a nucleotide sequence with at least 95% identical to the full length VP1 protein of any one of SEQ ID NOs: 12-127 and 361-362, wherein the recombinant nucleic acid molecule further comprises a heterologous sequence.
79. A cultured host cell, comprising: a recombinant nucleic acid molecule encoding an AAV VP2 capsid protein comprising a sequence comprising: (a) the full length VP2 capsid protein of any one of SEQ ID NOs: 12-127 and 361-362; or (b) an amino acid sequence with at least 95% identity to the full length VP2 protein of any one of SEQ ID NOs: 12-127 and 361-362, wherein the recombinant nucleic acid molecule further comprises a heterologous sequence.
80. A cultured host cell, comprising: a recombinant nucleic acid molecule encoding an AAV VP3 capsid protein comprising a sequence comprising: (a) the full length VP3 capsid protein of any one of SEQ ID NOs: 12-127 and 361-362; or (b) an amino acid sequence with at least 95% identical to the full length VP3 protein of any one of SEQ ID NOs: 12-127 and 361-362, wherein the recombinant nucleic acid molecule further comprises a heterologous sequence.
81. The cultured host cell of any one of embodiments 78 to 80, wherein the amino acid residues varied in the AAV VP1, VP2, or VP3 capsid protein with at least 95% identity to the full length VP1, VP2, or VP3 capsid protein of any one of SEQ ID NOs: 12-127 and 361-362 are selected from Table 2.
82. A cultured host cell containing a recombinant nucleic acid molecule, comprising: (a) nucleotides of a full length AAV VP1 capsid protein of any one of SEQ ID NOs: 192-307 and 363-364; or (b) a nucleotide sequence at least 95% identical to the nucleotide sequence of the full length VP1 capsid protein of any one of SEQ ID NOs: 192-307 and 363-364, wherein the recombinant nucleic acid molecule further comprises a heterologous sequence.
83. A cultured host cell containing a recombinant nucleic acid molecule, comprising: (a) nucleotides of a full length AAV VP2 capsid protein of any one of SEQ ID NOs: 192-307 and 363-364; or (b) a nucleotide sequence at least 95% identical to the nucleotide sequence of the full length VP2 capsid protein of any one of SEQ ID NOs: 192-307 and 363-364, wherein the recombinant nucleic acid molecule further comprises a heterologous sequence.
84. A cultured host cell containing a recombinant nucleic acid molecule, comprising: (a) nucleotides of a full length AAV VP3 capsid protein of any one of SEQ ID NOs: 192-307 and 363-364; or (b) a nucleotide sequence at least 95% identical to the nucleotide sequence of the full length VP3 capsid protein of any one of SEQ ID NOs: 192-307 and 363-364, wherein the recombinant nucleic acid molecule further comprises a heterologous sequence.
85. The cultured host cell of any one of embodiments 78 to 81, wherein nucleotides varied in the nucleotide sequence encoding the AAV VP1, VP2, or VP3 with at least 95% identity to the full length VP1, VP2, or VP3 capsid protein of any one of SEQ ID NOs: 12-127 and 361-362 are selected from nucleic acids encoding the amino acid residues that vary in Table 2.
86. A composition, comprising: a) the novel recombinant AAV viral particle of any one of embodiments 53 to 75; and (b) a physiologically acceptable carrier.
87. A method of delivering a biomolecule to a cell in vitro, comprising: transducing the cell with the novel recombinant AAV viral particle of any one of embodiments 53 to 75.
88. A method of delivering a biomolecule to a cell ex vivo, comprising: transducing the cell with the novel recombinant AAV viral particle of any one of embodiments 53 to 73.
89. A method of delivering a biomolecule to a cell in a subject, comprising: administering the novel recombinant AAV viral particle of any one of embodiments 53 to 75 to the cell in the subject.
90. The method of any one of embodiments 87 to 89, wherein the cell is one or more of a muscle, heart, brain, plasma, kidney, liver, or cancer cell.
91. A method of treating a disease or disorder, comprising: administering the novel recombinant AAV viral particle of any one of embodiments 53 to 75 to a subject.
92. The method of embodiments 89 or 91, wherein the subject is a human.
93. A method for producing a novel recombinant AAV viral particle, comprising: culturing a host cell comprising one or more vectors or rAAV vector genomes for generating the novel rAAV viral particle, wherein the one or more vectors or rAAV vector genomes comprises a nucleotide sequence encoding the capsid protein of any one of embodiments 35 to 45.
94. A method for producing a novel recombinant AAV viral particle, comprising: culturing a host cell comprising one or more vectors or rAAV vector genomes for generating the novel rAAV viral particle, wherein the one or more vectors or rAAV vector genomes comprises a nucleotide sequence encoding a VP1 protein of the AAV clade member of any one of embodiments 1 to 26 or 31 to 34.
95. A method for producing a novel rAAV viral particle, comprising: culturing a host cell comprising one or more vectors or rAAV vector genomes for generating the novel rAAV viral particle, wherein the one or more vectors or rAAV vector genomes comprises a nucleotide sequence encoding a VP1 protein of the AAV branch member of any one of embodiments 27 to 34.
96. The method of any one of embodiments 93 to 95, wherein the nucleotide sequence encoding the capsid protein or VP1 protein is operably linked to a heterologous regulatory sequence that controls expression of the nucleotide sequence in a host cell.
97. The method of any one of embodiments 93 to 95, wherein the one or more vectors or rAAV vector genomes further comprises a nucleotide sequence used by the host cell to generate an rAAV viral particle, and wherein the nucleotide sequence is operably linked to a heterologous regulatory sequence that controls expression of the nucleotide sequence in a host cell.
98. The method of any one of embodiments 93 to 97, wherein prior to the culturing step the host cell is transfected with the one or more vectors or rAAV genomes.
99. The method of any one of embodiments 93 to 98, wherein the novel rAAV viral particle is isolated from the host cell.

LENGTHY TABLES
The patent application contains a lengthy table section. A copy of the table is available in electronic form from the USPTO web site (https://seqdata.uspto.gov/?pageRequest=docDetail&DocID=US20240376496A1). An electronic copy of the table will also be available from the USPTO upon request and payment of the fee set forth in 37 CFR 1.19(b)(3).

We claim:

1. A member of an adeno-associated virus (AAV) clade, comprising:
(a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362.

2. The AAV clade member of claim 1, wherein (a) the VP1 amino acid sequence has at least 95% identity to the VP1 amino acid sequence of any one of SEQ ID NOs: 12-127 and 361-362, (b) the VP2 amino acid sequence has at least 95% identity to the VP2 amino acid sequence of any one of SEQ ID NOs: 12-127 and 361-362, or (c) the VP3 amino acid sequence has at least 95% identity to the VP3 amino acid sequence of any one of SEQ ID NOs: 12-127 and 361-362.

3. The AAV clade member of claim 1, wherein the VP1 amino acid sequence has at least 98% identity to the VP1 amino acid sequence of any one of SEQ ID NOs: 12-127 and 361-362.

4. The AAV clade member of claim 1, 2, or 3, wherein the VP1, VP2, or VP3 amino acid sequence comprises a variable region sequence, wherein the variable region sequence is selected from the variable region of at least one of SEQ ID NOs: 12-127 and 361-362.

5. The AAV clade member any one of claims 1 to 4, wherein the VP1 amino acid sequence further comprises a GBS region sequence, wherein the GBS region sequence is selected from the GBS region sequence of at least one of: SEQ ID NOs: 12-127 and 361-362.

6. The AAV clade member any one of claims 1 to 5, wherein the VP1 amino acid sequence further comprises a GH loop sequence, wherein the GH loop sequence is selected from the GH loop of at least one of: SEQ ID NOs: 12-127 and 361-362.

7. The AAV clade member any one of claims 1 to 6, wherein the VP2 amino acid sequence is the VP2 amino acid sequence of any one of SEQ ID NOs: 12-127 and 361-362.

8. The AAV clade member any one of claims 1 to 6, wherein the VP3 amino acid sequence is the VP3 amino acid sequence of any one of SEQ ID NOs: 12-127 and 361-362.

9. The AAV clade member any one of claims 1 to 6, wherein the VP1 amino acid sequence is the VP1 amino acid sequence of any one of SEQ ID NOs: 12-127 and 361-362.

10. The AAV clade member of any one of claims 1 to 8, wherein the VP1, VP2, or VP3 amino acid sequence comprises one or more of the amino acid modifications listed in Table 2.

11. The AAV clade member of claim 10, wherein the one or more of the amino acid modifications of the VP1, VP2, or VP3 amino acid sequence are limited to the ones listed in Table 2.

12. A member of an adeno-associated virus (AAV) clade, comprising:
a VP1 amino acid sequence that has a least 90% sequence identity to a representative VP1 amino acid sequence of a AAV clade, and wherein the representative sequence is selected from any one of: SEQ ID NOs: 28, 10, 91, 7, 9, 4, 27, 3, 1, and 23.

13. The AAV clade member of claim 12, wherein the VP1 amino acid sequence has at least 95% identity to any one of: SEQ ID NOs: SEQ ID NOs: 28, 10, 91, 7, 9, 4, 27, 3, 1, and 23.

14. The AAV clade member of claim 12, wherein the VP1 amino acid sequence has at least 98% identity to any one of: SEQ ID NOs: SEQ ID NOs: 28, 10, 91, 7, 9, 4, 27, 3, 1, and 23.

15. The AAV clade member of claim 12, wherein the VP1 amino acid sequence has at least 99% identity to any one of: SEQ ID NOs: SEQ ID NOs: 28, 10, 91, 7, 9, 4, 27, 3, 1, and 23.

16. The AAV clade member of any one of claims 12 to 15, wherein the VP1 amino acid sequence comprises one or more of the amino acid modifications listed in Table 2.

17. The AAV clade member of claim 16, wherein the VP1 amino acid sequence modifications are limited to the ones listed in Table 2.

18. A member of an adeno-associated virus (AAV) clade, comprising:
a VP1 amino acid sequence that has a variable region amino acid sequence, wherein the variable region amino acid sequence has substantial sequence identity to a variable region amino acid sequence in any one of: SEQ ID NOs: 12-127 and 361-362.

19. The AAV clade member of claim 18, wherein the variable region amino acid sequence is selected from any one of VRI-VRIX, a GBS region, or a GH loop, or a combination thereof.

20. The AAV clade member of claim 19, wherein the any one of VRI-VRIX sequence has at least 90% sequence identity to any one of VRI-VRIX of any one of SEQ ID NOs: 12-127 and 361-362.

21. The AAV clade member of claim 19, wherein the GBS region sequence has at least 90% sequence similarity or identity to the GBS region of any one of SEQ ID NOs: 12-127 and 361-362.

22. The AAV clade member of claim 19, wherein the GH loop sequence has at least 90% sequence similarity or identity to the GH loop of any one of SEQ ID NOs: 12-127 and 361-362.

23. A member of an adeno-associated virus (AAV) clade, comprising:
a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3.

24. The AAV clade member of claim 23, wherein the genetic distance is the mean genetic distance within the same AAV clade, as provided in Table 3.

25. The AAV clade member of claim 23, wherein the genetic distance is a range from about the min genetic distance within the same clade to about the max genetic distance within the same clade, as provided in Table 3.

26. The AAV clade member of any one of claims 23 to 25, wherein the second VP1 amino acid sequence comprises a VP1 amino acid sequence of any one of: SEQ ID NOs: 1-180 and 361-362.

27. A member of an adeno-associated virus (AAV) branch, comprising:
a first VP1 amino acid sequence that is phylogenetically related to a second VP1 amino acid sequence as determined by Neighbor-joining method, wherein the first VP1 amino acid sequence has a genetic distance to the second VP1 amino acid sequence as provided in Table 3.

28. The AAV branch member of claim 27, wherein the genetic distance is the mean genetic distance within the same branch as provided in Table 3.

29. The AAV branch member of claim 27, wherein the genetic distance is a range from about the min genetic distance within the same branch to about the max genetic distance within the same branch as provided in Table 3.

30. The AAV branch member of claim 27, wherein the second VP1 amino acid sequence comprises a VP1 amino acid sequence of any one of SEQ ID NOs: 1-180 and 361-362.

31. The AAV clade or AAV branch member of any of the preceding claims, further comprising the ability to evade AAV humoral immunity as determined by an in vitro assay.

32. The AAV clade or AAV branch member of claim 31, wherein the in vitro assay is an IVIg assay that determines a percent (%) transduction, and wherein the % transduction is about 2% to about 500% greater transduction as compared to a reference AAV at a given IVIg concentration.

33. The AAV clade or AAV branch member of claim 31, wherein the in vitro assay is an IVIg assay that determines a neutralizing antibody (Nab) titer, and wherein the NAb titer is reduced to about 1-fold to about 4,000-fold as compared to a reference AAV.

34. The AAV clade or AAV branch member of claim 31, wherein the in vitro assay is an IVIg assay that determines a NC₅₀, and wherein the NC₅₀ increases from about 1-fold to about 600-fold as compared to a reference AAV.

35. An AAV capsid protein, comprising:
(a) a VP1 amino acid sequence that has at least 90% identity to the VP1 amino acid sequence of any one of SEQ ID NOs: 12-127 and 361-362, (b) a VP2 amino acid sequence that has at least 90% identity to the VP2 amino acid sequence of the VP2 sequence of any one of SEQ ID NOs: 12-127 and 361-362, or (c) a VP3 amino acid sequence that has at least 90% identity to the VP3 amino acid sequence of any one of SEQ ID NOs: 12-127 and 361-362.

36. The AAV capsid protein of claim 35, wherein (a) the VP1 amino acid sequence has at least 95% identity to the VP1 amino acid sequence of any one of SEQ ID NOs: 12-127 and 361-362, (b) the VP2 amino acid sequence has at least 95% identity to the VP2 amino acid sequence of ay one of SEQ ID NOs: 12-127 and 361-362, or (c) the VP3 amino acid sequence has at least 95% identity to the VP3 amino acid sequence of any one of SEQ ID NOs: 12-127 and 361-362.

37. The AAV capsid protein of claim 35, wherein (a) the VP1 amino acid sequence has at least 98% identity to the VP1 amino acid sequence of any one of SEQ ID NOs: 12-127 and 361-362, (b) the VP2 amino acid sequence has at least 98% identity to the VP2 amino acid sequence of any one of SEQ ID NOs: 12-127 and 361-362, or (c) the VP3 amino acid sequence has at least 98% identity to the VP3 amino acid sequence of any one of SEQ ID NOs: 12-127 and 361-362.

38. The AAV capsid protein of claim 35, wherein the VP1, VP2, or VP3 amino acid sequence is a VP1, VP2 or VP3 amino acid sequence of any one of SEQ ID NOs: 12-127 and 361-362.

39. The AAV capsid protein of any one of claims 35 to 38, wherein the VP1, VP2, or VP3 amino acid sequence comprises a variable region amino acid sequence, and wherein the variable region amino acid sequence is a VRI-VRIX of any one of: SEQ ID NOs: 12-127 and 361-362.

40. The AAV capsid protein of any one of claims 35 to 39, wherein the VP1, VP2, or VP3 amino acid sequence comprises a GBS region amino acid sequence, and wherein the GBS region amino acid sequence is a GBS region of any one of: SEQ ID NOs: 12-127 and 361-362.

41. The AAV capsid protein of any one of claims 35 to 40, wherein the VP1, VP2, or VP3 amino acid sequence comprises a GH loop amino acid sequence, and wherein the GH loop amino acid sequence is a GH loop selected from any one of: SEQ ID NOs: 12-127 and 361-362.

42. The AAV capsid protein of any one of claims 35 to 41, further comprising the ability to evade AAV humoral immunity as determined by an in vitro assay.

43. The AAV capsid protein of any one of claims 35 to 42, wherein the in vitro assay is an IVIg assay that determines a percent (%) transduction, and wherein the % transduction is about 2% to about 500% greater transduction as compared to a reference AAV at a given IVIg concentration.

44. The AAV capsid protein of any one of claims 35 to 42, wherein the in vitro assay is an IVIg assay that determines a NC₅₀, and wherein the NAb titer is reduced to about 1-fold to about 4,000-fold as compared to a reference AAV.

45. The AAV capsid protein of any one of claims 35 to 42, wherein the in vitro assay is an IVIg assay that determines a NC₅₀, and wherein the NC₅₀ increases from about 1-fold to about 600-fold as compared to a reference AAV.

46. A vector, comprising:
(a) a nucleotide sequence encoding a VP1, VP2, or VP3 capsid protein that has at least 90% identity to the VP1, VP2, or VP3 of any one of SEQ ID NOs: 12-127 and 361-362; and
(b) a heterologous regulatory sequence that controls expression of the capsid protein in a host cell.

47. The vector of claim 46, wherein the nucleotide sequence encoding a VP1, VP2, or VP3 capsid protein has at least 95% identity to the VP1, VP2, or VP3 of any one of SEQ ID NOs: 12-127 and 361-362.

48. The vector of claim 46, wherein the nucleotide sequence encoding a VP1, VP2, or VP3 capsid protein has at least 98% identity to the VP1, VP2, or VP3 of any one of SEQ ID NOs: 12-127 and 361-362.

49. A vector, comprising:
(a) a nucleotide sequence encoding a VP1 amino acid sequence of the AAV clade member of any one of claim 1-26 or 31-34; and
(b) a heterologous regulatory sequence that controls expression of the capsid protein in a host cell.

50. A vector, comprising:
(a) a nucleotide sequence encoding a VP1 amino acid sequence of the AAV branch member of any one of claims 27-34; and
(b) a heterologous regulatory sequence that controls expression of the capsid protein in a host cell.

51. The vector of any one of claims 46 to 50, wherein the vector further comprises a transgene comprising a nucleotide sequence encoding a biomolecule operably linked to a heterologous regulatory sequence that controls expression of the capsid protein in a host cell.

52. An in vitro host cell, comprising the nucleotide sequence encoding a VP1, VP2, or VP3 capsid protein of any one of claims 35 to 45.

53. A novel recombinant AAV viral particle, comprising:
(a) a capsid, wherein the capsid comprises a VP1 amino acid sequence of a AAV clade member of any one of claims 1 to 26 or 31 to 34; and
(b) an rAAV vector genome comprising a nucleotide sequence encoding a biomolecule operably linked to a heterologous regulatory sequence that controls expression of the nucleotide sequence in a host cell.

54. A novel recombinant AAV viral particle, comprising:
(a) a capsid, wherein the capsid comprises a VP1 amino acid sequence of a AAV branch member of any one of claims 27 to 34; and
(b) an rAAV vector genome comprising a nucleotide sequence encoding a biomolecule operably linked to a heterologous regulatory sequence that controls expression of the nucleotide sequence in a host cell.

55. A novel recombinant AAV viral particle, comprising:
(a) the AAV capsid protein of any one of claims 35 to 45; and
(b) an rAAV vector genome comprising a nucleotide sequence encoding a biomolecule operably linked to a heterologous regulatory sequence that controls expression of the nucleotide sequence in a host cell.

56. The novel recombinant AAV viral particle of any one of claims 53 to 55, wherein the rAAV vector genome comprises an AAV inverted terminal repeat or a fragment thereof.

57. The novel recombinant AAV viral particle of any one of claims 53 to 55, wherein the rAAV vector genome comprises a 5′ AAV inverted terminal repeat and a 3′ AAV inverted terminal repeat.

58. The novel recombinant AAV viral particle of claim 56, wherein the AAV inverted terminal repeat is a 5′ AAV inverted terminal repeat selected from Table 4.

59. The novel recombinant AAV viral particle of claim 56, wherein the AAV inverted terminal repeat is a 3′ AAV inverted terminal repeat selected from Table 4.

60. The novel recombinant AAV viral particle of claim 57, wherein the 5′ AAV inverted terminal repeat and 3′ AAV inverted terminal repeat are selected from Table 4.

61. The novel recombinant AAV viral particle of any one of claims 53 to 58, wherein the biomolecule is selected from a therapeutic protein, an enzyme, a peptide, an RNA, a component of CRISPR gene editing system, an antisense oligonucleotides (AONs), an AON-mediated exon skipping, a poison exon, or a dominant negative mutant protein.

62. The novel recombinant AAV viral particle of claim 61, wherein the therapeutic protein is endogenously expressed in one or more of a muscle, heart, brain, plasma, kidney, liver or cancer cell of a subject.

63. The novel recombinant AAV viral particle of claim 62, wherein therapeutic protein is a functional version of the endogenously expressed protein.

64. The novel recombinant AAV viral particle of any one of claims 53 to 63, wherein the recombinant AAV viral particle has enhanced tropism to a muscle cell as compared to a reference AAV.

65. The novel recombinant AAV viral particle of any one of claims 53 to 63, wherein the recombinant AAV viral particle has enhanced tropism to a heart cell as compared to a reference AAV.

66. The novel recombinant AAV viral particle of any one of claims 53 to 63, wherein the recombinant AAV viral particle has enhanced tropism to a brain cell as compared to a reference AAV.

67. The novel recombinant AAV viral particle of any one of claims 53 to 63, wherein the recombinant AAV viral particle has enhanced tropism to a plasma cell as compared to a reference AAV.

68. The novel recombinant AAV viral particle of any one of claims 53 to 63, wherein the recombinant AAV viral particle has enhanced tropism to a kidney cell as compared to a reference AAV.

69. The novel recombinant AAV viral particle of any one of claims 53 to 63, wherein the recombinant AAV viral particle has enhanced tropism to a liver cell as compared to a reference AAV.

70. The novel recombinant AAV viral particle of any one of claims 53 to 69, wherein the novel recombinant AAV viral particle de-targets cells in a subject other than the cell for which the novel rAAV has enhanced tropism.

71. The novel recombinant AAV viral particle of claim 70, wherein the de-targeted cell is selected from one or more of a muscle, heart, brain, plasma, kidney, or liver cell.

72. The novel recombinant AAV viral particle of any one of claims 53 to 71, wherein the recombinant AAV viral particle has the ability to evade AAV humoral immunity as determined by an in vitro assay.

73. The novel recombinant AAV viral particle of claim 72, wherein the in vitro assay is an IVIg assay that determines a percent (%) transduction, and wherein the % transduction is about 2% to about 500% greater transduction as compared to a reference AAV at a given IVIg concentration.

74. The novel recombinant AAV viral particle of claim 72, wherein the in vitro assay is an IVIg assay that determines a NC₅₀, and wherein the NAb titer is reduced to about 1-fold to about 4,000-fold as compared to a reference AAV.

75. The novel recombinant AAV viral particle of claim 72, wherein the in vitro assay is an IVIg assay that determines a NC₅₀, and wherein the NC₅₀ increases from about 1-fold to about 600-fold as compared to a reference AAV.

76. An in vitro cell or tissue comprising: the novel recombinant AAV viral particle of any one of claims 53 to 75.

77. An ex vivo cell or tissue comprising: the novel recombinant AAV viral particle of any one of claims 53 to 75.

78. A cultured host cell, comprising:
a recombinant nucleic acid molecule comprising a nucleotide sequence encoding an AAV VP1 capsid protein comprising: (a) a sequence comprising the full length VP1 capsid protein of any one of SEQ ID NOs: 12-127 and 361-362; or (b) an amino acid sequence with at least 95% identity to the full length VP1 protein of any one of SEQ ID NOs: 12-127 and 361-362, wherein the recombinant nucleic acid molecule further comprises a heterologous sequence.

79. A cultured host cell, comprising:
a recombinant nucleic acid molecule comprising a nucleotide sequence encoding an AAV VP2 capsid protein comprising: (a) a sequence comprising the full length VP2 capsid protein of any one of SEQ ID NOs: 12-127 and 361-362; or (b) an amino acid sequence with at least 95% identity to the full length VP2 protein of any one of SEQ ID NOs: 12-127 and 361-362, wherein the recombinant nucleic acid molecule further comprises a heterologous sequence.

80. A cultured host cell, comprising:
a recombinant nucleic acid molecule comprising a nucleotide sequence encoding an AAV VP3 capsid protein comprising: (a) a sequence comprising the full length VP3 capsid protein of any one of SEQ ID Nos: 12-127 and 361-362; or (b) an amino acid sequence with at least 95% identity to the full length VP3 protein of any one of SEQ ID NOs: 12-127 and 361-362, wherein the recombinant nucleic acid molecule further comprises a heterologous sequence.

81. The cultured host cell of any one of claims 78 to 80, wherein the amino acid residues varied in the AAV VP1, VP2, or VP3 capsid protein with at least 95% identity to the full length VP1, VP2, or VP3 capsid protein of any one of SEQ ID NOs: 12-127 and 361-362 are selected from Table 2.

82. A cultured host cell containing a recombinant nucleic acid molecule, comprising:
(a) nucleotides of a full length AAV VP1 capsid protein of any one of SEQ ID Nos: 192-307 and 363-364; or (b) a nucleotide sequence at least 95% identical to the nucleotide sequence of the full length VP1 capsid protein of any one of SEQ ID NOs: 192-307 and 363-364, wherein the recombinant nucleic acid molecule further comprises a heterologous nucleotide sequence.

83. A cultured host cell containing a recombinant nucleic acid molecule, comprising:
(a) nucleotides of a full length AAV VP2 capsid protein of any one of SEQ ID NOs: 192-307 and 363-364; or (b) a nucleotide sequence at least 95% identical to the nucleotide sequence of the full length VP2 capsid protein of any one of SEQ ID NOs: 192-307 and 363-364, wherein the recombinant nucleic acid molecule further comprises a heterologous nucleotide sequence.

84. A cultured host cell containing a recombinant nucleic acid molecule, comprising:
(a) nucleotides of a full length AAV VP3 capsid protein of any one of SEQ ID NOs: 192-307 and 363-364; or (b) a nucleotide sequence at least 95% identical to the nucleotide sequence of the full length VP3 capsid protein of any one of SEQ ID NOs: 192-307 and 363-364, wherein the recombinant nucleic acid molecule further comprises a heterologous nucleotide sequence.

85. The cultured host cell of any one of claims 78 to 81, wherein nucleotides varied in the nucleotide sequence encoding the AAV VP1, VP2, or VP3 capsid protein with at least 95% identity to the full length VP1, VP2, or VP3 capsid protein of any one of SEQ ID NOs: 12-127 and 361-362 are selected from nucleotides encoding the amino acid residues that vary in Table 2.

86. A composition, comprising:
(a) the novel recombinant AAV viral particle of any one of claims 53 to 73; and
(b) a physiologically acceptable carrier.

87. A method of delivering a biomolecule to a cell in vitro, comprising:
transducing the cell with the novel recombinant AAV viral particle of any one of claims 53 to 75.

88. A method of delivering a biomolecule to a cell ex vivo, comprising:
transducing the cell with the novel recombinant AAV viral particle of any one of claims 53 to 75.

89. A method of delivering a biomolecule to a cell in a subject, comprising:
administering the novel recombinant AAV viral particle of any one of claims 53 to 73 to the cell in the subject.

90. The method of any one of claims 87 to 89, wherein the cell is one or more of a muscle, heart, brain, plasma, kidney, liver, or cancer cell.

91. A method of treating a disease or disorder, comprising:
administering the novel recombinant AAV viral particle of any one of claims 53 to 75 to a subject.

92. The method of claim 89 or 91, wherein the subject is a human.

93. A method for producing a novel recombinant AAV viral particle, comprising:
culturing a host cell comprising one or more vectors for generating the novel rAAV viral particle, wherein the one or more vectors comprise a nucleotide sequence encoding the capsid protein of any one of claims 35 to 45.

94. A method for producing a novel recombinant AAV viral particle, comprising:
culturing a host cell comprising one or more vectors for generating the novel rAAV viral particle, wherein the one or more vectors comprises a nucleotide sequence encoding a VP1 protein of the AAV clade member of any one of claims 1 to 26 or 31 to 34.

95. A method for producing a novel rAAV viral particle, comprising:
culturing a host cell comprising one or more vectors for generating the novel rAAV viral particle, wherein the one or more vectors comprises a nucleotide sequence encoding a VP1 protein of the AAV branch member of any one of claims 27 to 34.

96. The method of any one of claims 93 to 95, wherein the nucleotide sequence encoding the capsid protein or VP1 protein is operably linked to a heterologous regulatory sequence that controls the expression of the nucleotide sequence in a host cell.

97. The method of any one of claims 93 to 95, wherein the one or more vectors further comprises a nucleotide sequence used by the host cell to generate an rAAV viral particle, and wherein the nucleotide sequence is operably linked to a heterologous regulatory sequence that controls expression of the nucleotide sequence in a host cell.

98. The method of any one of claims 93 to 97, wherein prior to the culturing step the host cell is transfected with the one or more vectors.

99. The method of any one of claims 93 to 98, wherein the novel rAAV viral particle is isolated from the host cell.
